(19) United States
(12) Patent Application Publication
(10) Pub. No.: US 2016/0296464 A1
(43) Pub. Date: Oct. 13, 2016

(54) Sublingual Cannabis Dosage Form and Method of Making and Using the Same

(71) Applicant: FIRST WATERSIGN LLC, Boston, MA (US)

(72) Inventor: Shanel A. Lindsay, Auburn, MA (US)

(21) Appl. No.: 15/034,834

(22) PCT Filed: Nov. 5, 2014
(86) PCT No.: PCT/US14/64121

§ 371 Date: May 5, 2016

Related U.S. Application Data
(60) Provisional application No. 61/900,053, filed on Nov. 5, 2013.
(60) Provisional application No. 61/980,391, filed on Apr. 16, 2014.

(51) Int. Cl. A61K 9/00; B65B 11/48; A61K 36/185

(52) U.S. Cl. CPC A61K 9/009; A61K 9/006; A61K 36/185; A61K 9/0053; B65B 11/48

(57) ABSTRACT

The present invention relates to a sublingual dosage form comprising decarboxylated cannabis plant material, and a dispenser for delivering at least one pharmacologically active cannabinoid from the decarboxylated cannabis plant material contained inside the dispenser into the sublingual cavity of a subject when the dispenser is placed within the subject's sublingual cavity with the decarboxylated cannabis plant material contained therein. Also disclosed are a method and apparatus for preparing the sublingual dosage form, and compositions and kits comprising the sublingual dosage form.

### RELATED APPLICATIONS

This application claims the benefit of U.S. Provisional Application No. 61/900,053, filed on Nov. 5, 2013, and 61/980,391, filed on Apr. 16, 2014. The entire teachings of the above applications are incorporated herein by reference.

### BACKGROUND OF THE INVENTION

In its natural state, the tetrahydrocannabinol (THC) in cannabis is found as its biosynthesized precursor, cannabinolic acid. To activate this potential THC, a carboxyl group must be removed by heat. In practice, available cannabinolic acid in cannabis is “decarboxylated” to THC by the heat of smoking or cooking. The THC may then be delivered relatively rapidly through the lungs when smoked or more slowly in the gastrointestinal tract when eaten. Such conventional methods of preparing and ingesting cannabis result in imprecise and incomplete decarboxylation, and any preparation other than smoking requires extraction of the active cannabinoids into a secondary medium. Such conventional methods of preparing and ingesting cannabis suffer from other drawbacks as well. For example, smoking cannabis necessarily creates harmful carcinogens through destructive pyrolysis of numerous plant compounds, as well as irritation to the lung tissue. Similarly, eating cannabis requires a significant period of time before onset of effects, and the uptake through the gastrointestinal tract is uneven and incomplete.

### SUMMARY OF THE INVENTION

There is a need for a sublingual dosage form consisting of, consisting essentially of, or comprising decarboxylated cannabis plant material. There is also a need for a dispenser for delivering pharmacologically active cannabinoids (e.g., THC) from the sublingual dosage. The present invention is directed toward further solutions to address this need, in addition to having other desirable characteristics.

In accordance with an embodiment of the present disclosure, a sublingual dosage form comprising decarboxylated cannabis plant material is provided. The sublingual dosage form is not a conventional pharmaceutical dosage form formulated for sublingual or oral administration. The sublingual dosage form is not formulated as a conventional pharmaceutical dosage form such as an aerosol, a bead, a capsule, a cloth, a concentrate, an elixir, an emulsion, an extract, a fiber, a film, a gel, a globule, a granule, a chewing gum, an inhalant, a jelly, a liquid, a lozenge comprising a cannabinoid extract, an oil, a paste, a patch, a pellet, a pill, a poultice, a powder, a salve, a solution, a sponge, a spray, a strip, a suspension, a syrup, a tablet, a tape, a tincture, a trouche, and a wafer. The sublingual dosage form in its finished state does not contain a pharmaceutically acceptable binder, buffering agent, carrier, chelating agent, co-solvent, cross-linking agent, diluent, disintegrant, emulsifier, excipient, flavoring agent, permeability enhancer, preservative, propellant, solvent, or surfactant compressed into a cohesive solid dosage form. The total weight of the decarboxylated cannabis plant material accounts for substantially the entire weight of the sublingual dosage form.

In accordance with aspects of, the present disclosure, the total weight of the decarboxylated cannabis plant material ranges from about 0.05 grams to about 7 grams. The sublingual dosage form releases an effective amount of at least one pharmacologically active cannabinoid into the systemic circulation of a subject when the sublingual dosage form is placed into the sublingual cavity of the subject.

In accordance with aspects of the present disclosure, that the sublingual dosage form instantaneously releases an effective amount of at least one pharmacologically active cannabinoid into the sublingual cavity of a subject when placed in the subject's sublingual cavity. In accordance with aspects of the present disclosure, the sublingual dosage form begins to release an effective amount of at least one pharmacologically active cannabinoid into the systemic circulation of a subject within seconds of placing the sublingual dosage form in the subject's sublingual cavity. In accordance with aspects of the present disclosure, the sublingual dosage form induces a pharmacological effect in a subject within about 1 minute to about 20 minutes of placing the sublingual dosage form in the subject's sublingual cavity. In accordance with aspects of the present disclosure, after releasing an effective amount of at least one pharmacologically active cannabinoid the sublingual dosage form retains an amount of at least one pharmacologically active cannabinoid effective for enteral administration. The at least one pharmacologically active cannabinoid effective for enteral administration can be the same at least pharmacologically active cannabinoid or a different at least one pharmacologically active cannabinoid.

In accordance with aspects of the present disclosure, the sublingual dosage form is ingestible. In accordance with aspects of the present disclosure, the ingestible sublingual dosage form is ingested without first being placed in the sublingual cavity (i.e., the sublingual dosage form can be administered orally without first administering a sublingual dose). In accordance with aspects of the present disclosure, the sublingual dosage form is prepared for ingesting by removing all of the seeds and/or stems from the sublingual dosage form prior to placement in the sublingual cavity. In accordance with aspects of the present disclosure, when ingested after releasing an effective amount of at least one pharmacologically active cannabinoid the sublingual dosage form releases an effective amount of at least one pharmacologically active cannabinoid into the gastrointestinal tract of a subject. In accordance with aspects of the present disclosure, when ingested without first being placed in the sublingual cavity the sublingual dosage form releases an effective amount of at least one pharmacologically active cannabinoid into the gastrointestinal tract of a subject.

In accordance with aspects of the present disclosure, the decarboxylated cannabis plant material comprises at least one pharmacologically active cannabinoid present as an active ingredient. In accordance with aspects of the present disclosure, the at least one pharmacologically active cannabinoid comprises tetrahydrocannabinol. In accordance with aspects of the present disclosure, the tetrahydrocannabinol is present in an amount from about 1% to about 40% total weight of the decarboxylated cannabis plant material. In accordance with aspects of the present disclosure, the tetrahydrocannabinol is present in a dose range from about 2 mg to about 500 mg. In accordance with aspects of the present disclosure, the decarboxylated cannabis plant material contains at least 90% of the maximum amount of tetrahydrocannabinol that could theoretically be present as a result of decarboxylation of the maximum amount of cannabinolic acid present in the cannabis plant material.

In accordance with aspects of the present disclosure, the at least one pharmacologically active cannabinoid comprises cannabidiol. In accordance with aspects of the present disclosure, the cannabidiol is present in an amount from about 1% to about 40% total weight of the decarboxylated cannabis plant material. In accordance with aspects of the present disclosure, the cannabidiol is present in a dose range from about 2 mg to about 500 mg. In accordance with aspects of the present disclosure, the decarboxylated cannabis plant material contains at least 80% of the maximum amount of cannabidiol that could theoretically be present as a result of decarboxylation of the maximum amount of cannabidiolic acid present in the cannabis plant material.

In accordance with aspects of the present disclosure, the at least one pharmacologically active cannabinoid comprises tetrahydrocannabinol and cannabidiol. In accordance with aspects of the present disclosure, the tetrahydrocannabinol is present in an amount from about 1% to about 40% total weight of the decarboxylated cannabis plant material, and wherein the cannabidiol is present in an amount from about 1% to about 40% total weight of the decarboxylated cannabis plant material. In accordance with aspects of the present disclosure, the tetrahydrocannabinol is present in a dose range from about 2 mg to about 500 mg, and wherein the cannabidiol is present in a dose range from about 2 mg to about 500 mg. In accordance with aspects of the present disclosure, the decarboxylated cannabis plant material contains at least 90% of the maximum amount of tetrahydrocannabinol and at least 80% of the maximum amount of cannabidiol that could theoretically be present as a result of decarboxylation of the maximum amount of cannabinolic acid and cannabidiolic acid present, respectively, in the cannabis plant material.

In accordance with aspects of the present disclosure, an amount of cannabinol present in the dosage form is less than 0.5% of the total weight of the decarboxylated cannabis plant material.

In accordance with aspects of the present disclosure, the sublingual dosage form is administered in a dispenser containing the decarboxylated cannabis plant material, wherein the dispenser sublingually delivers at least one pharmacologically active cannabinoid into the sublingual cavity of a subject when the dispenser is placed within the subject's sublingual cavity. In accordance with aspects of the present disclosure, the decarboxylated cannabis plant material is infused with flavorant. In accordance with aspects of the present disclosure, the dispenser is infused with a flavorant.

In accordance with aspects of the present disclosure, the cannabis is selected from the group consisting of Cannibis sativa, Cannabis indica, Cannabis ruderalis, and combinations thereof. In accordance with aspects of the present disclosure, the cannabis plant material comprises a THC enriched strain. In accordance with aspects of the present disclosure, a THC enriched cannabis strain is selected for decarboxylation to form the sublingual dosage form. In accordance with aspects of the present disclosure, the cannabis plant material comprises a CBD enriched strain. In accordance with aspects of the present disclosure, a CBD enriched cannabis strain is selected for decarboxylation to form the sublingual dosage form. In accordance with aspects of the present disclosure, the cannabis plant material comprises a THC- and CBD-enriched strain. In accordance with aspects of the present disclosure, a THC- and CBD-enriched cannabis strain is selected for decarboxylation to form the sublingual dosage form.

In accordance with an embodiment of the present disclosure, a method of preparing a sublingual dosage form comprising decarboxylated cannabis plant material is provided. The method includes decarboxylating an amount of raw cannabis plant material sufficient to deliver an effective dose of at least one pharmacologically active cannabinoid present in the decarboxylated cannabis plant material into the systemic circulation of a subject when the decarboxylated cannabis plant material is placed into the sublingual cavity of the subject. In accordance with aspects of the present disclosure, decarboxylating the raw cannabis plant material activates at least one pharmacologically active cannabinoid present in the raw cannabis plant material. In accordance with aspects of the present disclosure, decarboxylating the raw cannabis plant material activates at least one pharmacologically active cannabinoid present in the raw cannabis plant material in the absence of subsequent processing of the decarboxylated cannabis plant material. In accordance with aspects of the present disclosure, decarboxylating the raw cannabis plant material converts at least one inactive precursor acid present in the raw cannabis plant material into at least one pharmacologically active cannabinoid. In accordance with aspects of the present disclosure, decarboxylating the raw cannabis plant material activates at least one pharmacologically active cannabinoid present in the raw cannabis plant material in the absence of subjecting the raw cannabis plant material to a processing technique selected from the group consisting of admixing, condensation, decoction, distillation, expression, extraction selected from the group consisting of aqueous extraction, solvent extraction, ethanolic extraction, gas extraction, CO2 extraction, extrusion, filtration, isolation, maceration, percolation, precipitation, pulverization, purification, solvent casting, spheronization, sublimation, volatilization, and winterization. In other words, the at least one pharmacologically active cannabinoid is available in its active form for sublingual administration without extracting the at least one pharmacologically active cannabinoid from the decarboxylated cannabis plant material.

In accordance with aspects of the present disclosure, decarboxylating the amount of raw cannabis plant material comprises heating the amount of raw cannabis plant material in an oxygen controlled environment at a temperature of from about 85° C. to about 125° C. for a period of from about 60 minutes to about 120 minutes, thereby decarboxylating the amount of raw cannabis plant material to decarboxylated cannabis plant material. In accordance with aspects of the present disclosure, decarboxylating the amount of raw cannabis plant material comprises heating the amount of raw cannabis plant material in an oxygen controlled environment at a temperature of from about 105° C. to about 115° C. for a period of from about 20 minutes to about 60 minutes, thereby decarboxylating the amount of raw cannabis plant material to decarboxylated cannabis plant material. In accordance with aspects of the present disclosure, the oxygen controlled environment contains an initial amount of oxygen present consisting of atmospheric oxygen that enters the oxygen controlled environment when the amount of raw cannabis plant material enters the oxygen controlled environment. In accordance with aspects of the present disclosure, the initial amount of oxygen present in the oxygen controlled environment minimizes oxidative degradation of the decarboxylated cannabis plant material. In accordance with aspects of the present disclosure, the oxygen controlled environment permits the initial amount of oxygen present to escape while preventing atmospheric oxygen from entering the oxygen controlled environment during decarboxylation of the cannabis plant material, thereby minimizing oxidative degradation of the decarboxylated cannabis plant material.

In accordance with aspects of the present disclosure, the heating is performed utilizing a heat source external to the oxygen controlled environment. In accordance with aspects of the present disclosure, the method includes a step of maximizing heat transmission from the heat source external to the oxygen controlled environment to the amount of raw cannabis plant material in the oxygen controlled environment.

In accordance with aspects of the present disclosure, decarboxylating the amount of raw cannabis plant material includes the steps of (i) placing the amount of raw cannabis plant material into an oxygen controlled environment; (ii) sealing the oxygen controlled environment, wherein sealing the oxygen controlled environment limits the oxygen content in the oxygen controlled environment to an initial amount of atmospheric oxygen present in air that enters the oxygen controlled environment during step (i) prior to sealing the oxygen controlled environment; and (Hi) heating the oxygen controlled environment indirectly with a heat source outside the oxygen controlled environment at a temperature of from about 85° C. to about 125° C. for a period of from about 60 minutes to about 120 minutes to decarboxylate the amount of raw cannabis plant material inside the oxygen controlled environment. The oxygen controlled environment minimizes oxidative degradation of the decarboxylated cannabis plant material by permitting the initial amount of atmospheric oxygen present in the oxygen controlled environment to escape while preventing atmospheric oxygen external to the oxygen controlled environment from entering the oxygen controlled environment during decarboxylation of the amount of raw cannabis plant material.

In accordance with aspects of the present disclosure, decarboxylating the amount of raw cannabis plant material includes the steps of: (i) placing the amount of raw cannabis plant material into an oxygen controlled environment; (ii) sealing the oxygen controlled environment, wherein sealing the oxygen controlled environment limits the oxygen content in the oxygen controlled environment to an initial amount of atmospheric oxygen present in air that enters the oxygen controlled environment during step (i) prior to sealing the oxygen controlled environment; and (iii) heating the oxygen controlled environment indirectly with a heat source outside the oxygen controlled environment at a temperature of from about 105° C. to about 115° C. for a period of from about 20 minutes to about 60 minutes to decarboxylate the amount of raw cannabis plant material inside the oxygen controlled environment. The oxygen controlled environment minimizes oxidative degradation of the decarboxylated cannabis plant material by permitting the initial amount of atmospheric oxygen present in the oxygen controlled environment to escape while preventing atmospheric oxygen external to the oxygen controlled environment from entering the oxygen controlled environment during decarboxylation of the amount of raw cannabis plant material.

In accordance with aspects of the present disclosure, decarboxylating the amount of raw cannabis plant material does not comprise exposing the oxygen controlled environment to steam created by water boiling in proximity to the oxygen controlled environment for up to 60 minutes.

In accordance with aspects of the present disclosure, the method includes, prior to step (ii), a step of maximizing heat transmission through the oxygen controlled environment to the amount of raw cannabis plant material inside the oxygen controlled environment. In accordance with aspects of the present disclosure, maximizing heat transmission through the oxygen controlled environment to the amount of raw cannabis plant material inside the oxygen controlled environment comprises pressing the amount of raw cannabis plant material present inside the oxygen controlled environment against one or more internal surfaces of the oxygen controlled environment. In accordance with aspects of the present disclosure, the amount of raw cannabis plant material is at least pressed against a surface of the oxygen controlled environment that is nearest to the external heat source.

In accordance with aspects of the present disclosure, the method includes shaping or forming the decarboxylated cannabis plant material. In accordance with aspects of the present disclosure, the shaping or forming the decarboxylated cannabis plant material is performed in the absence of compressing the decarboxylated cannabis plant material into a conventional cohesive pharmaceutical dosage form. In accordance with aspects of the present disclosure, the method includes flattening or spreading out the decarboxylated cannabis plant material. In accordance with aspects of the present disclosure, the method includes infusing the decarboxylated cannabis plant material with a flavorant. In accordance with aspects of the present disclosure, the method includes placing the decarboxylated cannabis plant material into a dispenser. In accordance with aspects of the present disclosure, the method includes placing the decarboxylated cannabis plant material into a storage medium. In accordance with aspects of the present disclosure, the storage medium prevents at least one pharmacologically active cannabinoid present in the decarboxylated cannabis plant material from degradation. In accordance with aspects of the present disclosure, the method includes packaging the dispenser containing the decarboxylated cannabis plant material or the storage medium containing the decarboxylated cannabis plant material into a kit for commercial distribution. In accordance with aspects of the present disclosure, the storage medium minimizes or prevents the decarboxylated cannabis plant material from being exposed to oxygen. In accordance with aspects of the present disclosure, the storage medium minimizes or prevents the decarboxylated cannabis plant material from being exposed to light.

In accordance with aspects of the present disclosure, the at least one pharmacologically active cannabinoid produced according to the method includes tetrahydrocannabinol, cannabidiol, or tetrahydrocannabinol and cannabidiol. In accordance with aspects of the present disclosure, such tetrahydrocannabinol is present in an amount from about 1% to about 40% total weight of the decarboxylated cannabis plant material, and wherein the cannabidiol is present in an amount from about 1% to about 40% total weight of the decarboxylated cannabis plant material. In accordance with aspects of the present disclosure, such tetrahydrocannabinol is present in a dose range from about 2 mg to about 500 mg, and wherein the cannabidiol is present in a dose range from about 2 mg to about 500 mg. In accordance with aspects of the present disclosure, such decarboxylated cannabis plant material contains at least 90% of the maximum amount of tetrahydrocannabinol and cannabidiol that could theoretically be present as a result of decarboxylation of the maximum amount of cannabinolic acid and cannabidiolic acid present in the cannabis plant material. In accordance with aspects of the present disclosure, such amount of cannabinol present in the dosage form is less than 0.5% of the total weight of the decarboxylated cannabis plant material.

In accordance with an embodiment of the present disclosure, a method of preparing a sublingual dosage form comprising decarboxylated cannabis plant material is provided. The method includes decarboxylating raw cannabis plant material at a temperature range suitable for and a period of time sufficient for the decarboxylated cannabis plant material to contain at least 70% of the maximum amount of at least one pharmacologically active cannabinoid that could theoretically be present as a result of decarboxylation of the maximum amount of the at least one pharmacologically active cannabinoid's inactive precursor present in the raw cannabis plant material. In accordance with aspects of the present disclosure, the raw cannabis plant material is decarboxylated at a temperature range suitable for and a period of time sufficient for the decarboxylated cannabis plant material to contain at least 80% of the maximum amount of at least one pharmacologically active cannabinoid that could theoretically be present as a result of decarboxylation of the maximum amount of the at least one pharmacologically active cannabinoid's inactive precursor present in the raw cannabis plant material. In accordance with aspects of the present disclosure, the raw cannabis plant material is decarboxylated at a temperature range suitable for and a period of time sufficient for the decarboxylated cannabis plant material to contain at least 90% of the maximum amount of at least one pharmacologically active cannabinoid that could theoretically be present as a result of decarboxylation of the maximum amount of the at least one pharmacologically active cannabinoid's inactive precursor present in the raw cannabis plant material. In accordance with aspects of the present disclosure, the raw cannabis plant material is decarboxylated at a temperature range suitable for and a period of time sufficient for the decarboxylated cannabis plant material to contain at least 80% of the maximum amount of a first at least one pharmacologically active cannabinoid and at least 70% of the maximum amount of a second at least one pharmacologically active cannabinoid that could theoretically be present as a result of decarboxylation of the maximum amount of the first and second at least one pharmacologically active cannabinoid's inactive precursors present in the raw cannabis plant material. In accordance with aspects of the present disclosure, the raw cannabis plant material is decarboxylated at a temperature range suitable for and a period of time sufficient for the decarboxylated cannabis plant material to contain at least 90% of the maximum amount of a first at least one pharmacologically active cannabinoid and at least 80% of the maximum amount of a second at least one pharmacologically active cannabinoid that could theoretically be present as a result of decarboxylation of the maximum amount of the first and second at least one pharmacologically active cannabinoid's inactive precursors present in the raw cannabis plant material. In accordance with aspects of the present disclosure, the raw cannabis plant material is decarboxylated at a temperature range suitable for and a period of time sufficient for the decarboxylated cannabis plant material to contain the maximum amount of a first at least one pharmacologically active cannabinoid and at least 90% of the maximum amount of a second at least one pharmacologically active cannabinoid that could theoretically be present as a result of decarboxylation of the maximum amount of the first and second at least one pharmacologically active cannabinoid's inactive precursors present in the raw cannabis plant material.

In accordance with aspects of the present disclosure, the at least one pharmacologically active cannabinoid is tetrahydrocannabinol and the at least one pharmacologically active cannabinoid's inactive precursor is cannabinolic acid. In accordance with aspects of the present disclosure, the cannabinolic acid is present in the raw cannabis plant material prior to decarboxylation in an amount from about 1% to about 40% total weight of the raw cannabis plant material. In accordance with aspects of the present disclosure, the tetrahydrocannabinol is present in the decarboxylated cannabis plant material after decarboxylation in an amount from about 1% to about 40% total weight of the decarboxylated cannabis plant material. In accordance with aspects of the present disclosure, the temperature range is from about 85° to about 125° and the period of time is between 60 minutes and 120 minutes. In accordance with aspects of the present disclosure, the decarboxylated cannabis plant material contains at least 80% of the maximum amount of tetrahydrocannabinol that could theoretically be present as a result of decarboxylation of the maximum amount of cannabinolic acid present in the raw cannabis plant material. In accordance with aspects of the present disclosure, the temperature range is from about 105° to about 115° and the period of time is between about 10 minutes and about 20 minutes. In accordance with aspects of the present disclosure, the decarboxylated cannabis plant material contains at least 90% of the maximum amount of tetrahydrocannabinol that could theoretically be present as a result of decarboxylation of the maximum amount of cannabinolic acid present in the raw cannabis plant material. In accordance with aspects of the present disclosure, the temperature range is from about 90° to about 100° and the period of time is at least 105 minutes. In accordance with aspects of the present disclosure, the temperature range is from about 105° to about 115° and the period of time is between about 20 minutes and about 60 minutes.

In accordance with aspects of the present disclosure, the at least one pharmacologically active cannabinoid is cannabidiol and the at least one pharmacologically active cannabinoid's inactive precursor is cannabidiolic acid. In accordance with aspects of the present disclosure, the cannabidiolic acid is present in the raw cannabis plant material prior to decarboxylation in an amount from about 1% to about 40% total weight of the raw cannabis plant material. In accordance with aspects of the present disclosure, the cannabidiol is present in the decarboxylated cannabis plant material after decarboxylation in an amount from about 1% to about 40% total weight of the decarboxylated cannabis plant material. In accordance with aspects of the present disclosure, the temperature range is from about 85° to about 125° and the period of time is between 60 minutes and 120 minutes. In accordance with aspects of the present disclosure, the decarboxylated cannabis plant material contains at least 80% of the maximum amount of cannabidiol that could theoretically be present as a result of decarboxylation of the maximum amount of cannabidiolic acid present in the raw cannabis plant material. In accordance with aspects of the present disclosure, the temperature range is from about 105° to about 115° and the period of time is between about 30 minutes and 60 minutes. In accordance with aspects of the present disclosure, the decarboxylated cannabis plant material contains at least 90% of the maximum amount of cannabidiol that could theoretically be present as a result of decarboxylation of the maximum amount of cannabidiolic acid present in the raw cannabis plant material and the temperature range is from about 105° to about 115° and the period of time is between about 60 minutes and about 120 minutes.

In accordance with aspects of the present disclosure, the at least one pharmacologically active cannabinoid comprises tetrahydrocannabinol and cannabidiol, and wherein the at least one pharmacologically active cannabinoid's inactive precursor is cannabinolic acid and cannabidiolic acid, respectively. In accordance with aspects of the present disclosure, the first at least one pharmacologically active cannabinoid comprises tetrahydrocannabinol and the second at least one pharmacologically active cannabinoid comprises cannabidiol, and wherein the first at least one pharmacologically active cannabinoid's inactive precursor is cannabinolic acid and the second at least one pharmacologically active cannabinoid's inactive precursor is cannabidiolic acid, respectively. In accordance with aspects of the present disclosure, the cannabinolic acid is present prior to decarboxylation in an amount from about 1% to about 40% total weight of the raw cannabis plant material, and wherein the cannabidiolic acid is present prior to decarboxylation in an amount from about 1% to about 40% total weight of the raw cannabis plant material. In accordance with aspects of the present disclosure, the tetrahydrocannabinol is present after decarboxylation in an amount from about 1% to about 40% total weight of the decarboxylated cannabis plant material, and wherein the cannabidiol is present after decarboxylation in an amount from about 1% to about 40% total weight of the decarboxylated cannabis plant material. In accordance with aspects of the present disclosure, the temperature range is from about 85° to about 125° and the period of time is between 60 minutes and 120 minutes. In accordance with aspects of the present disclosure, the decarboxylated cannabis plant material contains at least 80% of the maximum amount of tetrahydrocannabinol and cannabidiol that could theoretically be present as a result of decarboxylation of the maximum amount of cannabinolic acid and cannabidiolic acid, respectively, present in the raw cannabis plant material. In accordance with aspects of the present disclosure, the temperature range is from about 90° to about 100° and the period of time is at least 105 minutes. In accordance with aspects of the present disclosure, the temperature range is from about 105° to about 115° and the period of time is between about 20 minutes and about 60 minutes. In accordance with aspects of the present disclosure, an amount of cannabinol present in the dosage form is less than 0.5% of the total weight of the decarboxylated cannabis plant material.

In accordance with an embodiment of the present disclosure, an apparatus for preparing a sublingual dosage form comprising decarboxylated cannabis plant material is provided. The apparatus includes a first container having a first receptacle configured to retain a fluid medium, and a cap configured to sealably engage the first container, thereby providing the first container with a temperature controllable environment. The apparatus also includes a second container sealably engaged to and suspended from the cap. The second container is configured to provide an oxygen controlled environment for minimizing oxidation of cannabinoids during decarboxylation of cannabis plant material. The second container has a second receptacle configured to receive an amount of raw cannabis plant material, and an impervious barrier which prevents fluid present in the first container from penetrating the second container, thereby minimizing oxidation of cannabinoids during decarboxylation of the cannabis plant material. The impervious barrier includes a wall portion and a base portion. The wall portion extends circumferentially from a first open end of the impervious barrier to a second end of the impervious barrier which is closed by the base portion. The amount of raw cannabis plant material received in the second receptacle is sufficient for preparing a sublingual dosage form that delivers an effective amount of at least one pharmacologically active cannabinoid present in the decarboxylated cannabis plant material into the system circulation when the decarboxylated cannabis plant material is placed into the sublingual cavity.

In accordance with aspects of the present disclosure, the apparatus includes a disc. The disc reduces oxygen content in the oxygen controlled environment. The disc is configured to fit within the second container, and sealably engage the wall portion of the impervious barrier when pressed toward the base portion of the impervious barrier. In accordance with aspects of the present disclosure, an apparatus for heating contents in an oxygen controlled environment is provided. The apparatus includes a first container having a first receptacle configured to retain a fluid medium, and a cap configured to sealably engage the first container, thereby providing the first container with a temperature controllable environment. The apparatus also includes a second container sealably engaged to and suspended from the cap, the second container configured to provide an oxygen controlled environment for limiting the amount of oxygen present in the second container. The second container has a receptacle, and an impervious barrier which prevents fluid present in the first container from penetrating the second container. The impervious barrier includes a wall portion and a base portion. The wall portion extends circumferentially from a first open end of the impervious barrier to a second end of the impervious barrier which is closed by the base portion. The apparatus also includes a disc for reducing oxygen content in the oxygen controlled environment. The disc is configured to fit within the second container, and sealably engage the wall portion of the impervious barrier when pressed toward the base portion of the impervious barrier.

In accordance with aspects of the present disclosure, the disc includes a core member and a circumferential member. The core member is sized and dimensioned to cover the base portion of the impervious barrier when the disc is sealably engaged to the wall portion of the impervious barrier. The circumferential member is configured to sealably engage the wall portion of the impervious barrier while the disc is moved within the second container toward the base portion of the impervious barrier. In accordance with aspects of the present disclosure, the circumferential member is constructed of a pliable material that enables the disc to form a shallow cup configuration with the core member bowed into contact with contents in the receptacle. In accordance with aspects of the present disclosure, the circumferential member has a geometry selected from the group consisting of a tapered, square, rounded, and butterflied. In accordance with aspects of the present disclosure, the circumferential member comprises a ring that rolls relative to the core member. In accordance with aspects of the present disclosure, the ring enables the disc to move within the second container relative to the base portion while the disc is sealably engaged to the wall portion of the impervious barrier. In accordance with aspects of the present disclosure, when the disc is pressed toward the base portion of the impervious barrier the core member compresses contents present in the receptacle against the base portion of the impervious barrier. In accordance with aspects of the present disclosure, when the disc is pressed toward the base portion of the second container the core member compresses contents present in the receptacle against the wall portion of the impervious barrier. In accordance with aspects of the present disclosure, compression of the contents maximizes heat transmission from the temperature controlled environment to the contents present in the second receptacle. In accordance with aspects of the present disclosure, the apparatus includes a base configured to hold one or more control elements. In accordance with aspects of the present disclosure, a heating element is disposed at the bottom of the container adjacent to the base.

In accordance with aspects of the present disclosure, the heating element is configured to maintain the temperature controllable environment in the first container at a temperature for a time period sufficient to decarboxylate at least one inactive precursor acid in the raw cannabis plant material to the at least one pharmacologically active cannabinoid. In accordance with aspects of the present disclosure, the heating element is configured to maintain the temperature controllable environment in the first container at a temperature for a time period sufficient to decarboxylate at least 70% of the at least one inactive precursor acid in the raw cannabis plant material retained in the receptacle to the at least one pharmacologically active cannabinoid. In accordance with aspects of the present disclosure, the at least one inactive precursor is cannabinolic acid, cannabidiolic acid, or both cannabinolic acid and cannabidiolic acid, and the at least one pharmacologically active cannabinoid is tetrahydrocannabinol, cannabidiol or tetrahydrocannabinol and cannabidiol, respectively. In accordance with aspects of the present disclosure, the temperature comprises a range from about 85° C. to about 125° C., and the time period is from about 60 minutes to 120 minutes. In accordance with aspects of the present disclosure, the heating element is configured to maintain the temperature controllable environment in the first container at a temperature for a time period sufficient to decarboxylate at least 90% of the at least one inactive precursor acid in the raw cannabis plant material to the at least one pharmacologically active cannabinoid. In accordance with aspects of the present disclosure, the at least one inactive precursor is cannabinolic acid, cannabidiolic acid, or both cannabinolic acid and cannabidiolic acid, and the at least one pharmacologically active cannabinoid is tetrahydrocannabinol, cannabidiol or tetrahydrocannabinol and cannabidiol, respectively. In accordance with aspects of the present disclosure, the temperature comprises a range from about 90° C. to about 100° C., and the time period is at least 105 minutes. In accordance with aspects of the present disclosure, the temperature comprises a range from about 105° C. to about 115° C., and the time period is from about 20 minutes to about 60 minutes. In accordance with aspects of the present disclosure, an amount of cannabinol present in the decarboxylated cannabis plant material is less than 0.5% of the total weight of the decarboxylated cannabis plant material.

In accordance with aspects of the present disclosure, the apparatus includes an indicator light disposed in the base. In accordance with aspects of the present disclosure, the indicator light indicates that the decarboxylated cannabis plant material contains an effective amount of the at least one pharmacologically active cannabinoid for sublingual administration. In accordance with aspects of the present disclosure, a temperature sensitive switching element is disposed in parallel electrical connection with the indicator light. In accordance with aspects of the present disclosure, the temperature sensitive switching element maintains the temperature of the temperature controllable environment in a temperature range that maximizes decarboxylation of inactive precursor acids in the raw cannabis plant material while minimizing pyrolytic degradation of the decarboxylated cannabis plant material. In accordance with aspects of the present disclosure, the temperature range is from about 85° C. to about 125° C. In accordance with aspects of the present disclosure, the temperature sensitive switching element shuts off the heating element when the temperature in the temperature controllable environment exceeds the upper limit of the temperature range. In accordance with aspects of the present disclosure, the temperature sensitive switching element turns on the heating element when the temperature in the temperature controllable environment drops below the lower limit of the temperature range.

In accordance with aspects of the present disclosure, the cap comprises a valve. In accordance with aspects of the present disclosure, the valve comprises a one-way valve. In accordance with aspects of the present disclosure, the valve is situated in a portion of the cap that allows one-way fluid communication from the second container to the environment external to the apparatus. In accordance with aspects of the present disclosure, the valve relieves pressure during decarboxylation of the raw cannabis plant material by allowing fluid to escape the container. In accordance with aspects of the present disclosure, the pressure relieved by the valve maintains the cap sealably engaged to the container. In accordance with aspects of the present disclosure, the valve prevents air from entering the second container, thereby minimizing oxidation of the at least one pharmacologically active cannabinoid present in the decarboxylated plant material.

In accordance with an embodiment of the present disclosure, a sublingual composition for delivery of at least one pharmacologically active cannabinoid present in decarboxylated cannabis plant material into the systemic circulation of a subject when the composition is placed in the subject's sublingual cavity is provided. The composition includes: (a) an amount of decarboxylated cannabis plant material sufficient to sublingually deliver an effective dose of the at least one pharmacologically active cannabinoid into the systemic circulation of a subject when the sublingual composition is placed into the sublingual cavity; and (b) a flavorant.

In accordance with an embodiment of the present disclosure, a sublingual composition for delivery of at least one pharmacologically active cannabinoid present in decarboxylated cannabis plant material into the systemic circulation of a subject when the composition is placed in the subject's sublingual cavity is provided. The composition includes: (a) at least 70% of the maximum amount of at least one pharmacologically active cannabinoid that could theoretically be present as a result of decarboxylation of the maximum amount of the at least one pharmacologically active cannabinoid's inactive precursor present in the cannabis plant material; and (b) a flavorant.

The decarboxylated cannabis plant material is infused with the flavorant. In accordance with aspects of the present disclosure, the flavorant is present in an amount of from about 1% to about 20% by weight of the sublingual composition. In accordance with aspects of the present disclosure, the decarboxylated cannabis plant material is present in an amount of about 80% to about 99% by weight of the sublingual composition. In accordance with aspects of the present disclosure, the amount of decarboxylated cannabis plant material is from about 0.05 grams to about 7 grams. In accordance with aspects of the present disclosure, the effective dose of the at least one pharmacologically active cannabinoid is from about 2 mg to about 100 mg. In accordance with aspects of the present disclosure, the at least one pharmacologically active cannabinoid in the decarboxylated cannabis plant material of the composition is tetrahydrocannabinol and the at least one pharmacologically active cannabinoid's inactive precursor is cannabinolic acid. In accordance with aspects of the present disclosure, the cannabinolic acid is present in the cannabis plant material prior to decarboxylation in an amount from about 1% to about 40% total weight of the cannabis plant material. In accordance with aspects of the present disclosure, the tetrahydrocannabinol is present in the decarboxylated cannabis plant material in an amount from about 1% to about 40% total weight of the decarboxylated cannabis plant material. In accordance with aspects of the present disclosure, the decarboxylated cannabis plant material contains at least 90% of the maximum amount of tetrahydrocannabinol that could theoretically be present as a result of decarboxylation of the maximum amount of cannabinolic acid present in the cannabis plant material.

In accordance with aspects of the present disclosure, the at least one pharmacologically active cannabinoid in the decarboxylated cannabis plant material of the composition is cannabidiol and the at least one pharmacologically active cannabinoid's inactive precursor is cannabidiolic acid. In accordance with aspects of the present disclosure, the cannabidiolic acid is present prior to decarboxylation in an amount from about 1% to about 40% total weight of the raw cannabis plant material. In accordance with aspects of the present disclosure, the cannabidiol is present after decarboxylation in an amount from about 1% to about 40% total weight of the decarboxylated cannabis plant material. In accordance with aspects of the present disclosure, the decarboxylated cannabis plant material contains at least 80% of the maximum amount of cannabidiol that could theoretically be present as a result of decarboxylation of the maximum amount of cannabidiolic acid present in the raw cannabis plant material.

In accordance with aspects of the present disclosure, the at least one pharmacologically active cannabinoid in the decarboxylated cannabis plant material of the composition comprises tetrahydrocannabinol and cannabidiol, and the at least one pharmacologically active cannabinoid's inactive precursor is cannabinolic acid and cannabidiolic acid, respectively. In accordance with aspects of the present disclosure, the cannabinolic acid is present prior to decarboxylation in an amount from about 1% to about 40% total weight of the raw cannabis plant material, and wherein the cannabidiolic acid is present prior to decarboxylation in an amount from about 1% to about 40% total weight of the raw cannabis plant material. In accordance with aspects of the present disclosure, the tetrahydrocannabinol is present after decarboxylation in an amount from about 1% to about 40% total weight of the decarboxylated cannabis plant material, and wherein the cannabidiol is present after decarboxylation in an amount from about 1% to about 40% total weight of the decarboxylated cannabis plant material. In accordance with aspects of the present disclosure, the decarboxylated cannabis plant material contains at least 90% of the maximum amount of tetrahydrocannabinol and at least 80% of the maximum amount of cannabidiol that could theoretically be present as a result of decarboxylation of the maximum amount of cannabinolic acid and cannabidiolic acid, respectively, present in the raw cannabis plant material. In accordance with aspects of the present disclosure, an amount of cannabinol present in the composition is less than 0.5% of the total weight of the decarboxylated cannabis plant material.

In accordance with an embodiment of the present disclosure, a dispenser for delivering at least one pharmacologically active cannabinoid from decarboxylated cannabis plant material contained inside the dispenser into the sublingual cavity of a subject when the dispenser is placed within the subject's sublingual cavity with the decarboxylated cannabis plant material contained thereinis provided. In some embodiments, the dispenser is ingestible. In some embodiments, the dispenser is constructed from an ingestible material. In some embodiments, the dispenser is constructed from an insoluble material. In some embodiments, the dispenser is constructed from a fibrous plant material. In some embodiments, the dispenser is constructed from a fibrous plant material comprising cellulose. In some embodiments, the dispenser is constructed from a fibrous plant material comprising at least one of hemp, rice, bamboo, corn husk, silk husk, fruit skin, straw, flax, soy, wood, a pulp of any thereof, and combinations thereof. In some embodiments, the dispenser comprises a wrapping folded around the decarboxylated cannabis plant material. In some embodiments, the dispenser comprises at least one aperture configured to retain the decarboxylated cannabis plant material inside the dispenser while delivering the at least one pharmacologically active cannabinoid from the decarboxylated cannabis plant material into the sublingual cavity of the subject. In some embodiments, the dispenser comprises a plurality of apertures configured to retain the decarboxylated cannabis plant material inside the dispenser while delivering the at least one pharmacologically active cannabinoid from the decarboxylated cannabis plant material into the sublingual cavity of the subject. In some embodiments, the dispenser comprises a fastener on a first portion of an interior surface of the dispenser which secures the first portion of the interior surface of the dispenser to at least a second portion of the interior surface of the dispenser when the dispenser is folded around the decarboxylated cannabis plant material in such a way that the fastener contacts the at least the second portion of the interior surface, thereby retaining the decarboxylated cannabis plant material inside the dispenser. In some embodiments, the fastener comprises a wax or gelatin. In some embodiments, the fastener comprises at least one of beeswax, candelilla wax, carnauba, ceresin wax, japan wax, microcrystalline wax, paraffin wax, sugarcane wax, and combinations thereof. In some embodiments, the dispenser is configured to elicit a positive sensory response from the subject when the dispenser is placed in the subject's sublingual cavity with the decarboxylated cannabis plant material contained therein. In some embodiments, the dispenser is configured to elicit a positive sensory response from the subject when the dispenser is ingested by the subject with the decarboxylated cannabis plant material contained therein. In some embodiments, the dispenser comprises at least a first coating on at least a portion of an exterior surface of the dispenser. In some embodiments, the dispenser comprises at least a first coating on the entire exterior surface of the dispenser. In some embodiments, the at least the first coating elicits a positive sensory response from the subject when the dispenser is placed in the subject's sublingual cavity or ingested by the subject with the decarboxylated cannabis plant material contained therein. In some embodiments, the positive sensory response comprises a sensation of smoothness, comfort, pleasant taste, and/or softness in the subject's sublingual cavity or throat. In some embodiments, the at least the first coating comprises a wax. In some embodiments, the at least the first coating comprises at least one of beeswax, candelilla wax, carnauba, ceresin wax, japan wax, microcrystalline wax, paraffin wax, shellac wax, sugarcane wax, and combinations thereof. In some embodiments, the at least the first coating comprises a flavorant. In some embodiments, the at least the first coating comprises an oil. In some embodiments, the at least the first coating comprises a lubricious coating (e.g., an edible lubricious coating). In some embodiments, the at least a first coating comprises a lipophilic coating (e.g., to enhance absorption of the cannabinoids). In some embodiments, the dispenser is configured to deliver an effective amount of the at least one pharmacologically active cannabinoid from the decarboxylated cannabis plant material into the subject's sublingual cavity. In some embodiments, the dispenser is configured to deliver a first effective dose of the at least one pharmacologically active cannabinoid from the decarboxylated cannabis plant material into the subject's sublingual cavity when the dispenser is placed into the subject's sublingual cavity, and to deliver a second dose of the at least one pharmacologically active cannabinoid from the decarboxylated cannabis plant material into the subject's gastrointestinal tract when the dispenser is ingested by the subject after delivering the first effective dose. In some embodiments, the dispenser is configured to deliver a predetermined dose of the at least one pharmacologically active cannabinoid from the decarboxylated cannabis plant material into the subject's sublingual cavity. In some embodiments, the dispenser is configured to deliver a first predetermined dose of the at least one pharmacologically active cannabinoid from the decarboxylated cannabis plant material when the dispenser is placed into the subject's sublingual cavity, and to deliver a second predetermined dose of the at least one pharmacologically active cannabinoid from the decarboxylated cannabis plant material when the dispenser is swallowed by the subject after delivering the first predetermined dose.

In some aspects, the invention provides a wrapping comprising at least one aperture configured to retain cannabis plant material inside the wrapping while permitting an effective amount of at least one pharmacologically active agent released from the cannabis plant material upon exposure to saliva to flow with the saliva into a subject's sublingual cavity when the wrapping is folded around the cannabis plant material and placed in the subject's sublingual cavity.

In some aspects, the invention provides a wrapping comprising a plurality of apertures configured to retain cannabis plant material inside the wrapping while permitting an effective amount of at least one pharmacologically active agent released from the cannabis plant material upon exposure to saliva to flow with the saliva into a subject's sublingual cavity when the wrapping is folded around the cannabis plant material and placed in the subject's sublingual cavity.

In some embodiments, the wrapping is packaged in an unfolded form. In some embodiments, the wrapping is packaged together with a plurality of wrappings. In some embodiments, each adjacent wrapping is separated from each other by a sheet. In some embodiments, the sheet comprises cellophane, wax paper, parchment paper, or laminated aluminum. In some embodiments, the packaging comprises an aluminum foil bag comprising a zip top and a tear notch, wherein the tear notch permits the packaging to be opened thereby permitting a consumer to remove at least one wrapping from the packaging, and wherein the zip top permits the packaging to be sealed thereby permitting the consumer to store the remaining wrappings in the packaging. In some embodiments, the wrapping comprises an asymmetrical shape. In some embodiments, the wrapping comprises a symmetrical shape. In some embodiments, the wrapping is ingestible. In some embodiments, the wrapping is constructed from an ingestible material. In some embodiments, the wrapping is constructed from an insoluble material. In some embodiments, the wrapping is constructed from a fibrous plant material. In some embodiments, the wrapping is constructed from a fibrous plant material comprising cellulose. In some embodiments, the wrapping is constructed from a fibrous plant material comprising at least one of hemp, rice, bamboo, corn husk, silk husk, fruit skin, straw, flax, soy, wood, a pulp of any thereof, and combinations thereof. In some embodiments, the aperture is configured to minimize exposure of the plant material to saliva when the wrapping is placed in the subject's sublingual cavity. In some embodiments, the aperture is configured to minimize contact between the cannabis plant material and the subject's sublingual cavity. In some embodiments, the plurality of apertures are configured to maximize exposure of the cannabis plant material to saliva when the wrapping is placed in the subject's sublingual cavity. In some embodiments, the plurality of apertures are configured to maximize contact between the cannabis plant material and the subject's sublingual cavity. In some embodiments, the plurality of apertures are arranged on the wrapping in a uniform distribution. In some embodiments, the plurality of apertures are arranged on the wrapping in a random distribution. In some embodiments, the plurality of apertures are arranged on the wrapping in a regular pattern. In some embodiments, the plurality of apertures are arranged on the wrapping in an irregular pattern. In some embodiments, each of the plurality of apertures comprises the same geometric shape. In some embodiments, each of the plurality of apertures comprises a different geometric shape. In some embodiments, the plurality of apertures comprises at least two different geometric shapes. In some embodiments, the geometric shape or shapes comprise a regular shape. In some embodiments, the geometric shape or shapes comprise an irregular shape. In some embodiments, the plurality of apertures comprise at least 10% of the surface area of the wrapping. In some embodiments, the plurality of apertures are configured on only a portion of the wrapping. In some embodiments, each of the plurality of apertures comprises an area of between about 1936 μM and 1 in². In some embodiments, each of the plurality of apertures comprises a perimeter of between 148 μM and 4 inches. In some embodiments, each of the plurality of apertures comprises a circumference of between 132 μM and 3 inches. In some embodiments, the wrapping comprises a thickness of between 0.1 mm to about 3.0 mm. In some embodiments, the cannabis plant material comprises decarboxylated cannabis plant material. In some embodiments, the effective amount of at least one pharmacologically active agent present in the cannabis plant material is a function of the mass of the cannabis plant material. In some embodiments, the wrapping is sized and dimensioned to fold around the cannabis plant material.

In some aspects, the invention provides a plurality of dispensers formed into a continuous sheet, wherein each dispenser comprises a wrapping comprising at least one aperture configured to retain cannabis plant material inside the wrapping while permitting an effective amount of at least one pharmacologically active agent in the cannabis plant material to be released into a subject's sublingual cavity when the wrapping is folded around the cannabis plant material and placed in the subject's sublingual cavity.

In some aspects, the invention provides a plurality of dispensers formed into a continuous sheet, wherein each dispenser comprises a wrapping comprising a plurality of apertures configured to retain cannabis plant material inside the wrapping while permitting an effective amount of at least one pharmacologically active agent in the cannabis plant material to be released into a subject's sublingual cavity when the wrapping is folded around the cannabis plant material and placed in the subject's sublingual cavity.

In some embodiments, each dispenser is sized and dimensioned to fold around the cannabis plant material. In some embodiments, multiple dispensers are sized and dimensioned to fold around the cannabis plant material. In some embodiments, the mass of the cannabis plant material determines the effective amount of the at least one pharmacologically active agent released from the cannabis plant material. In some embodiments, each dispenser is configured to retain a mass of cannabis plant material sufficient to deliver a unit dosage of the pharmacologically activate agent. In some embodiments, a line perforation between each of the adjacent dispensers in the continuous sheet. In some embodiments, the line perforation permits adjacent dispensers in the continuous sheet to be separated from each other. In some embodiments, the line perforation permits any dispenser in the continuous sheet to be separated from the continuous sheet by enabling a subject to tear the continuous sheet along the line perforation without damaging the structural integrity of the dispenser or the sheet. In some embodiments, the line perforation permits a user to increase the dosage by separating a group of dispensers from the sheet by tearing the line perforation between the group of dispensers and the sheet, wherein the dosage is proportionate to the number of dispensers in the group. In some embodiments, the sheet is configured in a rolled configuration. In some embodiments, the sheet is configured in a planar configuration. In some embodiments, the sheet comprises a first surface, a second surface, and a perimeter. In some embodiments, the sheet includes at least one fastener disposed proximal the perimeter on the first surface of each dispenser in the sheet. In some embodiments, the at least one fastener is disposed proximal to the entire perimeter of each dispenser in the sheet. In some embodiments, the at least one fastener is configured to secure the dispenser to itself when the dispenser is separated from the sheet by tearing at least one line perforation, and the dispenser is folded around the cannabis plant material in such a way that the dispenser contacts the at least one fastener. In some embodiments, the sheet includes a plurality of fasteners disposed proximal the perimeter on the first surface of each dispenser in the sheet. In some embodiments, at least two of the plurality of fasteners are positioned opposite to each other proximal the perimeter. In some embodiments, the at least one fastener spans the entire portion proximal to a first edge of each dispenser in the sheet. In some embodiments, the at least one fastener spans the entire portion proximal to a second edge of each dispenser in the sheet. In some embodiments, the at least one fastener spanning the entire portion proximal to the first edge of each dispenser in the sheet and the at least one fastener spanning the entire portion proximal to the second edge of each dispenser in the sheet comprise the same material. In some embodiments, the at least one fastener spanning the entire portion proximal to the first edge of each dispenser in the sheet and the at least one fastener spanning the entire portion proximal to the second edge of each dispenser in the sheet comprise a different material. In some embodiments, the at least one fastener comprises a waxy. In some embodiments, the waxy material comprises an edible wax selected from the group consisting of beeswax, candelilla wax, carnauba, ceresin wax, japan wax, microcrystalline wax, paraffin wax, shellac wax, sugarcane wax, and combinations and mixtures thereof. In some embodiments, the at least one fastener is disposed proximal the perimeter on at least a portion of each side of the sheet. In some embodiments, the at least one fastener extends along the entire perimeter of the sheet in a continuous manner. In some embodiments, the at least one fastener extends along the entire perimeter of the sheet in a discontinuous manner. In some embodiments, the at least one fastener comprises a waxy material. In some embodiments, the at least one fastener comprises at least one of beeswax, candelilla wax, carnauba, ceresin wax, japan wax, microcrystalline wax, paraffin wax, shellac wax, sugarcane wax, and combinations and mixtures thereof. In some embodiments, the at least one fastener is configured to secure at least one dispenser to itself when the at least one dispenser is separated from the sheet by tearing at least one line perforation, and folded over the cannabis plant material in such a way that the at least one dispenser contacts the at least one fastener. In some embodiments, the sheet includes a coating on at least a portion of the second surface of each dispenser in the sheet. In some embodiments, the sheet includes a coating on the entire second surface of each dispenser in the sheet. In some embodiments, the coating is configured to elicit a positive sensory response in a subject. In some embodiments, the coating elicits a positive sensory response from the subject when the dispenser is placed in the subject's sublingual cavity or ingested by the subject with the cannabis plant material contained therein. In some embodiments, the positive sensory response comprises a sensation of smoothness, comfort, and/or softness in the subject's sublingual cavity or throat. In some embodiments, the coating comprises a wax, gelatin, a lipophilic material, a lubricious material, or combinations thereof. In some embodiments, the coating comprises at least one of beeswax, candelilla wax, carnauba, ceresin wax, japan wax, microcrystalline wax, paraffin wax, sugarcane wax, and combinations thereof. In some embodiments, the cannabis plant material comprises decarboxylated cannabis plant material. In some embodiments, the pharmacologically active agent comprises a pharmacologically active cannabinoid.

In some aspects, the dispenser comprises a) a wrapping having a first surface, a second surface, and a perimeter defined by a first side, a second side, a third side, and a fourth side, the wrapping comprising at least one aperture configured to retain a decarboxylated cannabis plant material inside the wrapping while permitting a maximum dosage of at least one pharmacologically active cannabinoid present in the decarboxylated cannabis plant material to be released into a subject's sublingual cavity when the wrapping is folded around the decarboxylated cannabis plant material and placed into the subject's sublingual cavity; and b) a first fastener on at least a portion of the first surface.

In some aspects, the dispenser comprises a) a wrapping having a first surface, a second surface, and a perimeter defined by a first side, a second side, a third side, and a fourth side, the wrapping comprising a plurality of apertures configured to retain decarboxylated cannabis plant material inside the wrapping while permitting a maximum dosage of at least one pharmacologically active cannabinoid present in the decarboxylated cannabis plant material to be released into a subject's sublingual cavity when the wrapping is folded around the decarboxylated cannabis plant material and placed into the subject's sublingual cavity; and b) a first fastener on at least a portion of the first surface.

In some embodiments, at least a portion of the first side of the first surface is folded around the decarboxylated cannabis plant material into at least a portion of any one of the second, third, or fourth sides of the first surface to retain the decarboxylated cannabis plant material inside the wrapping. In some embodiments, the fastener secures the at least the portion of the first side of the first surface to at least the portion of any one of the second, third, or fourth sides of the first surface, so as to retain the plant material inside the wrapping when the at least the portion of the first side is folded around the plant material into contact with the at least the portion of any one of the second, third, or fourth sides. In some embodiments, the dispenser includes a second, third, and/or fourth fastener on at least a portion of any one of the second, third and/or fourth sides. In some embodiments, each of the first, second, third, and/or fourth fasteners is constructed from the same material. In some embodiments, each of the first, second, third, and/or fourth fasteners is constructed from a different material. In some embodiments, any two of the first, second, third, and/or fourth fasteners is constructed from the same material. In some embodiments, any three of the first, second, third, and/or fourth fasteners is constructed from the same material. In some embodiments, any two of the first, second, third, and/or fourth fasteners is constructed from a different material. In some embodiments, any three of the first, second, third, and/or fourth fasteners is constructed from a different material. In some embodiments, the first, second, third, and/or fourth fasteners comprises at least one or any combination of beeswax, candelilla wax, carnauba, ceresin wax, japan wax, microcrystalline wax, paraffin wax, shellac wax, and sugarcane wax. In some embodiments, the dispenser includes a coating on at least a portion of the second surface. In some embodiments, the dispenser includes a coating on at least a portion of the first surface. In some embodiments, the dispenser includes a coating on approximately 50% of the second surface. In some embodiments, the dispenser includes a coating on the entire portion of the second surface. In some embodiments, the coating is configured to elicit a positive sensory response from the subject when the dispenser is placed in the subject's sublingual cavity. In some embodiments, the coating is configured to elicit a positive sensory response from the subject when the dispenser is ingested by the subject. In some embodiments, the positive sensory response comprises a sensation of at least one of smoothness, comfort, and/or softness in the subject's sublingual cavity or throat. In some embodiments, the coating is constructed from a material comprising at least one or any combination of beeswax, candelilla wax, carnauba, ceresin wax, japan wax, microcrystalline wax, paraffin wax, sugarcane wax, and gelatin. In some embodiments, the dispenser comprises a flavorant. In some embodiments, the dispenser is ingestible. In some embodiments, the dispenser is constructed from an ingestible material. In some embodiments, the dispenser is constructed from an insoluble material. In some embodiments, the dispenser is constructed from a fibrous plant material. In some embodiments, the dispenser is constructed from a fibrous plant material comprising cellulose. In some embodiments, the dispenser is constructed from a fibrous plant material comprising at least one of hemp, rice, bamboo, corn husk, silk husk, fruit skin, straw, flax, soy, wood, a pulp of any thereof, and combinations thereof.

In some aspects, the invention provides a method of administering at least one pharmacologically active cannabinoid to a subject in need thereof, comprising: providing a wrapping comprising at least one aperture configured to retain a decarboxylated cannabis plant material inside the wrapping while permitting an effective amount of at least one pharmacologically active cannabinoid to be released into a subject's sublingual cavity when the wrapping is folded around the decarboxylated cannabis plant material and placed in the subject's sublingual cavity; folding the wrapping around the decarboxylated cannabis plant material; and placing the dispenser in the subject's sublingual cavity.

In some aspects, the invention provides a method of administering at least one pharmacologically active cannabinoid to a subject in need thereof, comprising: providing a wrapping comprising a plurality of apertures configured to retain decarboxylated cannabis plant material inside the wrapping while permitting an effective amount of at least one pharmacologically active cannabinoid to be released into a subject's sublingual cavity when the wrapping is folded around the decarboxylated cannabis plant material and placed in the subject's sublingual cavity; folding the wrapping around the decarboxylated cannabis plant material; and placing the dispenser in the subject's sublingual cavity.

In some embodiments, the wrapping is placed in the subject's sublingual cavity for a period of time sufficient to release the effective amount of the at least one pharmacologically active cannabinoid into the subject's sublingual cavity. In some embodiments, the method includes ingesting the wrapping with the decarboxylated cannabis plant material inside the wrapping.

In some aspects, the invention provides a method of administering at least one pharmacologically active cannabinoid to a subject in need thereof, comprising: providing a wrapping comprising at least one aperture configured to retain decarboxylated cannabis plant material inside the wrapping while permitting an effective amount of at least one pharmacologically active cannabinoid to be released into a subject's sublingual cavity when the wrapping is folded around the decarboxylated cannabis plant material and placed in the subject's sublingual cavity; instructing the subject to fold the wrapping around the decarboxylated cannabis plant material; and instructing the subject to place the wrapping in the subject's sublingual cavity.

In some aspects, the invention provides a method of administering at least one pharmacologically active cannabinoid to a subject in need thereof, comprising: providing a wrapping comprising a plurality of apertures configured to retain decarboxylated cannabis plant material inside the wrapping while permitting an effective amount of at least one pharmacologically active cannabinoid to be released into a subject's sublingual cavity when the wrapping is folded around the decarboxylated cannabis plant material and placed in the subject's sublingual cavity; instructing the subject to fold the wrapping around the decarboxylated cannabis plant material; and instructing the subject to place the wrapping in the subject's sublingual cavity.

In some embodiments, the subject is instructed to place the wrapping in the subject's sublingual cavity for a period of time sufficient to release the effective amount of the at least one pharmacologically active cannabinoid into the subject's sublingual cavity. In some embodiments, the method includes instructing the subject to ingest the wrapping with the decarboxylated cannabis plant material inside the wrapping.

In some aspects, the invention provides a kit comprising a first wrapping and a second wrapping, wherein the first wrapping comprises at least one aperture configured to retain decarboxylated cannabis plant material inside the wrapping while permitting an effective amount of at least one pharmacologically active cannabinoid to be released into a subject's sublingual cavity when the wrapping is folded around the decarboxylated cannabis plant material and placed in the subject's sublingual cavity; and wherein the first wrapping is packaged inside the second wrapping.

In some aspects, the invention provides a kit comprising a first wrapping and a second wrapping, wherein the first wrapping comprises a plurality of apertures configured to retain decarboxylated cannabis plant material inside the wrapping while permitting an effective amount of at least one pharmacologically active cannabinoid to be released into a subject's sublingual cavity when the wrapping is folded around the decarboxylated cannabis plant material and placed in the subject's sublingual cavity; and wherein the first wrapping is packaged inside the second wrapping.

In some embodiments, the second wrapping comprises a wax sheet, a cellophane sheet, or a foil sheet.

In some aspects, the invention provides a wrapping, comprising: a sheet of ingestible material having a perimeter, a first surface, and a second surface on an opposite side of the sheet from the first surface; a plurality of apertures passing completely through from the first surface to the second surface and vice versa; and at least one wax fastener disposed proximal the perimeter and configured to secure the sheet of ingestible material to itself when the sheet is folded over and placed in contact with the at least one wax fastener.

In some aspects, the invention provides a wrapping, comprising: a sheet of ingestible material having a perimeter, a first surface, and a second surface on an opposite side of the sheet from the first surface; at least one aperture passing completely through from the first surface to the second surface and vice versa; and at least one wax fastener disposed proximal the perimeter and configured to secure the sheet of ingestible material to itself when the sheet is folded over and placed in contact with the at least one wax fastener.

In some embodiments, the sheet comprises a non-woven structure.

In some embodiments, the ingestible material comprises an insoluble material. In some embodiments, the ingestible material comprises a fibrous plant material. In some embodiments, the ingestible material comprises cellulose. In some embodiments, the ingestible material comprises at least one of hemp, rice, bamboo, corn husk, silk husk, fruit skin, straw, flax, soy, wood, a pulp of any thereof, and combinations thereof. In some embodiments, the at least one apertures comprise a plurality of apertures. In some embodiments, the plurality of apertures comprise at least 10% of the surface area of the wrapping. In some embodiments, the plurality of apertures comprise at least 50% of the surface area of the wrapping. In some embodiments, the plurality of apertures are sized and dimensioned in such a way that they are visible to the naked eye. In some embodiments, the at least one wax fastener comprises an edible wax. In some embodiments, the edible wax is selected from the group consisting of an animal wax, a vegetable wax, and a combination thereof. In some embodiments, the animal wax is selected from the group consisting of beeswax, and shellac wax. In some embodiments, the vegetable wax is selected from the group consisting of candelilla wax, carnauba wax, ceresin wax, japan wax, microcrystalline wax, paraffin wax, sugarcane wax, soy wax, and rice bran wax. In some embodiments, the at least one wax fastener comprises a colored wax. In some embodiments, the colored wax comprises a visual folding indicator. In some embodiments, the at least one wax fastener comprises a plurality of wax fasteners disposed proximal the perimeter at different locations. In some embodiments, at least one wax fastener comprises a material than at least one other wax fastener. In some embodiments, each wax fastener comprises the same material. In some embodiments, the wrapping includes a coating disposed on an opposite surface from the at least one wax fastener. In some embodiments, the wrapping includes a coating disposed on an the same surface as the at least one wax fastener, for example, on a different portion of the surface from the wax fastener(s). In some embodiments, the coating is configured to elicit a positive sensory response. In some embodiments, the wrapping includes a coating disposed on an opposite surface from the at least one wax fastener. In some embodiments, the coating is configured to elicit a sensation of smoothness, comfort, pleasant taste, and/or softness. In some embodiments, the coating comprises an edible material selected from the group consisting of an animal wax, a vegetable wax, a gelatinous material, and a combination thereof. In some embodiments, the animal wax is selected from the group consisting of beeswax and shellac wax. In some embodiments, the vegetable wax is selected from the group consisting of candelilla wax, carnauba wax, ceresin wax, japan wax, microcrystalline wax, paraffin wax, sugarcane wax, soy wax, and rice bran wax. In some embodiments, the gelatinous material comprises gelatin. In some embodiments, the wrapping comprises a plurality of line perforations. In some embodiments, the plurality of line perforations are parallel to each other. In some embodiments, at least one of the plurality of line perforations is perpendicular to at least one other line perforation. In some embodiments, the line perforations comprise a visual tearing indicator. In some embodiments, the line perforations comprise a visual dosing indicator.

In some aspects, the invention provides a wrapping, comprising: a sheet of ingestible material having a perimeter, a first surface, and a second surface on an opposite side of the sheet from the first surface; a plurality of apertures passing completely through from the first surface to the second surface and vice versa; at least one wax fastener disposed proximal the perimeter and configured to secure the sheet of ingestible material to itself when the sheet is folded over and placed in contact with the at least one wax fastener; wherein the sheet is configured to enclose decarboxylated cannabis plant material inside the wrapping when the sheet is folded over and placed in contact with the at least one wax fastener; wherein when the wrapping is placed in the sublingual cavity of a subject with decarboyxlated cannabis plant material inside the wrapping, the at least one aperture permits cannabinoids present in the decarboxylated cannabis plant material to exit the wrapping while retaining the decarboxylated cannabis plant material inside the wrapping.

In some aspects, the invention provides a wrapping, comprising: a sheet of ingestible material having a perimeter, a first surface, and a second surface on an opposite side of the sheet from the first surface; at least one aperture passing completely through from the first surface to the second surface and vice versa; at least one wax fastener disposed proximal the perimeter and configured to secure the sheet of ingestible material to itself when the sheet is folded over and placed in contact with the at least one wax fastener; wherein the sheet is configured to enclose decarboxylated cannabis plant material inside the wrapping when the sheet is folded over and placed in contact with the at least one wax fastener; wherein when the wrapping is placed in the sublingual cavity of a subject with decarboyxlated cannabis plant material inside the wrapping, the at least one aperture permits cannabinoids present in the decarboxylated cannabis plant material to exit the wrapping while retaining the decarboxylated cannabis plant material inside the wrapping.

In some embodiments, the sheet comprises a non-woven structure. In some embodiments, the sheet comprises a woven structure. In some embodiments, the ingestible material comprises an insoluble material. In some embodiments, the ingestible material comprises a fibrous plant material. In some embodiments, the ingestible material comprises cellulose. In some embodiments, the ingestible material comprises at least one of hemp, rice, bamboo, corn husk, silk husk, fruit skin, straw, flax, soy, wood, a pulp of any thereof, and combinations thereof. In some embodiments, the at least one apertures comprise a plurality of apertures. In some embodiments, the plurality of apertures comprise at least 10% of the surface area of the wrapping. In some embodiments, the plurality of apertures comprise at least 50% of the surface area of the wrapping. In some embodiments, the plurality of apertures are sized and dimensioned in such a way that they are visible to the naked eye. In some embodiments, the at least one wax fastener comprises an edible wax. In some embodiments, the edible wax is selected from the group consisting of an animal wax, a vegetable wax, and a combination thereof. In some embodiments, the animal wax is selected from the group consisting of beeswax, and shellac wax. In some embodiments, the vegetable wax is selected from the group consisting of candelilla wax, carnauba wax, ceresin wax, japan wax, microcrystalline wax, paraffin wax, sugarcane wax, soy wax, and rice bran wax. In some embodiments, the at least one wax fastener comprises a colored wax. In some embodiments, the colored wax comprises a visual folding indicator. In some embodiments, the at least one wax fastener comprises a plurality of wax fasteners disposed proximal the perimeter at different locations. In some embodiments, at least one wax fastener comprises a material than at least one other wax fastener. In some embodiments, each wax fastener comprises the same material. In some embodiments, the sheet includes a coating disposed on an opposite surface from the at least one wax fastener. In some embodiments, the coating is configured to elicit a positive sensory response. In some embodiments, the coating is configured to elicit a sensation of smoothness, comfort, pleasant taste, and/or softness. In some embodiments, the coating comprises an edible material selected from the group consisting of an animal wax, a vegetable wax, a gelatinous material, and a combination thereof. In some embodiments, the animal wax is selected from the group consisting of beeswax and shellac wax. In some embodiments, the vegetable wax is selected from the group consisting of candelilla wax, carnauba wax, ceresin wax, japan wax, microcrystalline wax, paraffin wax, sugarcane wax, soy wax, and rice bran wax. In some embodiments, the gelatinous material comprises gelatin. In some embodiments, the wrapping comprises a plurality of line perforations. In some embodiments, the plurality of line perforations are parallel to each other. In some embodiments, at least one of the plurality of line perforations is perpendicular to at least one other line perforation. In some embodiments, the line perforations comprise a visual tearing indicator. In some embodiments, the line perforations comprise a visual dosing indicator.

In an aspect, the present invention provides a sublingual dosage form consisting of decarboxylated cannabis plant material. In an aspect, the present invention provides a sublingual dosage form comprising decarboxyalted cannabis plant material. In an aspect, the present invention provides a sublingual dosage form comprising decarboxylated cannabis plant material.

In an embodiment, the sublingual dosage form is not a conventional pharmaceutical dosage form formulated for sublingual or oral administration. In an embodiment, the sublingual dosage form is not formulated as a conventional pharmaceutical dosage form selected from the group consisting of an aerosol, a bead, a capsule, a cloth, a concentrate, an elixir, an emulsion, an extract, a fiber, a film, a gel, a globule, a granule, a chewing gum, an inhalant, a jelly, a liquid, a lozenge comprising a cannabinoid extract, an oil, a paste, a patch, a pellet, a pill, a poultice, a powder, a salve, a solution, a sponge, a spray, a strip, a suspension, a syrup, a tablet, a tape, a tincture, a trouche, and a wafer. In an embodiment, the sublingual dosage form in its finished state does not contain a pharmaceutically acceptable binder, buffering agent, carrier, chelating agent, co-solvent, cross-linking agent, diluent, disintegrant, emulsifier, excipient, flavoring agent, permeability enhancer, preservative, propellant, solvent, or surfactant compressed into a cohesive solid dosage form.

In an embodiment, the total weight of the decarboxylated cannabis plant material accounts for substantially the entire weight of the sublingual dosage form. In an embodiment, the total weight of the decarboxylated cannabis plant material ranges from about 0.05 grams to about 7 grams.

In an embodiment, the sublingual dosage form releases an effective amount of at least one pharmacologically active cannabinoid into the systemic circulation of a subject when the sublingual dosage form is placed into the sublingual cavity of the subject. In an embodiment, the sublingual dosage form instantaneously releases an effective amount of at least one pharmacologically active cannabinoid into the sublingual cavity of a subject when placed in the subject's sublingual cavity. In an embodiment, the sublingual dosage form begins to release an effective amount of at least one pharmacologically active cannabinoid into the systemic circulation of a subject within seconds of placing the sublingual dosage form in the subject's sublingual cavity. In an embodiment, the sublingual dosage form induces a pharmacological effect in a subject within about 1 minute to about 20 minutes of placing the sublingual dosage form in the subject's sublingual cavity.

In an embodiment, after releasing an effective amount of at least one pharmacologically active cannabinoid the sublingual dosage form retains an amount of at least one pharmacologically active cannabinoid effective for enteral administration.

In an embodiment, the sublingual dosage form is ingestible. In an embodiment, when ingested after releasing an effective amount of at least one pharmacologically active cannabinoid the sublingual dosage form releases an effective amount of at least one pharmacologically active cannabinoid into the gastrointestinal tract of a subject.

In an embodiment, the decarboxylated cannabis plant material comprises at least one pharmacologically active cannabinoid present as an active ingredient. In an embodiment, the at least one pharmacologically active cannabinoid is selected from the group consisting of tetrahydrocannabinol and cannabidiol. In an embodiment, the tetrahydrocannabinol is present in an amount from about 1% to about 40% total weight of the decarboxylated cannabis plant material. In an embodiment, the tetrahydrocannabinol is present in a dose range from about 2 mg to about 500 mg. In an embodiment, the cannabidiol is present in an amount from about 1% to about 40% total weight of the decarboxylated cannabis plant material. In an embodiment, the cannabidiol is present in a dose range from about 2 mg to about 500 mg.

In an embodiment, the sublingual dosage form is administered in a dispenser containing the decarboxylated cannabis plant material, wherein the dispenser sublingually delivers at least one pharmacologically active cannabinoid into the sublingual cavity of a subject when the dispenser is placed within the subject's sublingual cavity.

In an embodiment, the decarboxylated cannabis plant material is infused with flavorant.

In an embodiment, the cannabis is selected from the group consisting of Cannibis sativa, Cannabis indica, Cannabis ruderalis, and combinations thereof.

In an aspect, the present invention provides a method of preparing a sublingual dosage form comprising decarboxylated cannabis plant material, the method comprising decarboxylating an amount of raw cannabis plant material sufficient to deliver an effective dose of at least one pharmacologically active cannabinoid present in the decarboxylated cannabis plant material into the systemic circulation of a subject when the decarboxylated cannabis plant material is placed into the sublingual cavity of the subject.

In an embodiment, decarboxylating the raw cannabis plant material activates at least one pharmacologically active cannabinoid present in the raw cannabis plant material. In an embodiment, decarboxylating the raw cannabis plant material activates at least one pharmacologically active cannabinoid present in the raw cannabis plant material in the absence of subsequent processing of the decarboxylated cannabis plant material. In an embodiment, decarboxylating the raw cannabis plant material converts at least one inactive precursor acid present in the raw cannabis plant material into at least one pharmacologically active cannabinoid. In an embodiment, decarboxylating the raw cannabis plant material activates at least one pharmacologically active cannabinoid present in the raw cannabis plant material in the absence of subjecting the raw cannabis plant material to a processing technique selected from the group consisting of admixing, condensation, decoction, distillation, expression, extraction selected from the group consisting of aqueous extraction, solvent extraction, ethanolic extraction, gas extraction, CO2 extraction, extrusion, filtration, isolation, maceration, percolation, precipitation, pulverization, purification, solvent casting, spheronization, sublimation, volatilization, and winterization.

In an embodiment, decarboxylating the amount of raw cannabis plant material comprises heating the amount of raw cannabis plant material in an oxygen controlled environment at a temperature of from about 85° C. to about 125° C. for a period of from about 60 minutes to about 90 minutes, thereby decarboxylating the amount of raw cannabis plant material to decarboxylated cannabis plant material.

In an embodiment, the oxygen controlled environment contains an initial amount of oxygen present consisting of atmospheric oxygen that enters the oxygen controlled environment when the amount of raw cannabis plant material enters the oxygen controlled environment. In an embodiment, the initial amount of oxygen present in the oxygen controlled environment minimizes oxidative degradation of the decarboxylated cannabis plant material. In an embodiment, the oxygen controlled environment permits the initial amount of oxygen present to escape while preventing atmospheric oxygen from entering the oxygen controlled environment during decarboxylation of the cannabis plant material, thereby minimizing oxidative degradation of the decarboxylated cannabis plant material.

In an embodiment, the heating is performed utilizing a heat source external to the oxygen controlled environment.

In an embodiment, the method further comprises maximizing heat transmission from the heat source external to the oxygen controlled environment to the amount of raw cannabis plant material in the oxygen controlled environment.

In an embodiment, decarboxylating the amount of raw cannabis plant material comprises: (i) placing the amount of raw cannabis plant material into an oxygen controlled environment; (ii) sealing the oxygen controlled environment, wherein sealing the oxygen controlled environment limits the oxygen content in the oxygen controlled environment to an initial amount of atmospheric oxygen present in air that enters the oxygen controlled environment during step (i) prior to sealing the oxygen controlled environment; and (iii) heating the oxygen controlled environment indirectly with a heat source outside the oxygen controlled environment at a temperature of from about 85° C. to about 125° C. for a period of from about 60 minutes to about 90 minutes to decarboxylate the amount of raw cannabis plant material inside the oxygen controlled environment; wherein the oxygen controlled environment minimizes oxidative degradation of the decarboxylated cannabis plant material by permitting the initial amount of atmospheric oxygen present in the oxygen controlled environment to escape while preventing atmospheric oxygen external to the oxygen controlled environment from entering the oxygen controlled environment during decarboxylation of the amount of raw cannabis plant material.

In an embodiment, the method further comprises, prior to step (ii), maximizing heat transmission through the oxygen controlled environment to the amount of raw cannabis plant material inside the oxygen controlled environment. In an embodiment, maximizing heat transmission through the oxygen controlled environment to the amount of raw cannabis plant material inside the oxygen controlled environment comprises pressing the amount of raw cannabis plant material present inside the oxygen controlled environment against one or more internal surfaces of the oxygen controlled environment.

In an embodiment, the amount of raw cannabis plant material is at least pressed against a surface of the oxygen controlled environment that is nearest to the external heat source.

In an embodiment, the method further comprises shaping or forming the decarboxylated cannabis plant material. In an embodiment, the method further comprises flattening or spreading out the decarboxylated cannabis plant material. In an embodiment, the method further comprises gathering the decarboxylated cannabis plant material together.

In an embodiment, the method further comprises infusing the decarboxylated cannabis plant material with a flavorant.

In an embodiment, the method further comprising placing the decarboxylated cannabis plant material into a dispenser.

In an embodiment, the method further comprises packaging the decarboxylated cannabis plant material for commercial distribution.

In an embodiment, the method further comprises placing the decarboxylated cannabis plant material into a storage medium. In an embodiment, the storage medium prevents at least one pharmacologically active cannabinoid present in the decarboxylated cannabis plant material from degradation. In an embodiment, the storage medium minimizes or prevents the decarboxylated cannabis plant material from being exposed to oxygen. In an embodiment, the storage medium minimizes or prevents the decarboxylated cannabis plant material from being exposed to light.

In an aspect, the present invention provides an apparatus for preparing a sublingual dosage form comprising decarboxylated cannabis plant material, the apparatus comprising: (a) a first container having a first receptacle configured to retain a fluid medium; (b) a cap configured to sealably engage the first container, thereby providing the first container with a temperature controllable environment; and (c) a second container sealably engaged to and suspended from the cap, the second container configured to provide an oxygen controlled environment for minimizing oxidation of cannabinoids during decarboxylation of cannabis plant material, the second container having a second receptacle configured to receive an amount of raw cannabis plant material, and an impervious barrier which prevents fluid present in the first container from penetrating the second container, thereby minimizing oxidation of cannabinoids during decarboxylation of the cannabis plant material, the impervious barrier comprising a wall portion and a base portion, the wall portion extending circumferentially from a first open end of the impervious barrier to a second end of the impervious barrier which is closed by the base portion; wherein the amount of raw cannabis plant material is sufficient for preparing a sublingual dosage form that delivers an effective amount of at least one pharmacologically active cannabinoid present in the decarboxylated cannabis plant material into the system circulation when the decarboxylated cannabis plant material is placed into the sublingual cavity. Those skilled in the art will appreciate that the fluid medium can be either a liquid or a gas or combination of gases (e.g., air).

In an embodiment, the apparatus includes a disc, for reducing oxygen content in the oxygen controlled environment, configured to fit within the second container, and sealably engage the wall portion of the impervious barrier when pressed toward the base portion of the impervious barrier.

In an aspect, the present invention provides an apparatus for heating contents in an oxygen controlled environment, the apparatus comprising: (a) a first container having a first receptacle configured to retain a fluid medium; (b) a cap configured to sealably engage the first container, thereby providing the first container with a temperature controllable environment; and (c) a second container sealably engaged to and suspended from the cap, the second container configured to provide an oxygen controlled environment for limiting the amount of oxygen present in the second container, the second container having a receptacle, and an impervious barrier which prevents fluid present in the first container from penetrating the second container, the impervious barrier comprising a wall portion and a base portion, the wall portion extending circumferentially from a first open end of the impervious barrier to a second end of the impervious barrier which is closed by the base portion; and (d) a disc, for reducing oxygen content in the oxygen controlled environment, configured to fit within the second container, and sealably engage the wall portion of the impervious barrier when pressed toward the base portion of the impervious barrier.

In an embodiment, the disc comprises: (i) a core member sized and dimensioned to cover the base portion of the impervious barrier when the disc is sealably engaged to the wall portion of the impervious barrier; and (ii) a circumferential member configured to sealably engage the wall portion of the impervious barrier while the disc is moved within the second container toward the base portion of the impervious barrier.

In an embodiment, the circumferential member is constructed of a pliable material that enables the disc to form a shallow cup configuration with the core member bowed into contact with contents in the receptacle. In an embodiment, the circumferential member has a geometry selected from the group consisting of a tapered, square, rounded, and butterflied. In an embodiment, the circumferential member comprises a ring that rolls relative to the core member. In an embodiment, the ring enables the disc to move within the second container relative to the base portion while the disc is sealably engaged to the wall portion of the impervious barrier. In an embodiment, when the disc is pressed toward the base portion of the impervious barrier the core member compresses contents present in the receptacle against the base portion of the impervious barrier. In an embodiment, when the disc is pressed toward the base portion of the second container the core member compresses contents present in the receptacle against the wall portion of the impervious barrier. In an embodiment, compression of the contents maximizes heat transmission from the temperature controlled environment to the contents present in the second receptacle.

In an embodiment, the apparatus includes a base configured to hold one or more control elements.

In an embodiment, the apparatus includes a heating element disposed at the bottom of the container adjacent to the base. In an embodiment, the heating element is configured to maintain the temperature controllable environment in the first container at a temperature for a time period sufficient to decarboxylate at least one inactive precursor acid in the raw cannabis plant material to the at least one pharmacologically active cannabinoid. In an embodiment, the temperature comprises a range from about 85° C. to about 125° C. In an embodiment, the at least one inactive precursor is cannabinolic acid. In an embodiment, the at least one pharmacologically active cannabinoid is selected from the group consisting of tetrahydrocannabinol and cannabidiol. In an embodiment, the time period is from about 60 minutes to about 90 minutes.

In an embodiment, the apparatus includes an indicator light disposed in the base. In an embodiment, the indicator light indicates that the decarboxylated cannabis plant material contains an effective amount of the at least one pharmacologically active cannabinoid for sublingual administration.

In an embodiment, the apparatus includes a temperature sensitive switching element disposed in parallel electrical connection with the indicator light. In an embodiment, the temperature sensitive switching element maintains the temperature of the temperature controllable environment in a temperature range that maximizes decarboxylation of inactive precursor acids in the raw cannabis plant material while minimizing pyrolytic degradation of the decarboxylated cannabis plant material. In an embodiment, the temperature range is from about 85° C. to about 125° C. In an embodiment, the temperature sensitive switching element shuts off the heating element when the temperature in the temperature controllable environment exceeds the upper limit of the temperature range. In an embodiment, the temperature sensitive switching element turns on the heating element when the temperature in the temperature controllable environment drops below the lower limit of the temperature range.

In an embodiment, the cap includes a valve. In an embodiment, the valve comprises a one-way valve. In an embodiment, the valve is situated in a portion of the cap that allows one-way fluid communication from the second container to the environment external to the apparatus. In an embodiment, the valve relieves pressure during decarboxylation of the raw cannabis plant material by allowing fluid to escape the container. In an embodiment, the pressure relieved by the valve maintains the cap sealably engaged to the container. In an embodiment, the valve prevents air from entering the second container, thereby minimizing oxidation of the at least one pharmacologically active cannabinoid present in the decarboxylated plant material.

In an aspect, the present invention provides a sublingual composition for delivery of at least one pharmacologically active cannabinoid present in decarboxylated cannabis plant material into the systemic circulation of a subject when the composition is placed in the subject's sublingual cavity, the composition comprising: (a) an amount of decarboxylated cannabis plant material sufficient to sublingually deliver an effective dose of the at least one pharmacologically active cannabinoid into the systemic circulation of a subject when the sublingual composition is placed into the sublingual cavity; and (b) a flavorant, wherein the decarboxylated decarboxylated cannabis plant material is infused with the flavorant. In an embodiment, the flavorant is present in an amount of from about 1% to about 20% by weight of the sublingual composition. In an embodiment, the decarboxylated cannabis plant material is present in an amount of about 80% to about 99% by weight of the sublingual composition. In an embodiment, the amount of decarboxylated cannabis plant material is from about 0.05 grams to about 7 grams. In an embodiment, the effective dose of the at least one pharmacologically active cannabinoid is from about 2 mg to about 100 mg.

In an aspect, the present invention provides a system for delivering a sublingual dose of at least one pharmacologically active cannabinoid present in decarboxylated cannabis plant material into the systemic circulation of a subject, the system comprising: (a) a sublingual dosage form comprising an amount of decarboxylated cannabis plant material sufficient to sublingually deliver an effective dose of the at least one pharmacologically active cannabinoid into the systemic circulation of the subject; (b) a dispenser containing the amount of decarboxylated cannabis plant material, wherein the dispenser releases the effective dose of the at least one pharmacologically active cannabinoid present in the decarboxylated cannabis plant material into contact with the subject's sublingual mucosa when the dispenser is placed in the subject's sublingual cavity, thereby sublingually delivering the sublingual dose of the at least one pharmacologically active cannabinoid into the subject's systemic circulation.

In accordance with aspects of the present disclosure, any dispenser, product, sheet, or wrapping disclosed herein can be used as the dispenser in the system. In accordance with aspects of the present disclosure, any sublingual dosage form, decarboxylated cannabis plant material, or composition according to, or produced in accordance with a method disclosed herein can be used as the sublingual dosage form in the system.

In an aspect, the present invention provides a system comprising: (a) an apparatus for heating contents in an oxygen controlled environment, the apparatus comprising: (i) a first container having a first receptacle configured to retain a fluid medium; (ii) a cap configured to sealably engage the first container, thereby providing the first container with a temperature controllable environment; and (iii) a second container sealably engaged to and suspended from the cap, the second container configured to provide an oxygen controlled environment for limiting the amount of oxygen present in the second container, the second container having a second receptacle, and an impervious barrier which prevents fluid present in the first container from penetrating the second container, the impervious barrier comprising a wall portion and a base portion, the wall portion extending circumferentially from a first open end of the impervious barrier to a second end of the impervious barrier which is closed by the base portion; and (b) a disc configured to fit within the second container and move toward the base portion of the second container while sealably engaging the wall portion of the impervious barrier.

In an embodiment, the disc comprises: (a) a core member sized and dimensioned to cover the base portion of the impervious barrier when the disc is sealably engaged to the wall portion of the impervious barrier; and (b) a circumferential member configured to sealably engage the wall portion of the impervious barrier while the disc is moved within the second container toward the base portion of the impervious barrier. In an embodiment, the circumferential member is constructed of a pliable material enabling the disc to form a shallow cup configuration with the core member bowed into contact with contents in the receptacle when the disc circumferential member sealably engages the wall portion of the impervious barrier. In an embodiment, the circumferential member has a geometry selected from the group consisting of a tapered, square, rounded, and butterflied. In an embodiment, the circumferential member comprises a ring that rolls relative to the core member. In an embodiment, the ring enables the disc to move within the container while the disc is sealably engaged with the wall portion of the impervious barrier. In an embodiment, when the disc is pressed toward the base portion of the second container the core member compresses contents present in the second receptacle against the base portion of the impervious barrier. In an embodiment, when the disc is pressed toward the base portion of the second container the core member compresses contents present in the receptacle against the wall portion of the impervious barrier. In an embodiment, compression of the contents maximizes heat transmission from the temperature controlled environment to the contents present in the second receptacle.

In an embodiment, the contents comprise food selected from the group consisting of vegetables, grains, legumes, pastries, confections, eggs, and bread. In an embodiment, the contents comprise herbaceous plant material. In an embodiment, the herbaceous plant material comprises raw cannabis plant material. In an embodiment, the apparatus is used to decarboxylate the raw cannabis plant material. In an embodiment, decarboxylation of the raw cannabis plant material activates at least one pharmacologically active cannabinoid in the decarboxylated cannabis plant material.

In an embodiment, the oxygen controlled environment minimizes oxidation of cannabinoids present in the amount of raw cannabis plant material. In an embodiment, the oxygen controlled environment minimizes oxidation of cannabinoids present in the decarboxylated cannabis plant material. In an embodiment, the second receptacle is configured to receive an amount of raw cannabis plant material. In an embodiment, the amount of raw cannabis plant material is sufficient for preparing a sublingual dosage form that delivers an effective amount of at least one pharmacologically active cannabinoid present in the decarboxylated cannabis plant material into the system circulation when the decarboxylated cannabis plant material is placed into the sublingual cavity.

In an embodiment, the apparatus includes a base configured to hold one or more control elements. In an embodiment, the apparatus includes a heating element disposed at the bottom of the container adjacent to the base. In an embodiment, the heating element is configured to maintain the temperature controllable environment in the first container at a temperature for a time period sufficient to decarboxylate at least one inactive precursor acid in the raw cannabis plant material to the at least one pharmacologically active cannabinoid. In an embodiment, the temperature comprises a range from about 85° C. to about 125° C. In an embodiment, the at least one inactive precursor is cannabinolic acid. In an embodiment, the at least one pharmacologically active cannabinoid is selected from the group consisting of tetrahydrocannabinol and cannabidiol. In an embodiment, the time period is from about 60 minutes to about 90 minutes.

In an embodiment, the apparatus includes an indicator light disposed in the base. In an embodiment, the indicator light indicates that the decarboxylated cannabis plant material contains an effective amount of the at least one pharmacologically active cannabinoid for sublingual administration. In an embodiment, the apparatus includes a temperature sensitive switching element disposed in parallel electrical connection with the indicator light. In an embodiment, the temperature sensitive switching element maintains the temperature of the temperature controllable environment in a temperature range that maximizes decarboxylation of inactive precursor acids in the raw cannabis plant material while minimizing pyrolytic degradation of the decarboxylated cannabis plant material. In an embodiment, the temperature range is from about 85° C. to about 125° C. In an embodiment, the temperature sensitive switching element shuts off the heating element when the temperature in the temperature controllable environment exceeds the upper limit of the temperature range. In an embodiment, the temperature sensitive switching element turns on the heating element when the temperature in the temperature controllable environment drops below the lower limit of the temperature range.

In an embodiment, the cap comprises a valve. In an embodiment, the valve comprises a one-way valve. In an embodiment, the valve is situated in a portion of the cap that allows one-way fluid communication from the second container to the environment external to the apparatus. In an embodiment, the valve relieves pressure while heating contents with the apparatus by allowing fluid to escape the second container. In an embodiment, the pressure relieved by the valve maintains the cap sealably engaged to the second container. In an embodiment, the valve prevents air from entering the second container, thereby minimizing oxygen present in the oxygen controlled environment.

In accordance with an embodiment of the present disclosure, a system for delivering a sublingual dose of at least one pharmacologically active cannabinoid present in decarboxylated cannabis plant material into the systemic circulation of a subject is provided. The system includes a sublingual dosage form and a dispenser. The sublingual dosage form comprises decarboxylated cannabis plant material containing at least 70% of the maximum amount of at least one pharmacologically active cannabinoid that could theoretically be present as a result of decarboxylation of the maximum amount of the at least one pharmacologically active cannabinoid's inactive precursor present in the cannabis plant material. The dispenser contains the decarboxylated cannabis plant material, and the dispenser releases an effective dose of the at least one pharmacologically active cannabinoid present in the decarboxylated cannabis plant material into contact with the subject's sublingual mucosa when the dispenser is placed in the subject's sublingual cavity, thereby sublingually delivering the effective dose of the at least one pharmacologically active cannabinoid into the subject's systemic circulation.

In accordance with aspects of the present disclosure, the at least one pharmacologically active cannabinoid is tetrahydrocannabinol and the at least one pharmacologically active cannabinoid's inactive precursor is cannabinolic acid.

In accordance with aspects of the present disclosure, the cannabinolic acid is present in the cannabis plant material prior to decarboxylation in an amount from about 1% to about 40% total weight of the cannabis plant material. In accordance with aspects of the present disclosure, the tetrahydrocannabinol is present in the decarboxylated cannabis plant material in an amount from about 1% to about 40% total weight of the decarboxylated cannabis plant material. In accordance with aspects of the present disclosure, the decarboxylated cannabis plant material contains at least 90% of the maximum amount of tetrahydrocannabinol that could theoretically be present as a result of decarboxylation of the maximum amount of cannabinolic acid present in the cannabis plant material. In accordance with aspects of the present disclosure, the at least one pharmacologically active cannabinoid is cannabidiol and the at least one pharmacologically active cannabinoid's inactive precursor is cannabidiolic acid. In accordance with aspects of the present disclosure, the cannabidiolic acid is present prior to decarboxylation in an amount from about 1% to about 40% total weight of the raw cannabis plant material. In accordance with aspects of the present disclosure, the cannabidiol is present after decarboxylation in an amount from about 1% to about 40% total weight of the decarboxylated cannabis plant material. In accordance with aspects of the present disclosure, the decarboxylated cannabis plant material contains at least 80% of the maximum amount of cannabidiol that could theoretically be present as a result of decarboxylation of the maximum amount of cannabidiolic acid present in the raw cannabis plant material. In accordance with aspects of the present disclosure, the at least one pharmacologically active cannabinoid comprises tetrahydrocannabinol and cannabidiol, and wherein the at least one pharmacologically active cannabinoid's inactive precursor is cannabinolic acid and cannabidiolic acid, respectively. In accordance with aspects of the present disclosure, the cannabinolic acid is present prior to decarboxylation in an amount from about 1% to about 40% total weight of the raw cannabis plant material, and wherein the cannabidiolic acid is present prior to decarboxylation in an amount from about 1% to about 40% total weight of the raw cannabis plant material. In accordance with aspects of the present disclosure, the tetrahydrocannabinol is present after decarboxylation in an amount from about 1% to about 40% total weight of the decarboxylated cannabis plant material, and wherein the cannabidiol is present after decarboxylation in an amount from about 1% to about 40% total weight of the decarboxylated cannabis plant material. In accordance with aspects of the present disclosure, the decarboxylated cannabis plant material contains at least 90% of the maximum amount of tetrahydrocannabinol and at least 80% of the maximum amount of cannabidiol that could theoretically be present as a result of decarboxylation of the maximum amount of cannabinolic acid and cannabidiolic acid, respectively, present in the raw cannabis plant material. In accordance with aspects of the present disclosure, an amount of cannabinol present in the decarboxylated cannabis plant material is less than 0.5% of the total weight of the decarboxylated cannabis plant material. In accordance with aspects of the present disclosure, any dispenser, product, sheet, or wrapping disclosed herein can be used as the dispenser in the system. In accordance with aspects of the present disclosure, any sublingual dosage form, decarboxylated cannabis plant material, or composition according to, or produced in accordance with a method of, the present disclosure can be used as the sublingual dosage form in the system.

In accordance with an embodiment of the present disclosure, a kit for preparing a sublingual dosage form comprising decarboxylated cannabis plant material is disclosed. The kit includes an apparatus for preparing a sublingual dosage form comprising decarboxylated cannabis plant material, the apparatus comprising: a first container having a first receptacle configured to retain a fluid medium; a cap configured to sealably engage the first container, thereby providing the first container with a temperature controllable environment; and a second container sealably engaged to and suspended from the cap, the second container configured to provide an oxygen controlled environment for minimizing oxidation of cannabinoids during decarboxylation of cannabis plant material, the second container having a second receptacle configured to receive an amount of raw cannabis plant material, and an impervious barrier which prevents fluid present in the first container from penetrating the second container, thereby minimizing oxidation of cannabinoids during decarboxylation of the cannabis plant material; and instructions for using the apparatus to prepare the sublingual dosage form comprising the decarboxylated cannabis plant material; wherein the amount of raw cannabis plant material is sufficient for preparing a sublingual dosage form that delivers an effective amount of at least one pharmacologically active cannabinoid present in the decarboxylated cannabis plant material into the system circulation when the decarboxylated cannabis plant material is placed into the sublingual cavity. In accordance with aspects of the present disclosure, the instructions comprise directions for decarboxylating at least 70% of the at least one inactive precursor acid present in the raw cannabis plant material into the at least one pharmacologically active cannabinoid present in the decarboxylated cannabis plant material. In accordance with aspects of the present disclosure, the instructions comprise directions for heating the raw cannabis plant material in the oxygen controlled environment at a temperature range from about 85° C. to about 125° C. for a period of time from about 60 minutes to 120 minutes. In accordance with aspects of the present disclosure, the instructions comprise directions for decarboxylating at least 90% of the at least one inactive precursor acid present in the raw cannabis plant material into the at least one pharmacologically active cannabinoid present in the decarboxylated cannabis plant material. In accordance with aspects of the present disclosure, the instructions comprise directions for heating the raw cannabis plant material in the oxygen controlled environment at a temperature range from about 90° C. to about 100° C. for a time period of at least 105 minutes. In accordance with aspects of the present disclosure, the instructions comprise directions for heating the raw cannabis plant material in the oxygen controlled environment at a temperature range from about 105° C. to about 115° C. for a time period of between 20 minutes and 60 minutes. In accordance with aspects of the present disclosure, the instructions comprise directions for limiting the amount of cannabinol present in the decarboxylated cannabis plant material to less than 0.5% of the total weight of the decarboxylated cannabis plant material. In accordance with aspects of the present disclosure, the at least one inactive precursor is cannabinolic acid, cannabidiolic acid, or both cannabinolic acid and cannabidiolic acid, and the at least one pharmacologically active cannabinoid is tetrahydrocannabinol, cannabidiol or tetrahydrocannabinol and cannabidiol, respectively. In accordance with aspects of the present disclosure, the instructions comprise directions for the sublingual administration of the at least one pharmacologically active cannabinoid into the systemic circulation. In accordance with aspects of the present disclosure, the instructions comprise directions for placing the decarboxylated cannabis plant material into the sublingual cavity. In accordance with aspects of the present disclosure, the instructions comprise directions for forming an amount of the decarboxylated cannabis plant material into a shape suitable for sublingual administration of the at least one pharmacologically active cannabinoid present in the decarboxylated cannabis plant material into the systemic circulation. In accordance with aspects of the present disclosure, the instructions comprise directions for flattening out the amount of the decarboxylated cannabis plant material prior to placing the decarboxylated cannabis plant material into the sublingual cavity. In accordance with aspects of the present disclosure, the instructions comprise directions for ingesting the amount of the decarboxylated cannabis plant material after the at least one pharmacologically active cannabinoid present in the decarboxylated cannabis plant material is delivered into the systemic circulation. In accordance with aspects of the present disclosure, the kit includes a dispenser for administering the at least one pharmacologically active cannabinoid present in the decarboxylated cannabis plant material to the sublingual mucosa when the dispenser is placed into the sublingual cavity. In accordance with aspects of the present disclosure, the kit includes a dispenser, product, wrapping, or sheet described herein. In accordance with aspects of the present disclosure, the instructions comprise directions for placing the decarboxylated cannabis plant material into the dispenser. In accordance with aspects of the present disclosure, the instructions direct a user to flatten out or spread out the decarboxylated cannabis plant material prior to placing the decarboxylated cannabis plant material into the dispenser. In accordance with aspects of the present disclosure, the instructions comprise directions for administering the sublingual dosage form to a subject having a prescription for medical marijuana use. In accordance with aspects of the present disclosure, the apparatus further comprises a disc. In accordance with aspects of the present disclosure, the kit includes a scale (e.g., a digital scale, e.g., for weighing an amount of decarboxylated cannabis plant material to be placed in the sublingual cavity and/or in the dispenser.

### BRIEF DESCRIPTION OF THE DRAWINGS

These and other characteristics of the present invention will be more fully understood by reference to the following detailed description in conjunction with the attached drawings, which are summarized below.

FIG. 1A is a photograph showing a sublingual dosage form composed of decarboxylated cannabis plant material in accordance with an example embodiment of the present invention. FIG. 1B is a photograph showing a sublingual dosage form composed of decarboxylated cannabis plant material in accordance with an example embodiment of the present invention.

FIG. 2A is a photograph illustrating a smallest effective dosage in accordance with an example embodiment of the present invention. FIG. 2B is a photograph illustrating an average effective dose of the sublingual dosage form in accordance with an example embodiment of the present invention.

FIG. 3A is a photograph showing the sublingual dosage form depicted in FIG. 1A shaped into a form suitable for sublingual administration in accordance with an example embodiment of the present invention. FIG. 3B is a photograph showing the sublingual dosage form depicted in FIG. 1B shaped into a form suitable for sublingual administration in accordance with an example embodiment of the present invention.

FIG. 4A is a schematic illustration showing a dispenser in accordance with an example embodiment of the present invention. FIG. 4B is a schematic illustration showing a sublingual dosage form contained within the dispenser depicted in FIG. 4A in accordance with an example embodiment of the present invention.

FIGS. 5A, 5B, 5C, 5D, 5E, 5F, 5G, 5H, 5I, 5J, 5K and 5L are top views depicting a dispenser comprising a wrapping and at least one aperture in accordance with various aspects of the present invention.

FIGS. 6A, 6B, 6C, 6D, 6E, 6F, 6G, 6H, 6I, 6J, 6K and 6L are top views depicting a dispenser comprising a wrapping and a plurality of apertures in accordance with various aspects of the present invention.

FIGS. 7A, 7B, 7C, 7D, 7E, 7F, 7G, 7H, 7I and 7J are top views depicting a dispenser comprising a wrapping and a plurality of apertures in accordance with various aspects of the present invention.

FIGS. 8A, 8B, 8C, 8D, 8E, 8F, 8G, 8H, 8I, 8J, 8K, 8L, 8M, 8N, 8O, 8P, 8Q, 8R, 8S, 8T, 8U, 8V, 8W, 8X, 8Y, 8Z, 8AA, 8BB, 8CC, 8DD, 8EE, 8FF, 8GG, 8HH, 8II, 8JJ and 8KK are top views depicting a dispenser comprising a wrapping and a plurality of apertures in accordance with various aspects of the present invention.

FIGS. 9A, 9B, 9C, 9D, 9E, 9F, 9G, 9H, 9I, 9J, 9K and 9L are top views depicting a dispenser comprising a wrapping, at least one aperture, and at least one wax fastener in accordance with various aspects of the present invention.

FIGS. 10A, 10B, 10C, 10D, 10E, 10F, 10G, 10H, 10I, 10J, 10K and 10L are top views depicting a dispenser comprising a wrapping, a plurality of apertures, and at least one wax fastener in accordance with various aspects of the present invention.

FIGS. 11A, 11B, 11C, 11D, 11E, 11F, 11G, 11H, 11I, 11J, 11K, 11L, 11M and 11N are top views depicting exemplary configurations of the at least one wax fastener in accordance with various aspects of the present invention.

FIGS. 12A, 12B, 12C and 12D demonstrate preparation of an exemplary embodiment of a dispenser of the present invention for administration of a sublingual dosage form of the present invention. FIG. 12A is a top view of a dispenser depicting cannabis plant material placed on a wrapping. FIG. 12B shows the wrapping depicted in FIG. 12A being folded over the cannabis plant material. FIG. 12C shows at least one fastener (e.g., wax fastener) securing the wrapping to itself to retain the cannabis plant material inside the dispenser. FIG. 12D shows the wrapping depicted in FIG. 12C folded around the cannabis plant material and secured to itself in such a way that the cannabis plant material is enclosed within the wrapping and the dispenser ready for sublingual use.

FIG. 13A is a side view showing an example embodiment of a dispenser in accordance with an aspect of the present invention.

FIG. 13B is a perspective view showing an example embodiment of a dispenser in accordance with an aspect of the present invention.

FIGS. 14A, 14B, 14C, 14D, 14E, 14F, 14G, 14H, 14I and 14J are top views depicting exemplary embodiments of a plurality of dispensers (e.g., formed into a sheet) in accordance with various aspects of the present invention.

FIGS. 15A and 15B demonstrate a dispenser being physically removed (FIG. 15B) from an exemplary embodiment of a plurality of dispensers (FIG. 15A), for example, by tearing along line perforations, in accordance with an aspect of the present invention.

FIG. 16 is a cross-sectional view depicting an oxygen controlled environment in accordance with an example embodiment of the present invention.

FIG. 17A is a perspective view illustrating a first side of an apparatus in accordance with an example embodiment of the present invention. FIG. 17B is a perspective view illustrating a second side of an apparatus in accordance with an example embodiment of the present invention. FIG. 17C is an exemplary embodiment of a circuit diagram for an apparatus of the present invention.

FIG. 18A is a cross-sectional view illustrating an apparatus for preparing a sublingual dosage form comprising decarboxylated cannabis plant material in accordance with an example embodiment of the present invention. FIG. 18B is a cross-sectional view of the apparatus shown in FIG. 18A depicting an amount of raw cannabis plant material disposed in an oxygen controlled environment of the apparatus in accordance with an example embodiment of the present invention. FIG. 18C is a cross-sectional view illustrating the apparatus shown in FIG. 18B depicting a disc for reducing oxygen content in the oxygen controlled environment in accordance with an example embodiment of the present invention.

FIG. 19A is cross-sectional view illustrating a disc for reducing oxygen content in an oxygen controlled environment in accordance with an example embodiment of the present invention. FIG. 19B is a top view of the disc shown in FIG. 19A. FIG. 19C is a cross-sectional view showing the disc depicted in FIGS. 19A and 19B sealably engaging a wall portion of an impervious barrier in accordance with an example embodiment of the present invention. FIG. 19D is a cross-sectional view showing the disc depicted in FIG. 19C compressing an amount of raw cannabis plant material in accordance with an example embodiment of the present invention. FIG. 19E is a cross-sectional view showing the disc depicted in FIG. 19C compressing an amount of raw cannabis plant material in accordance with an example embodiment of the present invention.

FIGS. 20A, 20B, 20C, 20D and 20E depict an example embodiment of a disc in accordance with the present invention. FIG. 20A is cross-sectional view illustrating a disc in accordance with an example embodiment of the present invention. FIG. 20B is a cross-sectional view illustrating a disc in accordance with an example embodiment of the present invention. FIG. 20C is a top view of the disc shown in FIG. 20A. FIG. 20D is a cross-sectional view showing the disc depicted in FIG. 20A displacing a volume of fluid present in an oxygen controlled environment in accordance with an example embodiment of the present invention. FIG. 20E is a cross-sectional view showing the disc depicted in FIG. 20A compressing an amount of raw cannabis plant material in accordance with an example embodiment of the present invention.

FIGS. 21A, 21B, and 21C depict an example embodiment of a disc in accordance with the present invention. FIG. 21A is a cross-sectional view illustrating a disc in accordance with an example embodiment of the present invention. FIG. 21B is a cross-sectional view of the disc depicted in FIG. 21A showing the disc displacing a volume of fluid present in an oxygen controlled environment in accordance with an example embodiment of the present invention. FIG. 21C is a cross-sectional view showing the disc depicted in FIG. 21A compressing an amount of raw cannabis plant material in accordance with an example embodiment of the present invention.

FIGS. 22A, 22B, and 22C depict an example embodiment of a disc in accordance with the present invention. FIG. 22A is a cross-sectional view illustrating a disc in accordance with an example embodiment of the present invention. FIG. 22B is a cross-sectional view of the disc depicted in FIG. 22A showing the disc displacing a volume of fluid present in an oxygen controlled environment in accordance with an example embodiment of the present invention. FIG. 22C is a cross-sectional view showing the disc depicted in FIG. 22A compressing an amount of raw cannabis plant material in accordance with an example embodiment of the present invention.

FIGS. 23A, 23B, 23C and 23D depict a system for heating contents in an oxygen controlled environment in accordance with an example embodiment of the present invention. FIG. 23A is a cross-sectional view illustrating an apparatus for heating contents in an oxygen controlled environment in accordance with an example embodiment of the present invention. FIG. 23B is a cross-sectional view illustrating a disc in accordance with an example embodiment of the present invention. FIG. 23C is a top view of the disc depicted in FIG. 23B in accordance with an embodiment of the present invention. FIG. 23D is a cross-sectional view of the disc depicted in FIGS. 23B and 23C showing the disc displacing a volume of fluid present in an oxygen controlled environment in accordance with an example embodiment of the present invention.

FIG. 24 is a photograph illustrating a working prototype of a dispenser constructed from a hemp material in accordance with an example embodiment of the present invention.

FIG. 25 is a photograph illustrating a working prototype of a dispenser constructed from a cellulose material (e.g., clear) in accordance with an example embodiment of the present invention.

FIG. 26 is a photograph illustrating a working prototype of a dispenser constructed from a rice material in accordance with an example embodiment of the present invention.

FIG. 27 is photograph illustrating a working prototype of a dispenser comprising a colored wax visual folding indicator in accordance with an example embodiment of the present invention.

FIG. 28 is a photograph illustrating a working prototype of a dispenser comprising a colored oil visual folding indicator in accordance with an example embodiment of the present invention.

The patent or application file contains at least one drawing executed in color. Copies of this patent or patent application publication with color drawings will be provided by the Office upon request and payment of the necessary fee.

### DETAILED DESCRIPTION OF THE INVENTION

The present invention relates to a sublingual dosage form consisting of, consisting essentially of, or comprising decarboxylated cannabis plant material, an apparatus, method and system for preparing the sublingual dosage form, and related compositions and kits. Accordingly, in an aspect, the disclosure provides a sublingual dosage form comprising decarboxylated cannabis plant material. Surprisingly, and unexpectedly, the present inventors have discovered that decarboxylated cannabis plant material may be used sublingually to systemically deliver pharmacologically active cannabinoids (e.g., tetrahydrocannabinol (THC)) present in the decarboxylated cannabis plant material without further processing. This discovery is significant, as sublingual use of decarboxylated cannabis provides efficient delivery of THC and other useful cannabinoids, such as cannabidiol, without the drawbacks of smoking cannabis or cooking cannabis to eat it. The onset of pharmacological effects is rapid, and their duration equivalent to or exceeding other forms of ingestion. The work described herein demonstrates that with sublingual use delivery of both THC, and other useful cannabinoids such as cannabidiol, is significantly improved.

Sublingual Dosage Form

Sublingual delivery of cannabis is a superior mode of administration considering the hazards of smoking cannabis, and the pharmacokinetic limitations of ingesting cannabis. Sublingual delivery provides rapid effects similar to smoking without exposing the lungs to heat, tar, or other unwanted collateral effects, including unpleasant smell, smoky taste, dry mouth, throat irritation and caused both by smoke and hot embers that often enter the user's mouth and lungs during administration. When, ignited, nearly 50% of the cannabinoids present in the cannabis literally “go up in smoke” that is not actually inhaled by the user. Sublingual delivery is discreet and more efficient, requiring smaller amounts of cannabis for the same effect, as more of the pharmacologically active cannabinoids are absorbed into the bloodstream. Sublingual administration allows the user to avoid heat, tar, potential kickback, and even the carbon dioxide created by the most popular smoking alternative, vaporizers. Most importantly, the beneficial effects of sublingual administration last on average twice as long as smoking.

Sublingual delivery is superior to ingestion of cannabis as well. While ingesting cannabis can provide long lasting therapeutic effects, and allow the body to reap the benefits provided by exposure to the entire cannabis plant profile, this method presents numerous drawbacks as well. Onset is significantly delayed, on average beginning over an hour after administration. This delay also makes it difficult to titrate doses as compared to sublingual administration. Decreased bioavailability in the digestive tract results in absorption of only 10% to 20% of cannabinoids, and oral administration subjects cannabis to “first pass metabolism,” whereby the cannabinoids are first processed by the liver before entering the bloodstream. Processing by the liver alters cannabinoids, leading to distinct therapeutic effects from those experienced with other forms of administration. While ingestion of cannabis may be unsatisfactory on its own, it can be an excellent complement to sublingual administration, and in conjunction with sublingual delivery ingestion can provide the user with a more comprehensive therapeutic experience as is described in more detail herein.

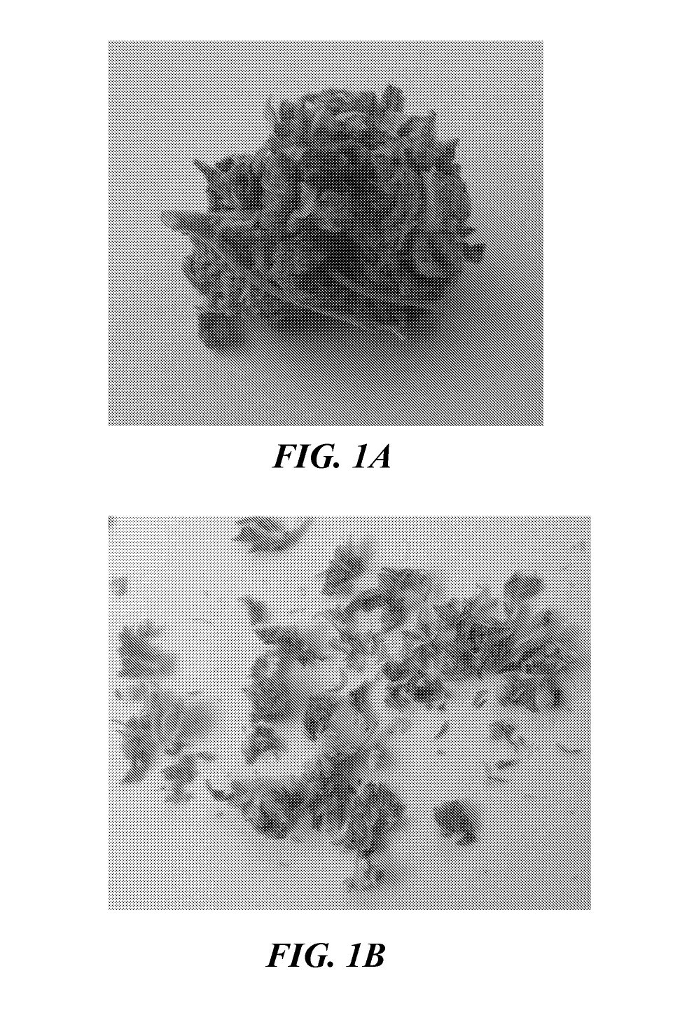

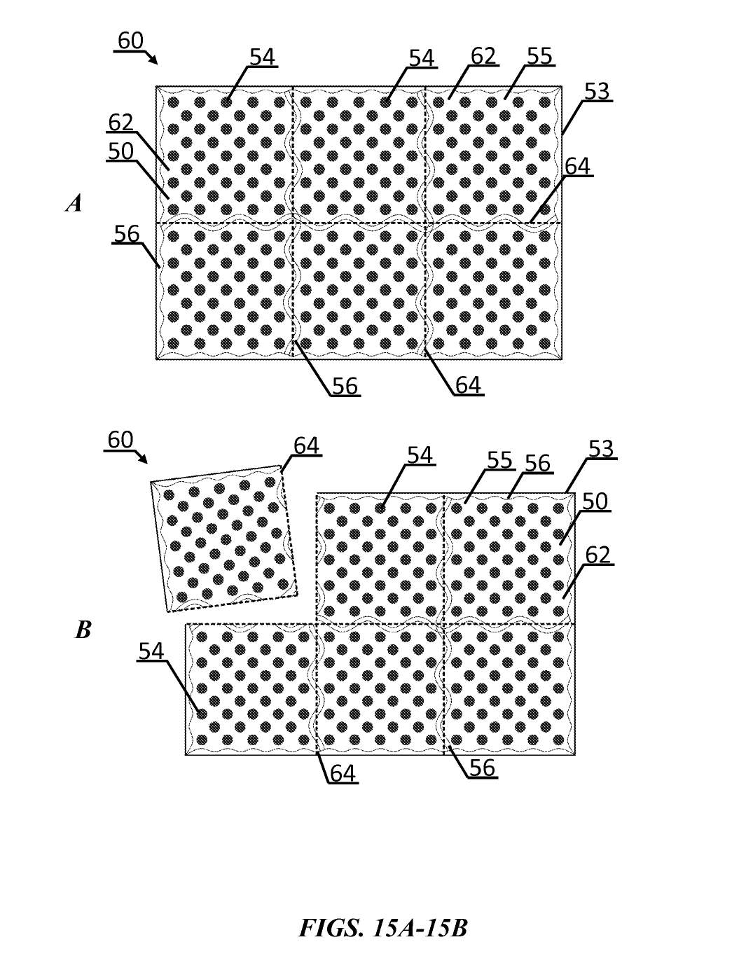

FIGS. 1 through 28, wherein like parts are designated by like reference numerals throughout, illustrate example embodiments of a sublingual dosage form of the present invention, a dispenser for administering the sublingual dosage form, related methods, apparatuses, and systems for preparing a sublingual dosage form of the present invention, related methods of using the dispenser to administer the sublingual dosage form, as well as related compositions and kits, according to various aspects of the present invention. Although the present invention will be described with reference to the figures, it should be understood that many alternative forms can embody the present invention. One of skill in the art will additionally appreciate different ways to alter the parameters disclosed, such as the size, shape, or type of elements or materials, in a manner still in keeping with the spirit and scope of the present invention.

Those skilled in the art will appreciate that the sublingual dosage form is not a conventional pharmaceutical dosage form formulated for sublingual or oral administration. That is, the sublingual dosage form is the decarboxylated cannabis plant material itself, not a cannabinoid extract formulated as a conventional pharmaceutical dosage form such as an aerosol, a bead, a capsule, a cloth, a concentrate, an elixir, an emulsion, an extract, a fiber, a film, a gel, a globule, a granule, a chewing gum, an inhalant, a jelly, a liquid, a lozenge comprising a cannabinoid extract, an oil, a paste, a patch, a pellet, a pill, a poultice, a powder, a salve, a solution, a sponge, a spray, a strip, a suspension, a syrup, a tablet, a tape, a tincture, a trouche, and a wafer. In further contrast to conventional pharmaceutical dosage forms, which typically include a pharmaceutically acceptable agent to facilitate administration of the active ingredient, the sublingual dosage form in its finished state does not contain a pharmaceutically acceptable binder, buffering agent, carrier, chelating agent, co-solvent, cross-linking agent, diluent, disintegrant, emulsifier, excipient, flavoring agent, permeability enhancer, preservative, propellant, solvent, or surfactant compressed into a cohesive solid dosage form.

FIGS. 1A and 1B illustrate example embodiments of a sublingual dosage form of the present invention. As is shown in FIGS. 1A and 1B, the total weight of the decarboxylated cannabis plant material accounts for substantially the entire weight of the sublingual dosage form. As is shown in the example embodiment in FIG. 1A, decarboxylated cannabis plant material can be administered sublingually in the form of a bud. As is shown in the example embodiment in FIG. 1B, decarboxylated cannabis plant material can be administered sublingually in the form of shake. It should be appreciated by those skilled in the art that the sublingual dosage form comprising the decarboxylated cannabis plant material is capable of delivering an effective amount of at least one pharmacologically active cannabinoid into the systemic circulation of a subject when the decarboxylated cannabis plant material itself is placed directly into the subject's sublingual cavity, without requiring a pharmaceutically acceptable carrier, diluent, buffer, or excipient to facilitate release of the at least one pharmacologically active cannabinoid into the sublingual cavity, or subsequent absorption through the sublingual mucosa into the systemic circulation of the subject.

As will be appreciated by those skilled in the art, the at least one pharmacologically active cannabinoid present in the decarboxylated cannabis plant material can be used in a variety of treatment applications, for example for treatment of glaucoma, amelioration of nausea, especially when associated with cancer chemotherapy, hypertension, pain, spasticity, neurogenic pain, movement disorders, asthma, premenstrual syndrome, unintentional weight loss, insomnia, lack of appetite, multiple sclerosis and spinal cord injuries (by exhibiting antispasmodic and muscle-relaxant properties). Other studies demonstrate that cannabis or cannabinoids may be useful in treating alcohol abuse, amyotrophic lateral sclerosis, collagen-induced arthritis, atherosclerosis, colorectal and other cancers, HIV-associated sensory neuropathy, dystonia, epilepsy, digestive diseases, gliomas, hepatitis C, Huntington's disease, leukemia, skin tumors, methicillin-resistant Staphylococcus aureus (MRSA), Parkinson's disease, pruritus, psoriasis, sickle-cell disease, sleep apnea, and Tourette syndrome as well as psychological conditions such as bipolar disorder, depression, posttraumatic stress disorder (PTSD), and anorexia nervosa. Accordingly, the present invention provides methods of treating preventing, or ameliorating a symptom of any of the diseases or conditions above in a subject in need thereof, comprising administering to the subject an effective amount of a sublingual dosage form described herein, for example, using a dispenser of the present invention. As used herein, “subject” and “user” are used interchangeably.

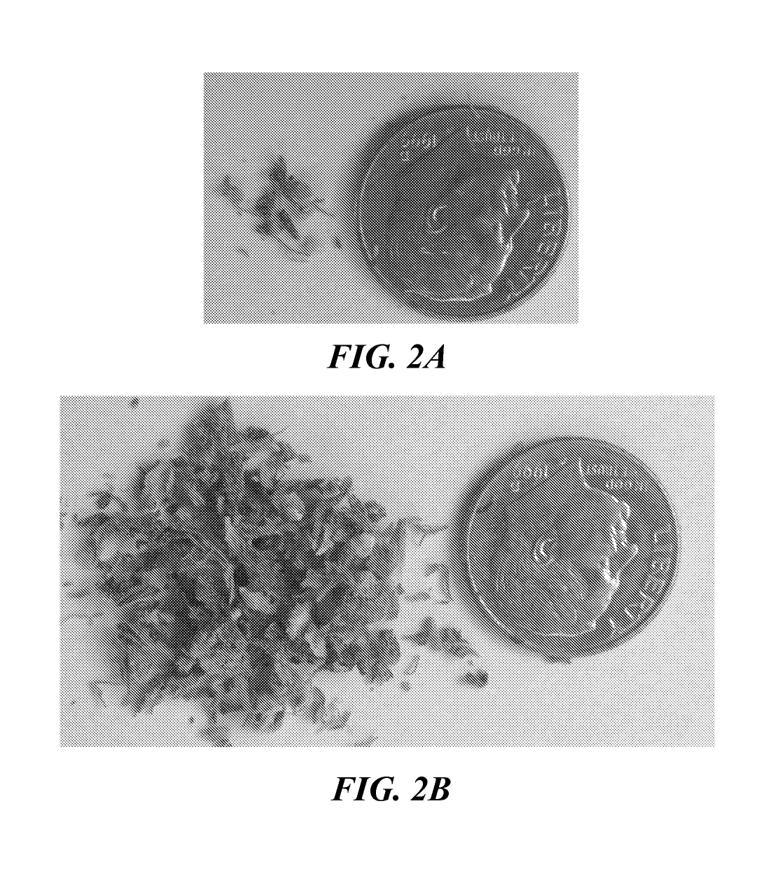

The total weight of the decarboxylated cannabis plant material for delivering an effective dose of at least one pharmacologically active cannabinoid present in the decarboxylated cannabis plant material into the systemic circulation of a subject may vary depending on a variety of factors, such as the subject's history, age, weight, gender, and metabolism, as will be appreciated by those skilled in the art. The present invention contemplates preparing sublingual dosage forms of any weight of decarboxylated cannabis plant material, as the invention is not intended to be limited in this manner. For example, the total weight of the decarboxylated cannabis plant material for a single dose can range from about 0.05 grams to about 7 grams inclusive. In an embodiment, the total weight of the decarboxylated cannabis plant material for a single dose is from about 0.10 grams to about 3 grams inclusive. In an embodiment, the total weight of the decarboxylated cannabis plant material for a single dose is about 1 gram. FIG. 2A depicts a smallest dose of decarboxylated cannabis plant material for delivering an effective amount of at least one pharmacologically active cannabinoid sublingually, in accordance with an example embodiment of the present invention. As shown in the example embodiment depicted in FIG. 2A, the smallest dose of decarboxylated cannabis plant material that can be used for delivering an effective amount of at least one pharmacologically active cannabinoid is approximately 0.05 grams. FIG. 2B depicts an average effective dose of decarboxylated cannabis plant material for delivering an effective amount of at least one pharmacologically active cannabinoid, in accordance with an example embodiment of the present invention. As shown in the example embodiment depicted in FIG. 2B, the average effective dose of decarboxylated cannabis plant material that can be used for delivering an effective amount of at least one pharmacologically active cannabinoid is approximately 0.5 grams. Of course, the actual effective dose of decarboxylated cannabis plant material that is required to deliver an effective amount of at least one pharmacologically active cannabinoid into the systemic circulation of a subject may vary, as will be appreciated by those skilled in the art.

The sublingual dosage form of the present invention releases an effective amount of at least one pharmacologically active agent (e.g., at least one pharmacologically active cannabinoid) into contact with the sublingual mucosa of a subject when the sublingual dosage form is placed into the sublingual cavity of the subject. As used herein, “effective amount” refers to an amount of the agent which is required to induce a detectable pharmacological and/or physiological effect in a subject. Determination of an effective amount is well within the capability of those skilled in the art. Generally, an effective amount can vary with the subject's history, age, condition, gender, as well as the type of medical condition in the subject and the severity of the medical condition, for example. As used herein, “pharmacologically active agent” refers to an agent that has a detectable pharmacological and/or physiological effect on a cell or organism. The present invention contemplates sublingual administration in any subject, e.g., human or animal subjects, for example, for medicinal or veterinary applications. Upon release of the effective amount of the at least one pharmacologically active cannabinoid into contact with a subject's sublingual mucosa, the at least one pharmacologically active cannabinoid is absorbed through the subject's sublingual mucosa and enters the systemic circulation of the subject. Studies conducted by the inventors have determined that the sublingual dosage form rapidly (e.g., instantaneously) releases an effective amount of at least one pharmacologically active cannabinoid into the sublingual cavity of a subject when placed in the subject's sublingual cavity. Studies conducted by the inventors have established that the sublingual dosage form begins to release an effective amount of at least one pharmacologically active cannabinoid into the systemic circulation of a subject within seconds of placing the sublingual dosage form in the subject's sublingual cavity. That is, the at least one pharmacologically active cannabinoid is absorbed rapidly.

In contrast to traditional methods, such as ingesting cannabis, for which onset of pharmacological effect typically takes between 30 minutes and 120 minutes, the sublingual dosage form of the present invention induces a pharmacological effect in a subject within about 30 seconds to about 20 minutes of placing the sublingual dosage form in the subject's sublingual cavity. In an embodiment, the sublingual dosage form induces a pharmacological effect in subject within about 30 seconds of placing the sublingual dosage form in the subject's sublingual cavity. In an embodiment, the sublingual dosage form induces a pharmacological effect in subject within about 1 minute of placing the sublingual dosage form in the subject's sublingual cavity. In an embodiment, the sublingual dosage form induces a pharmacological effect in subject within about 2 minutes of placing the sublingual dosage form in the subject's sublingual cavity. In an embodiment, the sublingual dosage form induces a pharmacological effect in subject within about 3 minutes of placing the sublingual dosage form in the subject's sublingual cavity. In an embodiment, the sublingual dosage form induces a pharmacological effect in subject within about 4 minutes of placing the sublingual dosage form in the subject's sublingual cavity. In an embodiment, the sublingual dosage form induces a pharmacological effect in subject within about 5 minutes of placing the sublingual dosage form in the subject's sublingual cavity. In an embodiment, the sublingual dosage form induces a pharmacological effect in subject within about 6 minutes of placing the sublingual dosage form in the subject's sublingual cavity. In an embodiment, the sublingual dosage form induces a pharmacological effect in subject within about 7 minutes of placing the sublingual dosage form in the subject's sublingual cavity. In an embodiment, the sublingual dosage form induces a pharmacological effect in subject within about 8 minutes of placing the sublingual dosage form in the subject's sublingual cavity. In an embodiment, the sublingual dosage form induces a pharmacological effect in subject within about 9 minutes of placing the sublingual dosage form in the subject's sublingual cavity. In an embodiment, the sublingual dosage form induces a pharmacological effect in subject within about 10 minutes of placing the sublingual dosage form in the subject's sublingual cavity. In an embodiment, the sublingual dosage form induces a pharmacological effect in subject within about 11 minutes of placing the sublingual dosage form in the subject's sublingual cavity. In an embodiment, the sublingual dosage form induces a pharmacological effect in subject within about 12 minutes of placing the sublingual dosage form in the subject's sublingual cavity. In an embodiment, the sublingual dosage form induces a pharmacological effect in subject within about 13 minutes of placing the sublingual dosage form in the subject's sublingual cavity. In an embodiment, the sublingual dosage form induces a pharmacological effect in subject within about 14 minutes of placing the sublingual dosage form in the subject's sublingual cavity. In an embodiment, the sublingual dosage form induces a pharmacological effect in subject within about 15 minutes of placing the sublingual dosage form in the subject's sublingual cavity. In an embodiment, the sublingual dosage form induces a pharmacological effect in subject within about 16 minutes of placing the sublingual dosage form in the subject's sublingual cavity. In an embodiment, the sublingual dosage form induces a pharmacological effect in subject within about 17 minutes of placing the sublingual dosage form in the subject's sublingual cavity. In an embodiment, the sublingual dosage form induces a pharmacological effect in subject within about 18 minutes of placing the sublingual dosage form in the subject's sublingual cavity. In an embodiment, the sublingual dosage form induces a pharmacological effect in subject within about 19 minutes of placing the sublingual dosage form in the subject's sublingual cavity. In an embodiment, the sublingual dosage form induces a pharmacological effect in subject within about 20 minutes of placing the sublingual dosage form in the subject's sublingual cavity.

The onset of pharmacological effect in any particular subject may depend on a variety of factors, as will be appreciated by those skilled in the art. Whereas conventional methods such as smoking or ingesting cannabis typically permit a single dose to be delivered via a single route of administration, such as inhalation, the sublingual dosage form of the present invention permits multiple doses of at least one pharmacologically active cannabinoid to be delivered via at least two routes of administration. For example, after releasing an effective amount of at least one pharmacologically active cannabinoid the sublingual dosage form retains an amount of at least one pharmacologically active cannabinoid effective for enteral administration. As used herein, “an amount effective for enteral administration” is an amount of agent that induces a desired pharmacological or physiological effect in a subject when absorbed through the gastrointestinal tract. In other words, the sublingual dosage form is ingestible. The skilled artisan will appreciate that when ingested after releasing an effective amount of at least one pharmacologically active cannabinoid the sublingual dosage form releases an effective amount of at least one pharmacologically active cannabinoid into the gastrointestinal tract of a subject. Studies conducted by the inventors establish that ingesting the sublingual dosage form of the present invention prolongs the peak effect, prolongs the overall duration of the pharmacological effect, and provides a broader spectrum of therapeutic application. When ingested, the THC is subject to processing by the liver, where it is metabolized into 11-hydroxy-delta-9-tetrahydrocannabinol (11-OH-THC). Increased levels of 11-OH-THC within the bloodstream following ingestion are associated with multiphasic effects, including peaking in successive waves and psychoactive effects at high doses. Increased levels of 11-OH-THC also result in a more sedative and physically relaxant effect as compared to effects experienced when cannabinoids are delivered directly into the bloodstream. In addition to cannabinoids, ingested cannabis provides the digestive tract a variety of other beneficial compounds, including essential amino acids, essential fatty acids, and fiber. Where the sublingual dosage form is administered and subsequently ingested, the user experiences the full spectrum of these effects. Direct absorption of cannabinoids into the bloodstream during sublingual administration is followed by processing of cannabinoids by the liver approximately 1 hour later. This synergy yields a therapeutic experience significantly longer and more comprehensive than either method provides on its own. The multifaceted nature of the sublingual dosage form is particularly significant because it allows for a single application to accomplish two distinct therapeutic goals. For example, a single sublingual dosage form would provide the rapid “cerebral” therapeutic relief required to treat anxiety and nausea, as well as the sedative “body” effects best suited for providing relief for conditions like spasticity and chronic pain.

The present invention contemplates any pharmacologically active agent present in decarboxylated cannabis plant material that is absorbed via the sublingual mucosa as an active ingredient. In an embodiment, the decarboxylated cannabis plant material comprises at least one pharmacologically active cannabinoid present as an active ingredient. In an embodiment, the at least one pharmacologically active cannabinoid is a psychoactive agent. In an embodiment, the at least one pharmacologically active cannabinoid is a non-psychoactive agent. In an embodiment, the at least one pharmacologically active cannabinoid is tetrahydrocannabinol. As used herein “tetrahydrocannabinol” refers to d9-tetrahydrocannabinol (THC), a decarboxylation product of its inactive precursor d9-tetrahydrocannabinolic acid (THCA; also referred to herein as cannabinolic acid). In an embodiment, the at least one pharmacologically active cannabinoid is an analog or derivative of tetrahydrocannabinol. In an embodiment, the at least one pharmacologically active cannabinoid is cannabidiol. As used herein “cannabidiol” (CBD) refers to the decarboxylation product of its inactive precursor cannabidiolic acid (CDBA). In an embodiment, the at least one pharmacologically active cannabinoid is an analog or derivative of cannabidiol. In an embodiment, the at least one pharmacologically active cannabinoid is cannabigerol. In an embodiment, the at least one pharmacologically active cannabinoid is an analog or derivative of cannabigerol. In an embodiment, the at least one pharmacologically active cannabinoid is cannabigevarin. In an embodiment, the at least one pharmacologically active cannabinoid is an analog or derivative of cannabigevarin. In an embodiment, the at least one pharmacologically active cannabinoid is tetrahydrocannabivarin. In an embodiment, the at least one pharmacologically active cannabinoid is an analog or derivative of tetrahydrocannabivarin. In an embodiment, the at least one pharmacologically active cannabinoid is cannabidivarin. In an embodiment, the at least one pharmacologically active cannabinoid is an analog or derivative of cannabidivarin.

Those skilled in the art will appreciate that the amount of active ingredient in the sublingual dosage form may vary, for example, depending on the variety of cannabis plant, the content of inactive precursors in the raw cannabis plant material, and/or the total weight of the raw cannabis plant material to be decarboxylated, among other things. In some embodiments, the cannabinolic acid precursor is present prior to decarboxylation in an amount up to about 40% total weight of the raw cannabis plant material, preferably from about 1% to about 35%, from about 5% to about 30%, from about 10% to about 30%, and from about 15% to about 25%. In an embodiment, the tetrahydrocannabinol is present in an amount up to about 40% total weight of the decarboxylated cannabis plant material, preferably from about 1% to about 35%, from about 5% to about 30%, from about 10% to about 30%, and from about 15% to about 25%. In an embodiment, the tetrahydrocannabinol is present the sublingual dosage form in a dose range from about 2 mg to about 500 mg inclusive. In an embodiment, the tetrahydrocannabinol is present in the sublingual dosage form in a dose of about 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, or about 500 mg.

The amount of tetrahydrocannabinol present in the decarboxylated cannabis plant material can be characterized as a percentage of the maximum amount of tetrahydrocannabinol that could theoretically be present as a result of decarboxylation of the maximum amount of its cannabinolic acid precursor present in the cannabis plant material. The methods described herein result in decarboxylated cannabis plant material that contains at least 70% of the maximum amount of tetrahydrocannabinol that could theoretically be present as a result of decarboxylation of the maximum amount of its cannabinolic acid precursor present in the cannabis plant material. In some embodiments, the decarboxylated cannabis plant material contains at least 80% of the maximum amount of tetrahydrocannabinol that could theoretically be present as a result of decarboxylation of the maximum amount of its cannabinolic acid precursor present in the cannabis plant material. In some embodiments, the decarboxylated cannabis plant material contains at least 81%, at least 82%, at least 83%, at least 84%, at least 85%, at least 86%, at least 87%, at least 88%, or at least 89% of the maximum amount of tetrahydrocannabinol that could theoretically be present as a result of decarboxylation of the maximum amount of its cannabinolic acid precursor present in the cannabis plant material. In some embodiments, the decarboxylated cannabis plant material contains at least 90% of the maximum amount of tetrahydrocannabinol that could theoretically be present as a result of decarboxylation of the maximum amount of its cannabinolic acid precursor present in the cannabis plant material. In some embodiments, the decarboxylated cannabis plant material contains at least 91%, at least 92%, at least 93%, at least 94%, at least 95%, at least 96%, at least 97%, at least 98%, or at least 99$ of the maximum amount of tetrahydrocannabinol that could theoretically be present as a result of decarboxylation of the maximum amount of its cannabinolic acid precursor present in the cannabis plant material. In some embodiments, the decarboxylated cannabis plant material contains the maximum amount of tetrahydrocannabinol that could theoretically be present as a result of decarboxylation of the maximum amount of its cannabinolic acid precursor present in the cannabis plant material.

In some embodiments, the cannabidiolic acid precursor is present prior to decarboxylation in an amount up to about 40% total weight of the raw cannabis plant material, preferably from about 1% to about 35%, from about 5% to about 30%, from about 10% to about 30%, and from about 15% to about 25%. In an embodiment, the cannabidiol is present in the in an amount up to about 40% total weight of the decarboxylated cannabis plant material, preferably from about 1% to about 35%, from about 5% to about 30%, from about 10% to about 30%, and from about 15% to about 25%. In an embodiment, the cannabidiol is present in the sublingual dosage form in a dose range from about 2 mg to about 500 mg. In an embodiment, the cannabidiol is present in the sublingual dosage form in a dose of about 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, or about 500 mg.

The amount of cannabidiol present in the decarboxylated cannabis plant material can be characterized as a percentage of the maximum amount of cannabidiol that could theoretically be present as a result of decarboxylation of the maximum amount of its cannabidiolic acid precursor present in the cannabis plant material. The methods described herein result in decarboxylated cannabis plant material that contains at least 70% of the maximum amount of cannabidiol that could theoretically be present as a result of decarboxylation of the maximum amount of its cannabidiolic acid precursor present in the cannabis plant material. In some embodiments, the decarboxylated cannabis plant material contains at least 71%, at least 72%, at least 73%, at least 74%, at least 75%, at least 76%, at least 77%, at least 78%, or at least 79% of the maximum amount of cannabidiol that could theoretically be present as a result of decarboxylation of the maximum amount of its cannabidiolic acid precursor present in the cannabis plant material. In some embodiments, the decarboxylated cannabis plant material contains at at least 80% of the maximum amount of cannabidiol that could theoretically be present as a result of decarboxylation of the maximum amount of its cannabidiolic acid precursor present in the cannabis plant material. In some embodiments, the decarboxylated cannabis plant material contains at least 81%, at least 82%, at least 83%, at least 84%, at least 85%, at least 86%, at least 87%, at least 88%, or at least 89% of the maximum amount of cannabidiol that could theoretically be present as a result of decarboxylation of the maximum amount of its cannabidiolic acid precursor present in the cannabis plant material. In some embodiments, the decarboxylated cannabis plant material contains at least 90% of the maximum amount of cannabidiol that could theoretically be present as a result of decarboxylation of the maximum amount of its cannabidiolic acid precursor present in the cannabis plant material. In some embodiments, the decarboxylated cannabis plant material contains at least 91%, at least 92%, at least 93%, at least 94%, at least 95%, at least 96%, at least 97%, at least 98%, or at least 99% of the maximum amount of cannabidiol that could theoretically be present as a result of decarboxylation of the maximum amount of its cannabidiolic acid precursor present in the cannabis plant material. In some embodiments, the decarboxylated cannabis plant material contains the maximum amount of cannabidiol that could theoretically be present as a result of decarboxylation of the maximum amount of its cannabidiolic acid precursor present in the cannabis plant material.

In some embodiments, the at least one pharmacologically active cannabinoid comprises tetrahydrocannabinol and cannabidiol. In some embodiments, the tetrahydrocannabinol is present in an amount from about 1% to about 40% total weight of the decarboxylated cannabis plant material, and wherein the cannabidiol is present in an amount from about 1% to about 40% total weight of the decarboxylated cannabis plant material. In some embodiments, the tetrahydrocannabinol is present in a dose range from about 2 mg to about 500 mg, and wherein the cannabidiol is present in a dose range from about 2 mg to about 500 mg. In some embodiments, the decarboxylated cannabis plant material contains at least 70% of the maximum amount of tetrahydrocannabinol and cannabidiol that could theoretically be present as a result of decarboxylation of the maximum amount of cannabinolic acid and cannabidiolic acid present in the cannabis plant material. the decarboxylated cannabis plant material contains at least 71%, at least 72%, at least 73%, at least 74%, at least 75%, at least 76%, at least 77%, at least 78%, or at least 79% of the maximum amount of tetrahydrocannabinol and cannabidiol that could theoretically be present as a result of decarboxylation of the maximum amount of its cannabinolic acid and cannabidiolic acid precursor, respectively, present in the cannabis plant material. In some embodiments, the decarboxylated cannabis plant material contains at least 80% of the maximum amount of tetrahydrocannabinol and cannabidiol that could theoretically be present as a result of decarboxylation of the maximum amount of cannabinolic acid and cannabidiolic acid present in the cannabis plant material. In some embodiments, the decarboxylated cannabis plant material contains at least 81%, at least 82%, at least 83%, at least 84%, at least 85%, at least 86%, at least 87%, at least 88%, or at least 89% of the maximum amount of tetrahydrocannabinol and cannabidiol that could theoretically be present as a result of decarboxylation of the maximum amount of cannabinolic acid and cannabidiolic acid present in the cannabis plant material. In some embodiments, the decarboxylated cannabis plant material contains at least 90% of the maximum amount of tetrahydrocannabinol and cannabidiol that could theoretically be present as a result of decarboxylation of the maximum amount of cannabinolic acid and cannabidiolic acid present in the cannabis plant material. In some embodiments, the decarboxylated cannabis plant material contains at least 91%, at least 92%, at least 93%, at least 94%, at least 95%, at least 96%, at least 97%, at least 98%, or at least 99% of the maximum amount of tetrahydrocannabinol and cannabidiol that could theoretically be present as a result of decarboxylation of the maximum amount of cannabinolic acid and cannabidiolic acid present in the cannabis plant material. In some embodiments, the decarboxylated cannabis plant material contains the maximum amount of tetrahydrocannabinol and cannabidiol that could theoretically be present as a result of decarboxylation of the maximum amount of cannabinolic acid and cannabidiolic acid present in the cannabis plant material.

It is worth noting that decarboxylation of cannabis plant material can result in reactions in which THC is degraded to cannabinol (CBN), which has only a fraction of the psychoactive effects of THC and causes drowsiness as well as fellings of fatigue and disorientation. The methods described herein result in a decarboxylated cannabis plant material that contains low amounts of cannabinol. In some embodiments, the amount of cannabinol present in the decarboxylated cannabis plant material is less than 0.5% of the total weight of the decarboxylated cannabis plant material. In some embodiments, the amount of cannabinol present in the decarboxylated cannabis plant material is less than 0.4% of the total weight of the decarboxylated cannabis plant material. In some embodiments, the amount of cannabinol present in the decarboxylated cannabis plant material is less than 0.3% of the total weight of the decarboxylated cannabis plant material. In some embodiments, the amount of cannabinol present in the decarboxylated cannabis plant material is less than 0.2% of the total weight of the decarboxylated cannabis plant material. In some embodiments, the amount of cannabinol present in the decarboxylated cannabis plant material is less than 0.1% of the total weight of the decarboxylated cannabis plant material. In some embodiments, the amount of cannabinol present in the decarboxylated cannabis plant material is below quantifiable limits (BQL) with respect to the total weight of the decarboxylated cannabis plant material.

The present invention contemplates using any cannabis variety or combination of cannabis varieties to prepare a sublingual dosage form of the present invention. In an embodiment, the cannabis comprises Cannibis sativa. In an embodiment, the cannabis comprises Cannabis indica. In an embodiment, the cannabis comprises Cannabis ruderalis. As will be appreciated by those skilled in the art, the cannabis used to prepare the sublingual dosage form can be a genetically modified variety, which has been genetically modified to include a higher content of an inactive acid precursor which can be decarboxylated into a pharmacologically active cannabinoid that is suitable for sublingual administration. For example, the cannabis can be genetically modified utilizing techniques well known to the skilled artisan to increase the cannabinolic acid precursor to THC content in the cannabis.

The present invention also contemplates selecting cannabis strains that are enriched for THC, CBD, THC and CBD, and other pharmacologically active cannabinoids for decarboxylation according to the methods described herein. As used herein, “enriched” means that strain contains a relative high amount of the particular inactive precursor such that subjecting the strain to the decarboxylation methods described herein results in a decarboxylated cannabis plant material that contains a relative high amount of the pharmacologically active cannabinoid that results from decarboxylation of the inactive precursor. As used herein, a “THC enriched strain” is any cannabis strain in which the maximum theoretical amount of THC makes up 80% of total cannabinoids and the maximum theoretical amount of CBD is less than 1% total weight. As used herein, a “CBD enriched strain” is any cannabis strain in which the maximum theoretical amount of CBD makes up more than 20% total cannabinoids or in which the maximum theoretical CBD is more than 1%. It should be evident that a THC and CDB enriched strain is a strain that has a relative high amount of THCA and CDBA such that decarboxylation of the strain as described herein results in a relative high amount of THC and CBD in the decarboxylated cannabis plant material. It should also be evident that a THC- and CBD-enriched strain any cannabis strain tin which the maximum theoretical amount of THC makes up to 80% of total cannabinoids and the maximum theoretical amount of CBD makes up to at least 20% total cannabinoids or in which the maxim theoretical CBD is more than 1%. Selecting cannabis strains that are enriched for THC, CBD, THC and CBD, and other pharmacologically active cannabinoids can be accomplished in a variety of ways. For example, certain commercially available hybrid strains are known to be enriched for both THC and CBD. In such examples, selecting a THC- and CBD-enriched strain can be achieved by obtaining the commercially available strain with the desired characteristics. In situations in which the content of the THCA and/or CBDA inactive precursors in a particular strain are unknown, selecting a strain that is enriched for THC, CBD, or THC and CBD can be achieved by measuring the THCA, CBDA, and/or THCA and CBDA content in the strain, e.g., using high-performance liquid chromatography (e.g., HPLC-UV), and then selecting a strain which has the desired characteristics as measured.

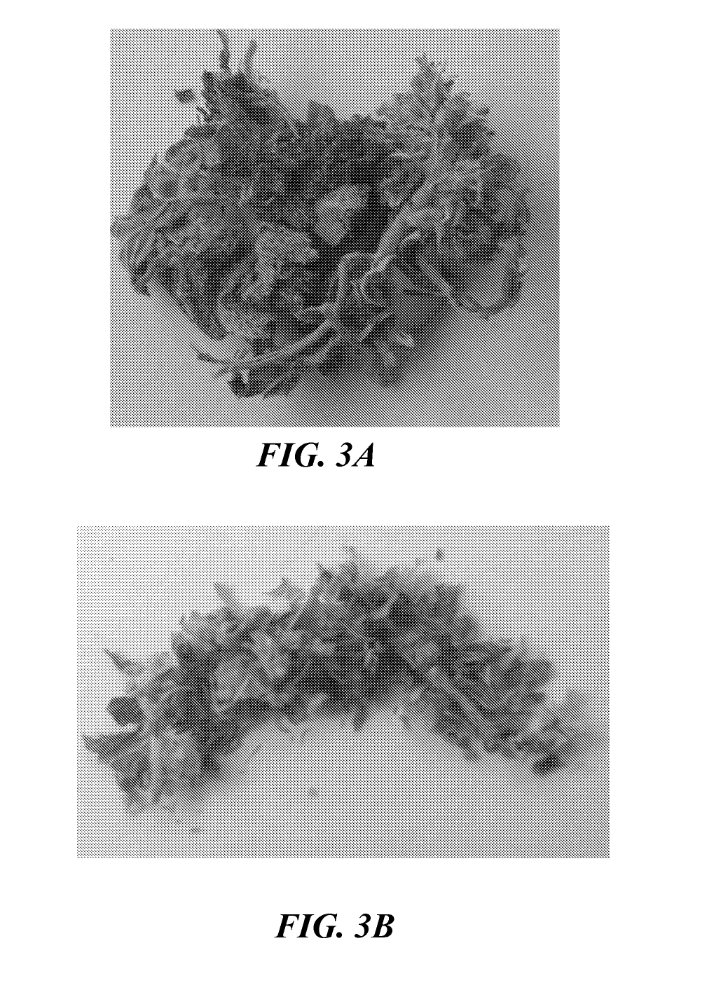

Decarboxylated cannabis in its raw plant form is often unsuited for direct sublingual use. Such cannabis is often amassed in dense buds, which may cause discomfort within the mouth and provide inefficient delivery. In other instances, the cannabis particles may be small, causing them to be easily displaced from under the tongue and dispersed throughout the oral cavity. These circumstances limit effectiveness and can create a barrier to the application of administration of at least one pharmacologically active cannabinoid via the sublingual dosage form of the present invention. Accordingly, the decarboxylated cannabis plant material can be shaped or formed, for example, to facilitate maximum absorption of the cannabinoids present in the decarboxylated cannabis plant material by increasing the surface area of the decarboxylated cannabis plant material that is in direct contact with the sublingual mucosa. FIGS. 3A and 3B are photographs illustrating example embodiments of a sublingual dosage form of the present invention shaped or formed to facilitate maximum absorption of the sublingual dosage form. As shown in the exemplary embodiment depicted in FIG. 3A, the sublingual dosage form depicted in FIG. 1A can be flattened out (e.g., compressed), for example into a fanned configuration, before being placed directly in the sublingual cavity of a subject for sublingual administration of at least one pharmacologically active cannabinoid. As shown in the exemplary embodiment depicted in FIG. 3B, the sublingual dosage form depicted in FIG. 1B can be gathered together before being placed directly in the sublingual cavity of a subject for sublingual administration of at least one pharmacologically active cannabinoid.

In some instances, it may be desirable to remove any stems and/or seeds present in the decarboxylated cannabis plant material prior to, during, or after shaping or forming the decarboxylated cannabis plant material, as will be appreciated by those skilled in the art. Those skilled in the art will also appreciate that the stems and/or seeds present in the an amount of raw cannabis to be decarboxylated according to the present invention can be removed before decarboxylating the raw cannabis in accordance with the methods described herein. The present invention contemplates any method of removing stems and/or seeds from an amount of raw cannabis plant material or decarboxylated cannabis plant material, as the invention is not intended to be limited by the manner in which stems and/or seeds are removed.

Dispenser

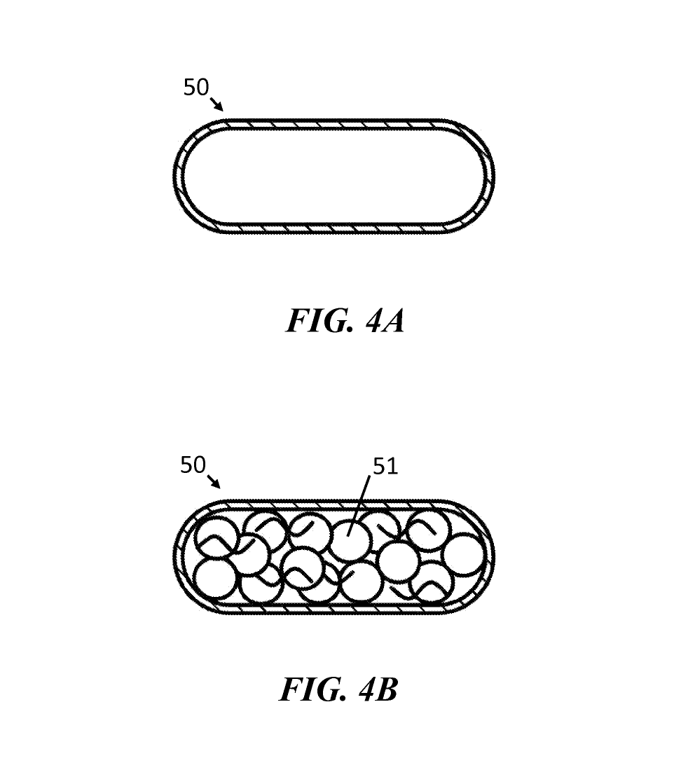

Aspects of the invention involve administering the sublingual dosage form (e.g., decarboxylated cannabis plant material) in a dispenser containing the decarboxylated cannabis plant material. The dispenser sublingually delivers at least one pharmacologically active cannabinoid into the sublingual cavity of a subject when the dispenser is placed within the user's sublingual cavity, where the at least one pharmacologically active cannabinoid is absorbed via the user's sublingual mucosa and enters the user's systemic circulation. FIGS. 4A and 4B depict a dispenser 50 (FIG. 4A) and a dispenser 50 containing a sublingual dosage form of the present invention contained therein (FIG. 4B).

In an aspect, the invention provides a dispenser 50 for delivering at least one pharmacologically active cannabinoid from decarboxylated cannabis plant material contained inside the dispenser into the sublingual cavity of a subject when the dispenser is placed within the subject's sublingual cavity. Examples of suitable dispensers include, but are not limited to, a coating as described in U.S. Patent Application Publication No. 2011/0232662 (incorporated by reference herein in its entirety), or a pouch as described in U.S. Pat. No. 8,387,625 (incorporated by reference herein in its entirety).

Coatings, such as the coating described in the '662 publication, are often constructed from a combination of insoluble and soluble components, which are designed to deliver nicotine from completely disintegrable tobacco material so that once the soluble component of the coating dissolves and tobacco material has disintegrated, a user must chew and either spit out or ingest the remaining insoluble component. In either case, the structural integrity the insoluble component of such coatings is inadequate for the coating to retain the tobacco material itself in inside the coating causing the tobacco material to be released into the user's mouth as the soluble component dissolves.

In contrast to such coatings which require both the soluble component to dissolve and the remaining insoluble component to be chewed before it can be ingested by the user, a dispenser 50 of the present invention is ingestible without modification. The dispenser 50 can be constructed completely free of soluble components or materials. The dispenser 50 can be ingested by a user without first chewing the dispenser materials. The dispenser 50 can be ingested by a user with the decarboxylated cannabis plant material inside it. The dispenser 50 can be ingested by a user with the decarboxylated cannabis plant material inside it without chewing the dispenser before swallowing it. The dispenser 50 can be ingested in the absence of dissolution of a soluble component. The dispenser 50 can be immediately ingested by a user upon placement of the dispenser in the user's mouth, or preferably after sublingual administration of at least one pharmacologically active cannabinoid.

Similarly, in contrast to such coatings which contain dissolvable components and therefore lack the structural integrity to retain the tobacco material inside the coating (i.e., they release the tobacco material into the user's mouth), the dispenser 50 of the present invention retains its structural integrity in the user's sublingual cavity so that the decarboxylated cannabis plant material contained in the dispenser is retained in the dispenser and is not released into the user's mouth. The dispensers 50 of the present invention are configured to deliver an effective amount of at least one pharmacologically active cannabinoid from decarboxylated cannabis plant material contained in the dispenser into a user's sublingual cavity while retaining the decarboxylated cannabis plant material itself inside the dispenser.

Pouches are often constructed in a pillow configuration from moisture permeable materials, such as non woven fabrics, which are typically sealed with a pre-portioned amount of tobacco during the manufacturing process. The seal configuration for many pouches makes it difficult for a user to unseal and reseal the pouch to remove or add tobacco at the user's desire. In addition, the sizes of the pouches are predetermined by the manufacturer. Thus the user has little or no control over selecting the size of the pouch or the amount of tobacco material contained in the pouch, and must consume tobacco in amounts predetermined by the manufacturer. If a user wants to consume less tobacco or obtain a smaller dose of nicotine the user must retain the pouch in the user's mouth for less time, or reduce the pre-portioned amount of tobacco (e.g., by cutting the pouch in half) in a way that is likely to damage the structural integrity of the pouch and cause tobacco material to be dispersed into the user's mouth. Similarly, if a user wants to consume more tobacco or obtain a larger dose of nicotine, the user must insert multiple pouches into the user's mouth either at the same time or consecutively leaving the user little or no choice of controlling the actual amount of tobacco consumed or dosage of nicotine obtained. Moreover, because pouches are pre-packaged with tobacco material it is difficult for a user to know how long the tobacco material has been sitting on the shelf prior to usage. Pre-packaging of the tobacco material into pouches also requires the manufacturer to perform additional processing steps of the tobacco material itself and seal the pouch in special packaging to ensure that the tobacco material itself appears suitable for consumption and actually is suitable for consumption by a user when the user ultimately opens the packaging. For example, the tobacco material often is processed to include a higher water content which can cause the pouch to appear stained or used if the pouch is not packaged appropriately prior to distribution. Moreover, because the tobacco material sealed within each pouch is typically unsafe for ingestion, the pouches containing the tobacco material cannot be ingested by a user after obtaining a dosage of nicotine, and must be discarded by the user.

In contrast to pouches, the dispensers 50 of the present invention can be constructed in a sheet or planar configuration. The dispensers 50 of the present invention can be made and distributed in a non-sealed configuration. The dispensers 50 of the present invention can be distributed for use without a pre-portioned amount of cannabis plant material sealed within the dispensers prior to use. The dispensers 50 of the present invention give a user total control over selecting both the dispenser size and the amount of cannabis plant material to be placed within the selected dispenser. The dispensers 50 of the present invention enable the user to minimize or maximize the cannabinoid dosage as desired. The dispensers 50 of the present invention enable a user to select a desired amount of cannabis for consumption and to enclose the amount of cannabis selected in the dispenser. The sizes of the dispensers 50 of the present invention can be customized by a user without damaging the structural integrity of the dispensers. The dispensers 50 of the present invention do not require special processing steps to ensure that they appear suitable for consumption or actually suitable for consumption by a user when the user attempts to use the dispenser. The dispensers 50 of the present invention are ingestible. The dispensers 50 of the present invention are ingestible with the cannabis plant material contained inside. The dispensers 50 of the present invention are safe for ingestion by a user. The dispensers 50 of the present invention can be swallowed by a user after delivering a sublingual dosage of cannabinoid and therefore do not need to be discarded by the user after use. The dispensers 50 of the present invention are configured to deliver an effective amount of at least one pharmacologically active cannabinoid from decarboxylated cannabis plant material inside the dispenser into a user's sublingual cavity. The dispensers 50 of the present invention can be folded around decarboxylated cannabis plant material for subsequent sublingual administration of pharmacologically active cannabinoids. The dispenser 50 can be configured to elicit a positive sensory response from a user when the dispenser is placed in the user's sublingual cavity with the decarboxylated cannabis plant material contained therein. The dispenser 50 can be configured to elicit a positive sensory response from the user when the dispenser is ingested by the user with the decarboxylated cannabis plant material contained therein.

The dispensers 50 of the present invention can be configured to deliver a first effective dose of at least one pharmacologically active cannabinoid from the decarboxylated cannabis plant material into the subject's sublingual cavity when the dispenser is placed into the subject's sublingual cavity, and to deliver a second dose of the at least one pharmacologically active cannabinoid from the decarboxylated cannabis plant material into the subject's gastrointestinal tract when the dispenser is ingested by the subject after delivering the first effective dose. The dispensers 50 of the present invention can be configured to deliver a maximum sublingual dose of at least one pharmacologically active cannabinoid from decarboxylated cannabis plant material contained inside the dispenser when the dispenser is placed in a user or subject's sublingual cavity with the decarboxylated cannabis plant material therein. The dispensers 50 of the present invention can be configured to deliver a user selected dose of the at least one pharmacologically active cannabinoid from the decarboxylated cannabis plant material into the user's sublingual cavity. The dispensers 50 of the present invention are configured to deliver a first user selected dose of the at least one pharmacologically active cannabinoid from the decarboxylated cannabis plant material when the dispenser is placed into a user's sublingual cavity, and to deliver a second user selected dose of the at least one pharmacologically active cannabinoid from the decarboxylated cannabis plant material when the dispenser is swallowed by the user after delivering the first user selected dose. The dispensers 50 of the present invention are configured to deliver a first controlled dose of the at least one pharmacologically active cannabinoid from the decarboxylated cannabis plant material when the dispenser is placed into the subject's sublingual cavity, and to deliver a second controlled dose of the at least one pharmacologically active cannabinoid from the decarboxylated cannabis plant material when the dispenser is swallowed by the subject after delivering the first controlled dose.

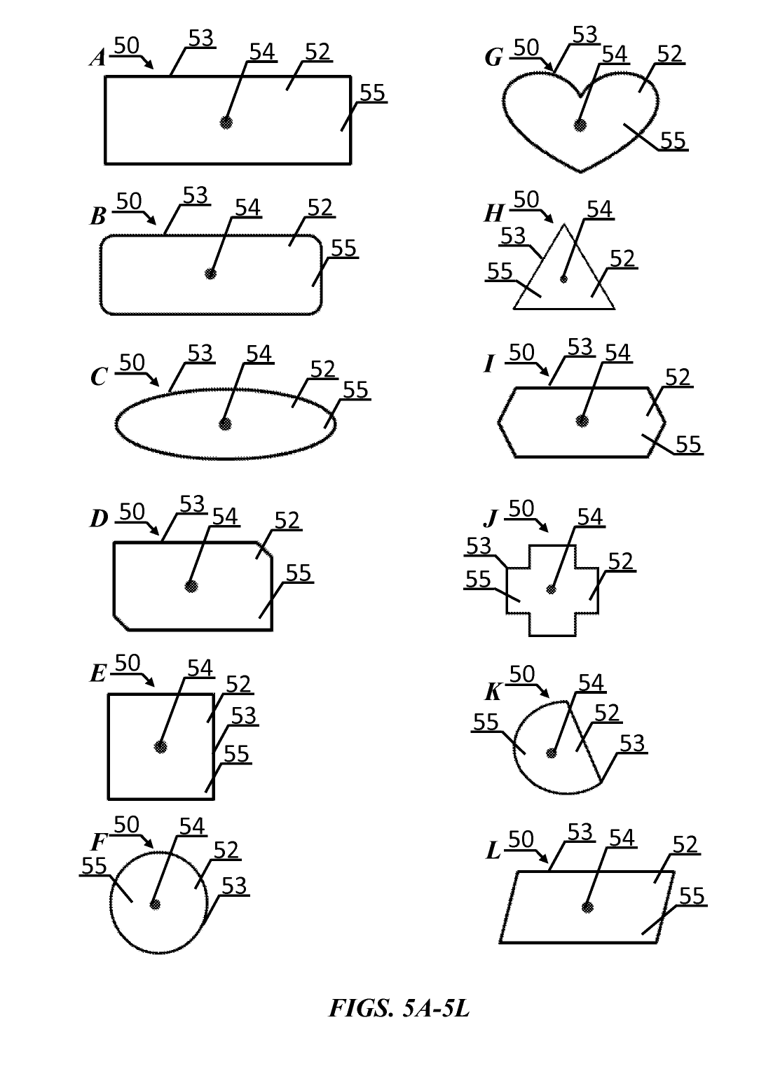

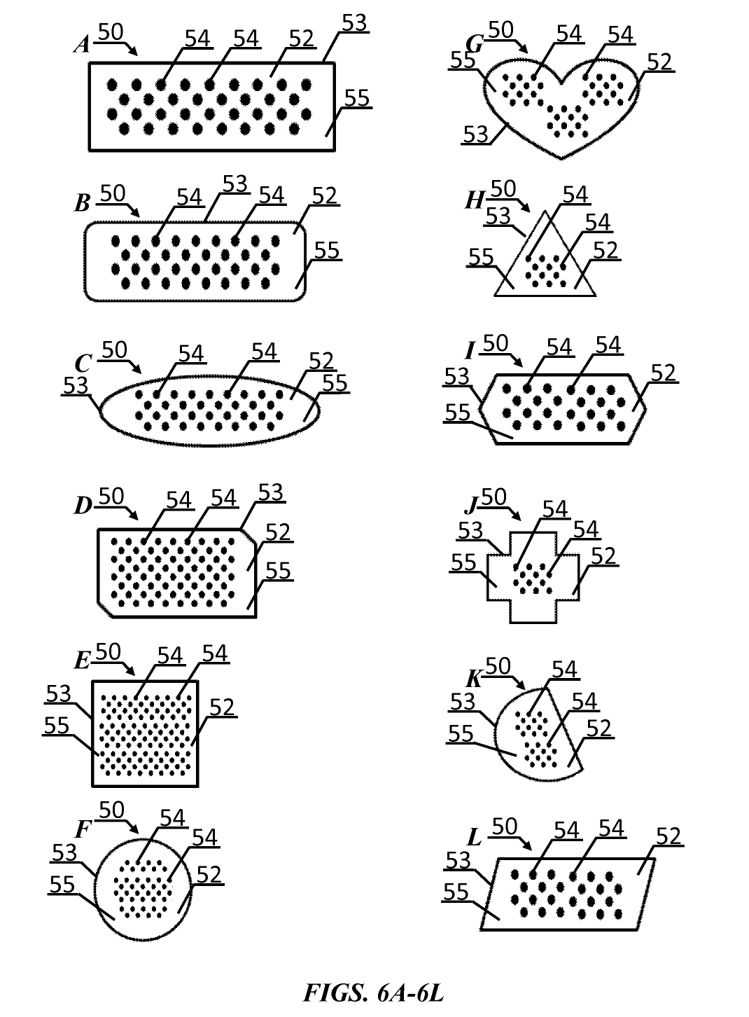

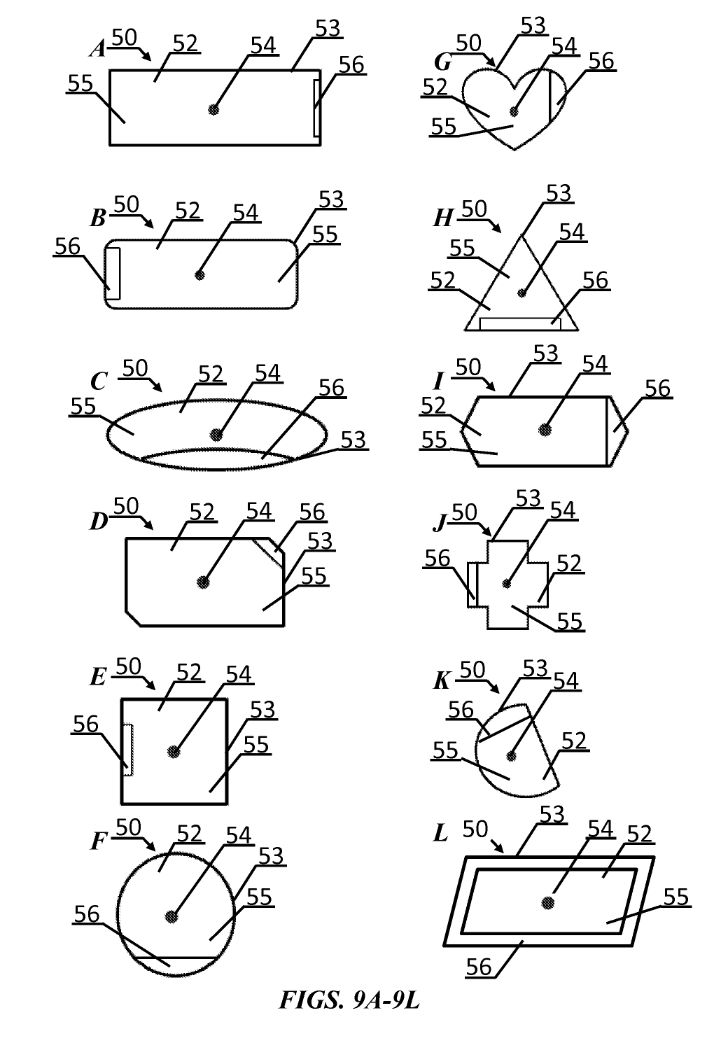

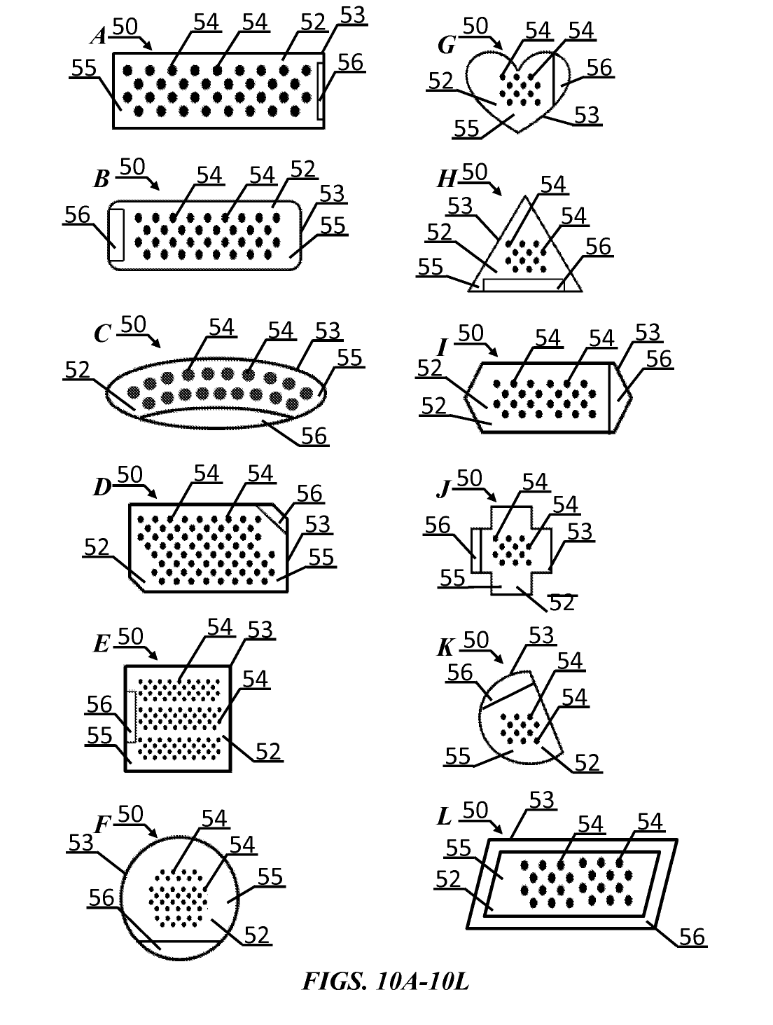

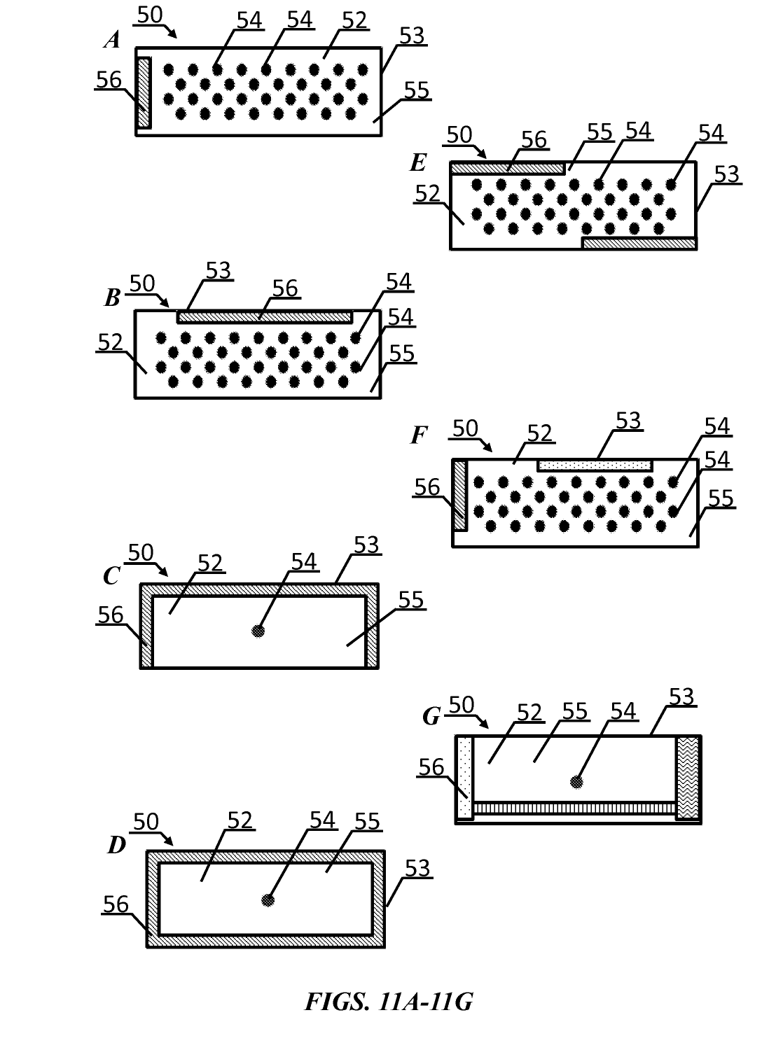

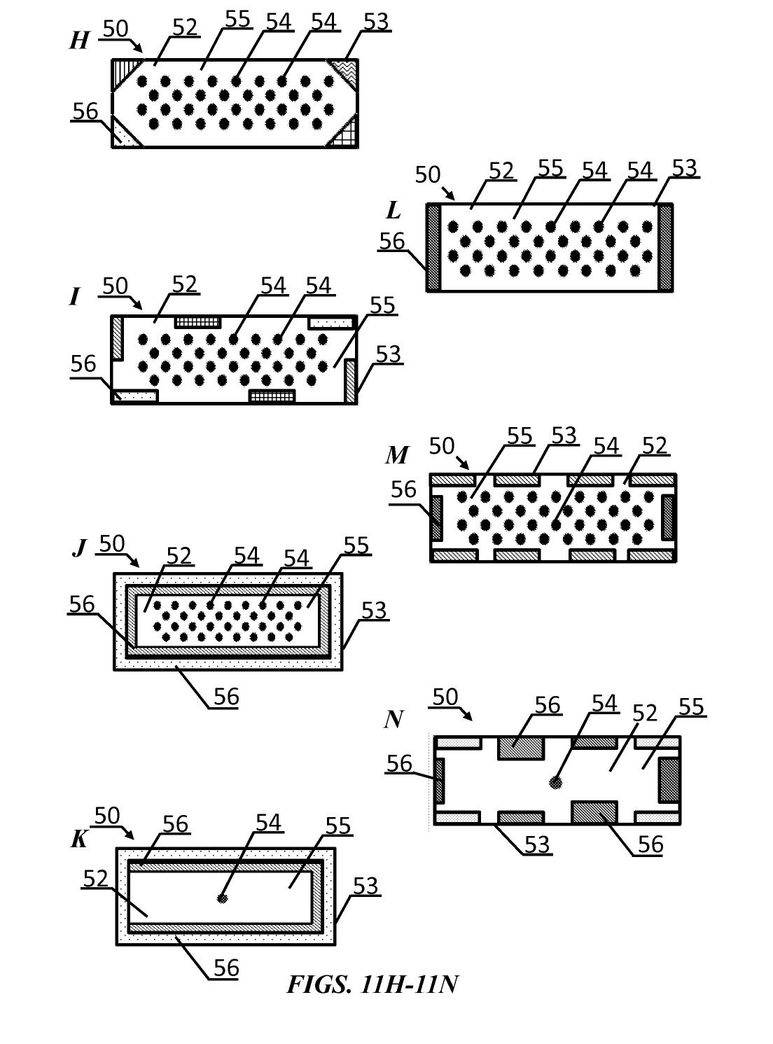

In some aspects, the present invention provides a dispenser 50 comprising a wrapping 52, and at least one aperture 54, as depicted in the exemplary embodiments shown in FIGS. 5A-5L. In some aspects, the present invention provides a dispenser 50 comprising a wrapping 52, and a plurality of apertures 54, as depicted in the exemplary embodiments shown in FIGS. 6A-6L. In some aspects, the present invention provides a dispenser 50 comprising a wrapping 52, at least one aperture 54, and at least one fastener (e.g., a wax fastener), as depicted in the exemplary embodiments shown in FIGS. 9A-9L, 11F and 11L. In some aspects, the present invention provides a dispenser 50 comprising a wrapping 52, a plurality of apertures 54, and at least one fastener (e.g., a wax fastener), as depicted in the exemplary embodiments shown in FIGS. 10A-10L, 11A and 11B. In some aspects, the present invention provides a dispenser 50 comprising a wrapping 52, a plurality of apertures 54, and a plurality of fasteners 56 (e.g., wax fasteners), as depicted in the exemplary embodiments shown in FIGS. 11C, 11D, 11E, 11I, 11J, 11K, 11M and 11O. It is to be understood that the following description is generally applicable to each of the above aspects.

Generally, the wrapping 52 is sized and dimensioned to fold around a user selected amount of cannabis plant material. As used herein, a “user selected amount” means an amount of cannabis plant material selected by a user for sublingual administration of cannabinoids. A user can select an amount of cannabis plant material to be wrapped inside a dispenser 50 of the present invention based on a desired cannabinoid dosage (e.g., a prescribed amount of cannabinoids, e.g., an amount of cannabinoids prescribed by a physician to treat a disease, condition, or disorder the user is suffering from). Table 1 below provides exemplary information regarding cannabis weight, dispenser size, and cannabinoid content to assist a user in selecting an appropriate amount of cannabis plant material for sublingual administration of cannabinoids using a dispenser of the present invention. The information in Table 1 below can included in a kit or packaging comprising a sublingual dosage form of the present invention and/or dispenser, for example, to instruct a user how to obtain an effective amount of at least one pharmacologically active cannabinoid. For example, a kit or packaging comprising a dispenser can include a label comprising dosing information insofar as it relates to the amount of cannabis plant material. Generally, a user selects an amount of cannabis plant material that contains enough cannabinoid content when the cannabis plant material is decarboxylated to deliver an effective amount of at least one pharmacologically active cannabinoid, and wraps the decarboxylated cannabis plant material inside the dispenser by folding the wrapping around the decarboxylated cannabis plant material to retain the decarboxylated cannabis plant material inside the dispenser thus preparing the dispenser for sublingual use.

TABLE 1
Exemplary Dispenser Sizes

| Cannabis | | | Cannabinoid | |
|---|---|---|---|---|
| Weight | | Exemplary Dispenser Size Range | Content | |
| 0.1 | gm | .25 in. × .25 in.-.5 in. × .75 in. | 1-40 | mg |
| 0.2 | gm | .25 in. × .25 in.-.5 in. × 1 in. | 1-80 | mg |
| 0.3 | gm | .5 in. × .5 in.-.75 in. × .75 in. | 1-120 | mg |
| 0.4 | gm | .5 in. × .5 in.-1 in. × 1 in. | 1-160 | mg |
| 0.5 | gm | .5 in. × .5 in.-1.25 in. × 1.25 in. | 1-200 | mg |
| 1 | gm | .75 in. × .75 in.-2 in. × 2 in. | 1-400 | mg |
| 2 | gm | 1 in. × 1 in.-2.5 in. × 2.5 in. | 1-800 | mg |
| 3 | gm | 1.25 in. × 1.25 in.-3 in. × 3 in. | 1-1200 | mg |

Those skilled in the art will appreciate that actual dispenser sizes can vary, for example, depending on the shape of wrapping 52, the number, size, or distribution of the at least one apertures 54 passing through wrapping 52, and/or the thickness of wrapping 52, as the invention is not intended to be limited by dispenser size and/or dimension.

The wrapping 52 can be formed into any shape which permits a user to fold the wrapping 52 around a user selected amount of cannabis plant material, as the invention is not intended to be limited by the shape into which the wrapping 52 is formed. In accordance with example embodiments, the wrapping 52 comprises an asymmetrical shape. In accordance with example embodiments, the wrapping 52 comprises a symmetrical shape. In some embodiments, the wrapping 52 comprises a rectangular shape as is shown in FIG. 5A. In some embodiments, the wrapping 52 comprises a rectangular shape having rounded corners as is shown in FIG. 5B. In some embodiments, the wrapping 52 comprises an ovular shape as is shown in FIG. 5C. In some embodiments, the wrapping 52 comprises a rectangular shape having at least one beveled corner as is shown in FIG. 5D. In some embodiments, the wrapping 52 comprises a square shape as is shown in FIG. 5E. In some embodiments, the wrapping 52 comprises a circular shape as is shown in FIG. 5F. In some embodiments, the wrapping 52 comprises a heart shape as is shown in FIG. 5G. In some embodiments, the wrapping 52 comprises a triangular shape as is shown in FIG. 5H. Although an equilateral triangle is shown in FIG. 5H, it should be appreciated that other triangles can be used, such as isosceles and scalene triangles, for example. In some embodiments, the wrapping 52 comprises a hybrid rectangular triangular shape as is shown in FIG. 5I. In some embodiments, the wrapping 52 comprises a cross shape as is shown in FIG. 5J. In some embodiments, the wrapping 52 comprises a modified circle as is shown in FIG. 5K. In some embodiments, the wrapping 52 comprises a parallelogram as is shown in FIG. 5L. In some embodiments, the wrapping 52 comprises a combination of the foregoing shapes. Other suitable shapes would be apparent to the skilled artisan.

The manner in which wrapping 52 can be folded around cannabis plant material can vary, for example, depending on the shape of the wrapping. It is expected that symmetrical shapes can be folded once about each axis of symmetry to sufficiently enclose the cannabis plant material inside the dispenser. For example, cannabis plant material (e.g., decarboxylated) can be disposed proximal to the axis of symmetry of a heart shaped wrapping 52 (FIG. 5G), and the heart shaped wrapping can be folded about its sole axis of symmetry to enclose the cannabis plant material inside the dispenser 50. In another illustrative example, cannabis plant material can be disposed proximal an intersection of two axes of symmetry of a rectangular shaped wrapping 52 (FIGS. 5A and 5B), and the rectangular shaped wrapping can be folded once about its latitudinal axis of symmetry, and then a second time about its longitudinal axis of symmetry to enclose the cannabis plant material inside the dispenser 50. In another illustrative example, cannabis plant material can be disposed proximal an intersection of three axes of symmetry of a triangular shaped wrapping 52 (FIG. 5H), and the triangular shaped wrapping can be folded at least once about a first axis of symmetry, and optionally at least a second time about a second axis of symmetry, and optionally at least a third time about a third axis of symmetry, to enclose the cannabis plant material inside the dispenser 50. In yet another illustrative example, cannabis plant material can be disposed proximal an intersection of four axes of symmetry of a cross-shaped wrapping 52 (FIG. 5J), and the cross-shaped wrapping can be folded in such a way that each opposing edge portion is folded proximal an axis of symmetry so that a first pair of opposing edge portions contact each other and enclose the cannabis plant material inside the dispenser 50, and the second pair of opposing edge portions is folded over the first pair of opposing edge portion. The skilled person can envision a variety of other ways to fold the wrapping 52 depending on its shape, however, the invention is not intended to be limited by the manner in which the wrapping 52 is folded around the decarboxylated cannabis plant material.

The wrapping 52 can be constructed having any thickness which permits the wrapping to maintain its structural integrity when at least one aperture 54 passes through the wrapping. Preferably, the wrapping 52 has a thickness which facilitates ingestion of the wrapping with decarboxylated cannabis plant material inside the wrapping, for example, so that a user can swallow the dispenser 50 with the decarboxylated cannabis plant material contained therein after receiving a sublingual dose of at least one pharmacologically active cannabinoid. In accordance with an example embodiment, the wrapping 54 comprises a thickness of at least 0.1 mm. In accordance with an example embodiment, the wrapping 54 comprises a thickness of between 0.1 mm to about 3.0 mm. In accordance with an example embodiment, the wrapping 54 comprises a thickness of about 0.2 mm, 0.3 mm, 0.4 mm, 0.5 mm, 0.6 mm, 0.7 mm, 0.8 mm, 0.9 mm, 1.0 mm, 1.1 mm, 1.2 mm, 1.3 mm, 1.4 mm, 1.5 mm, 1.6 mm, 1.7 mm, 1.8 mm, 1.9 mm, 2.0 mm, mm, 2.2 mm, 2.3 mm, 2.4 mm, 2.5 mm, 2.6 mm, 2.7 mm, 2.8 mm, 2.9 mm, or 3.0 mm.

The wrapping 52 can be constructed from any ingestible material as long as the material retains its structural integrity when it is formed with at least one aperture 54 passing through the wrapping. In accordance with an example embodiment, the wrapping 52 is constructed from an ingestible material. As used herein, “ingestible” material means any material which can safely be ingested by a user, preferably without first chewing the material before swallowing the material. In other words, the ingestible material can be swallowed immediately by a user. Ingestible materials of use herein can be ingested by a user without inducing an immunological reaction in the user. Ingestible materials of use herein can be ingested by a user without physically damaging any portion of the gastrointestinal tract of the user. Ingestible materials of use herein are biocompatible. In some instances, ingestible materials of use herein are biodegradable. In accordance with an example embodiment, the wrapping 52 is constructed from an insoluble material. In accordance with an example embodiment, the wrapping 52 is constructed from an ingestible, insoluble material. In accordance with an example embodiment, the wrapping 52 is constructed from a fibrous plant material. In accordance with an example embodiment, the wrapping 52 is constructed from a fibrous plant material comprising cellulose. In accordance with an example embodiment, the wrapping 52 is constructed from cellulose. In accordance with an example embodiment, the wrapping 52 is constructed from a fibrous plant material comprising at least one of hemp, rice, bamboo, corn husk, silk husk, fruit skin, straw, flax, soy, wood, a pulp of any thereof, and combinations thereof.

Wrapping 52 can be constructed in any structural form in which at least one aperture 54 passes completely through the wrapping (e.g., from a first surface of the sheet to a second surface opposite to the first surface). In accordance with an example embodiment, the wrapping 52 is constructed in the form of a woven sheet. In accordance with an example embodiment, the wrapping 52 is constructed in the form of a non-woven sheet.

As is shown in the example embodiments in FIGS. 5A-5L, the wrapping 52 comprises at least one aperture 54 configured to retain cannabis plant material inside the wrapping while permitting an effective amount of at least one pharmacologically active cannabinoid released from the decarboxylated cannabis plant material upon exposure to saliva to flow with the saliva into a user's sublingual cavity when the wrapping is folded around the cannabis plant material and placed in the user's sublingual cavity. In other words, the apertures 54 are large enough for saliva and cannabinoids to pass through but not large enough for decarboxylated cannabis plant material to pass through (e.g., bud, shake, or flake form). In accordance with an example embodiment, the dispenser 50 comprises at least one aperture 54 configured to retain the decarboxylated cannabis plant material inside the dispenser while delivering the at least one pharmacologically active cannabinoid from the decarboxylated cannabis plant material into the sublingual cavity of the user.

The at least one aperture 54 can be configured to control the exposure of the decarboxylated cannabis plant material to saliva, as well as the exposure of the decarboxylated cannabis plant material to the sublingual mucosa of a user. For example, exposure of the decarboxylated cannabis plant material to saliva can be decreased by decreasing the number and/or size of the at least one aperture 54. Similarly, the surface area of the decarboxylated cannabis plant material which contacts a user's sublingual mucosa can be decreased by decreasing the number and/or size of the at least one aperture 54. Conversely, exposure of the decarboxylated cannabis plant material to saliva can be increased by increasing the number and/or size of the at least one aperture 54. Similarly, the surface area of the decarboxylated cannabis plant material which contacts a user's sublingual mucosa can be increased by increasing the number and/or size of the at least one aperture 54.

It is believed that release of the at least one pharmacologically active cannabinoid from the dispenser 50 can be modulated by controlling the exposure of the decarboxylated cannabis plant material to saliva and/or controlling the surface area of the decarboxylated cannabis plant material which contacts a user's sublingual mucosa. It is expected that release of the at least one pharmacologically active cannabinoid from the dispenser 50 can be delayed or sustained by limiting the number and/or size of the at least one aperture 54 passing through wrapping 52, or reducing the surface area of the decarboxylated cannabis plant material which contacts a user's sublingual mucosa. Similarly, it is expected that release of the at least one pharmacologically active cannabinoid from the dispenser 50 can be expedited or sped up by increasing the number and/or size of the at least one aperture 54 on the dispenser, or increasing the surface area of the decarboxylated cannabis plant material which contacts the user's sublingual mucosa. Those skilled in the art will appreciate that the surface area which contacts a user's sublingual mucosa can be modulated by adjusting the number and/or size of the at least one aperture 54. In accordance with an example embodiment, the dispenser 50 is configured to minimize exposure of the decarboxylated cannabis plant material to saliva when the wrapping 52 is placed in the subject's sublingual cavity. In accordance with an example embodiment, the dispenser 50 is configured to minimize contact between the cannabis plant material and the subject's sublingual mucosa when the wrapping 52 is placed in the subject's sublingual cavity. In accordance with an example embodiment, the dispenser 50 is configured with a single at least one aperture 54 to minimize exposure of the cannabis plant material to saliva and minimize contact between the cannabis plant material and a user's sublingual mucosa when the wrapping 52 is placed in the subject's sublingual cavity with the wrapping 52 folded around the cannabis plant material.

Turning now to FIGS. 6A-6L. In accordance with the example embodiments shown therein, the wrapping 52 comprises a plurality of apertures 54 configured to retain decarboxylated cannabis plant material inside the wrapping while permitting an effective amount of at least one pharmacologically active cannabinoid released from the decarboxylated cannabis plant material upon exposure to saliva to flow with the saliva into a user's sublingual cavity when the wrapping is folded around the decarboxylated cannabis plant material and placed in the subject's sublingual cavity. In accordance with an example embodiment, the dispenser 50 comprises a plurality of apertures 54 configured to retain the decarboxylated cannabis plant material inside the dispenser while delivering the at least one pharmacologically active cannabinoid from the decarboxylated cannabis plant material into the sublingual cavity of the subject. As is evident from FIGS. 6A-6L, any shape which is suitable for a wrapping 52 having a single at least one aperture 54 is suitable for a wrapping 52 having a plurality of apertures 54.

The dispenser 50 can be provided with any number, size, and/or pattern of apertures 54, as the invention is not intended to be limited by the number, size, and/or pattern of apertures 54 on wrapping 52. In accordance with an example embodiment, the plurality of apertures 54 are configured to maximize exposure of the decarboxylated cannabis plant material to saliva when the wrapping 52 is placed in a user's sublingual cavity. In accordance with an example embodiment, the plurality of apertures 54 are configured to maximize contact between the cannabis plant material and a user's sublingual mucosa when the wrapping 52 is placed in the user's sublingual cavity while it is folded around the cannabis plant material. Without wishing to be bound by theory, it is believed that maximizing exposure of the cannabis plant material to saliva and maximizing contact between the cannabis plant material and the user's sublingual mucosa maximizes the concentration or amount of the at least one pharmacologically active cannabinoid released from the dispenser and absorbed sublingually into the user's systemic circulation.

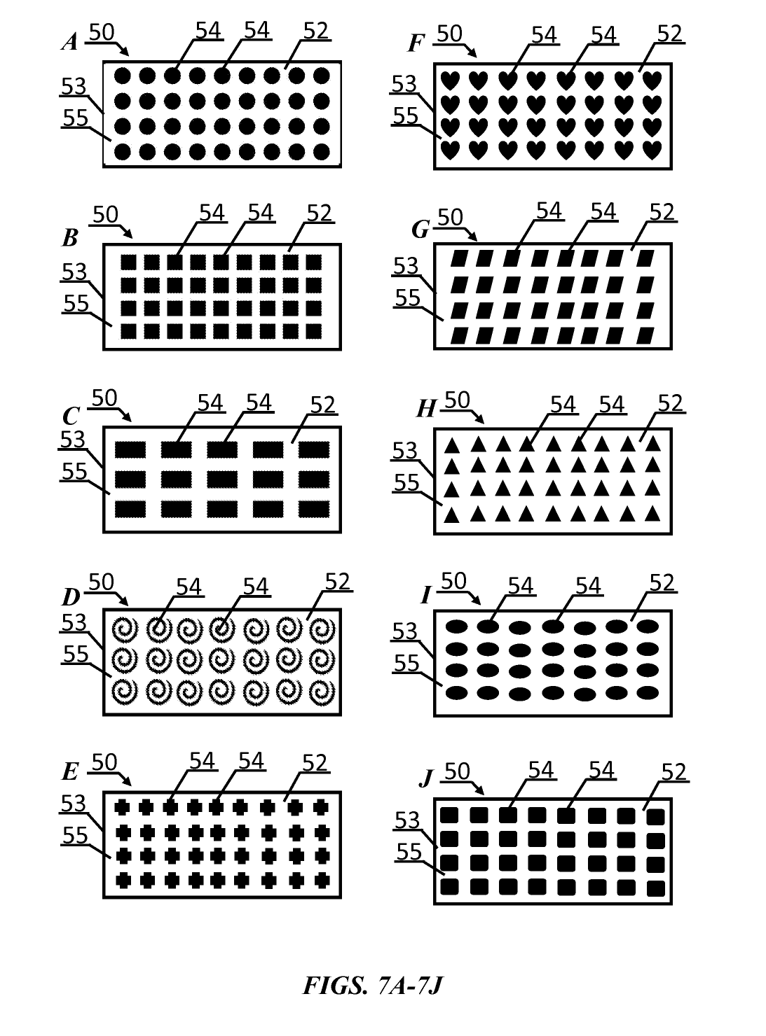

Generally, the present invention contemplates that the plurality of apertures 54 passing through wrapping 52 can be configured in any shape that is suitably sized and dimensioned to retain cannabis plant material inside the wrapping 52 when the wrapping is folded around the cannabis plant material, as the invention is not intended to be limited by the shape of the apertures. Exemplary shapes for the plurality of apertures include, without limitation, circles, squares, rectangles, swirls, crosses, hearts, diamonds, triangles, ovals, and rounded squares or rectangles, as shown in FIGS. 7A-7J. Aperture 54 shapes can similarly be patterned after real-world objects (e.g., animals, structures, plants, etc.) as long as they are sized and dimensioned to retain cannabis plant material inside the wrapping 54 when the wrapping is folded around the cannabis plant material and placed sublingually.

In accordance with example embodiments, each of the plurality of apertures 54 comprises the same geometric shape. In accordance with example embodiments, each of the plurality of apertures 54 comprises a different geometric shape. In accordance with an example embodiment, the plurality of apertures 54 comprises at least two different geometric shapes. In accordance with an example embodiment, the plurality of aperture's 54 comprises at least three different geometric shapes. In accordance with an example embodiment, the plurality of apertures 54 comprises at least four different geometric shapes. In accordance with an example embodiment, the plurality of apertures 54 comprises at least five different geometric shapes. In accordance with an example embodiment, the geometric shape or shapes comprise a regular shape. In accordance with an example embodiment, the geometric shape or shapes comprise an irregular shape.

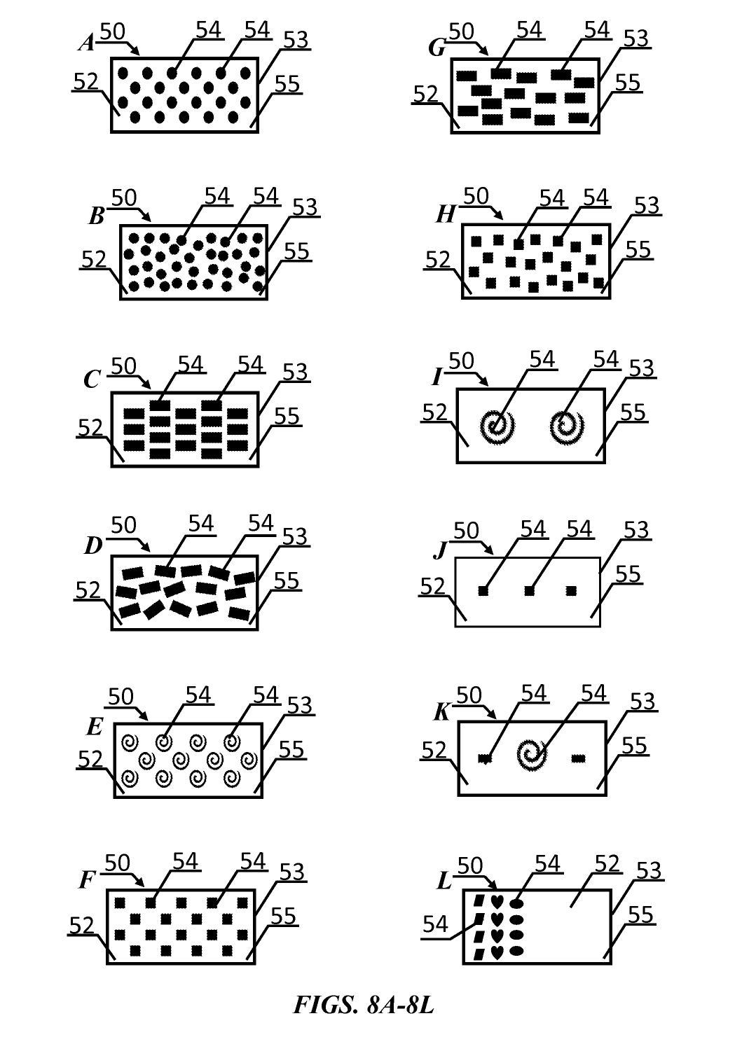

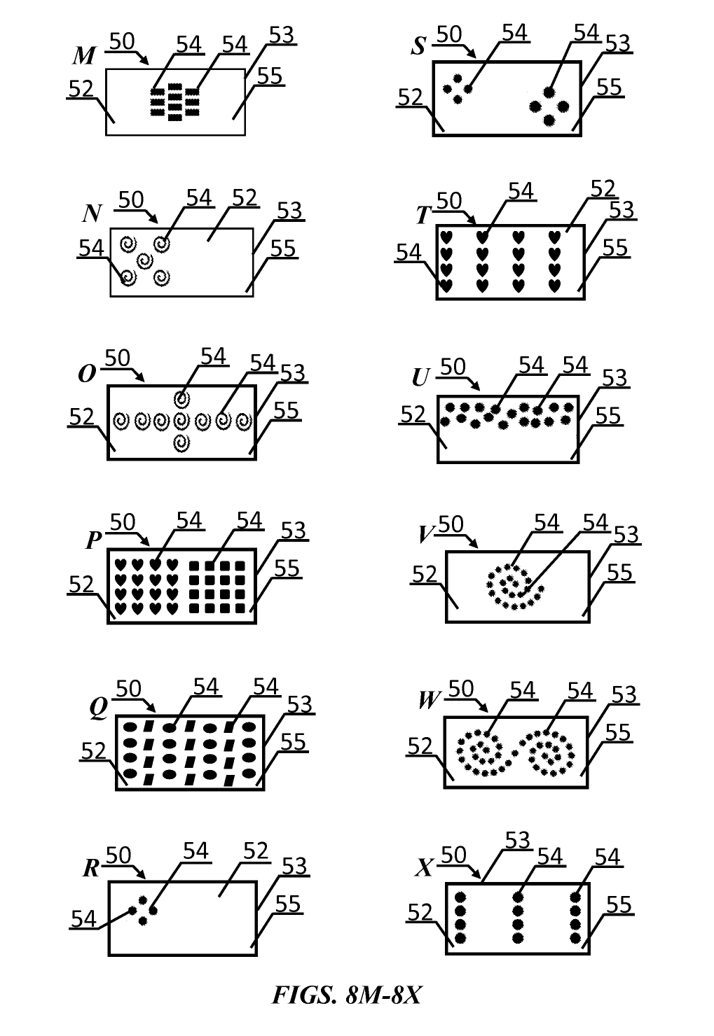

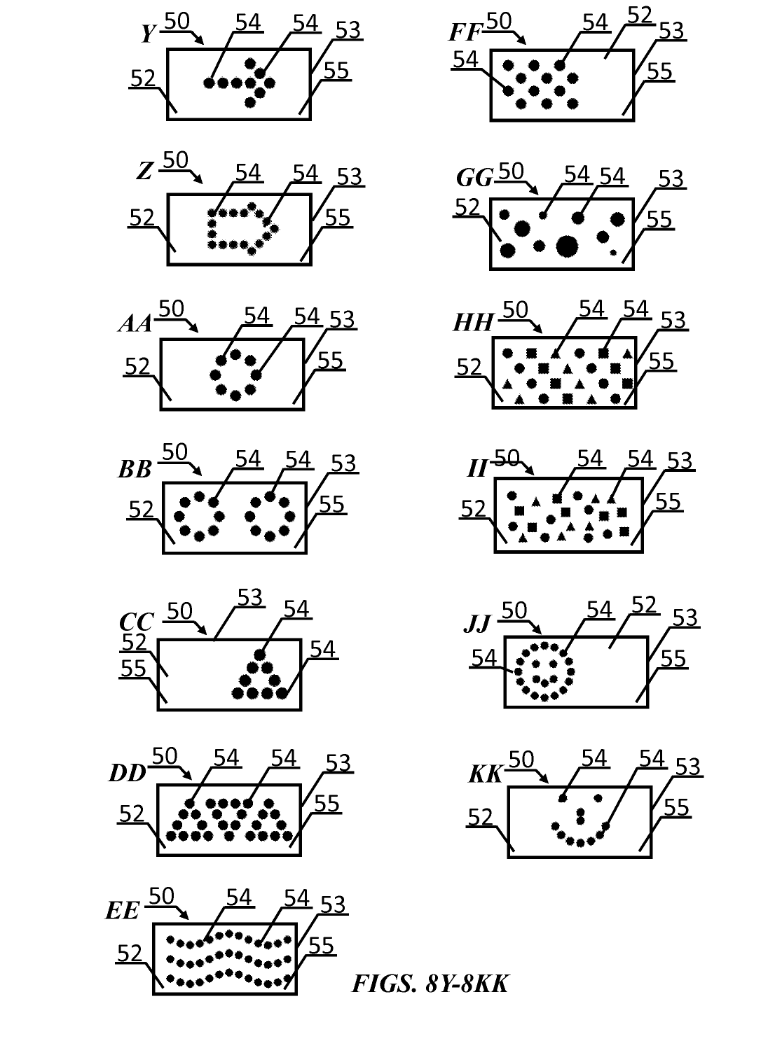

The plurality of apertures 54 can be arranged on the wrapping 52 in any desirable configuration or distribution, as the invention is not intended to be limited by the manner in which the apertures 54 are configured or distributed. In accordance with an example embodiment, the plurality of apertures 54 are arranged on the wrapping 52 in a uniform distribution. In accordance with an example embodiment, the plurality of apertures 54 are arranged on the wrapping 52 in a random distribution. In accordance with an example embodiment, the plurality of apertures 54 are arranged on the wrapping 52 in a regular pattern. Exemplary patterns and distributions of apertures 54 are shown in FIGS. 8A-8KK.

Generally, the plurality of apertures 54 can comprise any percentage of the surface area of wrapping 52 which permits the wrapping to retain its structural integrity when the apertures pass through the wrapping. In accordance with an example embodiment, the plurality of apertures 54 comprise at least 10% of the surface area of the wrapping 52. In accordance with an example embodiment, the plurality of apertures 54 comprise at least 15%, at least 20%, at least 22%, at least 25%, at least 33%, at least 40%, or at least 44% of the surface area of the wrapping 52. Preferably, the plurality of apertures comprise between 50% and 90% of the surface area of the wrapping 52. In accordance with an example embodiment, the plurality of apertures 54 comprise at least 55%, at least 60%, at least 62%, at least 65%, at least 63%, at least 70%, or at least 74% of the surface area of the wrapping 52. In accordance with an example embodiment, the plurality of apertures 54 are configured to pass through only a portion of the wrapping 52. The plurality of apertures 54 can be configured to pass through any portion of wrapping 52. In accordance with an example embodiment, each of the plurality of apertures 54 comprises an area of between about 1936 μM² and 1 in². In accordance with an example embodiment, each of the plurality of apertures 54 comprises a perimeter of between 148 μM and 4 inches. In accordance with an example embodiment, each of the plurality of apertures 54 comprises a circumference of between 132 μM and 3 inches. In an embodiment, each of the plurality of apertures 54 comprises a circumference of 12 mm. In an embodiment, each of the plurality of apertures 54 comprises an area of 3 mm². In an embodiment, the at least one aperture 54 comprises a width of at least 44 microns in one dimension. In an embodiment, the at least one aperture 54 comprises a perimeter of at least 148 μM. In an embodiment, the at least one aperture 54 comprises a circumference of 12 mm.

In accordance with an example embodiment, each of the plurality of apertures is visible to the naked eye. As used herein, “visible to the naked eye” means that the aperture 54 can be seen without the aid of a microscope. In accordance with an example embodiment, each of the plurality of apertures is visible to the naked eye (i.e., the plurality of apertures are sized and dimensioned in such a way that they are visible to the naked eye). In accordance with an example embodiment, the apertures 54 are machine formed. In accordance with an example embodiment, the apertures 54 comprise apertures resulting from a transverse applied force. In accordance with an example embodiment, the apertures 54 are not formed as a result of the manufacturing process of the wrapping 52 itself. In accordance with an example embodiment, the apertures 54 are formed on wrapping 52 after the wrapping is constructed in a desired shape and/or size. In accordance with an example embodiment, the apertures 54 are formed as a final step in constructing a dispenser 50.

Referring now to FIGS. 9A-10L, there is shown a dispenser 50 in accordance with various aspects of the invention. As is shown in the exemplary embodiments depicted in FIGS. 9A-10L, a dispenser 50 can include at least one fastener 56 disposed proximal the perimeter 53 of wrapping 52. The at least one fastener 56 can be disposed proximal any portion of the perimeter 53 of wrapping 52. Of course, the at least one fastener 56 can be disposed distal any portion of the perimeter 53 of wrapping 52. In accordance with example embodiments, the at least one fastener 56 can be disposed proximal the perimeter spanning only a portion of an edge of the wrapping 52, for example, as shown in FIGS. 9A, 9B, 9C, 9E, 9H, 9L, 10A, 10B, 10C, 10E, 10H, and 10L. In accordance with example embodiments, the at least one fastener 56 can be disposed proximal the perimeter 53 spanning an entire portion of an edge of the wrapping 52, for example, as shown in FIGS. 9D, 9J, 10D and 10J. In accordance with example embodiments, the at least one fastener 56 can be disposed proximal the perimeter 53 spanning an entire portion of multiple edges of the wrapping 52, for example, as shown in FIGS. 91 and 10I. In accordance with example embodiments, the at least one fastener 56 can be disposed proximal the perimeter 53 spanning a curved portion of the wrapping 52, for example, as shown in FIGS. 9C, 9F, 9G, 10C, 10F, and 10G. In accordance with example embodiments, the at least one fastener 56 can be disposed proximal the perimeter 53 spanning a curvilinear portion of the wrapping 52, for example, as shown in FIGS. 9K and 10K. In accordance with example embodiments shown in FIGS. 9L and 10L, the at least one fastener 56 can be disposed proximal the perimeter 53 spanning the entire perimeter the wrapping 52.

The at least one fastener 56 can be used to secure the surface (e.g., first surface 55) of the wrapping 52 to itself when the wrapping is folded over itself, for example around decarboxylated cannabis plant material, into contact with itself. That is, the at least one fastener 56 secures a first portion of the wrapping 52 to a second portion of the wrapping when the wrapping is folded such that the first portion proximal the at least one fastener 56 is folded into contact with the second portion.

The at least one fastener 56 can be constructed from any ingestible material which is capable of securing the surface of the wrapping to itself when the fastener contacts the surface of the wrapping. It should be appreciated that the at least one fastener 56 is configured to secure the surface of the wrapping 52 to itself regardless of whether the at least one fastener 56 contacts another fastener. That is, the at least one fastener 56 can secure the wrapping 52 to itself by contacting a surface of the wrapping on which at least one fastener is not disposed, as well as by contacting a surface of the wrapping on which at least one fastener is disposed. The skilled artisan will appreciate that the cannabis material will be more securely contained inside dispenser 50 when at least one fastener 56 secures the wrapping 52 to itself by contacting another at least one fastener 56, or when multiple at least one fasteners 56 are employed.

Preferably, the at least one fastener 56 comprises at least one wax fastener 56. The at least one wax fastener 56 can be constructed from any wax or wax-like material, preferably an edible wax or wax-like material, even more preferably an edible animal or vegetable wax or wax-like material. In accordance with an example embodiment, the at least one wax fastener 56 is constructed from beeswax. In accordance with an example embodiment, the at least one wax fastener 56 is constructed from candelilla wax. In accordance with an example embodiment, the at least one wax fastener 56 is constructed from carnauba. In accordance with an example embodiment, the at least one wax fastener 56 is constructed from ceresin wax. In accordance with an example embodiment, the at least one wax fastener 56 is constructed from Japan wax. In accordance with an example embodiment, the at least one wax fastener 56 is constructed from microcrystalline wax. In accordance with an example embodiment, the at least one wax fastener 56 is constructed from paraffin wax. In accordance with an example embodiment, the at least one wax fastener 56 is constructed from sugarcane wax. In accordance with an example embodiment, the at least one wax fastener 56 is constructed from a combination of waxes or wax-like materials. In accordance with an example embodiment, the at least one wax fastener 56 is constructed from an edible wax. In accordance with an example embodiment, the edible wax is selected from the group consisting of an animal wax, a vegetable wax, and a combination thereof. In accordance with an example embodiment, the animal wax is selected from the group consisting of beeswax, and shellac wax. In accordance with an example embodiment, the vegetable wax is selected from the group consisting of candelilla wax, carnauba wax, ceresin wax, japan wax, microcrystalline wax, paraffin wax, sugarcane wax, soy wax, and rice bran wax.

FIGS. 11A-11N demonstrate that the at least one fastener (e.g., wax fastener) 56 can be configured in a variety of ways in accordance with the spirit and scope of the present invention, as the invention is not intended to be limited by the manner in which the at least one fastener 56 is disposed on wrapping 52. For example, FIGS. 11A-11D demonstrate that the at least one wax fastener 56 can be disposed on any side or edge of wrapping 52. In accordance with the example embodiment shown in FIGS. 11E-11N, the at least one wax fastener 56 can comprise a plurality of wax fasteners 56 disposed proximal the perimeter 53 at different locations. In accordance with the example embodiments shown in FIGS. 11E and 11L, each of the plurality of wax fasteners 56 can comprise the same material, e.g., the same wax or waxy material. In accordance with the example embodiment shown in FIG. 11F, at least one of the plurality of wax fasteners 56 can comprise a different wax or wax-like material from at least one other of the plurality of wax fasteners. In accordance with the example embodiments shown in FIGS. 11E, 11F, 11J and 11K, the wrapping 52 can comprise at least two wax fasteners 56 disposed proximal the perimeter 53. In accordance with the example embodiment shown in FIG. 11G, the wrapping 52 can comprise at least three wax fasteners 56 disposed proximal the perimeter 53. In accordance with the example embodiment shown in FIG. 11H, the wrapping 52 can comprise at least four wax fasteners 56 disposed proximal the perimeter 53. In accordance with the example embodiment shown in FIG. 11I, the wrapping 52 can comprise at least six wax fasteners 56 disposed proximal the perimeter 53. In accordance with the example embodiments shown in FIGS. 11M and 11N, the wrapping 52 can comprise at least ten wax fasteners 56 disposed proximal the perimeter 53. Of course, although not shown, the wrapping 52 can comprise at least five, at least seven, at least eight, at least nine, or more wax fasteners 56 disposed proximal perimeter 53. The at least one, at least two, at least three, at least four, at least five, at least six, at least seven, at least eight, at least nine, or at least ten wax fasteners 56 can comprise the same or different wax or waxy materials. In accordance with the example embodiment shown in FIG. 11G, any of the at least one wax fasteners 56 can be disposed distal from the perimeter 53. In some embodiments, at least a first wax fastener 56 can be disposed proximal the perimeter 53, and at least a second wax fastener 56 can be disposed adjacent to the first wax fastener, as shown in the example embodiment depicted in FIGS. 11J and 11K.

In accordance with an example embodiment, a dispenser 50 includes a wrapping 52 having a first surface 55, and a perimeter 53, defined by a first side, a second side, a third side, and a fourth side, the wrapping 52 comprising at least one aperture 54 configured to retain a decarboxylated cannabis plant material 51 inside the wrapping 52 while permitting a maximum dosage of at least one pharmacologically active cannabinoid present in the decarboxylated cannabis plant material to be released into a subject's sublingual cavity when the wrapping is folded around the decarboxylated cannabis plant material and placed into the subject's sublingual cavity; and a first fastener 56 on at least a portion of the first surface 55.

In accordance with an example embodiment, a dispenser 50 includes a wrapping 52 having a first surface 55, and a perimeter 53 defined by a first side, a second side, a third side, and a fourth side, the wrapping 52 comprising a plurality of apertures 54 configured to retain decarboxylated cannabis plant material inside the wrapping while permitting a maximum dosage of at least one pharmacologically active cannabinoid present in the decarboxylated cannabis plant material to be released into a subject's sublingual cavity when the wrapping is folded around the decarboxylated cannabis plant material and placed into the subject's sublingual cavity; and a first fastener 56 on at least a portion of the first surface 55.

At least a portion of the first side of the first surface 55 can be folded around the decarboxylated cannabis plant material into at least a portion of any one of the second, third, or fourth sides of the first surface to retain the decarboxylated cannabis plant material inside the wrapping 52. The fastener 56 secures the at least the portion of the first side of the first surface 55 to at least the portion of any one of the second, third, or fourth sides of the first surface, so as to retain the plant material inside the wrapping when the at least the portion of the first side is folded around the plant material into contact with the at least the portion of any one of the second, third, or fourth sides. The dispenser 50 can include a second, third, and/or fourth fastener 56 on at least a portion of any one of the second, third and/or fourth sides. Each of the first, second, third, and/or fourth fasteners 56 can be constructed from the same material or from a different material. In accordance with an example embodiment, any two of the first, second, third, and/or fourth fasteners 56 is constructed from the same material. In accordance with an example embodiment, any three of the first, second, third, and/or fourth fasteners 56 is constructed from the same material. In accordance with an example embodiment, any two of the first, second, third, and/or fourth fasteners 56 can be constructed from a different material. In accordance with an example embodiment, any three of the first, second, third, and/or fourth fasteners 56 is constructed from a different material. In accordance with an example embodiment, the first, second, third, and/or fourth fasteners 56 comprises at least one or any combination of beeswax, candelilla wax, carnauba, ceresin wax, japan wax, microcrystalline wax, paraffin wax, shellac wax, and/or sugarcane wax.

In accordance with the example embodiments shown in FIGS. 11L-11N, the at least one wax fastener 56 can comprise a colored wax. The colored wax can be naturally or artificially colored, as long as the wax is safe for human consumption. In accordance with an example embodiment, the colored wax comprises a coloring approved by a regulatory agency (e.g., FDA), for example a food coloring. The colored wax can be used as a visual folding indicator. In some embodiments, an oil can be used as a visual folding indicator. As used herein, “visual folding indicator” in the context of a colored wax means that the color of the at least one wax fastener 56 provides a visual cue to instruct a user of the dispenser 50 how to fold the wrapping 52 to retain the cannabis plant material inside the dispenser to prepare the dispenser for sublingual use. Non-limiting examples of visual folding indicators are shown in FIGS. 11L-11N. As shown in FIGS. 11L-11N, the colored wax fasteners 56 cue a user to fold the wrapping 52 over cannabis plant material so that wax fasteners of the same color contact each other to secure the wrapping to itself around the cannabis plant material to contain the cannabis plant material inside the dispenser 50. For example, the red wax fasteners 56 shown in FIG. 11L can be folded over cannabis plant material into contact with each other to secure the opposing edges of the wrapping 52 to itself to wrap the cannabis plant material inside the dispenser 50. Similarly, the red wax fasteners 56 shown in FIG. 11M can be folded over cannabis plant material into contact with each other to secure the opposing edges of the wrapping 52 to itself to wrap the cannabis plant material inside the dispenser 50. It should be appreciated that by folding the red wax fasteners 56 into contact with each other over cannabis plant material the remaining wax fasteners 56 on the wrapping 52 will further secure the wrapping 52 to itself to better retain the cannabis plant material inside the dispenser 50. The remaining wax fasteners 56 need not be colored as the remaining wax fasteners are positioned in such a way that they will automatically secure the wrapping to itself when the user folds the colored wax fasteners into contact with each other. Optionally, additional wax fasteners 56 can be colored, for example as shown in FIG. 11N, to indicate which wax fasteners are to be folded into contact with each other to optimally wrap the cannabis plant material and retain the cannabis plant material inside dispenser 50. It should be appreciated that the colors featured in FIGS. 11L-11N can be any color that exists in a natural or artificial form.

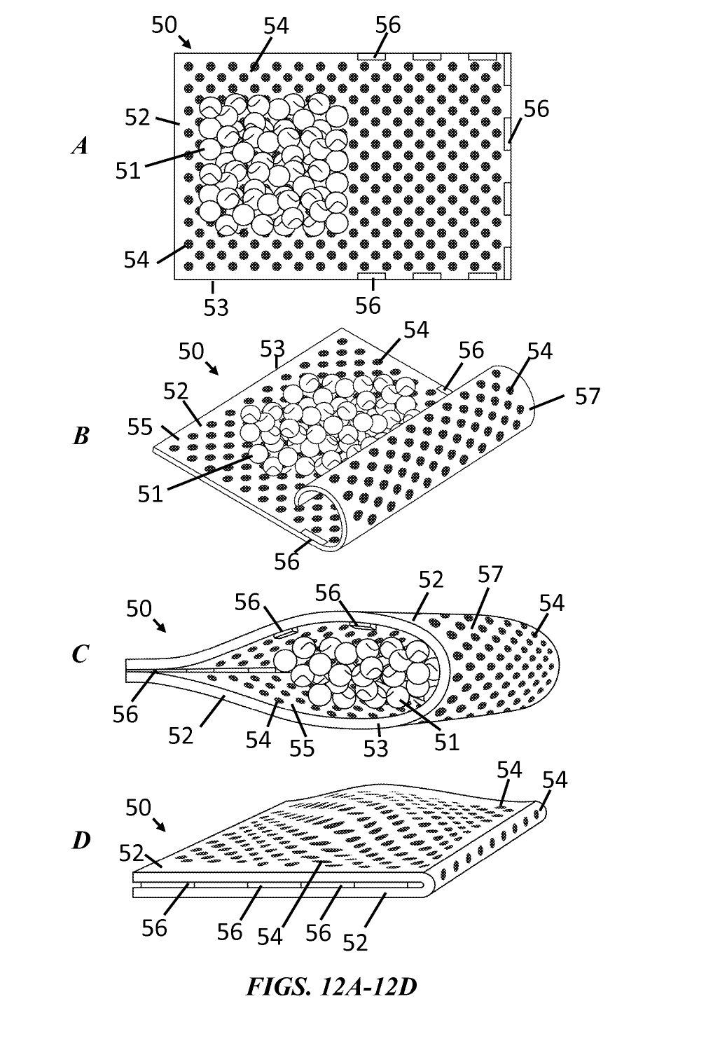

Referring now to FIGS. 12A-12D, there is shown a dispenser 50 in accordance with various aspects of the present invention. In accordance with an aspect of the present invention, a wrapping 52 comprises a sheet of ingestible material having a perimeter 53, a first surface 55, and a second surface 57 on an opposite side of the sheet from the first surface 55, at least one aperture 54 passing completely through from the first surface 55 to the second surface 57 and vice versa, and at least one wax fastener 56 disposed proximal the perimeter 53 and configured to secure the sheet of ingestible material to itself when the sheet is folded over and placed in contact with the at least one wax fastener 56.

In accordance with an aspect of the present invention, a wrapping 52 comprises a sheet of ingestible material having a perimeter 53, a first surface 55, and a second surface 57 on an opposite side of the sheet from the first surface 55, a plurality of apertures 54 passing completely through from the first surface 55 to the second surface 57 and vice versa, and at least one wax fastener 56 disposed proximal the perimeter 53 and configured to secure the sheet of ingestible material to itself when the sheet is folded over and placed in contact with the at least one wax fastener 56.

In accordance with an aspect of the present invention, a wrapping 52 comprises a sheet of ingestible material having a perimeter 53, a first surface 55, and a second surface 57 on an opposite side of the sheet from the first surface 55, a plurality of apertures 54 passing completely through from the first surface 55 to the second surface 57 and vice versa, at least one wax fastener 56 disposed proximal the perimeter 53 and configured to secure the sheet of ingestible material to itself when the sheet is folded over and placed in contact with the at least one wax fastener 56, wherein the sheet is configured to enclose decarboxylated cannabis plant material 51 inside the wrapping 52 when the sheet is folded over and placed in contact with the at least one wax fastener 56, wherein when the wrapping 52 is placed in the sublingual cavity of a subject with decarboyxlated cannabis plant material 51 inside the wrapping 52, the at least one aperture 54 permits cannabinoids present in the decarboxylated cannabis plant material 51 to exit the wrapping 52 while retaining the decarboxylated cannabis plant material 51 inside the wrapping 52.

In accordance with an aspect of the present invention, a wrapping 52 comprises a sheet of ingestible material having a perimeter 53, a first surface 55, and a second surface 57 on an opposite side of the sheet from the first surface 55, at least one aperture 54 passing completely through from the first surface 55 to the second surface 57 and vice versa, at least one wax fastener 56 disposed proximal the perimeter 53 and configured to secure the sheet of ingestible material to itself when the sheet is folded over and placed in contact with the at least one wax fastener 56, wherein the sheet is configured to enclose decarboxylated cannabis plant material 51 inside the wrapping 52 when the sheet is folded over and placed in contact with the at least one wax fastener 56; wherein when the wrapping is placed in the sublingual cavity of a subject with decarboyxlated cannabis plant material inside the wrapping, the at least one aperture permits cannabinoids present in the decarboxylated cannabis plant material to exit the wrapping while retaining the decarboxylated cannabis plant material 51 inside the wrapping.

The wrapping 52 can be folded around decarboxylated cannabis plant material to prepare the dispenser 50 for sublingual administration of at least one pharmacologically active cannabinoid in the decarboxylated cannabis plant material. The cannabis plant material can be decarboxylated in accordance with a method described herein, and then the wrapping 52 can be folded around the decarboxylated cannabis plant material as described herein, and then placed under a user's tongue for sublingual administration of the at least one pharmacologically active cannabinoid from the dispenser 50. Preferably, the wrapping 52 is folded around decarboxylated cannabis plant material in such a way that the decarboxylated cannabis plant material is retained inside the wrapping when the dispenser 50 resides in a user's sublingual cavity. FIGS. 12A-12D demonstrate folding wrapping 52 around cannabis plant material using an exemplary dispenser 50 of the present invention. As is shown in FIG. 12A, a user selected amount of cannabis plant material 51 (e.g., decarboxylated cannabis plant material) is placed onto the surface of wrapping 52. Next, wrapping 52 is folded over the cannabis plant material, for example, in such a way that the at least one wax fastener (e.g., plurality of wax fasteners) 56 secure the cannabis plant material 51 inside the dispenser 50, as is shown in the example embodiment depicted in FIGS. 12B and 12C. As is shown in FIG. 12D, the at least one wax fastener 56 secures the cannabis plant material inside the dispenser 50 when the at least one wax fastener 56 contacts the wrapping 52, thereby retaining the cannabis plant material inside the dispenser 50. In instances in which the cannabis plant material has been decarboxylated prior to placement on the surface of the wrapping 52, the dispenser thus folded around the cannabis plant material is ready for sublingual use.

In accordance with an example embodiment, the wrapping 52 is folded once over the decarboxylated cannabis plant material in such a way that the decarboxylated cannabis plant material is sandwiched between opposing sides of the same surface (e.g., first surface) of the dispenser 50. In such embodiment, the wrapping 52 comprises two layers composed of the same sheet of material enclosing the cannabis plant material. In accordance with an example embodiment, the wrapping 52 is folded twice over the decarboxylated cannabis plant material. In accordance with an example embodiment, the wrapping 52 is folded thrice over the decarboxylated cannabis plant material. In accordance with an example embodiment, the wrapping 52 a first edge portion of the wrapping is folded over the cannabis plant material, and an opposite second edge portion of the wrapping is folded over the cannabis plant material into contact with the first edge portion to retain the decarboxylated cannabis plant material inside the dispenser.

It should be appreciated that the sheet can be formed with any structure which permits the sheet to retain its structural integrity when at least one aperture 54 passes completely through from the first surface 55 to the second surface 57 and vice versa, so that the cannabis plant material is retained inside the wrapping 52 when folded around the cannabis plant material and placed sublingually. In accordance with an example embodiment, the sheet comprises a woven structure. In accordance with an example embodiment, the sheet comprises a non-woven structure. In accordance with an example embodiment, the sheet is not configured in a pillow configuration. In accordance with an example embodiment, the sheet is not configured as a pouch. In accordance with an example embodiment, the dispenser 50 is not a coating. In accordance with an example embodiment, the dispenser 50 is not a pouch.

It is contemplated that any ingestible material can be used to form the sheet. In accordance with an example embodiment, the ingestible material comprises an insoluble material. In accordance with an example embodiment, the ingestible material comprises a fibrous plant material. In accordance with an example embodiment, the ingestible material comprises cellulose. In accordance with an example embodiment, the ingestible material comprises at least one of hemp, rice, bamboo, corn husk, silk husk, fruit skin (e.g., dried or cured fruit skin), straw, flax, soy, wood, a pulp of any thereof, and combinations thereof.

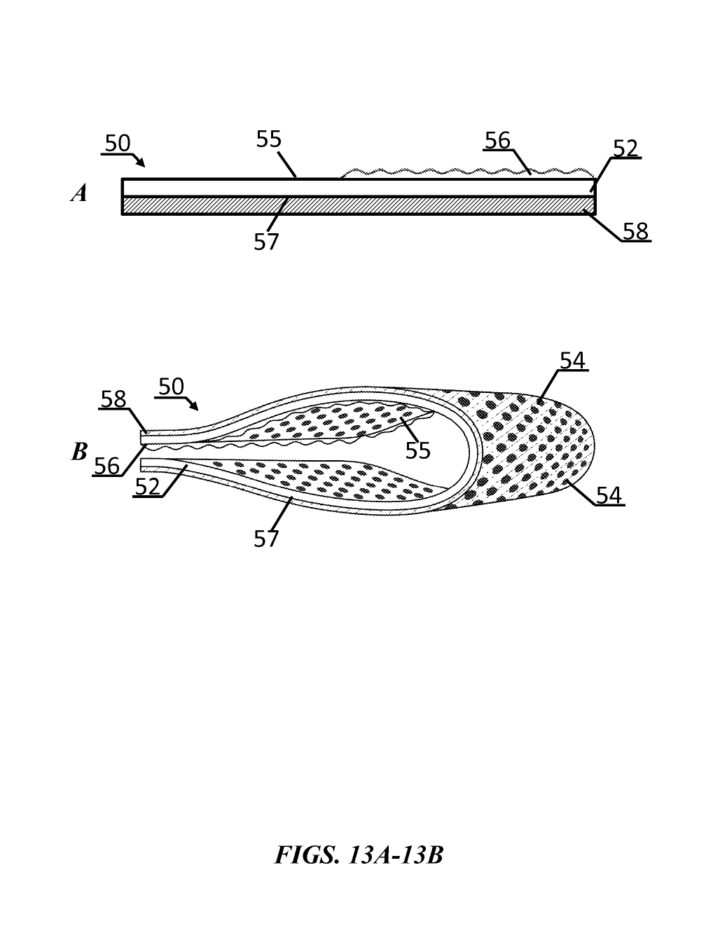

Optionally, a dispenser 50 of the present invention can include a coating 58, as is shown in the exemplary embodiments depicted in FIGS. 13A and 13B. FIG. 13A shows a side view of a dispenser 50 of the present invention. As shown in FIG. 13A, a dispenser 50 can include at least one wax fastener 56 on at least a portion of a first surface 55 of wrapping 52, and a coating 58. In other words, coating 58 is disposed on an opposite surface from the at least one wax fastener 56. Although shown as covering the entire second surface 57 of wrapping 52, it should be appreciated that the coating may be disposed on only a portion of the second surface 57 of wrapping 52 or on a portion of the first surface 55. In accordance with an example embodiment, coating 58 can be disposed on at least a portion of the second surface 57 of wrapping 52. In accordance with an example embodiment, coating 58 can be disposed on approximately 10%, 15%, 20%, 25%, 30%, 35%, 40%, 50%, 55%, 60%, 70%, 75%, 80%, 85%, 86%, 90%, 95% or more of second surface 57 of wrapping 52. In accordance with an example embodiment, coating 58 can be disposed on approximately 50% of the second surface. In accordance with an example embodiment, coating 58 is disposed on the entire portion of the second surface 57. FIG. 13B shows a perspective view of a dispenser 50 of the present invention. As is shown in FIG. 13B, the plurality of apertures 54 are generally free of coating 58. In other words, the coating 58 is not intended to obstruct or cover the apertures 58. The coating 58 can be disposed on second surface 57 using any suitable technique, e.g., dipped or sprayed. The coating 58 can be disposed in a uniform layer on surface 57. The coating 58 can be disposed in an uneven layer on surface 57. The coating can be disposed on surface 57 in any desired thickness. It is expected that certain properties imparted by coating 58 may be enhanced by increasing the thickness of coating 58. For example, it is believed that a thicker coating of waxy or gelatinous material can enhance a user's sensation of comfort, smoothness and/or softness in a user's sublingual cavity and/or throat. It should be appreciated that although only a single layer coating 58 is shown in FIGS. 13A and 13B, multiple layers of coatings 58 are contemplated by the invention. The multiple layers of coatings 58 can comprise different materials and/or impart different properties or characteristics to the dispenser 50. For example, a dispenser 50 can include at least two coatings comprising a first coating 58 comprising a comfort, smoothness, and or softness enhancing coating as described herein, and a second coating (not shown) comprising a flavorant and/or oil (e.g., a lipophilic absorption enhancing oil). As another example, dispenser 50 can include at least three coatings comprising a first coating 58 comprising a comfort, smoothness, and/or softness enhancing coating as described herein, a second coating comprising an oil, and a third coating comprising a flavorant. Those skilled in the art will appreciate that the order in which the various coatings are disposed on second surface 57 can be interchanged as desired, and multiple materials can be mixed to form any particular coating layer to impart multiple desired properties.

Generally, coating 58 can be configured to impart desired physical properties and performance characteristics to dispenser 50 (e.g., elicit a positive sensory response from a user, ease swallowing or ingestion, improve cannabinoid absorption, etc.). In accordance with an example embodiment, coating 58 is configured to elicit a positive sensory response from a user of dispenser 50. Preferably, coating 58 is configured to elicit a sensation of smoothness, comfort, pleasant taste, and/or softness. In accordance with an example embodiment, the positive sensory response comprises a sensation of smoothness, comfort, pleasant taste, and/or softness in the subject's mouth, sublingual cavity and/or throat. In accordance with an example embodiment, coating 58 is configured to ease swallowing or ingestion of dispenser 50 containing decarboxylated cannabis plant material inside it. In accordance with an example embodiment, coating 58 is configured to enhance absorption of cannabinoids released from decarboxylated cannabis plant material inside the dispenser 50.

The coating 58 can be configured to enhance absorption of the at least one pharmacologically active cannabinoid, for example, by disposing a lipophilic coating on second surface 57 of wrapping 52. It is expected that a lipophilic coating would aid in the transmission and absorption of cannabinoids within the sublingual cavity and digestive tract. The coating 58 can be configured to ease swallowing or ingestion of dispenser 50, for example, by disposing a lubricious coating on second surface 57 of wrapping 52.

It should be appreciated that coating 58 is disposed on an exterior surface 57 of wrapping 52 when the wrapping is folded around cannabis plant material to retain the cannabis plant material inside the dispenser 50. Similarly, the at least one wax fastener 56 (or plurality of wax fasteners 56) are disposed proximal the perimeter 53 on an interior surface 55 of wrapping 52 when the wrapping is folded around cannabis plant material to retain the cannabis plant material inside the dispenser 50. In accordance with an example embodiment, a dispenser 50 comprises a fastener 56 on a first portion of an interior surface 55 of the dispenser 50 which secures the first portion of the interior surface 55 of the dispenser to at least a second portion of the interior surface 55 of the dispenser when the dispenser is folded around cannabis plant material in such a way that the fastener contacts the at least the second portion of the interior surface, thereby retaining the decarboxyalted cannabis plant material inside the dispenser. In accordance with an example embodiment, the dispenser 50 comprises at least a first coating 58 on at least a portion of an exterior surface 57 of the dispenser 50. In accordance with an example embodiment, the at least the first coating 58 on the exterior surface 57 elicits a positive sensory response from a user when the dispenser 50 is placed in a user's sublingual cavity and/or ingested by the user with the decarboxylated cannabis plant material contained inside the dispenser. In accordance with an example embodiment, the at least the first coating 58 on the exterior surface 57 enhances sublingual absorption of cannabinoids released from decarboxylated cannabis plant material into a user's sublingual mucosa when the dispenser 50 is placed in a user's sublingual cavity with the decarboxylated cannabis plant material inside the dispenser. In accordance with an example embodiment, the at least the first coating 58 on the exterior surface 57 facilitates swallowing or ingestion of the dispenser with decarboxylated cannabis plant material retained inside the dispenser.

In some aspects, the coating comprises 58 an edible material which elicits the positive sensory response. For example, the coating 58 can be an edible waxy or gelatinous material which elicits a sensation of smoothness, comfort, and/or softness when the dispenser 50 contacts a user's oral cavity. In accordance with an exemplary embodiment, the edible material comprises an animal wax. Exemplary animal waxes include, without limitation, beeswax and shellac wax. In accordance with an exemplary embodiment, the edible material comprises a vegetable wax. Exemplary vegetable waxes include, without limitation, candelilla wax, carnauba wax, ceresin wax, japan wax, microcrystalline wax, paraffin wax, sugarcane wax, soy wax, and rice bran wax. In accordance with an exemplary embodiment, the gelatinous material comprises gelatin. In accordance with an example embodiment, the at least the first coating 58 comprises a wax or gelatin. In accordance with an example embodiment, the at least the first coating 58 comprises at least one of beeswax, candelilla wax, carnauba, ceresin wax, japan wax, microcrystalline wax, paraffin wax, shellac wax, sugarcane wax, and combinations thereof. In accordance with an example embodiment, the at least the first coating 58 comprises a lubricious coating. In accordance with an example embodiment, the at least the first coating 58 comprises a lipophilic coating. In accordance with an example embodiment, the at least the first coating 58 comprises a lipophilic, lubricious coating.

In some aspects, coating 58 comprises a flavorant described herein.

In some aspects, coating 58 comprises an oil described herein.

In some aspects, coating 58 comprises a combination or mixture of an edible material described herein, a flavorant, and an oil. In some aspects, the coating 58 comprises a combination or mixture of an edible material described herein, a flavorant, an oil, a lubricant, and/or a lipophilic substance.

As another example, dispenser 50 can include at least one coating 58 comprising a lubricant, for example, to facilitate swallowing or ingestion of dispenser 50 after sublingual administration of at least one pharmacologically active cannabinoid.

The dispenser 50 of the present invention can generally be distributed and/or sold in any packaging. The skilled artisan will appreciate that the form in which the dispenser 50 is packaged or distributed can depend on the type of packaging. In accordance with an example embodiment, the dispenser 50 is packaged in such a way that wrapping 52 is in an unfolded form. In accordance with an example embodiment, the dispenser 50 is packaged in such a way that wrapping 52 is in an unfolded form. For example, a dispenser 50 can be packaged in such a way that wrapping 52 is in an unfolded form so that a user can unfold the wrapping 52 and refold wrapping 52 around cannabis plant material in exactly the same way that it was folded. In this way, the folded form of wrapping 52 serves as a visual cue to instruct a user of a suggested way to fold wrapping 52 around cannabis plant material to prepare the dispenser 52 for sublingual use.

A plurality of individual dispensers 50 can be packaged together in a single packaging, or individually packaged. When a plurality of individual dispensers 50 are packaged together in a single packaging, each adjacent wrapping 52 can be separated from each other by a sheet. Examples of suitable sheets to separate dispensers 50 include, without limitation, cellophane, wax paper, parchment paper, and/or laminated aluminum. In accordance with an example embodiment, the packaging comprises an aluminum foil bag comprising a zip top and a tear notch, wherein the tear notch permits the packaging to be opened thereby permitting a consumer to remove at least one wrapping from the packaging, and wherein the zip top permits the packaging to be sealed thereby permitting the consumer to store the remaining wrappings in the packaging.

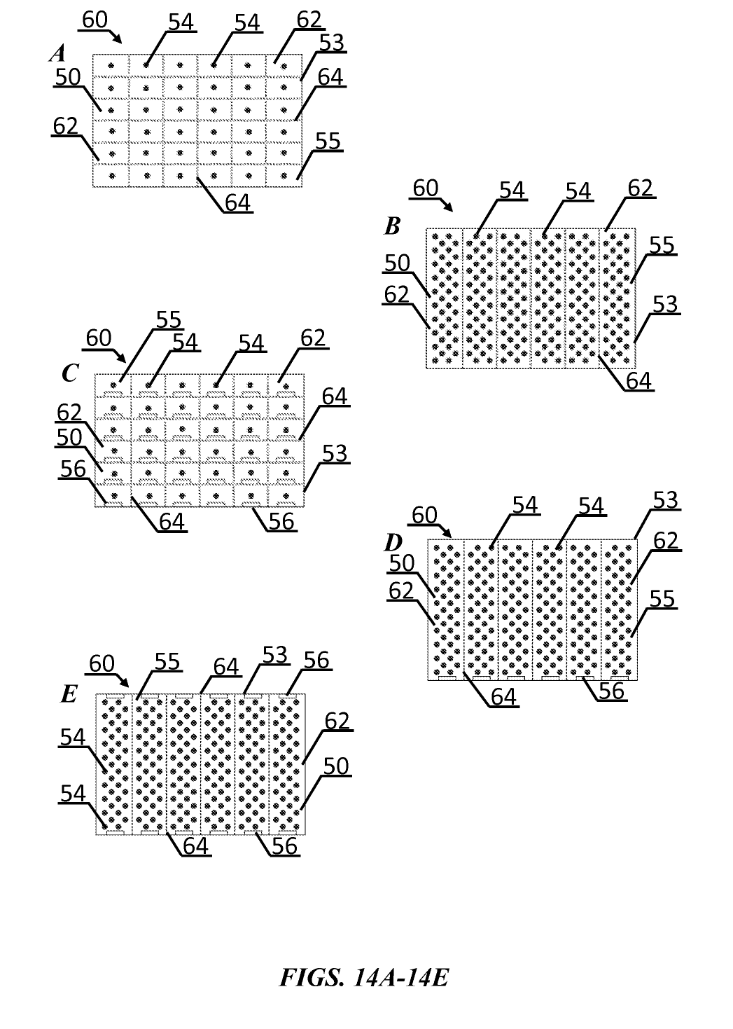

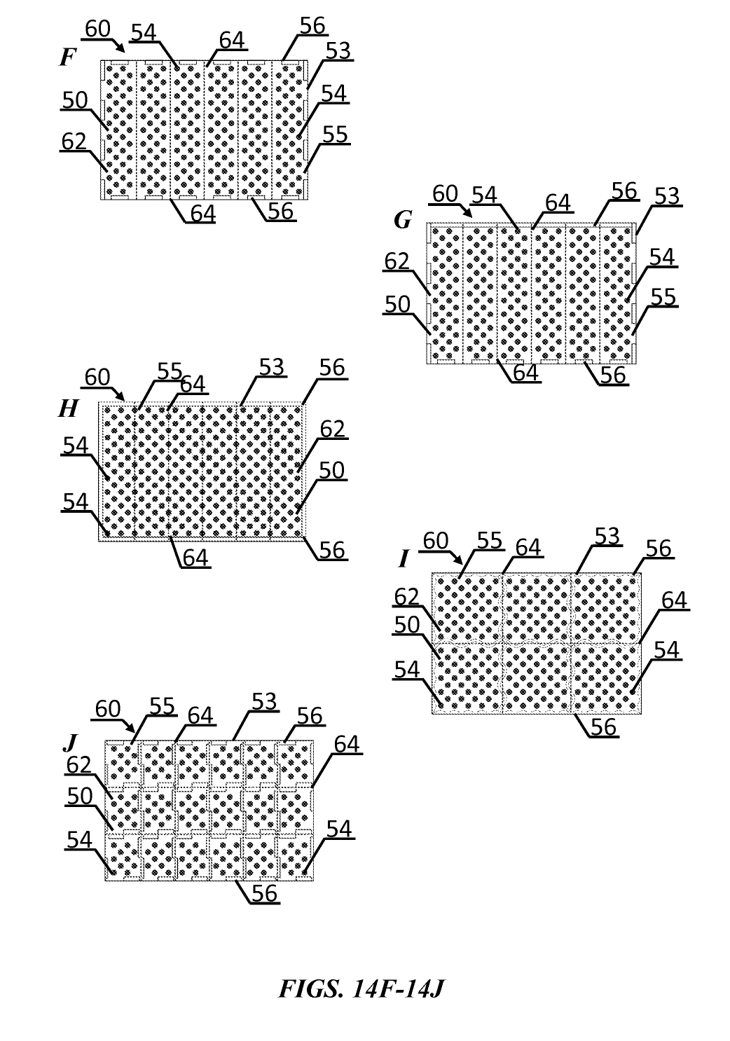

In some aspects, the invention provides a plurality of dispensers 60 comprising a plurality of wrappings 62 separated by line perforations 64, each wrapping 62 comprising at least one aperture 54. An exemplary such plurality of dispensers 60 is shown in FIG. 14A. In some aspects, the invention provides a plurality of dispensers 60 comprising a plurality of wrappings 62 separated by line perforations 64, each wrapping 62 comprising a plurality of apertures 54. An exemplary such plurality of dispensers 60 is shown in FIG. 14B. In some aspects, the invention provides a plurality of dispensers 60 comprising a plurality of wrappings 62 separated by line perforations 64, each wrapping comprising at least one aperture 54 and at least one fastener (e.g., wax fastener) 56. An exemplary such plurality of dispensers 60 is shown in FIG. 14C. In some aspects, the invention provides a plurality of dispensers 60 comprising a plurality of wrappings 62 separated by line perforations 64, each wrapping comprising a plurality of apertures 54 and at least one fastener (e.g., wax fastener) 56. An exemplary such plurality of wrappings is shown in FIG. 14D. In some aspects, the invention provides a plurality of dispensers 60 comprising a plurality of wrappings 62 separated by line perforations 64, each wrapping comprising a plurality of apertures 54 and a plurality of fasteners (e.g., wax fasteners) 56. Exemplary such plurality of dispensers 60 are shown in FIGS. 14E, 14F, 14G, 14H, 14I and 14J.

In accordance with an example embodiment, the invention provides a plurality of dispensers 60 formed into a continuous sheet, wherein each dispenser 50 comprises a wrapping 62 comprising at least one aperture 54 configured to retain cannabis plant material (e.g., decarboxylated cannabis plant material) inside the wrapping while permitting an effective amount of at least one pharmacologically active cannabinoid in the cannabis plant material to be released into a user's sublingual cavity when the wrapping is folded around the cannabis plant material and placed in the user's sublingual cavity.

In accordance with an example embodiment, the invention provides a plurality of dispensers 60 formed into a continuous sheet, wherein each dispenser 50 comprises a wrapping 62 comprising a plurality of apertures 54 configured to retain cannabis plant material (e.g., decarboxylated cannabis plant material) inside the wrapping while permitting an effective amount of at least one pharmacologically active cannabinoid in the cannabis plant material to be released into a user's sublingual cavity when the wrapping is folded around the cannabis plant material and placed in the user's sublingual cavity.

The plurality of dispensers 60 formed into a sheet can designed to include any number of dispensers 50, as the invention is not intended to be limited by the number of dispensers in a sheet. In accordance with example embodiments, the plurality of dispensers 60 include at least two, at least three, at least four, at least five, at least six, at least seven, at least eight, at least nine, at least 10, at least 12, at least 14, at least 16, at least 18, at least 20, at least 23, at least 25, at least 30, at least 40, at least 45, at least 50, at least 55, at least 60, at least 64, at least 75, at least 80, at least 90, or at least 100 or more dispensers.

The plurality of dispensers 60 in each sheet are physically separated by line perforations 64, which provide a visual tearing indicator to a user. As used herein, “visual tearing indicator” refers to a visual cue that signals a user to tear along the sheet so that the user can physically remove at least one dispenser 50 from the plurality of dispensers 60 in the sheet without damaging the structural integrity of adjacent dispensers 60 in the sheet. As is shown in FIGS. 14A-14J, the plurality of dispensers 60 include a line perforation 64 between each of the adjacent dispensers 50 in the sheet. The line perforation 64 permits adjacent dispensers 50 in the sheet to be separated from each other. The line perforation 64 permits any dispenser 50 in the sheet to be separated from the sheet by enabling a user to tear the sheet along the line perforation without damaging the structural integrity of the dispenser being removed from the sheet or any other dispenser in the sheet.

As is shown in the example embodiments in FIGS. 14A-14J, line perforations 64 can be spaced apart from each other to provide each dispenser 50 with a desired dimension and/or shape. Although the line perforations 64 are spaced apart from each other in such a way that the resulting dimensions and/or shapes of each dispenser 50 are the same, it should be appreciated that the line perforations 64 can be spaced apart from each other to provide the each dispenser 50 with any desired dimension and/or shape, including different dimensions and/or shapes. As such, the rectangular shapes of the plurality of dispensers 60 depicted in the figures are merely exemplary. The line perforations 64 can be aligned parallel to each other and/or perpendicular to each other. Such alignment can be at 90 degrees and 180 degrees as shown, or at any other desired angle. If desired, line perforations 64 can intersect each other, for example, at one or multiple locations on the sheet 60.

The line perforation 64 permits a user to increase the dosage by separating a group of dispensers 50 from the sheet, for example, by tearing the line perforations between the group of dispensers and the sheet, wherein the dosage is proportionate to the number of dispensers in the group separated from the sheet. The number of dispensers 50 in a sheet can be configured to reflect a prescribed dosage regimen, for example, a daily dosage regimen, a twice daily dosage regimen, a thrice daily dosage regimen, a weekly dosage regimen, etc. For example, a plurality of dispensers 60 can be designed to include seven dispensers 50 to provide a once daily dosage regimen that is expected to last the user a week. A plurality of dispensers 60 can be designed to include 14 dispensers 50 to provide a twice daily dosage regimen that is expected to last the user a week. A plurality of dispensers 60 can be designed to include 21 dispensers 50 to provide a thrice daily dosage regimen that is expected to last the user a week. A plurality of dispensers 60 can be designed to include 28, 29, 30 or 31 dispensers to provide a once daily dosage regimen that is expected to last the user a month. A plurality of dispensers 60 can be designed to include 56, 58, 60 or 62 dispensers to provide a twice daily dosage regimen that is expected to last the user a month. A plurality of dispensers 60 can be designed to include 84, 87, 90 or 93 dispensers to provide a thrice daily dosage regimen that is expected to last the user a month.

Each dispenser 50 can be sized and dimensioned to fold around a selected amount of cannabis plant material. In accordance with an example embodiment, each dispenser 50 is sized and dimensioned to fold around a prescribed amount of cannabis plant material, for example, to provide a prescribed dosage of at least one pharmacologically active cannabinoid. Generally, the mass of the cannabis plant material determines the effective amount of the at least one pharmacologically active agent released from the cannabis plant material. In accordance with an example embodiment, each dispenser is configured to retain a mass of plant material sufficient to deliver a unit dosage of the pharmacologically activate agent. In accordance with an example embodiment, the line perforations 60 comprise a visual dosing indicator. As used herein, a “visual dosing indicator” means that the line perforations are configured to indicate to a user the maximum dosage expected when the user separates one or more dispenser 50 from a plurality of dispensers 60 within a perimeter defined by the line perforations, i.e., the surface area of the dispensers 60 removed by tearing along the perimeter defined by the line perforations indicates the maximum amount of a user selected amount of cannabis plant material which can be retained within the dispenser by folding the removed dispensers 60 around the cannabis plant material and placing the dispensers 60 sublingually.

The plurality of dispensers 60 formed into the sheet 60 can be configured in any way. In accordance with an example embodiment, the sheet is configured in a rolled configuration (i.e., the sheet is packaged for distribution rolled up like a roll of toilet paper). In accordance with an example embodiment, the sheet is configured in a planar configuration.

Generally, the sheet 60 includes a first surface (shown) a second opposite surface (not shown), and a perimeter. In accordance with an example embodiment, the sheet includes at least one fastener 56 disposed proximal the perimeter on the first surface of each dispenser 50 in the sheet 60. The at least one fastener 56 is configured to secure at least one dispenser 50 to itself when the at least one dispenser is separated from the sheet 60 by tearing at least one line perforation, and folded over the cannabis plant material in such a way that the at least one dispenser contacts the at least one fastener.

In accordance with an example embodiment, the at least one fastener 56 is disposed proximal to the entire perimeter of each dispenser 50 in the sheet 60. In accordance with an example embodiment, the at least one fastener 56 is configured to secure the dispenser 50 to itself when the dispenser is separated from the sheet 60 by tearing at least one line perforation, and the dispenser is folded around cannabis plant material in such a way that the dispenser contacts the at least one fastener. In accordance with an example embodiment, the sheet 60 includes a plurality of fasteners 54 disposed proximal the perimeter on the first surface of each dispenser 50 in the sheet. In accordance with an example embodiment, the sheet 60 includes at least two of the plurality of fasteners 54 positioned opposite to each other proximal the perimeter. In accordance with an example embodiment, the at least one fastener 56 spans the entire portion proximal to a first edge of each dispenser in the sheet. In accordance with an example embodiment, the at least one fastener 56 spans the entire portion proximal to a second edge of each dispenser 54 in the sheet.

It should be appreciated that the at least one fastener 56 spanning the entire portion proximal to the first edge of each dispenser 50 in the sheet 60 and the at least one fastener 56 spanning the entire portion proximal to the second edge of each dispenser 50 in the sheet 60 can comprise the same material. Alternatively, the at least one fastener 56 spanning the entire portion proximal to the first edge of each dispenser 50 in the sheet 60 and the at least one fastener 56 spanning the entire portion proximal to the second edge of each dispenser 50 in the sheet 60 can comprise a different material.

In accordance with an example embodiment, the at least one fastener 56 is disposed proximal the perimeter on at least a portion of each side of the sheet 60. In accordance with an example embodiment, the at least one fastener 56 extends along the entire perimeter of the sheet 60 in a continuous manner. In accordance with an example embodiment, the at least one fastener 56 extends along the entire perimeter of the sheet 60 in a discontinuous manner.

Preferably, the at least one fastener 56 comprises a waxy material. Exemplary waxy materials include, without limitation, an edible wax selected from the group consisting of beeswax, candelilla wax, carnauba, ceresin wax, japan wax, microcrystalline wax, paraffin wax, shellac wax, sugarcane wax, and combinations and mixtures thereof. In accordance with an example embodiment, the plurality of dispensers 60 includes a coating as described herein (not shown) on at least a portion of the second surface of each dispenser 50 in the sheet 60. The coating 58 can be disposed on the entire second surface of each dispenser in the sheet, or alternatively only on a portion (e.g., approximately 10%, 15%, 20%, 25%, 30%, 35%, 40%, 45%, 50%, 55%, 60%, 65%, 70%, 75%, 80%, 85%, 90%, etc.) of the second surface of each dispenser 50 in the sheet 60.

Decarboxylated cannabis plant material generally has a bitter/sour taste which may result in a subject experiencing discomfort associated with clenching under the sides of the jaw when initially placing the decarboxylated cannabis plant material under the tongue due to hyper secretion and attendant pumping of saliva through the salivary glands in response to the taste (e.g., bitter and/or sour) of the decarboxylated cannabis plant material.

In an embodiment, the decarboxylated cannabis plant material is infused with a flavorant, for example, to improve a subject's taste sensation of the sublingual dosage form. The decarboxylated cannabis plant material can be infused with any suitable flavorant. Examples of such flavorants include, but are not limited to, synthetic flavor oils, flavoring aromatics, natural oils, extracts from plants other than cannabis, leaves, flowers, fruits, for example, anise oil, oil of wintergreen, clove oil, bay oil, anise oil, cocoa, cinnamon oil, eucalyptus, menthol, peppermint oil, thyme oil, cedar leaf oil, oil of nutmeg, oil of sage, oil of bitter almonds, cassia oil, vanilla, citrus oils including lemon, orange, lime, grapefruit, and fruit essences, including grape, apple, pear, peach, strawberry, raspberry, cherry, plum, and apricot, to name more than a few. Those skilled in the art will appreciate, however, that the decarboxylated cannabis plant material can be infused with other flavorants, such as coffee, smoke, and meat, for example, as the present invention is not intended to be limited in this manner.

Methods

The present invention contemplates methods of preparing a sublingual dosage form consisting of consisting essentially of or comprising decarboxylated cannabis plant material. The present invention also contemplates method of administering cannabinoids using a dispenser containing the sublingual dosage form. In an aspect, a method preparing a sublingual dosage form described herein comprises decarboxylating an amount of raw cannabis plant material sufficient to deliver an effective dose of at least one pharmacologically active cannabinoid present in the decarboxylated cannabis plant material into the systemic circulation of a subject when the decarboxylated cannabis plant material is placed into the sublingual cavity of the subject.

It is to be understood that decarboxylating the raw cannabis plant material activates at least one pharmacologically active cannabinoid present in the raw cannabis plant material. As used herein, “activates” refers to converting an inactive precursor into a pharmacologically active ingredient. In an embodiment, decarboxylating the raw cannabis plant material converts at least one inactive precursor acid present in the raw cannabis plant material into at least one pharmacologically active cannabinoid (e.g., conversion of cannabinolic acid precursor to THC).

In contrast to conventional methods of preparing cannabis dosage forms which subsequently process decarboxylated cannabis, for example by extracting the pharmacologically active THC from the plant material itself, decarboxylating the raw cannabis plant material is the only processing step that is required to prepare a sublingual dosage form in accordance with the present invention. That is, decarboxylating the raw cannabis plant material activates at least one pharmacologically active cannabinoid in the raw cannabis plant material so that the decarboxylated cannabis plant material is suitable for sublingual administration in its plant form in the absence of subsequent processing of the decarboxylated cannabis plant material. In particular, decarboxylating the raw cannabis plant material in accordance with the methods described herein activates at least one pharmacologically active cannabinoid present in the raw cannabis plant material so that the decarboxylated cannabis plant material can be used sublingually in the absence of subjecting the raw cannabis plant material or the decarboxylated cannabis plant material to a processing technique, such as admixing, condensation, decoction, distillation, expression, extraction selected from the group consisting of aqueous extraction, solvent extraction, ethanolic extraction, gas extraction, CO2 extraction, extrusion, filtration, isolation, maceration, percolation, precipitation, pulverization, purification, solvent casting, spheronization, sublimation, volatilization, and winterization. For the avoidance of doubt, the methods described herein prepare a sublingual dosage form comprising the decarboxylated cannabis plant material for immediate sublingual use to administer at least one pharmacologically active cannabinoid present in the decarboxylated cannabis plant material without having to subject the decarboxylated cannabis plant material to further processing, such as admixing, condensation, decoction, distillation, expression, extraction selected from the group consisting of aqueous extraction, solvent extraction, ethanolic extraction, gas extraction, CO2 extraction, extrusion, filtration, isolation, maceration, percolation, precipitation, pulverization, purification, solvent casting, spheronization, sublimation, volatilization, and winterization. It is to be understood that none of the above processing techniques are required by the methods, apparatuses or systems disclosed herein at any time during the preparation of a sublingual dosage form of the present invention.

The present invention contemplates any suitable method of decarboxylating an amount of raw cannabis plant material which results in the formation of an amount of at least one pharmacologically active cannabinoid in the resulting decarboxylated cannabis plant material effective for sublingual administration. The methods of decarboxylating raw (i.e., pre-decarboxylated) cannabis plant material described herein result in the formation of decarboxylated cannabis plant material that contains at least 70% of the maximum amount of pharmacologically active cannabinoids (e.g., tetrahydrocannabinol and cannabidiol) that could theoretically be present as a result of decarboxylation of the maximum amount of their pharmacologically inactive precursor acids (e.g., cannabinolic acid and cannabidiolic acid) present in the cannabis plant material (i.e., at least 70% of the pharmacologically inactive precursor acids present in the cannabis plant material are decarboxylated to their pharmacologically active cannabinoid form). Preferably, the methods of decarboxylating raw cannabis plant material described herein result in the formation of decarboxylated cannabis plant material contains at least 71%, at least 72%, at least 73%, at least 74%, 75%, at least 76%, at least 77%, at least 78%, at least 79%, at least 80%, at least 81%, at least 82%, at least 83%, at least 84%, at least 85%, at least 86%, at least 87%, at least 88%, or at least 89% of the maximum amount of pharmacologically active cannabinoids that could theoretically be present as a result of decarboxylation of the maximum amount of their pharmacologically inactive precursor acids present in the cannabis plant material. More preferably, the methods of decarboxylating raw cannabis plant material described herein result in the formation of decarboxylated cannabis plant material contains at least 90% of the maximum amount of pharmacologically active cannabinoids that could theoretically be present as a result of decarboxylation of the maximum amount of their pharmacologically inactive precursor acids present in the cannabis plant material. Even more preferably, the methods of decarboxylating raw cannabis plant material described herein result in the formation of decarboxylated cannabis plant material contains at least 91%, at least 92%, at least 93%, at least 94%, at least 95%, at least 96%, at least 97%, at least 98%, or at least 99% of the maximum amount of pharmacologically active cannabinoids that could theoretically be present as a result of decarboxylation of the maximum amount of their pharmacologically inactive precursor acids present in the cannabis plant material. Still even more preferably, the methods of decarboxylating raw cannabis plant material described herein result in the formation of decarboxylated cannabis plant material contains the maximum amount (i.e., 100% less any minor or negligible loss of product due to undesirable degradation (e.g., to cannabinol, oxidation/pyrolytic degradation)) of pharmacologically active cannabinoids that could theoretically be present as a result of decarboxylation of the maximum amount of their pharmacologically inactive precursor acids present in the cannabis plant material (i.e., at least 100% of the pharmacologically inactive precursor acids present in the cannabis plant material are decarboxylated to their pharmacologically active cannabinoid form regardless of minor or negligible loss of product due to undesirable reactions or unwanted degradation (e.g., degradation to cannabinol, oxidation/pyrolytic degradation)). Those skilled in the art will appreciate that one way in which the maximum amount of THC that could theoretically be available assuming full decarboxylation under ideal conditions can be calculated using the formula: Max THC=THC+THCA*0.877. Those skilled in the art will appreciate that the same formula can be used to calculate the Max CBD, e.g., by replacing THC and THCA with CBD and CBDA, respectively. For the avoidance of double, when calculating the maximum amount of THC that could theoretically be available using this formula the phrase “at least X % of the maximum amount of pharmacologically active cannabinoids that could theoretically be present as a result of decarboxylation of the maximum amount of their pharmacologically inactive precursor acids present in the cannabis plant material” refers to at least X % of the Max THC value calculated using the formula. The methods of decarboxylating raw cannabis plant material described herein result in the formation of decarboxylated cannabis plant material that contains cannabinol in an amount less than 0.5%, less than 0.4%, less than 0.3%, less than 0.2%, less than 0.1 total weight of the decarboxylated cannabis plant material. Preferably, the methods described herein result in the formation of decarboxylated cannabis plant material that contains cannabinol in an amount below quantifiable limits with respect to the total weight of the decarboxylated cannabis plant material. Preferably, decarboxylation of the amount of raw cannabis plant material results in the simultaneous pasteurization of the decarboxylated cannabis plant material, for example, to inactivate live pathogens (e.g., Aspergillus fungus) which would render the cannabis unsuitable for sublingual use or human consumption. The methods, apparatuses, and systems of the present invention are well adapted to simultaneously carry out decarboxylation and pasteurization of raw cannabis so that the resulting decarboxylated cannabis plant material is immediately suitable for suitable for sublingual administration, without requiring a pre- or post-decarboxylation pasteurization step, for example using an autoclave.

In some embodiments, decarboxylating the amount of raw cannabis plant material comprises heating an amount of raw cannabis plant material in an oxygen controlled environment at a temperature of from about 85° C. to about 125° C. for a period of from about 60 minutes to about 120 minutes, thereby decarboxylating the amount of raw cannabis plant material. Decarboxylating the amount of raw cannabis plant material in this manner results in the formation of decarboxylated cannabis plant material that contains at least 70% of the maximum amount of pharmacologically active cannabinoids that could theoretically be present as a result of decarboxylation of the maximum amount of their pharmacologically inactive precursor acids. In some embodiments, decarboxylating the amount of raw cannabis plant material comprises heating an amount of raw cannabis plant material in an oxygen controlled environment at a temperature of from about 90° C. to about 100° C. for a time period of at least 105 minutes. Selecting an amount of raw THC-enriched cannabis plant material and decarboxylating it in this manner results in the formation of decarboxylated cannabis plant material that contains at least 90% of the maximum amount of THC that could theoretically be present as a result of decarboxylation of the maximum amount of THCA present in the raw THC-enriched cannabis plant material.

In some embodiments, decarboxylating the amount of raw cannabis plant material comprises heating the amount of raw cannabis plant material in an oxygen controlled environment at a temperature of from about 105° C. to about 115° C. for a period of from about 20 to 60 minutes. Surprisingly and unexpectedly, work described herein demonstrates that decarboxylating an amount of raw cannabis plant material by heating the amount of raw cannabis plant material in an oxygen controlled environment at a temperature from about 105° C. to about 115° C. for a period of from about 20 to 60 minutes results in the formation of decarboxylated cannabis plant material that contains at least 90% of the maximum amount of THC and/or at least 80% of the maximum amount of CBD that could theoretically present as a result of decarboxylation of the maximum amount of both THCA and/or CBDA, respectively, present in the amount of raw cannabis plant material. As described more in Example 11, in contrast to the apparatus described in U.S. Pat. No. 4,279,824 which only achieves a decarboxylation completeness for THC of 64% when operated according to the parameters described therein (decarboxylation using boiling water as a heat source for about 60 minutes) and does not exceed a decarboxylation completeness of 67% even after 4 hours of decarboxylation using that apparatus, decarboxylating the cannabis plant material in this manner surprisingly and unexpectedly achieves at least 90% decarboxylation completeness for THC and at least 80% decarboxylation completeness for CBD within 20 to 60 minutes.

In some aspects, disclosed herein is a method of preparing a sublingual dosage form comprising decarboxylated cannabis plant material, the method comprising decarboxylating raw cannabis plant material at a temperature range suitable for and a period of time sufficient for the decarboxylated cannabis plant material to contain at least 70% of the maximum amount of at least one pharmacologically active cannabinoid that could theoretically be present as a result of decarboxylation of the maximum amount of the at least one pharmacologically active cannabinoid's inactive precursor present in the raw cannabis plant material.

In some embodiments, the at least one pharmacologically active cannabinoid is tetrahydrocannabinol and the at least one pharmacologically active cannabinoid's inactive precursor is cannabinolic acid. In some embodiments, the cannabinolic acid is present prior to decarboxylation in an amount from about 1% to about 40% total weight of the raw cannabis plant material. In some embodiments, the tetrahydrocannabinol is present after decarboxylation in an amount from about 1% to about 40% total weight of the decarboxylated cannabis plant material. In some embodiments, the temperature range is from about 85° to about 125° and the period of time is between 60 minutes and 120 minutes. In some embodiments, the decarboxylated cannabis plant material contains at least 90% of the maximum amount of tetrahydrocannabinol that could theoretically be present as a result of decarboxylation of the maximum amount of cannabinolic acid present in the raw cannabis plant material. In some instances, this can be achieved with a temperature range from about 90° to about 100° and a period of time of at least 105 minutes. In other instances, this can be achieved with a temperature range from about 105° to about 115° and a period of time between about 20 minutes and about 60 minutes.

In some embodiments, the at least one pharmacologically active cannabinoid is cannabidiol and the at least one pharmacologically active cannabinoid's inactive precursor is cannabidiolic acid. In some embodiments, the cannabidiolic acid is present prior to decarboxylation in an amount from about 1% to about 40% total weight of the raw cannabis plant material. In some embodiments, the cannabidiol is present after decarboxylation in an amount from about 1% to about 40% total weight of the decarboxylated cannabis plant material. In some embodiments, the temperature range is from about 85° to about 125° and the period of time is between 60 minutes and 120 minutes. In some embodiments, the decarboxylated cannabis plant material contains at least 80% of the maximum amount of cannabidiol that could theoretically be present as a result of decarboxylation of the maximum amount of cannabidiolic acid present in the raw cannabis plant material. In some instances, the temperature range is 105° C. to 115° C. and the period of time is between 30 minutes and 60 minutes. In some embodiments, the decarboxylated cannabis plant material contains at least 90% of the maximum amount of cannabidiol that could theoretically be present as a result of decarboxylation of the maximum amount of cannabidiolic acid present in the raw cannabis plant material. In some embodiments, this can be achieved with a temperature range from about 105° to about 115° and the period of time is between about 60 to 120 minutes.

It should be appreciated that the lower limits of the recited temperature ranges correspond to the upper limits of the recited time periods and that the upper limits of the recited temperature ranges correspond to the lower limits of the recited time periods.

In some embodiments, the at least one pharmacologically active cannabinoid comprises tetrahydrocannabinol and cannabidiol, and wherein the at least one pharmacologically active cannabinoid's inactive precursor is cannabinolic acid and cannabidiolic acid, respectively. In some embodiments, the cannabinolic acid is present prior to decarboxylation in an amount from about 1% to about 40% total weight of the raw cannabis plant material, and wherein the cannabidiolic acid is present prior to decarboxylation in an amount from about 1% to about 40% total weight of the raw cannabis plant material. In some embodiments, the tetrahydrocannabinol is present after decarboxylation in an amount from about 1% to about 40% total weight of the decarboxylated cannabis plant material, and wherein the cannabidiol is present after decarboxylation in an amount from about 1% to about 40% total weight of the decarboxylated cannabis plant material. In some embodiments, the temperature range is from about 85° to about 125° and the period of time is between 60 minutes and 120 minutes. In some embodiments, the decarboxylated cannabis plant material contains at least 90% of the maximum amount of tetrahydrocannabinol and at least 80% of the maximum amount of cannabidiol that could theoretically be present as a result of decarboxylation of the maximum amount of cannabinolic acid and cannabidiolic acid, respectively, present in the raw cannabis plant material. In some instances, this can be achieved with a temperature range from about 105° to about 115° and a period of time between about 20 minutes and about 60 minutes. In some embodiments, the decarboxylated cannabis plant material contains 100% of the maximum amount of tetrahydrocannabinol and at least 90% of the cannabidiol that could theoretically be present as a result of decarboxylation of the maximum amount of cannabinolic acid and cannabidiolic acid, respectively, present in the raw cannabis plant material. In some instances, this can be achieved with a temperature range from about 105° to about 115° and the period of time is between about 60 minutes and about 120 minutes. In some embodiments, an amount of cannabinol present in the dosage form is less than 0.5% of the total weight of the decarboxylated cannabis plant material.

The oxygen controlled environment may contain an initial amount of oxygen present consisting of atmospheric oxygen that enters the oxygen controlled environment when the amount of raw cannabis plant material enters the oxygen controlled environment. The initial amount of oxygen present in the oxygen controlled environment is generally not enough to cause significant oxidative degradation of the decarboxylated cannabis plant material. In an embodiment, the oxygen controlled environment permits the initial amount of oxygen present to escape while preventing atmospheric oxygen from entering the oxygen controlled environment during decarboxylation of the cannabis plant material, thereby minimizing oxidative degradation of the decarboxylated cannabis plant material. In an embodiment, the oxygen controlled environment displaces an amount of fluid (e.g., air) that entered the oxygen controlled environment upon placing the amount of raw cannabis plant material in the oxygen controlled environment to reduce the oxygen content in the oxygen controlled environment, without permitting additional atmospheric oxygen to enter the oxygen controlled environment.

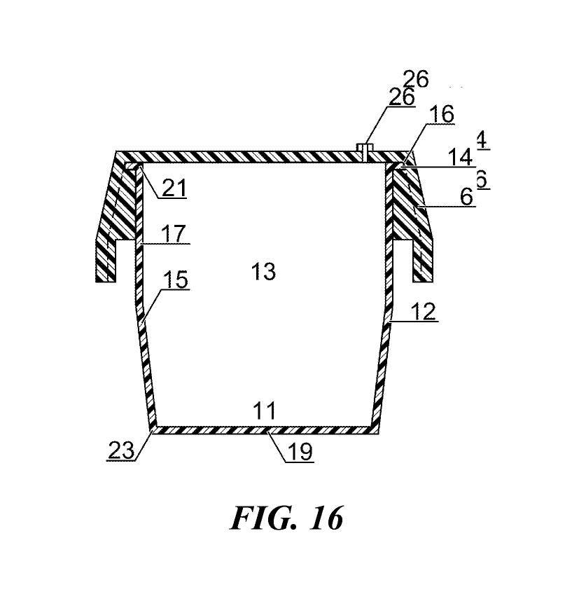

The present invention contemplates any oxygen controlled environment that limits the initial oxygen content present in the oxygen controlled environment or permits the initial oxygen content present in the oxygen controlled environment to be reduced. In an embodiment, the oxygen controlled environment comprises an apparatus or system described herein. In an embodiment, as is shown in FIG. 16, the oxygen controlled environment comprises a container suspended from and sealably engaged to a cap as described herein. In an embodiment, the oxygen controlled environment comprises an airtight container (e.g., a vacuum sealed container) as described herein. In an embodiment, the oxygen controlled environment comprises a vacuum sealed bag as described herein. In an embodiment, the oxygen controlled environment comprises a microwave safe zip-lock bag. It should be appreciated that the oxygen controlled environment may be a combination of oxygen controlled environments described herein. For example, an amount of raw cannabis plant material to be decarboxylated can be placed in a first oxygen controlled environment (e.g., a vacuum sealed bag), and then the first oxygen controlled environment containing the amount of raw cannabis plant material can be placed directly into a second oxygen controlled environment (e.g., such as the oxygen controlled environment depicted in FIG. 16) for subsequent decarboxylation of the cannabis to be carried out. Other oxygen controlled environments suitable for use in the methods of the present invention would be apparent to the skilled artisan.

In an embodiment, the heating is performed utilizing a heat source external to the oxygen controlled environment. In such embodiments, the method contemplates maximizing heat transmission from the heat source external to the oxygen controlled environment to the amount of raw cannabis plant material in the oxygen controlled environment.

In an embodiment, decarboxylating the amount of raw cannabis plant material comprises: (i) placing the amount of raw cannabis plant material into an oxygen controlled environment; (ii) sealing the oxygen controlled environment, wherein sealing the oxygen controlled environment limits the oxygen content in the oxygen controlled environment to an initial amount of atmospheric oxygen present in air that enters the oxygen controlled environment during step (i) prior to sealing the oxygen controlled environment; and (iii) heating the oxygen controlled environment indirectly with a heat source outside the oxygen controlled environment at a temperature of from about 85° C. to about 125° C. for a period of from about 60 minutes to about 120 minutes to decarboxylate the amount of raw cannabis plant material inside the oxygen controlled environment; wherein the oxygen controlled environment minimizes oxidative degradation of the decarboxylated cannabis plant material by permitting the initial amount of atmospheric oxygen present in the oxygen controlled environment to escape while preventing atmospheric oxygen external to the oxygen controlled environment from entering the oxygen controlled environment during decarboxylation of the amount of raw cannabis plant material.

In an embodiment, decarboxylating the amount of raw cannabis plant material comprises: (i) placing the amount of raw cannabis plant material into an oxygen controlled environment; (ii) sealing the oxygen controlled environment, wherein sealing the oxygen controlled environment limits the oxygen content in the oxygen controlled environment to an initial amount of atmospheric oxygen present in air that enters the oxygen controlled environment during step (i) prior to sealing the oxygen controlled environment; and (iii) heating the oxygen controlled environment indirectly with a heat source outside the oxygen controlled environment at a temperature of from about 90° C. to about 100° C. for a period of at least about 105 minutes to decarboxylate the amount of raw cannabis plant material inside the oxygen controlled environment; wherein the oxygen controlled environment minimizes oxidative degradation of the decarboxylated cannabis plant material by permitting the initial amount of atmospheric oxygen present in the oxygen controlled environment to escape while preventing atmospheric oxygen external to the oxygen controlled environment from entering the oxygen controlled environment during decarboxylation of the amount of raw cannabis plant material.

In an embodiment, decarboxylating the amount of raw cannabis plant material comprises: (i) placing the amount of raw cannabis plant material into an oxygen controlled environment; (ii) sealing the oxygen controlled environment, wherein sealing the oxygen controlled environment limits the oxygen content in the oxygen controlled environment to an initial amount of atmospheric oxygen present in air that enters the oxygen controlled environment during step (i) prior to sealing the oxygen controlled environment; and (iii) heating the oxygen controlled environment indirectly with a heat source outside the oxygen controlled environment at a temperature of from about 105° C. to about 115° C. for a period of from about 20 minutes to about 60 minutes to decarboxylate the amount of raw cannabis plant material inside the oxygen controlled environment; wherein the oxygen controlled environment minimizes oxidative degradation of the decarboxylated cannabis plant material by permitting the initial amount of atmospheric oxygen present in the oxygen controlled environment to escape while preventing atmospheric oxygen external to the oxygen controlled environment from entering the oxygen controlled environment during decarboxylation of the amount of raw cannabis plant material.

In some embodiments, decarboxylating the amount of raw cannabis plant material does not comprise exposing the oxygen controlled environment to steam created by water boiling in proximity to the oxygen controlled environment for up to 60 minutes.

In an embodiment, the method includes, prior to step (ii), maximizing heat transmission through the oxygen controlled environment to the amount of raw cannabis plant material inside the oxygen controlled environment. The prevent invention contemplates any suitable technique for maximizing heat transmission through the oxygen controlled environment. In an embodiment, maximizing heat transmission through the oxygen controlled environment to the amount of raw cannabis plant material inside the oxygen controlled environment comprises pressing the amount of raw cannabis plant material present inside the oxygen controlled environment against one or more internal surfaces of the oxygen controlled environment. In an embodiment, the amount of raw cannabis plant material is at least pressed against a surface of the oxygen controlled environment that is nearest to the external heat source.

In an embodiment, the method includes, prior to step (ii), reducing oxygen content present in the oxygen controlled environment. The present invention contemplates any suitable technique for reducing oxygen present in the oxygen controlled environment before the oxygen controlled environment is sealed. In an embodiment, reducing oxygen content present in the oxygen controlled embodiment comprises limiting the amount of initial oxygen content present in the oxygen controlled environment by sealing off the amount of raw cannabis plant material present in the oxygen controlled environment from the ambient air, for example by utilizing a disc described herein, and causing at least a portion of the initial oxygen content present in the oxygen controlled environment to escape, for example, by pressing a disc described herein against the amount of raw cannabis plant material present in the oxygen controlled environment. It should be appreciated that pressing the disc against the amount of raw cannabis plant material compresses the cannabis plant material against an interior surface of the oxygen controlled environment, thereby maximizing heat transmission to the cannabis plant material.

In an embodiment, reducing oxygen content present in the oxygen controlled environment comprises disposing the amount of raw cannabis plant material into an airtight container (e.g., a vacuum sealed bag) prior to being placed in the oxygen controlled environment. When placed into the airtight container, the exposure to ambient air can be eliminated almost entirely, for example, where the container is vacuum sealed prior to being placed into the oxygen controlled environment. It should be appreciated that employing a vacuum-sealed container restricts exposure to ambient air both during and after the cannabis plant material is decarboxylated. Any airtight container can be used. In an embodiment, the airtight container includes a container composed of a heat resistant high quality borosilicate glass, and a lid composed of a non-corrosive metal material with a silicone button disposed in the lid that creates an airtight seal when the silicone button is pressed. As will be appreciated by those skilled in the art, the container and the lid can be sized and dimensioned in any way that permits an amount of raw cannabis plant material to be placed within the container with the lid vacuum sealed to the container to reduce the oxygen content present in the oxygen controlled environment. An exemplary such airtight container is the commercially available square airtight glass container with 18/8 stainless steel lid (Onyx, Cranbury, N.J.). In an embodiment, the airtight container includes a container constructed from a nonporous material (e.g., glass) and a silicone-rimmed, self-venting lid (e.g., constructed from glass) that creates an airtight seal when the lid is secured to the container for reducing the oxygen content present. As will be appreciated by those skilled in the art, the container and the lid can be sized and dimensioned in any way that permits an amount of raw cannabis plant material to be placed within the container with the lid sealed to the container to reduce the oxygen content present in the oxygen controlled environment. Other suitable airtight containers are apparent to the skilled artisan.

In an embodiment, the method includes shaping or forming the decarboxylated cannabis plant material, for example, to facilitate maximum absorption of the cannabinoids present in the decarboxylated cannabis plant material by increasing the surface area of the decarboxylated cannabis plant material in direct contact with the sublingual mucosa. The present invention contemplates shaping or forming the decarboxylated cannabis plant material in any way which would facilitate administration of the decarboxylated cannabis plant material sublingually. In an embodiment, the decarboxylated cannabis plant material is flattened out (e.g., into a “fanned” configuration) before being administered sublingually. In an embodiment, the decarboxylated cannabis plant material is spread out before being administered sublingually. In an embodiment, the decarboxylated plant material is flattened out and spread out before being administered sublingually. Without wishing to be bound by theory, it is believed that spreading the plant material out and/or flattening the plant material out allows the cannabis to fit comfortably in a subject's oral cavity, prevents excess saliva buildup, and permits the user to speak, eat, or drink during administration of the sublingual dosage form of the present invention. Those skilled in the art will appreciate that shaping or forming the decarboxylated cannabis plant material can be achieved in a variety of ways during the preparation of a sublingual dosage form comprising decarboxylated cannabis plant material. For example, a sublingual dosage form comprising the decarboxylated cannabis plant material can be preshaped or preformed and individually packaged for distribution.

In an embodiment, the method includes infusing the decarboxylated cannabis plant material with a flavorant, such as a flavorant described herein. Infusing the decarboxylated cannabis with a flavorant can be achieved in a variety of ways, for example, by contacting the decarboxylated cannabis plant material with an amount of flavorant and for a period of time sufficient to infuse the decarboxylated cannabis plant material with the flavoring. Preferably, the decarboxylated cannabis plant material is infused with a flavorant in a way that minimizes oxidative degradation of pharmacologically active cannabinoids in the cannabis. In an embodiment, the decarboxylated cannabis plant material is infused with flavorant prior to decarboxylating the cannabis plant material. For example, an amount of raw cannabis plant material can be contacted with flavorant in an inert, oxygen controlled environment for a period of time sufficient for the raw cannabis plant material to be infused with the flavorant, and the raw cannabis plant material infused with flavorant can be decarboxylated in accordance with the methods described herein. In an embodiment, the decarboxylated cannabis plant material is infused with flavorant during decarboxylation of the raw cannabis plant material. For example, an amount of raw cannabis plant material can be placed into an oxygen controlled environment along with the flavorant, and decarboxylation of the raw cannabis plant material can be carried out so that the resulting decarboxylated cannabis plant material is infused with the flavorant. In an embodiment, the decarboxylated cannabis plant material can be infused with flavorant after decarboxylating the raw cannabis plant material. For example, decarboxylated cannabis plant material can be placed into a storage medium (e.g., an inert storage medium) in contact with the flavorant until the decarboxylated cannabis plant material is infused with the flavorant.

In an embodiment, the method includes placing the decarboxylated cannabis plant material into a dispenser as described herein.

In an embodiment, the method includes placing the decarboxylated cannabis plant material into a storage medium, for example an inert storage medium, to prevent at least one pharmacologically active cannabinoid present in the decarboxylated cannabis plant material from degradation. In an embodiment, the storage medium minimizes or prevents the decarboxylated cannabis plant material from being exposed to oxygen. In an embodiment, the storage medium minimizes or prevents the decarboxylated cannabis plant material from being exposed to light.

Methods of Use

Aspects of the disclosure involve using a dispenser (e.g., comprising a wrapping 52 as described herein) for sublingually administering at least one pharmacologically active cannabinoid from decarboxylated cannabis plant material. Generally, administering at least one pharmacologically active cannabinoid (e.g., THC) using a wrapping described herein involves folding the wrapping 52 around the decarboxylated cannabis plant material 51, and placing the wrapping 52 under the tongue with the decarboxylated cannabis plant material inside the wrapping 52.

In some aspects, the disclosure provides a method of administering at least one pharmacologically active cannabinoid to a subject in need thereof. The method includes providing a wrapping described herein; folding the wrapping around the decarboxylated cannabis plant material; and placing the wrapping in the subject's sublingual cavity.

Generally, the wrapping is placed in the subject's sublingual cavity for a period of time sufficient to release an effective amount of the at least one pharmacologically active cannabinoid into the subject's sublingual cavity, where it is absorbed into the subject's systemic circulation. The effective amount of the at least one pharmacologically active cannabinoid actually released into the sublingual cavity is a function of the residence time of the dispenser 50 in a user's sublingual cavity. It is expected that the amount of the at least one pharmacologically active cannabinoid released into the subject's sublingual cavity generally increases as the residence time of the wrapping in the subject's sublingual cavity increases. Table 2 below lists exemplary release profiles of cannabinoids from the dispenser 50 depending on the residence time of the dispenser in a user's sublingual cavity.

TABLE 2
Exemplary Cannabinoid Release Profiles

| | | Cannabinoids Released | Cannabinoids Remaining |
|---|---|---|---|
| Dispenser | | From Sublingual Dosage | in Sublingual Dosage |
| Residence Time | | Form in Dispenser | Form in Dispenser |
| 1 | minute | 5% to 100% | 0% to 95% |
| 5 | minutes | 25% to 100% | 0% to 75% |
| 10 | minutes | 50% to 100% | 0% to 50% |
| 15 | minutes | 75% to 100% | 0% to 25% |

Based on the exemplary release profiles featured in Table 2, the skilled artisan will glean that the amount of pharmacologically active cannabinoids remaining in the sublingual dosage form of the present invention (e.g., decarboxylated cannabis plant material retained in the dispenser), is inversely proportionate to the residence time, i.e., the longer the residence time, the less cannabinoid content remaining in the sublingual dosage form. Accordingly, a dispenser 50 of the present invention can deliver a first dosage comprising an effective amount of at least one pharmacologically active cannabinoid into a user's sublingual cavity by retaining the dispenser 50 in the user's sublingual cavity with the decarboxylated cannabis plant material contained within it, and deliver a second dosage comprising an effective amount of at least one pharmacologically active cannabinoid into a user's gastrointestinal tract by swallowing the dispenser 50 with the decarboxylated cannabis plant material inside it after the first dosage is delivered.

In accordance with an example embodiment, the method of administering at least one pharmacologically active cannabinoid includes ingesting the wrapping 52 with the decarboxylated cannabis plant material inside the wrapping. Based on the exemplary residence times listed in Table 2, a user can obtain a larger first dosage sublingually and a smaller dosage orally by increasing the residence time of dispenser 50 in the user's sublingual cavity before swallowing the dispenser 50. Similarly, a user can obtain a smaller second dosage sublingually and a larger dosage orally by decreasing the residence time of dispenser 50 in the user's sublingual cavity before swallowing the dispenser 50.

In accordance with an example embodiment, the dispenser 50 is retained in a user's sublingual cavity for a period of 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, or 15 minutes, or more, to obtain a first dosage of at least one pharmacologically active cannabinoid before swallowing the dispenser with the decarboxylated cannabis plant material inside it to obtain a second dosage of at least one pharmacologically active cannabinoid. In accordance with an example embodiment, the dispenser 50 is retained in a user's sublingual cavity for a period of 16 minute, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes, or 30 minutes, or more, to obtain a first dosage of at least one pharmacologically active cannabinoid before swallowing the dispenser with the decarboxylated cannabis plant material inside it to obtain a second dosage of at least one pharmacologically active cannabinoid.

In some aspects, a method of administering at least one pharmacologically active cannabinoid to a subject in need thereof includes providing a wrapping comprising at least one aperture configured to retain decarboxylated cannabis plant material inside the wrapping while permitting an effective amount of at least one pharmacologically active cannabinoid to be released into a subject's sublingual cavity when the wrapping is folded around the decarboxylated cannabis plant material and placed in the subject's sublingual cavity, instructing the subject to fold the wrapping around the decarboxylated cannabis plant material; and instructing the subject to place the wrapping in the subject's sublingual cavity.

In some aspects, a method of administering at least one pharmacologically active cannabinoid to a subject in need thereof includes providing a wrapping comprising a plurality of apertures configured to retain decarboxylated cannabis plant material inside the wrapping while permitting an effective amount of at least one pharmacologically active cannabinoid to be released into a subject's sublingual cavity when the wrapping is folded around the decarboxylated cannabis plant material and placed in the subject's sublingual cavity; instructing the subject to fold the wrapping around the decarboxylated cannabis plant material; and instructing the subject to place the wrapping in the subject's sublingual cavity.

The subject can instructed to place the wrapping in the subject's sublingual cavity for a period of time sufficient to release the effective amount of the at least one pharmacologically active cannabinoid into the subject's sublingual cavity. For example, based on a prescribed dosage of at least one pharmacologically active cannabinoid, a user is instructed to select an amount of cannabis plant material which is sufficient to deliver the prescribed dosage and fold the wrapping around the decarboxylated cannabis plant material, and place the wrapping in their sublingual cavity for a period of time during which the effective amount of the at least one pharmacologically active cannabinoid is released into the subject's sublingual cavity. The skilled artisan will be able to determine the appropriate amount of cannabis and residence time, for example, based on the information provided in Tables 1 and 2.

The subject can be instructed to ingest the wrapping with the decarboxylated cannabis plant material inside the wrapping.

Aspects of the disclosure involve using a dispenser (e.g., comprising a wrapping 52 as described herein) for sublingually administering at least one pharmacologically active cannabinoid from decarboxylated cannabis plant material. Generally, administering at least one pharmacologically active cannabinoid (e.g., THC) using a wrapping described herein involves folding the wrapping 52 around the decarboxylated cannabis plant material 51, and placing the wrapping 52 under the tongue with the decarboxylated cannabis plant material inside the wrapping 52.

In some aspects, the disclosure provides a method of administering at least one pharmacologically active cannabinoid to a subject in need thereof. The method includes providing a wrapping described herein; folding the wrapping around the decarboxylated cannabis plant material; and placing the wrapping in the subject's sublingual cavity.

Generally, the wrapping 52 is placed in the subject's sublingual cavity for a period of time sufficient to release an effective amount of the at least one pharmacologically active cannabinoid into the subject's sublingual cavity, where it is absorbed into the subject's systemic circulation. The effective amount of the at least one pharmacologically active cannabinoid actually released into the sublingual cavity is a function of the residence time of the dispenser 50 in a user's sublingual cavity. It is expected that the amount of the at least one pharmacologically active cannabinoid released into the subject's sublingual cavity generally increases as the residence time of the wrapping 52 in the subject's sublingual cavity increases. Table 2 below lists exemplary release profiles of cannabinoids from the dispenser 50 depending on the residence time of the dispenser in a user's sublingual cavity.

TABLE 2
Exemplary Cannabinoid Release Profiles

| | | Cannabinoids Released | Cannabinoids Remaining |
|---|---|---|---|
| Dispenser | | From Sublingual Dosage | in Sublingual Dosage |
| Residence Time | | Form in Dispenser | Form in Dispenser |
| 1 | minute | 5% to 100% | 0% to 95% |
| 5 | minutes | 25% to 100% | 0% to 75% |
| 10 | minutes | 50% to 100% | 0% to 50% |
| 15 | minutes | 75% to 100% | 0% to 25% |

Based on the exemplary release profiles featured in Table 2, the skilled artisan will glean that the amount of pharmacologically active cannabinoids remaining in the sublingual dosage form of the present invention (e.g., decarboxylated cannabis plant material retained in the dispenser), is inversely proportionate to the residence time, i.e., the longer the residence time, the less cannabinoid content remaining in the sublingual dosage form. Accordingly, a dispenser 50 of the present invention can deliver a first dosage (e.g., sublingually) comprising an effective amount of at least one pharmacologically active cannabinoid into a user's sublingual cavity by retaining the dispenser 50 in the user's sublingual cavity with the decarboxylated cannabis plant material contained within it, and deliver a second dosage (e.g., orally) comprising an effective amount of at least one pharmacologically active cannabinoid into a user's gastrointestinal tract by swallowing the dispenser 50 with the decarboxylated cannabis plant material inside it after the first dosage is delivered.

In accordance with an example embodiment, the method of administering at least one pharmacologically active cannabinoid includes ingesting the wrapping 52 with the decarboxylated cannabis plant material inside the wrapping. Based on the exemplary residence times listed in Table 2, a user can obtain a larger first dosage sublingually and a smaller dosage orally by increasing the residence time of dispenser 50 in the user's sublingual cavity before swallowing the dispenser 50. Similarly, a user can obtain a smaller second dosage sublingually and a larger dosage orally by decreasing the residence time of dispenser 50 in the user's sublingual cavity before swallowing the dispenser 50.

In accordance with an example embodiment, the dispenser 50 is retained in a user's sublingual cavity for a period of 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, or 15 minutes, or more, to obtain a first dosage of at least one pharmacologically active cannabinoid before swallowing the dispenser with the decarboxylated cannabis plant material inside it to obtain a second dosage of at least one pharmacologically active cannabinoid. In accordance with an example embodiment, the dispenser 50 is retained in a user's sublingual cavity for a period of 16 minute, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes, or 30 minutes, or more, to obtain a first dosage of at least one pharmacologically active cannabinoid before swallowing the dispenser with the decarboxylated cannabis plant material inside it to obtain a second dosage of at least one pharmacologically active cannabinoid.

In some aspects, a method of administering at least one pharmacologically active cannabinoid to a subject in need thereof includes providing a wrapping comprising at least one aperture configured to retain decarboxylated cannabis plant material inside the wrapping while permitting an effective amount of at least one pharmacologically active cannabinoid to be released into a subject's sublingual cavity when the wrapping is folded around the decarboxylated cannabis plant material and placed in the subject's sublingual cavity, instructing the subject to fold the wrapping around the decarboxylated cannabis plant material; and instructing the subject to place the wrapping in the subject's sublingual cavity.

In some aspects, a method of administering at least one pharmacologically active cannabinoid to a subject in need thereof includes providing a wrapping comprising a plurality of apertures configured to retain decarboxylated cannabis plant material inside the wrapping while permitting an effective amount of at least one pharmacologically active cannabinoid to be released into a subject's sublingual cavity when the wrapping is folded around the decarboxylated cannabis plant material and placed in the subject's sublingual cavity; instructing the subject to fold the wrapping around the decarboxylated cannabis plant material; and instructing the subject to place the wrapping in the subject's sublingual cavity.

The subject can instructed to place the wrapping in the subject's sublingual cavity for a period of time sufficient to release the effective amount of the at least one pharmacologically active cannabinoid into the subject's sublingual cavity. For example, based on a prescribed dosage of at least one pharmacologically active cannabinoid, a user is instructed to select an amount of cannabis plant material which is sufficient to deliver the prescribed dosage and fold the wrapping around the decarboxylated cannabis plant material, and place the wrapping in their sublingual cavity for a period of time during which the effective amount of the at least one pharmacologically active cannabinoid is released into the subject's sublingual cavity. The skilled artisan will be able to determine the appropriate amount of cannabis and residence time, for example, based on the information provided in Tables 1 and 2.

The subject can be instructed to ingest the wrapping with the decarboxylated cannabis plant material inside the wrapping.

Apparatus

In an aspect, the present invention provides an oxygen controlled environment for minimizing oxidative degradation of cannabinoids present in decarboxylated or decarboxylating cannabis plant material while preparing a sublingual dosage form of the present invention. FIG. 16 shows an example embodiment of an oxygen controlled environment. With reference now to FIG. 16, the oxygen controlled environment 11 includes a container 12 and a cap 6 configured to sealably engage the container 12. The cap 6 is shown in snap-fittable engagement with container 12. The container 12 can be suspended from cap 6 by snap-fittable engagement of circumferential rim 14 of container 12 with corresponding recess 16 disposed as indicated within cap 6. Those skilled in the art will appreciate that although the mechanism of releasably engaging cap 6 and container 12 is shown, it is to be understood that alternate engagement mechanisms may be employed, such as regularly spaced projections radiating inwardly from the inner surface of cap 6 to mate with corresponding detents disposed in the outer upper surface of container 12. The disclosure is therefore merely illustrative of such engagement mechanisms, and the invention is therefore not limited to the specific construction illustrated herein. The container 12 has a receptacle 13, and an impervious barrier 15 which prevents fluid (e.g., air) from penetrating the container 12, thereby minimizing oxygen present in the oxygen controlled environment 11. The impervious barrier IS includes a wall portion 17 and a base portion 19. The wall portion 17 extends circumferentially from a first end 21, which is open, to a second end 23, which is closed by the base portion 19.

The container 12 and cap 6 can be constructed of a variety of materials which are suitable for providing an oxygen controllable environment 11, including, but not limited to, plastic, glass, silicone, food-grade butyl rubber, latex, aliphatic polyesters, natural rubber, metal, metal foils, polytetrafluoroethylene, biopolymers such as liquid wood, modified casein, polyhydroxyalkanoate polyesters, including polyhydroxybutrate, polyhydroxyvalerate, polylactic acid, starch based polyesters, keratin processed with methyl acrylate, hemp polymers, hemp plastic, hemp composite polymers, and combinations thereof. The container 12 is preferably constructed of a cylindrical shape, as shown in FIG. 16. As is shown in FIG. 16, at least a portion of the wall portion 17 of the impervious barrier 15 can be designed to taper inwardly as the wall portion 17 extends toward the base portion 19, for example, so that an amount of raw cannabis plant material to be decarboxylated in the oxygen controlled environment 11 can aggregate together in the receptacle 13, for example, to ensure consistent heating of the amount of raw cannabis plant material in the oxygen controlled environment 11. Those skilled in the art will appreciate that the length of the wall portion 17 of the impervious barrier 15 which tapers inwardly can vary, as the invention is not intended to be limited in this manner. It should also be appreciated, however, that the wall portion 17 can be designed so that the wall portion 17 does not taper.

As is shown in FIG. 16, the cap 6 can be configured with a valve 26, such as a one-way valve that allows fluid (e.g., air) to escape the oxygen controlled environment 11 without permitting fluid (e.g., air) to enter the oxygen controlled environment through the valve 26. The valve 26 relieves pressure while heating contents with the apparatus by allowing fluid to escape the container 12. The pressure relieved by the valve 6 helps to maintain the cap 6 sealably engaged to the container 12 during operation. In an embodiment, the valve 26 prevents air from entering the container, thereby minimizing oxygen present in the oxygen controlled environment 11.

Those skilled in the art will appreciate that an oxygen controlled environment 11 can be configured in other ways than as depicted in FIG. 16. In an embodiment, the oxygen controlled environment comprises an airtight container, as described herein.

In operation, an amount of raw cannabis plant material (not shown) can be placed into the receptacle 13 of oxygen controlled environment 11, and then the receptacle 13 can be heated in accordance with the methods described herein, for example, utilizing a heat source (e.g., steam) external to the oxygen controlled environment 11. In an embodiment, the oxygen controlled environment 11 can include a disc 30 for reducing oxygen as described herein.

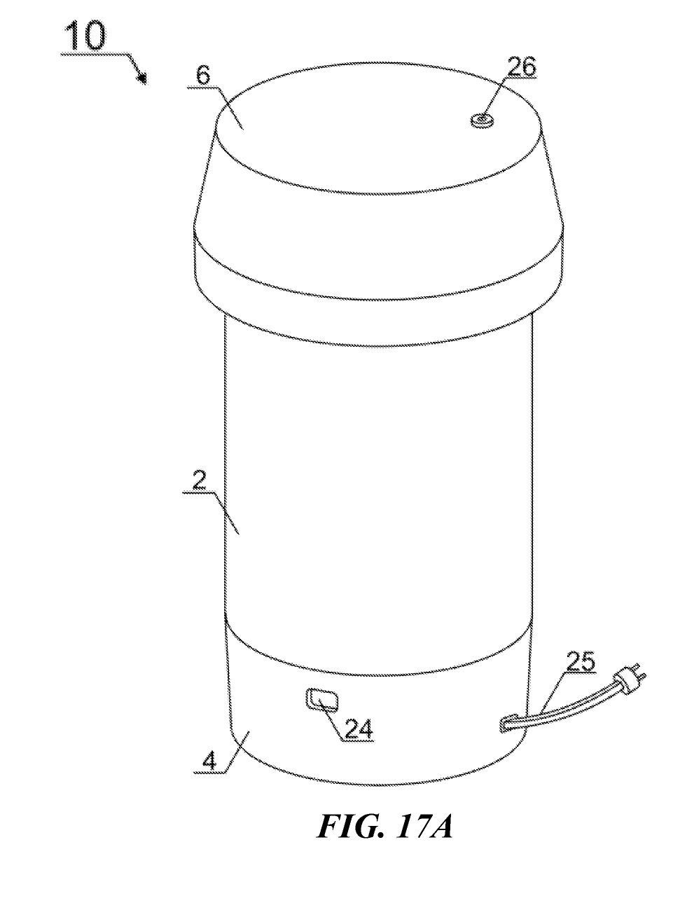

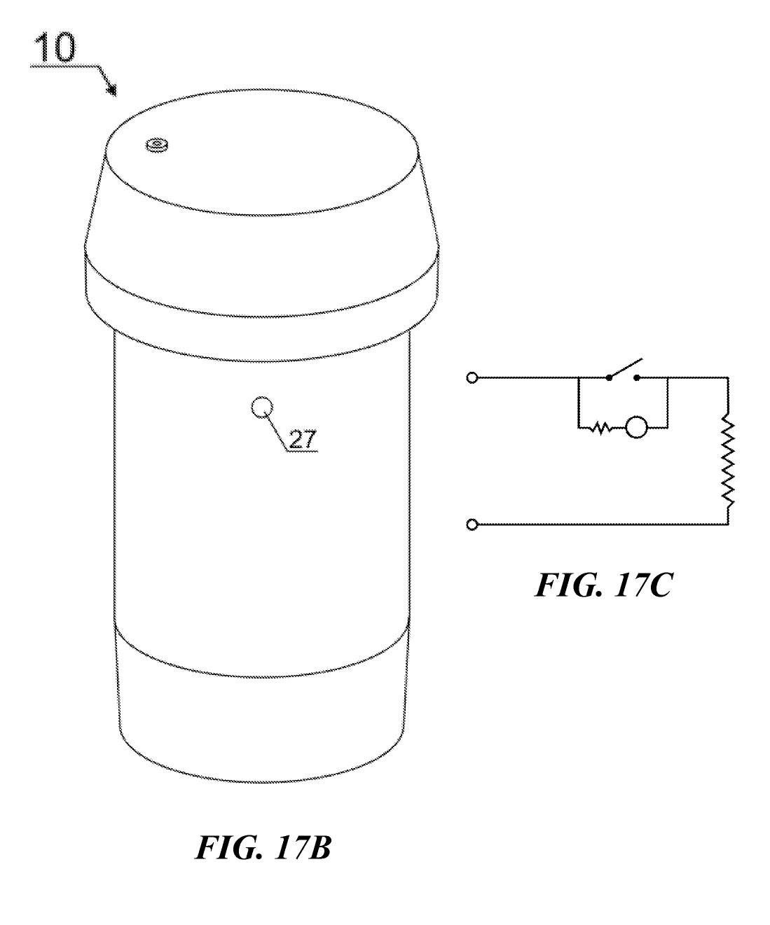

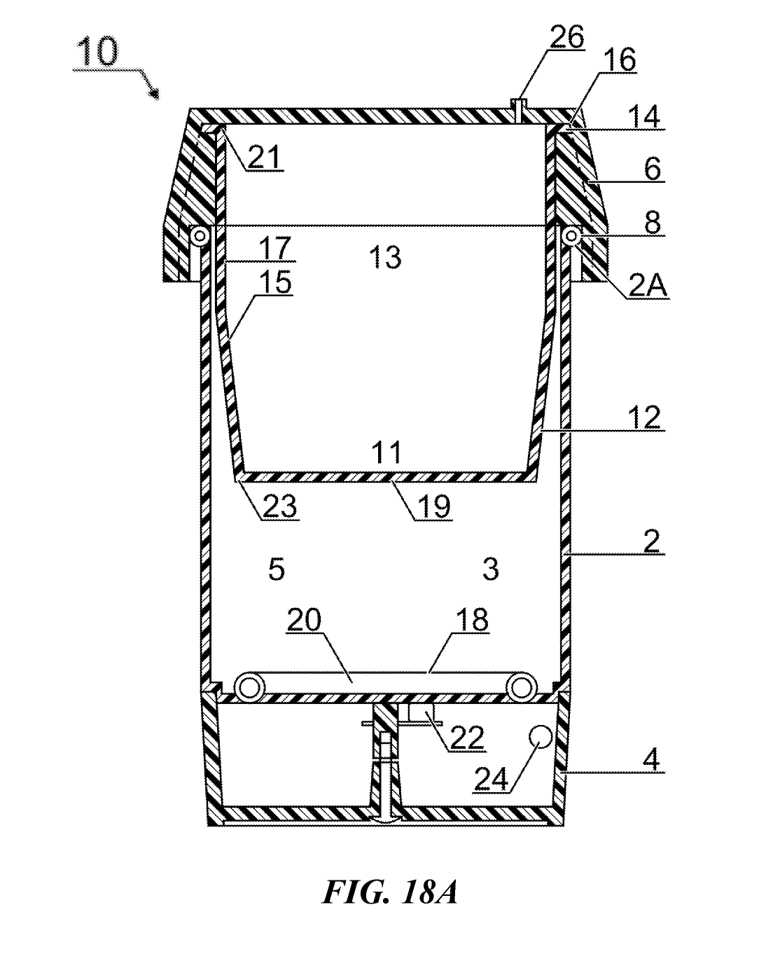

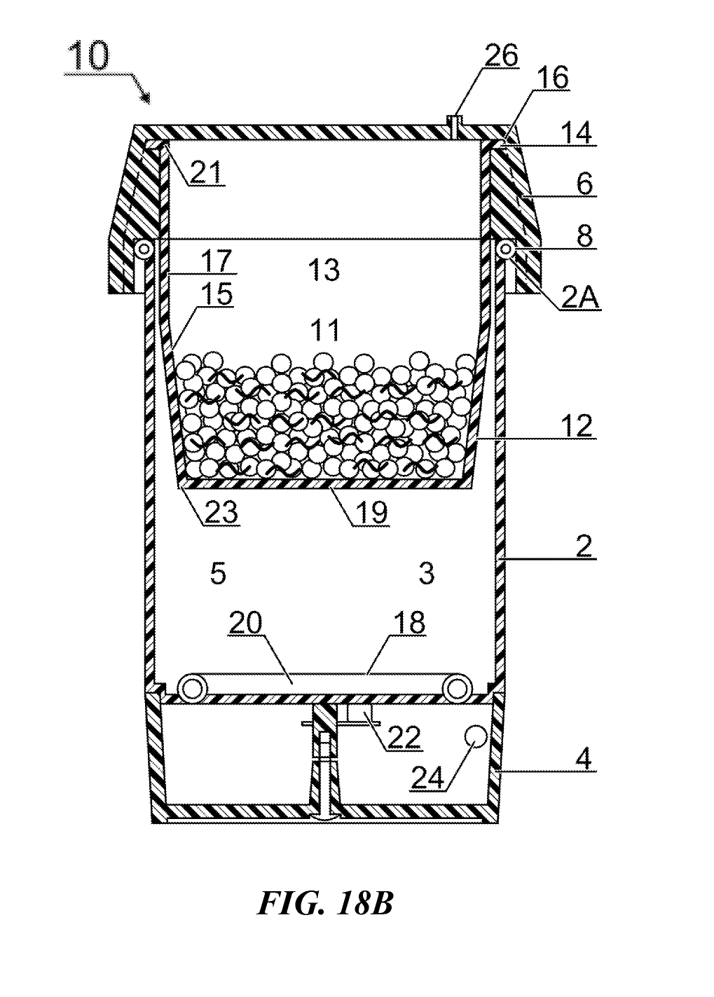

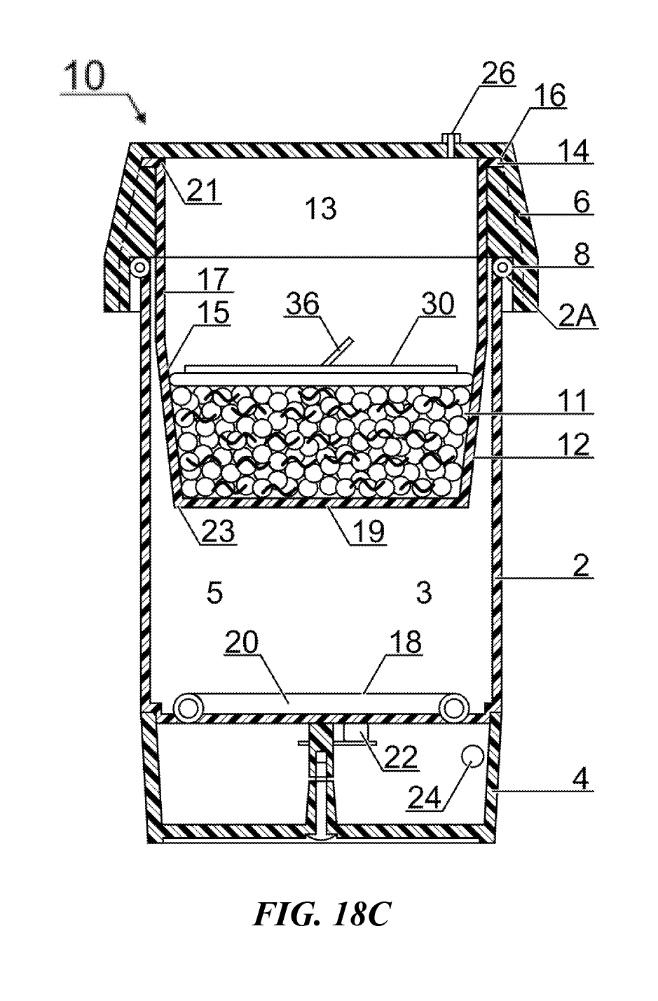

In an aspect, the present invention provides an apparatus for heating contents in an oxygen controlled environment, e.g., to prepare a sublingual dosage form consisting of, consisting essentially of, or comprising decarboxylated cannabis plant material. FIGS. 17A and 17B illustrate a first side perspective view (FIG. 17A) and a second side perspective view (FIG. 17B) of an apparatus for heating contents in an oxygen controlled environment, for example, to decarboxylate an amount of raw cannabis plant material sufficient to prepare a sublingual dosage form of the present invention. Referring now to FIG. 18A, there is shown a cross-sectional view of an exemplary embodiment of an apparatus 10 for heating contents in an oxygen controlled environment 11, which includes a first container 2 providing a temperature controllable environment 3, a cap 6 configured to sealably engage the first container 2, and a second container 12 configured to provide an oxygen controlled environment 11 for limiting the exposure of contents in the second container 12 to oxygen. The first container 2 has a first receptacle 5 configured to retain a fluid medium (not shown). The second container 12 has a second receptacle 13, and an impervious barrier 15 which prevents fluid present in the first container 2 from penetrating the second container 12. The impervious barrier 15 includes a wall portion 17 and a base portion 19. The wall portion 17 extends circumferentially from a first end 21, which is open, to a second end 23, which is closed by the base portion 19.

The first container 2 can be constructed of a variety of materials which are suitable for providing a temperature controllable environment 3, including, but not limited to, plastic, glass, silicone, food-grade butyl rubber, latex, aliphatic polyesters, natural rubber, metal, metal foils, polytetrafluoroethylene, biopolymers such as liquid wood, modified casein, polyhydroxyalkanoate polyesters, including polyhydroxybutrate, polyhydroxyvalerate, polylactic acid, starch based polyesters, keratin processed with methyl acrylate, hemp polymers, hemp plastic, hemp composite polymers, and combinations thereof. The first container 2 is preferably constructed of a cylindrical shape as shown in FIG. 18A. The first container 2 is open at its upper end to receive the second container 12, and is closed at its lower end by a base 4. A cap 6 is configured to be fitted over a relieved upper edge 2A of the container in a position to be sealed by a sealing element 8. The sealing element 8, for example, can be an O-ring disposed within the perimeter of cap 6 or a bead-like lip as shown. Of course, cap 6 can be designed to be sealably engagable with the first container 2 in a variety of ways which are within the spirit and scope of the present invention, and the invention is therefore not limited to the specific embodiments illustrated and described herein.

The cap 6 is shown in snap-fittable engagement with a second container 12 which is provided with an impervious barrier 15 to prevent fluid (not shown) present in the first container 2 from penetrating the second container 12. The second container 12 can be constructed of a variety of materials which are suitable for providing an oxygen controllable environment 11, including, but not limited to, plastic, glass, silicone, food-grade butyl rubber, latex, aliphatic polyesters, natural rubber, metal, metal foils, polytetrafluoroethylene, biopolymers such as liquid wood, modified casein, polyhydroxyalkanoate polyesters, including polyhydroxybutrate, polyhydroxyvalerate, polylactic acid, starch based polyesters, keratin processed with methyl acrylate, hemp polymers, hemp plastic, hemp composite polymers, and combinations thereof. The second container 12 is preferably constructed of a cylindrical shape, as shown in FIG. 18A, which depicts at least a portion of the wall portion 17 of the impervious barrier 15 designed to taper inwardly as the wall portion 17 extends toward the base portion 19, for example, so that contents to be heated in the oxygen controlled environment 11 can aggregate together in the second receptacle 13, for example, to ensure consistent heating of the contents in the oxygen controlled environment 11. Those skilled in the art will appreciate that the length of the wall portion 17 of the impervious barrier 15 which tapers inwardly can vary, as the invention is not intended to be limited in this manner. It should also be appreciated, however, that the wall portion 17 can be designed so that the wall portion 17 does not taper.

The base 4 of the apparatus 10 can be configured to hold one or more control elements. In an embodiment, the apparatus 10 includes a heating element 18 disposed at the bottom of the first container 2 adjacent to the base 4. In an embodiment, the apparatus 10 further includes an indicator light 24 disposed in the base 4. In an embodiment, the apparatus 10 includes a temperature sensitive switching element 22 disposed in parallel electrical connection with the indicator light 24.

Heating element 18 as illustrated may comprise a circular heating coil 20 which is provided with electrical connection to a temperature responsive switching element 22 which is disposed in parallel electrical connection with an indicator light 24, as schematically shown in FIG. 17C. Heating element 18 may be adapted for alternating current or direct current operation, including battery power, and is connected to the appropriate current supply by power cord 25 shown in FIG. 17A. Referring briefly to FIG. 17C, the circuitry of heating element 18 is schematically represented, and it can be seen that thermostatic switching element 22 is located in parallel displacement astride indicator light 24, whereby an increase in the temperature of the temperature controllable environment 3 of the first container 2 beyond a predetermined threshold of the switching element 22 causes switching means 22 to open with the result that heating element 20 is cycled off and indicator light 24 can be activated and visibly glow to notify the user that the heating process has been completed. In an embodiment, the thermostat may be calibrated e.g., to implement any of the temperature ranges and time periods described herein. For example, in instances in which water is the fluid medium the thermostat may be calibrated for activation at 110° Celsius so that shut off will not occur until all water has been boiled away. In an embodiment, the heating element 18 is configured to maintain the temperature controllable environment 3 in the first container 2 at a temperature for a time period sufficient to decarboxylate at least one inactive precursor acid (e.g., cannabinolic acid) in the raw cannabis plant material to the at least one pharmacologically active cannabinoid (e.g., tetrahydrocannabinol). It should be appreciated that the temperature range can be from about 85° C. to about 125° C., preferably from about 90° C. to about 110° C., more preferably from about 95° C. to 105° C., even more preferably from about 98° C. to about 102° C., and still more preferably at about 100° C. Those skilled in the art will appreciate that the time frame sufficient for decarboxylating the at least one inactive precursor acid in the raw cannabis plant material to the at least one pharmacologically active cannabinoid can vary, depending on a variety of factors, such as the amount of raw cannabis plant material to be decarboxylated, the amount of inactive precursor present in the raw cannabis plant material, and the desired amount of at least one pharmacologically active cannabinoid desired in the resulting decarboxylated cannabis plant material, for example. In an embodiment, the time period is from about 60 minutes to 120 minutes. In an embodiment, the time period is from about 70 to 80 minutes. In an embodiment, the time period is about 75 minutes. In some embodiments, the temperature range is 90° C. to 100° C. and the time period is at least 105 minutes. In some embodiments, the temperature range is 105° C. to 115° C. and the time period is between about 20 minutes and about 60 minutes.

The switching element 22, which may comprise a thermostatic switch, can be constructed to interrupt the supply of electrical current to the heating element 20 when the temperature in the first container 2 reaches or exceeds a threshold level, for example, a level in excess of that proper for the efficient non-destructive heating of the contents (e.g., raw cannabis plant material). In an embodiment, the temperature sensitive switching element 22 maintains the temperature of the temperature controllable environment 3 in a temperature range that maximizes decarboxylation of inactive precursor acids in the raw cannabis plant material while minimizing pyrolytic degradation of the decarboxylated cannabis plant material. A temperature range which is expected to minimize pyrolytic degradation of the decarboxylated cannabis plant material is from about 85° C. to about 125° C. In an embodiment, the temperature sensitive switching element 22 shuts off the heating element 18 when the temperature in the temperature controllable environment 3 exceeds the upper limit of the temperature range. In an embodiment, the temperature sensitive switching element 22 turns on the heating element 18 when the temperature in the temperature controllable environment drops below the lower limit of the temperature range.

In some embodiments, the heating element is configured to maintain the temperature controllable environment in the first container at a temperature for a time period sufficient to decarboxylate at least 70% of the at least one inactive precursor acid in the raw cannabis plant material to the at least one pharmacologically active cannabinoid. In some embodiments, the at least one inactive precursor is cannabinolic acid, cannabidiolic acid, or both cannabinolic acid and cannabidiolic acid, and the at least one pharmacologically active cannabinoid is tetrahydrocannabinol, cannabidiol or tetrahydrocannabinol and cannabidiol, respectively. In some embodiments, the temperature comprises a range from about 85° C. to about 125° C., and the time period is from about 60 minutes to 120 minutes.

In some embodiments, the heating element is configured to maintain the temperature controllable environment in the first container at a temperature for a time period sufficient to decarboxylate at least 80% of the at least one inactive precursor acid in the raw cannabis plant material to the at least one pharmacologically active cannabinoid. In some embodiments, the heating element is configured to maintain the temperature controllable environment in the first container at a temperature for a time period sufficient to decarboxylate at least 90% of the at least one inactive precursor acid in the raw cannabis plant material to the at least one pharmacologically active cannabinoid. In some embodiments, the at least one inactive precursor is cannabinolic acid, cannabidiolic acid, or both cannabinolic acid and cannabidiolic acid, and the at least one pharmacologically active cannabinoid is tetrahydrocannabinol, cannabidiol or tetrahydrocannabinol and cannabidiol, respectively. In some embodiments, the temperature comprises a range from about 90° C. to about 100° C., and the time period is at least 105 minutes. In some embodiments, the temperature comprises a range from about 105° C. to about 115° C., and the time period is from about 20 minutes to about 60 minutes. In some embodiments, the temperature comprises a range from about 105° C. to about 115° C., and the time period is from about 60 minutes to about 120 minutes. In some embodiments, an amount of cannabinol present in the decarboxylated cannabis plant material is less than 0.5% of the total weight of the decarboxylated cannabis plant material. In some embodiments, an amount of cannabinol present in the decarboxylated cannabis plant material is less than 0.4% of the total weight of the decarboxylated cannabis plant material. In some embodiments, an amount of cannabinol present in the decarboxylated cannabis plant material is less than 0.2% of the total weight of the decarboxylated cannabis plant material. In some embodiments, an amount of cannabinol present in the decarboxylated cannabis plant material is less than 0.1% of the total weight of the decarboxylated cannabis plant material. In some embodiments, an quantifiable amount of cannabinol is not present in the decarboxylated cannabis plant material.

In an embodiment, the indicator light 24 indicates that the decarboxylated cannabis plant material contains an effective amount of the at least one pharmacologically active cannabinoid for sublingual administration.

In an embodiment, the cap 6 comprises a valve 26. In an embodiment, the valve 26 comprises a one-way valve. In embodiment, the valve 26 is situated in a portion of the cap that allows one-way fluid communication from the second container 12 to the environment external to the apparatus 10. The valve 26 relieves pressure while heating contents with the apparatus by allowing fluid to escape the second container 12. The pressure relieved by the valve 6 helps to maintain the cap 6 sealably engaged to the second container 12 during operation. Without wishing to be bound by theory, it is believed that absent pressure relief by the valve 26 pressure building up while the apparatus 10 is heating contents in the second receptacle 13 may cause the cap 6 to disengage from the second container 12, thereby undesirably exposing the contents (e.g., cannabis plant material) in the second container 12 to oxygen. In an embodiment, the valve 26 prevents air from entering the second container, thereby minimizing oxygen present in the oxygen controlled environment 11.

Generally, the apparatus can be used to heat any contents desired. Preferably, the apparatus 10 can be used to heat contents for which heating in an oxygen controlled, moisture free environment is desired. In an embodiment, the contents comprise food. Exemplary foods include, but are not limited to, vegetables, grains, legumes, pastries, confections, eggs, and breads. Other suitable foods which can be heated in the oxygen controlled environment are apparent to the skilled artisan.

In an embodiment, the contents comprise herbaceous plant material. The present invention contemplates heating any herbaceous plant material. In an embodiment, the herbaceous plant material comprises raw cannabis plant material. In an embodiment, the apparatus is used to decarboxylate the raw cannabis plant material, for example, to activate at least one pharmacologically active cannabinoid in the decarboxylated cannabis plant material. Referring now to FIG. 18B, an amount of raw cannabis plant material can be placed within the second receptacle 13 of the oxygen controlled environment 11, and then the second container 12 can be suspended from cap 6 by snap-fittable engagement of circumferential rim 14 of the second container 12 with corresponding recess 16 disposed as indicated within cover 6. When the amount of raw cannabis plant material is within the oxygen controlled environment 11, decarboxylation of the raw cannabis plant material can be carried out in accordance with the methods described herein. Any amount of raw cannabis plant material which fits into the second receptacle 13 can be placed within the second receptacle 13 so that the amount of raw cannabis plant material can be retained in an oxygen controlled environment 11 for decarboxylation of the raw cannabis plant material in accordance with the methods described herein. In an embodiment, the amount of raw cannabis plant material placed within the second receptacle 13 is sufficient for preparing a sublingual dosage form of decarboxylated cannabis plant material that delivers an effective amount of at least one pharmacologically active cannabinoid present in the decarboxylated cannabis plant material into the system circulation when the decarboxylated cannabis plant material is placed into the sublingual cavity. During decarboxylation of the cannabis plant material, the oxygen controlled environment 11 provided by the second container 12 minimizes oxidation of cannabinoids present in the cannabis plant material, thereby minimizing or preventing oxidative degradation of cannabinoids and maximizing the amount of the at least one pharmacologically active cannabinoid in the decarboxylated cannabis plant material. In an embodiment, the oxygen controlled environment decreases oxidation of cannabinoids in the decarboxylating cannabis plant material by at least 1%, 5%, 10%, 15%, 20%, 25%, 30%, 40%, 50%, or more. In an embodiment, the oxygen controlled environment decreases oxidation of cannabinoids in the decarboxylating cannabis plant material by at least 50%, at least 60%, at least 70%, at least 80%, or at least 90%. In an embodiment, the oxygen controlled environment prevents oxidation of cannabinoids in the decarboxylating cannabis plant material.

Turning now to FIG. 18C, an amount of raw cannabis plant material can be placed within the second receptacle 13 of the oxygen controlled environment 11 provided by the second container 12, compressed against an interior surface of the impervious barrier 15 by a disc 30, and then the second container 12 can be suspended from cap 6 by snap-fittable engagement of circumferential rim 14 of the second container 12 with corresponding recess 16 disposed as indicated within cover 6. As shown in FIG. 18C, the disc 30 reduces oxygen content in the oxygen controlled environment 11, as is described in detail herein. The disc 30 is configured to fit within the second container 12, and to sealably engage the wall portion 17 of the impervious barrier 15 so that when the disc 30 is pressed toward the base portion 19 of the impervious barrier 15 a volume of fluid (e.g., air) in the oxygen controlled environment 11 is displaced as the disc 30 compresses contents in the second receptacle 13. In an embodiment, as is shown in FIG. 7C, the disc 30 includes a handle 36 that allows the disc 30 to be disengaged from the wall portion 17, e.g., by pulling the handle 36, so that the decarboxylated cannabis plant material can be removed from the second receptacle 13. In an embodiment, the decarboxylated cannabis plant material can be placed directly into the sublingual cavity of a subject for use as a sublingual dosage form of the present invention. In an embodiment, the decarboxylated cannabis plant material can be shaped or formed in accordance with the description herein before being placed into the sublingual cavity of a subject for use as a sublingual dosage form of the present invention. In an embodiment, the decarboxylated cannabis plant material can be placed into a dispenser for sublingual administration of the sublingual dosage form utilizing the dispenser. In an embodiment, the decarboxylated cannabis plant material can placed into a storage medium (not shown). In an embodiment, the decarboxylated cannabis plant material can be packaged for commercial distribution.

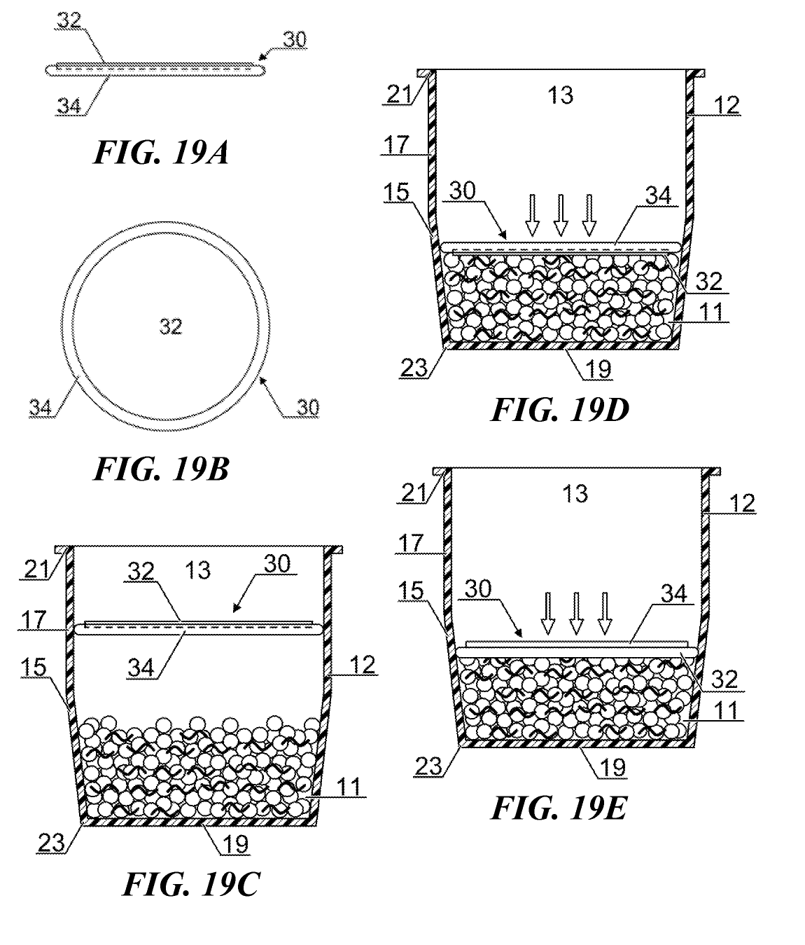

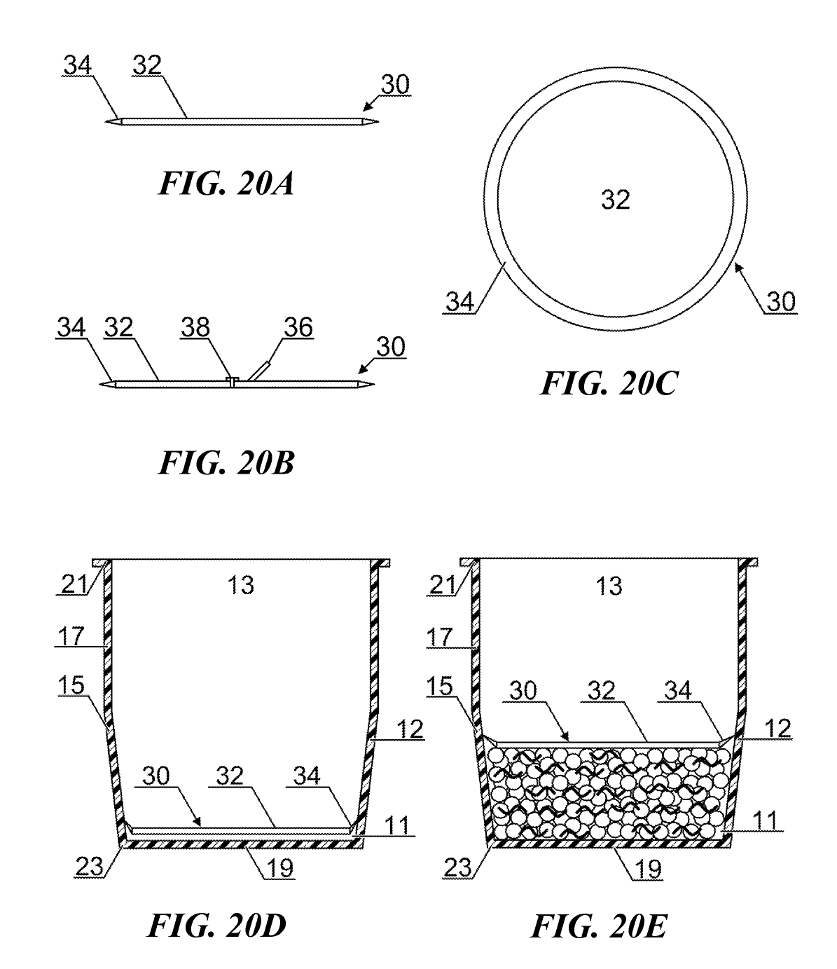

Those skilled in the art will appreciate that a variety of disc 30 configurations can be employed to permit the disc 30 to sealably engage the wall portion 17 of the impervious barrier 15 while the disc 30 is pressed toward the base portion of the impervious barrier 15, as is depicted in the example embodiments shown in FIGS. 19A-19E, 20A-20E, 21A-21C, and 22A-22C. Generally, the disc 30 includes a core member 32 and a circumferential member 34. The core member 32 can be sized and dimensioned to cover the base portion 19 of the impervious barrier 15 when the disc 30 is sealably engaged to the wall portion 17 of the impervious barrier 15. For example, FIGS. 20D, 21B, and 22B show the core member 32 sized and dimensioned to cover the base portion 19 of the impervious barrier 15 when the disc 30 is sealably engaged to the wall portion 17 of the impervious barrier 15 adjacent to the base portion 19. The circumferential member 34 can be configured to sealably engage the wall portion 17 of the impervious barrier 15 while the disc 30 is moved within the second container 12 toward the base portion 19 of the impervious barrier 15.

The core member 32 can be constructed of any material which enables the disc 30 to move within the second container 12 when pressed while compressing contents within the second receptacle 13. Examples of suitable materials include, but are not limited to, plastic, glass, silicone, food-grade butyl rubber, latex, aliphatic polyesters, natural rubber, metal, metal foils, polytetrafluoroethylene, biopolymers such as liquid wood, modified casein, polyhydroxyalkanoate polyesters, including polyhydroxybutrate, polyhydroxyvalerate, polylactic acid, starch based polyesters, keratin processed with methyl acrylate, hemp polymers, hemp plastic, hemp composite polymers, and combinations thereof. In an embodiment, the core member 32 is constructed from a rigid material, such as plastic, glass, metal, polytetrafluoroethylene, biopolymers such as liquid wood, modified casein, polyhydroxyalkanoate polyesters, including polyhydroxybutrate, polyhydroxyvalerate, polylactic acid, starch based polyesters, and keratin processed with methyl acrylate, hemp polymers, hemp plastic, hemp composite polymers, and combinations thereof. In an embodiment, the core member 32 is constructed from a flexible material, for example a flexible material that remains sealed or engaged with an inner surface of the second container 12, such as plastic, silicone, food-grade butyl rubber, latex, aliphatic polyesters, natural rubber, metal foils, polytetrafluoroethylene, biopolymers such as liquid wood, modified casein, polyhydroxyalkanoate polyesters, including polyhydroxybutrate, polyhydroxyvalerate, polylactic acid, starch based polyesters, keratin processed with methyl acrylate, hemp polymers, hemp plastic, hemp composite polymers, and combinations thereof.

The circumferential member 34 can be constructed in a variety of different ways as long as the circumferential member 34 enables the disc to sealably engage the wall portion 17 of the impervious barrier 15 while the disc 30 is moved within the second container 12.

Looking at the example embodiments shown in FIGS. 19A-19E, the circumferential member 34 can be constructed in the form of a ring that enables the circumferential member 34 to roll relative to the core member 32, thereby enabling the disc 30 to be pressed toward the base portion 19 of the impervious barrier 15 while the disc 30 is sealably engaged to the wall portion 17 of the impervious barrier 15. It is to be understood that the circumferential ring member 34 may roll in any direction relative to the core member 32. For example, when the disc 30 is pressed toward the base portion 19 of the impervious barrier 15, the ring 34 may roll above the plane of the core member 32 as is shown in FIG. 19D, or below the plane of the core member 32 as is shown in FIG. 19E.

Referring now to the example embodiments shown in FIGS. 20A-20E, 21A-21C, and 22A-22C, the circumferential member 34 can be constructed from a pliable material that enables the disc 30 to reconfigure into a “shallow cup” configuration with the core member 32 bowed into contact with contents in the second receptacle 13 when the disc 30 is pressed toward the base portion 19 of the impervious barrier 15. Examples of suitable materials include, but are not limited to, plastic, silicone, food-grade butyl rubber, latex, aliphatic polyesters, natural rubber, metal foils, PTFE (Teflon) tape, resin string, and wax (for sealant) grease (to seal), biopolymers such as liquid wood, modified casein, polyhydroxyalkanoate polyesters, including polyhydroxybutrate, polyhydroxyvalerate, polylactic acid, starch based polyesters, keratin processed with methyl acrylate, hemp polymers, hemp plastic, hemp composite polymers, and combinations thereof.

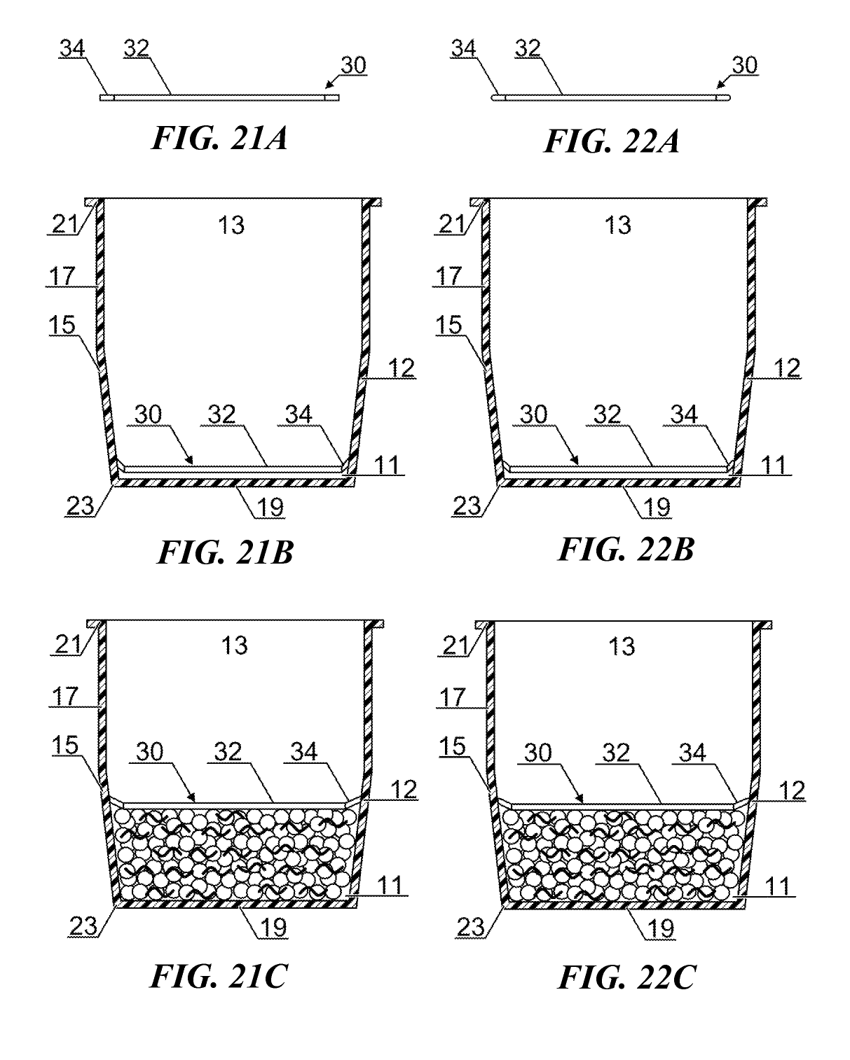

It should be appreciated that as the disc 30 is pressed toward the base portion 19, the pliable circumferential member 34 enables the disc 30 to move with relative ease while remaining sealably engaged to the wall portion 17 of the impervious barrier 15. In such embodiments, the circumferential member 34 can be provided with any geometry that enables the disc 30 to sealably engage the wall portion 17 of the impervious barrier 15 while the disc 30 is pressed toward the base portion 19 within the second container 12. Exemplary such geometries include a tapered configuration as shown in FIGS. 9A and 9B, a squared configuration as shown in FIG. 21A, and a rounded configuration as shown in FIG. 22A, and a butterflied configuration (not shown).

It is to be understood that although the disc 30 is generally shown with the core member 32 and circumferential member 34 as distinct components, the disc 30 can be constructed such that the core member 32 and circumferential member 34 are constructed as an integrated unit, as would be appreciated by those skilled in the art.

As is shown in the example embodiments depicted in FIGS. 19D, 19E, 20E, 21C and 22C, when the disc 30 is pressed toward the base portion 19 of the impervious barrier 15 the core member 32 compresses an amount of raw cannabis plant material present in the oxygen controlled environment 11 against the base portion 19 of the impervious barrier 15. In some instances, when the disc 30 is pressed toward the base portion 19 of the impervious barrier 15 the core member 32 compresses an amount of raw cannabis present in the oxygen controlled environment 11 against the wall portion 17 of the impervious barrier 15. It should be appreciated that compression of the amount of raw cannabis plant material against the wall portion 17 or base portion 19 of the impervious barrier 15 maximizes heat transmission from the temperature controlled environment 3 to the raw cannabis plant material present in the oxygen controlled environment 11 by increasing the surface area of the cannabis that is in contact with the interior surface of the impervious barrier 115.

It should be appreciated that when the disc 30 is pressed toward the base portion 19 of the impervious barrier 15 a volume of fluid (e.g., air) in the oxygen controlled environment 11 can be displaced as the disc 30 compresses an amount of raw cannabis plant material in the oxygen controlled environment 11. Those skilled in the art will appreciate that as the volume of fluid in the oxygen controlled environment 11 decreases, pressure in the oxygen controlled environment 11 may increase. Accordingly, the disc 30 can be provided with a one way valve 38 to enable the volume of fluid in the oxygen controlled environment 11 to decrease without causing the pressure in the oxygen controlled environment 11 to increase, as is shown in the example embodiment depicted in FIG. 20B. Although the valve 38 is only depicted in FIG. 20B, those skilled in the art will appreciate that other configurations of the disc 30 can be designed to include valve 38. Without wishing to be bound by theory, it is believed that the valve 38 enables the disc to minimize oxygen content in the oxygen controlled environment 11. For example, it is believed that oxygen present in ambient air that enters the oxygen controlled environment 11 of the second receptacle 13 upon placement of an amount of raw cannabis plant material into the second receptacle 13 can escape through the valve 38 without allowing air to penetrate the oxygen controlled environment 11, which is sealed off from the ambient atmosphere by sealable engagement of the disc 30 to the wall portion 17 of the impervious barrier 15. In this way, air initially present in the oxygen controlled environment 11 escapes without permitting additional air to enter the oxygen controlled environment 11 as the disc 30 is pressed toward the base portion 19, and compresses the amount of raw cannabis plant material in the oxygen controlled environment 11 against the interior surface of the impervious barrier 15.

Compositions

The present invention contemplates compositions consisting of, consisting essentially of, or comprising decarboxylated cannabis plant material. In an aspect, a sublingual composition for delivery of at least one pharmacologically active cannabinoid present in decarboxylated cannabis plant material into the systemic circulation of a subject when the composition is placed in the subject's sublingual cavity comprises; (a) an amount of decarboxylated cannabis plant material sufficient to sublingually deliver an effective dose of the at least one pharmacologically active cannabinoid into the systemic circulation of a subject when the sublingual composition is placed into the sublingual cavity; and (b) a flavorant, wherein the decarboxylated cannabis plant material is infused with the flavorant.

In an aspect, a sublingual composition for delivery of at least one pharmacologically active cannabinoid present in decarboxylated cannabis plant material into the systemic circulation of a subject when the composition is placed in the subject's sublingual cavity comprises: (a) at least 70% of the maximum amount of at least one pharmacologically active cannabinoid that could theoretically be present as a result of decarboxylation of the maximum amount of the at least one pharmacologically active cannabinoid's inactive precursor present in the cannabis plant material; and (b) a flavorant, wherein the decarboxylated cannabis plant material is infused with the flavorant.

The ratio of the amount of decarboxylated cannabis plant material to the amount of flavorant present in the composition can vary, as will be appreciated by those skilled in the art. The flavorant can be present in the sublingual composition in an amount of from about 1% to about 20% inclusive by weight of the sublingual composition. The decarboxylated cannabis plant material can be present in an amount of about 80% to about 99% inclusive by weight of the sublingual composition. In an embodiment, the amount of decarboxylated cannabis plant material is from about 0.05 grams to about 7 grams inclusive. In an embodiment, the effective dose of the at least one pharmacologically active cannabinoid is from about 2 mg to about 500 mg inclusive.

In some embodiments, the at least one pharmacologically active cannabinoid is tetrahydrocannabinol and the at least one pharmacologically active cannabinoid's inactive precursor is cannabinolic acid. In some embodiments, the cannabinolic acid is present in the cannabis plant material prior to decarboxylation in an amount from about 1% to about 40% total weight of the cannabis plant material. In some embodiments, the tetrahydrocannabinol is present in the decarboxylated cannabis plant material in an amount from about 1% to about 40% total weight of the decarboxylated cannabis plant material. In some embodiments, the decarboxylated cannabis plant material contains at least 80% of the maximum amount of tetrahydrocannabinol that could theoretically be present as a result of decarboxylation of the maximum amount of cannabinolic acid present in the cannabis plant material. In some embodiments, the decarboxylated cannabis plant material contains at least 90% of the maximum amount of tetrahydrocannabinol that could theoretically be present as a result of decarboxylation of the maximum amount of cannabinolic acid present in the cannabis plant material. In some embodiments, the decarboxylated cannabis plant material contains 100% of the maximum amount of tetrahydrocannabinol that could theoretically be present as a result of decarboxylation of the maximum amount of cannabinolic acid present in the cannabis plant material. In some embodiments, the at least one pharmacologically active cannabinoid is cannabidiol and the at least one pharmacologically active cannabinoid's inactive precursor is cannabidiolic acid. In some embodiments, the cannabidiolic acid is present prior to decarboxylation in an amount from about 1% to about 40% total weight of the raw cannabis plant material. In some embodiments, the cannabidiol is present after decarboxylation in an amount from about 1% to about 40% total weight of the decarboxylated cannabis plant material. In some embodiments, the decarboxylated cannabis plant material contains at least 80% of the maximum amount of cannabidiol that could theoretically be present as a result of decarboxylation of the maximum amount of cannabidiolic acid present in the raw cannabis plant material. In some embodiments, the decarboxylated cannabis plant material contains at least 90% of the maximum amount of cannabidiol that could theoretically be present as a result of decarboxylation of the maximum amount of cannabidiolic acid present in the raw cannabis plant material. In some embodiments, the at least one pharmacologically active cannabinoid comprises tetrahydrocannabinol and cannabidiol, and wherein the at least one pharmacologically active cannabinoid's inactive precursor is cannabinolic acid and cannabidiolic acid, respectively. In some embodiments, the cannabinolic acid is present prior to decarboxylation in an amount from about 1% to about 40% total weight of the raw cannabis plant material, and wherein the cannabidiolic acid is present prior to decarboxylation in an amount from about 1% to about 40% total weight of the raw cannabis plant material. In some embodiments, the tetrahydrocannabinol is present after decarboxylation in an amount from about 1% to about 40% total weight of the decarboxylated cannabis plant material, and wherein the cannabidiol is present after decarboxylation in an amount from about 1% to about 40% total weight of the decarboxylated cannabis plant material. In some embodiments, the decarboxylated cannabis plant material contains at least 70% of the maximum amount of tetrahydrocannabinol and at least 70% of the maximum amount of cannabidiol that could theoretically be present as a result of decarboxylation of the maximum amount of cannabinolic acid and cannabidiolic acid, respectively, present in the raw cannabis plant material. In some embodiments, the decarboxylated cannabis plant material contains at least 90% of the maximum amount of tetrahydrocannabinol and at least 80% of the maximum amount of cannabidiol that could theoretically be present as a result of decarboxylation of the maximum amount of cannabinolic acid and cannabidiolic acid, respectively, present in the raw cannabis plant material. In some embodiments, the decarboxylated cannabis plant material contains the maximum amount of tetrahydrocannabinol and at least 90% of the maximum amount of cannabidiol that could theoretically be present as a result of decarboxylation of the maximum amount of cannabinolic acid and cannabidiolic acid, respectively, present in the raw cannabis plant material. In some embodiments, an amount of cannabinol present in the composition less than 0.5%, less than 0.4%, less than 0.3%, less than 0.2%, or less than 0.1% of the total weight of the decarboxylated cannabis plant material. In some embodiments, a quantifiable amount of cannabinol is not present in the composition. Kits

A sublingual dosage form described herein or apparatus for preparing the same described herein can be provided in a kit. The kit can include any combination of i) the sublingual dosage form or a composition thereof, ii) an apparatus described herein, iii) a dispenser described herein, iv) a storage medium described herein v) a disc described herein, and vi) informational material. The informational material can be descriptive, instructional, marketing or other material that relates to the methods described herein and/or the use of the apparatuses or systems for the methods described herein. For example, the informational material describes methods for preparing a sublingual dosage form described herein (e.g., decarboxylated cannabis plant material), or a composition thereof.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In an embodiment, the informational material of the kit is a link or contact information, e.g., a physical address, email address, hyperlink, website, or telephone number, where a user of the kit can obtain substantive information about the sublingual dosage form and/or its methods of preparing the sublingual dosage form described herein. Of course, the informational material can also be provided in any combination of formats.

The kit can include one or more containers containing the sublingual dosage form. In an embodiment, the kit contains separate containers, dividers or compartments for the sublingual dosage form and informational material. For example, the sublingual dosage form (e.g., in a composition) can be contained in a storage medium, and the informational material can be contained in a plastic sleeve or packet. In an embodiment, the separate elements of the kit can be contained within a single, undivided container. For example, the sublingual dosage form is contained in a storage medium that has attached thereto the informational material in the form of a label. In an embodiment, kit includes a plurality (e.g., a pack) of individual containers, each containing one or more unit dosage forms of the sublingual dosage form described herein. The containers of the kits can be air tight and/or waterproof.

In an aspect, a kit for preparing a sublingual dosage form comprising decarboxylated cannabis plant material includes an apparatus described herein, and instructions for using the apparatus to prepare the sublingual dosage form. In an embodiment, the apparatus includes i) a first container having a first receptacle configured to retain a fluid medium; ii) a cap configured to sealably engage the first container, thereby providing the first container with a temperature controllable environment; and iii) a second container sealably engaged to and suspended from the cap, the second container configured to provide an oxygen controlled environment for minimizing oxidation of cannabinoids during decarboxylation of cannabis plant material, the second container having a second receptacle configured to receive an amount of raw cannabis plant material, and an impervious barrier which prevents fluid present in the first container from penetrating the second container, thereby minimizing oxidation of cannabinoids during decarboxylation of the cannabis plant material; wherein the amount of raw cannabis plant material is sufficient for preparing a sublingual dosage form that delivers an effective amount of at least one pharmacologically active cannabinoid present in the decarboxylated cannabis plant material into the system circulation when the decarboxylated cannabis plant material is placed into the sublingual cavity.

In an embodiment, the instructions comprise directions for decarboxylating at least one inactive precursor acid present in the raw cannabis plant material into the least one pharmacologically active cannabinoid present in the decarboxylated cannabis plant material.

In an embodiment, the instructions comprise directions for heating the raw cannabis plant material in the oxygen controlled environment at a temperature range of from about 85° C. to about 125° C. for a period of from about 60 minutes to about 120 minutes to decarboxylate the raw cannabis plant material.

In an embodiment, the instructions comprise directions for heating the oxygen controlled environment at a temperature range of from about 85° C. to about 125° C. for a period of from about 60 minutes to about 120 minutes to decarboxylate the raw cannabis plant material.

In some embodiments, the instructions comprise directions for decarboxylating at least 70% of the at least one inactive precursor acid present in the raw cannabis plant material into the at least one pharmacologically active cannabinoid present in the decarboxylated cannabis plant material. For example, the instructions comprise directions for heating the raw cannabis plant material in the oxygen controlled environment at a temperature range from about 85° C. to about 125° C. for a period of time from about 60 minutes to 120 minutes.

In some embodiments, the instructions comprise directions for decarboxylating at least 80% of the at least one inactive precursor acid present in the raw cannabis plant material into the at least one pharmacologically active cannabinoid present in the decarboxylated cannabis plant material. As an example, the instructions comprise directions for heating the raw cannabis plant material in the oxygen controlled environment at a temperature range from about 105° C. to about 115° C. for a period of between about 30 minutes to about 60 minutes, e.g., to achieve at least 80% decarboxylation completeness for a CBD-enriched cannabis strain. In some embodiments, the instructions comprise directions for decarboxylating at least 90% of the at least one inactive precursor acid present in the raw cannabis plant material into the at least one pharmacologically active cannabinoid present in the decarboxylated cannabis plant material. As an example, the instructions comprise directions for heating the raw cannabis plant material in the oxygen controlled environment at a temperature range from about 90° C. to about 100° C. for a time period of at least 105 minutes, e.g., to achieve at least 90% decarboxylation completeness for a THC-enriched cannabis strain. As another example, the instructions comprise directions for heating the raw cannabis plant material in the oxygen controlled environment at a temperature range from about 105° C. to about 115° C. for a time period of between 20 minutes and 60 minutes, e.g., to achieve up to 90% decarboxylation completeness for a THC and at least 80% and decarboxylation completeness for CBD in a THC- and CBD-enriched cannabis strain.

In some embodiments, the instructions comprise directions for limiting the amount of cannabinol present in the decarboxylated cannabis plant material to less than 1% of the total weight of the decarboxylated cannabis plant material. In some embodiments, the at least one inactive precursor is cannabinolic acid, cannabidiolic acid, or both cannabinolic acid and cannabidiolic acid, and the at least one pharmacologically active cannabinoid is tetrahydrocannabinol, cannabidiol or tetrahydrocannabinol and cannabidiol, respectively. In some embodiments, the instructions comprise directions for selecting a THC-enriched cannabis strain for decarboxylation, and decarboxylating it to achieve at least 70%, at least 80%, at least 90% or up to 100% decarboxylation completeness. In some embodiments, the instructions comprise directions for selecting a CBD-enriched cannabis strain for decarboxylation, and decarboxylating it to achieve at least 70%, at least 80%, or at least 90% decarboxylation completeness. In some embodiments, the instructions comprise directions for selecting a THC- and CBD-enriched cannabis strain for decarboxylation, and decarboxylating it to achieve at least 70% decarboxylation completeness for both THC and CBD, at least 90% decarboxylation completeness for THC and at least 80% decarboxylation completeness for THC and CBP, respectively, or 100% decarboxylation completeness for THC and at least 90% decarboxylation completeness for CBD.

It should be appreciated that the instructions can include directions for the sublingual administration of the at least one pharmacologically active cannabinoid into the systemic circulation. In an embodiment, the instructions comprise directions for administering the sublingual dosage form to a subject having a prescription for medical marijuana use. In an embodiment, the instructions comprise a label for administering the decarboxylated cannabis plant material for prescription use of the decarboxylated cannabis plant material, such as a label approved by a federal regulatory authority (e.g., FDA). In an embodiment, the instructions comprise directions for placing the decarboxylated cannabis plant material into the sublingual cavity. In an embodiment, the instructions comprise directions for forming an amount of the decarboxylated cannabis plant material into a shape suitable for sublingual administration of the at least one pharmacologically active cannabinoid present in the decarboxylated cannabis plant material into the systemic circulation. In an embodiment, the instructions comprise directions for flattening out the amount of the decarboxylated cannabis plant material prior to placing the decarboxylated cannabis plant material into the sublingual cavity. In an embodiment, the instructions comprise directions for spreading out the amount of decarboxylated cannabis plant material prior to placing the decarboxylated plant material into the sublingual cavity. In an embodiment, the instructions comprise directions for flattening out and spreading out the amount of decarboxylated cannabis plant material prior to placing the decarboxylated plant material into the sublingual cavity. In an embodiment, the instructions comprise directions for gathering the decarboxylated cannabis plant material prior to placing the decarboxylated cannabis plant material into the sublingual cavity. In an embodiment, the instructions comprise directions for ingesting the amount of the decarboxylated cannabis plant material after the at least one pharmacologically active cannabinoid present in the decarboxylated cannabis plant material is delivered into the systemic circulation.

The kit can include a dispenser for administering the at least one pharmacologically active cannabinoid present in the decarboxylated cannabis plant material to the sublingual mucosa when the dispenser is placed into the sublingual cavity. In an embodiment, the instructions comprise directions for placing the decarboxylated cannabis plant material into the dispenser. The instructions can direct the user to shape or form the decarboxylated cannabis plant material into a geometry optimal for sublingual administration of the decarboxylated cannabis plant material in the dispenser. In an embodiment, the instructions direct a user to flatten out the decarboxylated cannabis plant material prior to placing the decarboxylated cannabis plant material into the dispenser. In an embodiment, the instructions direct a user to spread out the decarboxylated cannabis plant material prior to placing the decarboxylated cannabis plant material into the dispenser. In an embodiment, the instructions direct the user to flatten and spread out the decarboxylated cannabis plant material prior to placing the decarboxylated cannabis plant material into the dispenser.

In some embodiments, the kit includes a scale (e.g., digital scale).

In an embodiment, the apparatus of the kit includes a disc as described herein.

Aspects of the invention involve kits comprising a dispenser of the present invention in a suitable packaging.

In an aspect, a kit includes a first wrapping 52 and a second wrapping, wherein the first wrapping 52 comprises at least one aperture 54 configured to retain decarboxylated cannabis plant material 51 inside the wrapping while permitting an effective amount of at least one pharmacologically active cannabinoid to be released into a subject's sublingual cavity when the wrapping is folded around the decarboxylated cannabis plant material and placed in the subject's sublingual cavity; and wherein the first wrapping is packaged inside the second wrapping.

In an aspect, a kit includes a first wrapping 52 and a second wrapping, wherein the first wrapping 52 comprises a plurality of apertures 54 configured to retain decarboxylated cannabis plant material 51 inside the wrapping while permitting an effective amount of at least one pharmacologically active cannabinoid to be released into a subject's sublingual cavity when the wrapping is folded around the decarboxylated cannabis plant material and placed in the subject's sublingual cavity; and wherein the first wrapping is packaged inside the second wrapping.

The second wrapping can comprise any suitable packaging to distribute a dispenser 50 of the present invention. Exemplary second wrappings include, without limitation, a wax sheet, a cellophane sheet, or a foil sheet.

Systems

The present invention contemplates various systems for preparing and delivering decarboxylated cannabis plant material sublingually. In an aspect, the present invention provides a system for delivering a sublingual dose of at least one pharmacologically active cannabinoid present in decarboxylated cannabis plant material into the systemic circulation of a subject, the system comprising: (a) a sublingual dosage form comprising an amount of decarboxylated cannabis plant material sufficient to sublingually deliver an effective dose of the at least one pharmacologically active cannabinoid into the systemic circulation of the subject; (b) dispenser containing the amount of decarboxylated cannabis plant material, wherein the dispenser releases the effective dose of the at least one pharmacologically active cannabinoid present in the decarboxylated cannabis plant material into contact with the subject's sublingual mucosa when the dispenser is placed in the subject's sublingual cavity, thereby sublingually delivering the sublingual dose of the at least one pharmacologically active cannabinoid into the subject's systemic circulation.

In an aspect, disclosed herein is a system for delivering a sublingual dose of at least one pharmacologically active cannabinoid present in decarboxylated cannabis plant material into the systemic circulation of a subject, the system comprising: (a) a sublingual dosage form comprising decarboxylated cannabis plant material containing at least 70% of the maximum amount of at least one pharmacologically active cannabinoid that could theoretically be present as a result of decarboxylation of the maximum amount of the at least one pharmacologically active cannabinoid's inactive precursor present in the cannabis plant material; and (b) a dispenser containing the decarboxylated cannabis plant material, wherein the dispenser releases an effective dose of the at least one pharmacologically active cannabinoid present in the decarboxylated cannabis plant material into contact with the subject's sublingual mucosa when the dispenser is placed in the subject's sublingual cavity, thereby sublingually delivering the effective dose of the at least one pharmacologically active cannabinoid into the subject's systemic circulation. In some embodiments, the at least one pharmacologically active cannabinoid is tetrahydrocannabinol and the at least one pharmacologically active cannabinoid's inactive precursor is cannabinolic acid. In some embodiments, the cannabinolic acid is present in the cannabis plant material prior to decarboxylation in an amount from about 1% to about 40% total weight of the cannabis plant material. In some embodiments, the tetrahydrocannabinol is present in the decarboxylated cannabis plant material in an amount from about 1% to about 40% total weight of the decarboxylated cannabis plant material. In some embodiments, the decarboxylated cannabis plant material contains at least 80% of the maximum amount of tetrahydrocannabinol that could theoretically be present as a result of decarboxylation of the maximum amount of cannabinolic acid present in the cannabis plant material. In some embodiments, the decarboxylated cannabis plant material contains at least 90% of the maximum amount of tetrahydrocannabinol that could theoretically be present as a result of decarboxylation of the maximum amount of cannabinolic acid present in the cannabis plant material. In some embodiments, the decarboxylated cannabis plant material contains the maximum amount of tetrahydrocannabinol that could theoretically be present as a result of decarboxylation of the maximum amount of cannabinolic acid present in the cannabis plant material. In some embodiments, the at least one pharmacologically active cannabinoid is cannabidiol and the at least one pharmacologically active cannabinoid's inactive precursor is cannabidiolic acid. In some embodiments, the cannabidiolic acid is present prior to decarboxylation in an amount from about 1% to about 40% total weight of the raw cannabis plant material. In some embodiments, the cannabidiol is present after decarboxylation in an amount from about 1% to about 40% total weight of the decarboxylated cannabis plant material. In some embodiments, the decarboxylated cannabis plant material contains at least 80% of the maximum amount of cannabidiol that could theoretically be present as a result of decarboxylation of the maximum amount of cannabidiolic acid present in the raw cannabis plant material. In some embodiments, the decarboxylated cannabis plant material contains at least 90% of the maximum amount of cannabidiol that could theoretically be present as a result of decarboxylation of the maximum amount of cannabidiolic acid present in the raw cannabis plant material. In some embodiments, the at least one pharmacologically active cannabinoid comprises tetrahydrocannabinol and cannabidiol, and wherein the at least one pharmacologically active cannabinoid's inactive precursor is cannabinolic acid and cannabidiolic acid, respectively. In some embodiments, the cannabinolic acid is present prior to decarboxylation in an amount from about 1% to about 40% total weight of the raw cannabis plant material, and wherein the cannabidiolic acid is present prior to decarboxylation in an amount from about 1% to about 40% total weight of the raw cannabis plant material. In some embodiments, the tetrahydrocannabinol is present after decarboxylation in an amount from about 1% to about 40% total weight of the decarboxylated cannabis plant material, and wherein the cannabidiol is present after decarboxylation in an amount from about 1% to about 40% total weight of the decarboxylated cannabis plant material. In some embodiments, the decarboxylated cannabis plant material contains at least 90% of the maximum amount of tetrahydrocannabinol and at least 80% of the maximum amount of cannabidiol that could theoretically be present as a result of decarboxylation of the maximum amount of cannabinolic acid and cannabidiolic acid, respectively, present in the raw cannabis plant material. In some embodiments, the decarboxylated cannabis plant material contains the maximum amount of tetrahydrocannabinol and at least 90% of the maximum amount of cannabidiol that could theoretically be present as a result of decarboxylation of the maximum amount of cannabinolic acid and cannabidiolic acid, respectively, present in the raw cannabis plant material. In some embodiments, an amount of cannabinol present in the decarboxylated cannabis plant material consisting of less than 0.5%, less than 0.4%, less than 0.3%, less than 0.2%, or less than 0.1% of the total weight of the decarboxylated cannabis plant material. In some embodiments, a quantifiable amount of cannabinol is not present in the decarboxylated cannabis plant material. It should be appreciated that the dispenser can be any dispenser, product, sheet, or wrapping described herein. It should also be appreciated that the sublingual dosage form can be any sublingual dosage form, decarboxylated cannabis plant material, or composition described herein, or produced in accordance with a method of the present invention.

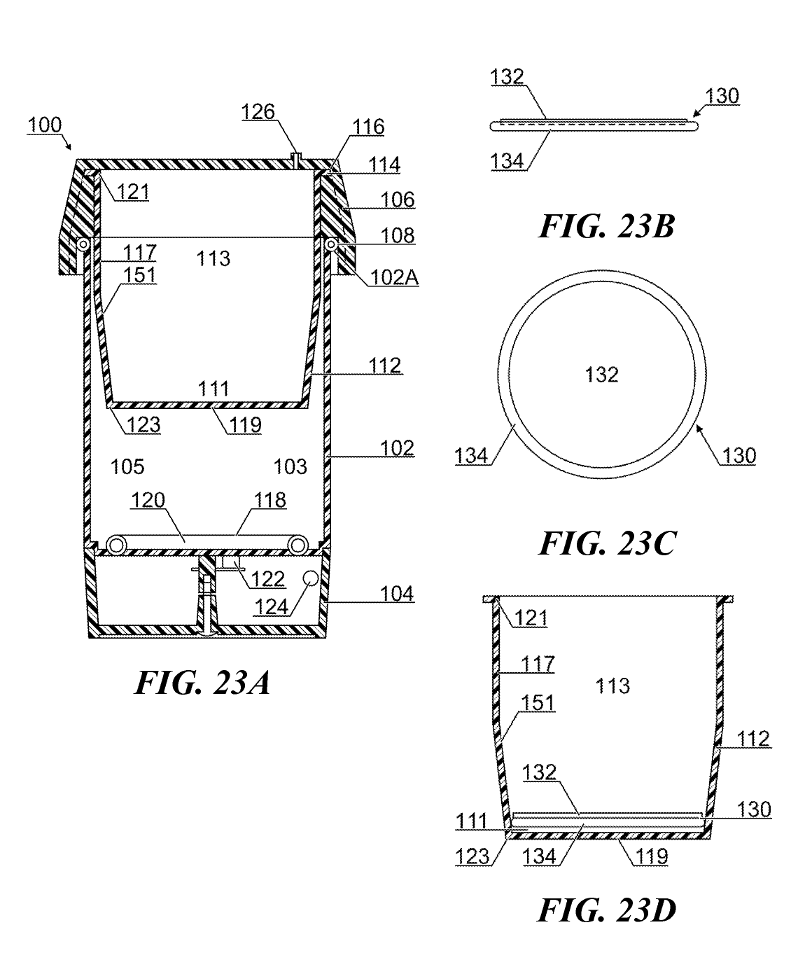

In an aspect, the present invention provides a system 100 for heating contents in an oxygen controlled environment. In an aspect, a system 100 of the present invention includes an apparatus 110 for heating contents in an oxygen controlled environment 111, and a disc 130 for reducing oxygen content in the oxygen controlled environment 111, as are shown in FIGS. 23A, 23B, 23C and 23D. With reference now to FIG. 23A, the apparatus 110 includes a first container 102 providing a temperature controllable environment 103, a cap 106 configured to sealably engage the first container 102, and a second container 112 configured to provide an oxygen controlled environment 111 for limiting the exposure of contents in the second container 112 to oxygen. The first container 102 has a first receptacle 105 configured to retain a fluid medium. The second container 112 has a second receptacle 113, and an impervious barrier 115 which prevents fluid present in the first container 102 from penetrating the second container 112. The impervious barrier 115 includes a wall portion 117 and a base portion 119. The wall portion 117 extends circumferentially from a first end 121, which is open, to a second end 123, which is closed by the base portion 119.

The first container 102 can be constructed of a variety of materials which are suitable for providing a temperature controllable environment 103, including, but not limited to, plastic, glass, silicone, food-grade butyl rubber, latex, aliphatic polyesters, natural rubber, metal, metal foils, polytetrafluoroethylene, biopolymers such as liquid wood, modified casein, polyhydroxyalkanoate polyesters, including polyhydroxybutrate, polyhydroxyvalerate, polylactic acid, starch based polyesters, keratin processed with methyl acrylate, hemp polymers, hemp plastic, hemp composite polymers, and combinations thereof. The first container 102 is preferably constructed of a cylindrical shape as shown in FIG. 23A. The first container 102 is open at its upper end to receive the second container 112, and is closed at its lower end by a base 104. A cap 106 is configured to be fitted over a relieved upper edge 102A of the container in a position to be sealed by a sealing element 108. The sealing element 108, for example, can be an O-ring disposed within the perimeter of cap 6 or a bead-like lip as shown. Of course, cap 6 can be designed to be sealably engagable with the first container 102 in a variety of ways which are within the spirit and scope of the present invention, and the invention is therefore not limited to the specific embodiments illustrated and described herein.

The cap 106 is shown in snap-fittable engagement with a second container 112 which is provided with an impervious barrier 115 to prevent fluid (not shown) present in the first container 102 from penetrating the second container 112. The second container 112 can be constructed of a variety of materials which are suitable for providing an oxygen controllable environment 111, including, but not limited to, plastic, glass, silicone, food-grade butyl rubber, latex, aliphatic polyesters, natural rubber, metal, metal foils, polytetrafluoroethylene, biopolymers such as liquid wood, modified casein, polyhydroxyalkanoate polyesters, including polyhydroxybutrate, polyhydroxyvalerate, polylactic acid, starch based polyesters, keratin processed with methyl acrylate, hemp polymers, hemp plastic, hemp composite polymers, and combinations thereof. The second container 112 is preferably constructed of a cylindrical shape, as shown in FIGS. 23A and 23D. As is shown in FIGS. 23A and 23D, at least a portion of the wall portion 117 of the impervious barrier 115 can be designed to taper inwardly as the wall portion 117 extends toward the base portion 119, for example, so that contents to be heated in the oxygen controlled environment 111 can aggregate together in the second receptacle 113, for example, to ensure consistent heating of the contents in the oxygen controlled environment 111. Those skilled in the art will appreciate that the length of the wall portion 117 of the impervious barrier 115 which tapers inwardly can vary, as the invention is not intended to be limited in this manner. It should also be appreciated, however, that the wall portion 117 can be designed so that the wall portion 117 does not taper.

Turning now to FIG. 23B, there is shown a disc 130 for reducing oxygen content in the oxygen controlled environment 111. The disc 130 is configured to fit within the second container 112 (see FIG. 23D), and to sealably engage the wall portion 117 of the impervious barrier 115 so that when the disc 130 is pressed toward the base portion 119 of the impervious barrier 115 a volume of fluid (e.g., air) in the oxygen controlled environment 111 is displaced as the disc 130 compresses contents in the second receptacle 113. Those skilled in the art will appreciate that as the volume of fluid in the oxygen controlled environment 111 decreases, pressure in the oxygen controlled environment 111 may increase. Accordingly, the disc 130 can be provided with a valve (e.g., a one-way valve) (not shown) to enable the volume of fluid in the oxygen controlled environment 111 to decrease without causing the pressure in the oxygen controlled environment 111 to increase. Without wishing to be bound by theory, it is believed that the valve enables the disc to minimize oxygen content in the oxygen controlled environment 111. For example, it is believed that oxygen present in ambient air that enters the oxygen controlled environment 111 of the second receptacle 113 upon placement of contents into the second receptacle 113 can escape through the valve without allowing air to penetrate the oxygen controlled environment which is sealed off from the ambient atmosphere by sealable engagement of the disc 130 to the wall portion 117 of the impervious barrier 115. In this way, air initially present in the oxygen controlled environment 111 escapes without permitting additional air to enter the oxygen controlled environment 111 as the disc 130 is pressed toward the base portion 119, and compresses the contents (not shown) placed into the second receptacle 113 against the interior surface of the impervious barrier 115.

Those skilled in the art will appreciate that any disc configuration contemplated herein can be employed to permit the disc 130 to sealably engage the wall portion 117 of the impervious barrier 115 while the disc 130 is pressed toward the base portion 119 of the impervious barrier 115. In an embodiment, the disc 130 includes a core member 132 and a circumferential member 134, as contemplated herein.

In an embodiment, when the disc 130 is pressed toward the base portion 119 of the impervious barrier 115 the core member 132 compresses contents present in the second receptacle 113 against the base portion 119 of the impervious barrier 115. In an embodiment, when the disc 130 is pressed toward the base portion 119 of the second container 112 the core member 132 compresses contents present in the second receptacle 113 against the wall portion 117 of the impervious barrier 115. It should be appreciated that compression of the contents against the wall portion 117 or base portion 119 of the impervious barrier 115 maximizes heat transmission from the temperature controlled environment 103 to the contents present in the second receptacle 113 by increasing the surface area of the contents that are in contact with the interior surface of the impervious barrier 115.

Referring to FIG. 23A, contents (e.g., an amount of raw cannabis plant material), can be placed within the second receptacle 113, compressed against the surface of the impervious barrier 115 by disc 130 (shown in FIGS. 23B, 23C and 23D), and then the second container can be suspended from cap 106 by snap-fittable engagement of circumferential rim 114 of the second container 112 with corresponding recess 116 disposed as indicated within cover 106. Those skilled in the art will appreciate that although the mechanism of releasably engaging cap 106 and second container 112 has been shown, it is to be understood that alternate engagement mechanisms may be employed, such as regularly spaced projections radiating inwardly from the inner surface of cap 106 to mate with corresponding detents disposed in the outer upper surface of second container 112. The disclosure is therefore merely illustrative of such engagement mechanisms, and the invention is therefore not limited to the specific construction illustrated herein.

The base 104 of the apparatus 110 of the system 100 can be configured to hold one or more control elements. In an embodiment, the apparatus 110 includes a heating element 118 disposed at the bottom of the first container 102 adjacent to the base 104. In an embodiment, the apparatus 110 of the system 100 further includes an indicator light 124 disposed in the base 104. In an embodiment, the apparatus 110 of the system 100 includes a temperature sensitive switching element 122 disposed in parallel electrical connection with the indicator light 124.

Heating element 118 as illustrated may comprise a circular heating coil 120 which is provided with electrical connection to a temperature responsive switching element 122 which is disposed in parallel electrical connection with an indicator light 124, as schematically shown in FIG. 17C. Heating element 118 may be adapted for alternating current or direct current operation, including battery power, and is connected to the appropriate current supply by power cord 125 shown in FIG. 17.

Referring briefly to FIG. 17C, the circuitry of heating element 118 is schematically represented, and it can be seen that thermostatic switching element 122 is located in parallel displacement astride indicator light 124, whereby an increase in the temperature of the temperature controllable environment 103 of the first container 102 beyond a predetermined threshold of the switching element 122 causes switching means 122 to open with the result that heating element 120 is cycled off and indicator light 124 can be activated and visibly glow to notify the user that the heating process has been completed. In an embodiment, the thermostat may be calibrated for activation e.g., to implement any of the temperature ranges and time periods described herein. For example, in instances in which water is the fluid medium the thermostat may be calibrated for activation at 110° Celsius so that shut off will not occur until all water has been boiled away at which point the temperature rises rapidly and the apparatus will shut off. In an embodiment, the heating element 118 is configured to maintain the temperature controllable environment 103 in the first container 102 at a temperature for a time period sufficient to decarboxylate at least one inactive precursor acid (e.g., cannabinolic acid) in the raw cannabis plant material to the at least one pharmacologically active cannabinoid (e.g., tetrahydrocannabinol and cannabidiol). It should be appreciated that the temperature range can be from about 85° C. to about 125° C., preferably from about 90° C. to about 110° C., more preferably from about 95° C. to 105° C., even more preferably from about 98° C. to about 102° C., and still more preferably at about 100° C. Those skilled in the art will appreciate that the time frame sufficient for decarboxylating the at least one inactive precursor acid in the raw cannabis plant material to the at least one pharmacologically active cannabinoid can vary, depending on a variety of factors, such as the amount of raw cannabis plant material to be decarboxylated, the amount of inactive precursor present in the raw cannabis plant material, and the desired amount of at least one pharmacologically active cannabinoid desired in the resulting decarboxylated cannabis plant material, for example. In an embodiment, the time period is from about 60 minutes to 120 minutes. In an embodiment, the time period is from about 70 to 80 minutes. In an embodiment, the time period is about 75 minutes. In some embodiments, the temperature range is from 90° C. to 100° C. and the time period is at least 105 minutes. In some embodiments, the temperature range is from 105° C. to 115° C. and the time period is between 20 minutes and 60 minutes. In some embodiments, the temperature range is from 105° C. to 115° C. and the time period is between 30 minutes and 60 minutes. In some embodiments, the temperature range is from 105° C. to 115° C. and the time period is between 60 minutes and 120 minutes.

The switching element 122, which may comprise a thermostatic switch, can be constructed to interrupt the supply of electrical current to the heating element 120 when the temperature in the first container 102 reaches or exceeds a threshold level, for example, a level in excess of that proper for the efficient non-destructive heating of the contents (e.g., raw cannabis plant material). In an embodiment, the temperature sensitive switching element 122 maintains the temperature of the temperature controllable environment 103 in a temperature range that maximizes decarboxylation of inactive precursor acids in the raw cannabis plant material while minimizing pyrolytic degradation of the decarboxylated cannabis plant material. A temperature range which is expected to minimize pyrolytic degradation of the decarboxylated cannabis plant material is from about 85° C. to about 125° C. In an embodiment, the temperature sensitive switching element 122 shuts off the heating element 118 when the temperature in the temperature controllable environment 103 exceeds the upper limit of the temperature range. In an embodiment, the temperature sensitive switching element 122 turns on the heating element 118 when the temperature in the temperature controllable environment drops below the lower limit of the temperature range.

In an embodiment, the indicator light 124 indicates that the decarboxylated cannabis plant material contains an effective amount of the at least one pharmacologically active cannabinoid for sublingual administration.

In an embodiment, the cap 106 comprises a valve 126. In an embodiment, the valve 126 comprises a one-way valve. In embodiment, the valve 126 is situated in a portion of the cap that allows one-way fluid communication from the second container 112 to the environment external to the apparatus 110. The valve 126 relieves pressure while heating contents with the apparatus by allowing fluid to escape the second container 112. The pressure relieved by the valve 126 helps to maintain the cap 106 sealably engaged to the second container 112 during operation. Without wishing to be bound by theory, it is believed that absent pressure relief by the valve 126 pressure building up while the apparatus 110 is heating contents in the second receptacle 113 may cause the cap 106 to disengage from the second container 112, thereby undesirably exposing the cannabis plant material in the second container 112 to oxygen. In an embodiment, the valve 126 prevents air from entering the second container, thereby minimizing oxygen present in the oxygen controlled environment 111.

Generally, the apparatus can be used to heat any contents desired. Preferably, the apparatus 110 can be used to heat contents for which heating in an oxygen controlled, moisture free environment is desired. In an embodiment, the contents comprise food. Exemplary foods include, but are not limited to, vegetables, grains, legumes, pastries, confections, eggs, and breads.

In an embodiment, the contents comprise herbaceous plant material. The present invention contemplates heating any herbaceous plant material. In an embodiment, the herbaceous plant material comprises raw cannabis plant material. In an embodiment, the apparatus is used to decarboxylate the raw cannabis plant material, for example, to activate at least one pharmacologically active cannabinoid in the decarboxylated cannabis plant material. In such embodiment, an amount of raw cannabis plant material is placed within the second receptacle 113 so that decarboxylation of the cannabis plant material can proceed in accordance with the methods described herein. Any amount of raw cannabis plant material which fits into the second receptacle 113 can be placed within the second receptacle 113. In an embodiment, the amount of raw cannabis plant material placed within the second receptacle 113 is sufficient for preparing a sublingual dosage form of decarboxylated cannabis plant material that delivers an effective amount of at least one pharmacologically active cannabinoid present in the decarboxylated cannabis plant material into the system circulation when the decarboxylated cannabis plant material is placed into the sublingual cavity. During decarboxylation of the cannabis plant material, the oxygen controlled environment 111 provided by the second container 112 minimizes oxidation of cannabinoids present in the cannabis plant material, thereby minimizing or preventing oxidative degradation of cannabinoids and maximizing the amount of the at least one pharmacologically active cannabinoid in the decarboxylated cannabis plant material.

### EXAMPLES

### Example 1

### Comparing Effectiveness of the Sublingual Dosage Form of the Present Invention to Conventional Routes of Cannabis Administration

The studies described in the following experiments were conducted to determine the effective dosage, onset of effects, peak effects, and duration of effects from various routes of administration of cannabis, for example, to compare the effectiveness of the sublingual dosage form of the present invention to conventional methods of administering cannabis, such as ingestion and inhalation.

Experiment 1—Cannabis Infused Butter

This experiment was conducted to determine the effective dosage, onset of effect, peak effect, and duration of effect from ingesting cannabis. The cannabis was prepared using a conventional preparation method whereby the cannabis is broken apart, mixed with butter and water, and heated at approximately 100 C for approximately 2 hours, after which the entire mixture is cooled so that the butter separates from the water and the cannabis residue is removed. For a period of two months, a subject was administered twice daily doses of 0.9 grams of cannabis with food via ingesting a tablespoon of cannabis infused butter. The subject experienced an inconsistent onset of effect ranging anywhere between within from about 30 minutes to about 90 minutes of cannabis ingestion. Onset of effect via ingestion appeared to occur more rapidly after fasting.

The subject experienced a duration of effect averaging from about 4 hours to about 6 hours, with a mild to moderate warm, numbing, sedative effect felt throughout the limbs and body, an experience distinctly different from the more cerebral effects experienced from smoking or sublingual use. The subject generally experienced cyclical peaking from approximately hour 2 through hour 4, marked by the ebb and flow of mild to moderate effects. Notably, the user rarely experienced a modest therapeutic effect despite ingesting what was otherwise expected to be an effective dose.

Experiment 2—Sublingual Dosage Form of the Present Invention

This experiment was conducted to determine the effective dosage, onset of effect, peak effect, and duration of effects from sublingual administration of cannabis via a sublingual dosage form of the present invention. The raw cannabis was decarboxylated in accordance with the specifications herein, after which the cannabis was gently flattened and placed directly in the sublingual cavity. The dosage range was 0.3 grams to 0.5 grains of cannabis. The subjects retained the cannabis in the sublingual cavity for an average of between about 5 minutes and about 10 minutes, and then removed.

First Subject

The subject often experienced clenching of the jaw when first placing the decarboxylated cannabis plant material within the sublingual cavity. Within approximately 1-2 minutes, the subject began to experience the onset of effects including increased heart rate, lightheaded feeling, and a measured “rush” followed by a feeling of relief, calmness, relaxation, pain diffusion, “fogginess,” and slight time/space distortion. Strong effects of the sublingual dosage form manifested within approximately 10 minutes of placing the decarboxylated cannabis plant material within the sublingual cavity. The nature of the effects was similar to those experienced when smoking, rather than ingesting cannabis. The average dose of 0.5 grams of decarboxylated cannabis plant material, however, produced significantly longer peak effects than if the 0.5 grams dose had been smoked or vaporized. For example, the peak effect lasted as many as 3 hours whereas the typical peak effect lasts for less than an hour when cannabis is smoked or vaporized. The total duration of effects ranged from between about 4 hours to about 6 hours with a steady, gradual decrease in effects after peak. The subject did not experience a “crash” after peak effects as was sometimes experienced during smoking. Surprisingly and unexpectedly, even a dose of less than 0.10 grams of decarboxylated cannabis plant material placed directly into the sublingual cavity of the subject produced a moderate amount of the effects described above for a period of as long as 3 hours.

Second Subject

A second subject was administered a single dose of approximately 0.40 grams of decarboxylated cannabis plant material. The decarboxylated cannabis plant material was prepared in accordance with the description herein, after which the decarboxylated cannabis plant material was gently flattened and placed directly into the subject's sublingual cavity. The subject retained the decarboxylated cannabis plant material in the sublingual cavity for about 20 minutes before it was removed.

Within two minutes, the subject noticed effects when rising from his chair, in the form of a very slight instability as if he had drunk a small amount of wine. Within five minutes his heartbeat was starting to increase. Within 10 minutes the effects were very similar to those experienced when the subject smoked cannabis, but more intense for the amount of cannabis administered. The subject remarked that in contrast to smoking where the subject often experienced an “elevator” effect or sudden “rush,” the effects the subject experienced via sublingual administration employing a sublingual dosage form of the present invention appeared and increased in a smooth and gentle fashion. The smooth and gradual incremental increase in sensation felt more like a natural transition rather than a dramatic alteration experienced when smoking. The subject also noted a lack of any throat or lung irritation, as well as a pleasing “total body” effect similar to effects experienced after ingesting cannabis, but much lighter in nature. The effects continued to increase in intensity and peaked at approximately 25 minutes. The effects remained strong for approximately two hours and then decreased gradually over the next 2 hours.

Experiment 3—Smoking

This experiment was conducted to determine the effective dosage, onset of effect, peak effect, and duration of effects from smoking cannabis. The raw cannabis was not treated in any manner prior to administration. The cannabis was rolled into commercial cigar wraps and smoked. The dosage range was 0.5 grams to 0.7 grams of cannabis.

First Subject

The subject often experienced throat irritation, including coughing, and sometimes experienced unwanted effects as a result of combustion, including inhaling pieces of cannabis which subsequently became lodged in the subject's throat, irritation to the eye from smoke rising from the cannabis cigar while the subject was smoking, and unpleasant residual smell after administration. Within seconds of inhaling the cannabis smoke, the user began to experience effects, including a lightheaded feeling, followed by a strong “rush” and feeling of relief and relaxation. Peak effects including pain relief, mild time and space distortion, and “foggy” feeling were experienced from 10 minutes to forty five minutes, with a gradual decrease over the next hour. The subject sometimes experienced a “crash” including a mild headache and irritable disposition approximately 2 hours after administration.

Second Subject

A second subject was administered a dose of approximately 1 gram of dried cannabis. The cannabis was prepared for smoking by passing it through a strainer to create an evenly textured rough powder, which was placed in a pipe, and smoked. The smoke was held in the lungs for 20 seconds and expelled. Within a minute the subject began to experienced effects such as lightheadedness, accelerated heartbeat and moments of unusual focus or attention to thought or sensations. These effects increased during the next five minutes, peaking within fifteen minutes. The effects then gradually diminished during the next two hours.

### Example 2

### A Single Sublingual Dosage Form of the Present Invention Delivers Effective Amounts of Cannabinoids Via Sublingual and Enteral Modes of Administration

This experiment was conducted to determine the effective dosage, onset of effect, peak effect, and duration of effects from sublingual administration of cannabinoids via a sublingual dosage form of the present invention followed by subsequent ingestion of the sublingual dosage form. Raw cannabis was decarboxylated in accordance with the description herein to create a sublingual dosage form of from about 0.3 grams to about 0.5 grams of decarboxylated cannabis plant material, which was gently flattened and placed directly in the sublingual cavity. The subject retained the sublingual dosage form in the sublingual cavity for an average of between about 5 minutes and about 10 minutes, before swallowing (without chewing) the sublingual dosage form.

The subject experienced tightening or clenching of the jaw when first placing the decarboxylated cannabis plant material within the sublingual cavity. The subject began to experience strong effects within 1-2 minutes of placing the sublingual dosage form in the subject's sublingual cavity, including a measured “rush” followed by feelings of relief, wellbeing, comfort, and time/space distortion. Peak effects, including significant pain relief, manifested within approximately 15-25 minutes of the subject placing the decarboxylated cannabis plant material within the sublingual cavity. Though similar, the peak effects lasted significantly longer than the subject had experienced when smoking the same amount of cannabis. While still experiencing the effects from the sublingual administration, approximately one hour after ingestion of the sublingual dosage form, the subject experienced a second “rush” followed by warm “numb” and/or “tingling” feeling throughout the body and limbs. These effects continued in cycles or “waves” of repeated peak feelings and releases, and ranged from moderate to strong, with the strongest effects observed after fasting. Altogether the duration of effects lasted from about 4 hours to about 10 hours, suggesting that subsequent to delivery of the at least one pharmacologically active cannabinoid sublingually via the sublingual dosage form, the sublingual dosage form retains an amount of at least one pharmacologically active cannabinoid that is effective for enteral administration, and that such dual mode of administering at least one pharmacologically active cannabinoid via a single dosage form of the present invention induces synergistic effects in the subject so that the subject experiences the beneficial effects of both sublingual and enteral administration over an extended duration.

### Example 3

### Smoking Decarboxylated Cannabis

This experiment was conducted to determine the effective dosage, onset of effect, peak effect, and duration of effects from smoking, rather than sublingual administration of, decarboxylated cannabis. The decarboxylated cannabis was rolled into commercial cigar wraps and smoked. The dosage range was 0.3 grams to 0.5 grams of decarboxylated cannabis.

First Subject

The subject often experienced throat irritation, including coughing, and sometimes experienced unwanted effects as a result of combustion, including inhaling pieces of cannabis which subsequently became lodged in the subject's throat, irritation to the eye from smoke rising from the cannabis cigar while the subject was smoking, and an unpleasant residual smell after administration. Within seconds of inhaling the cannabis smoke, the user began to experience effects, including a very strong “rush,” followed by a lightheaded sensation and feeling of relief and relaxation. The subject noticed a more rapid onset of peak effects including pain relief, mild time and space distortion, and “foggy” feeling, which were experienced within 5 to 10 minutes, with a gradual decrease over the next 2 hours. The subject noted that less cannabis was required to achieve the desired effect than if non-decarboxylated was used. The subject often experienced a “crash” including a mild headache and irritable disposition after the effects had subsided.

Second Subject

A second subject was administered a dose of approximately 1 gram of dried cannabis which had been fully decarboxylated using an apparatus described herein. The cannabis was prepared for smoking by passing it through a strainer to create an evenly textured rough powder, which was placed in a pipe and inhaled. The subject retained the smoke in the lungs for less than 20 seconds as the onset of effects was very rapid.

The subject experienced a wave of light-headedness nearly immediately, as soon as the smoke was expelled, much more rapidly than with conventional cannabis. This light-headedness increased along with accelerated heartbeat and unusual focus on thoughts and sensations that was more noticeable than the same effects from conventional cannabis. These effects peaked after twenty minutes, and gradually subsided over the next 2 hours.

### Example 4

### Cannabinoid Content of Sublingual Dosage Form

High-performance liquid chromatography (HPLC) tests were performed on raw cannabis plant material (control) and a sublingual dosage form of the present invention comprising decarboxylated cannabis plant material produced by processing the control cannabis plant material (flower) for about 75 minutes utilizing an apparatus of the present invention, and a sublingual dosage form of the present invention comprising decarboxylated cannabis plant material produced by processing cannabis trim for about 75 minutes utilizing an apparatus of the present invention. The results of the HPLC tests are shown in Tables 3 and 4 below.

TABLE 3
Percent Cannabinoid Content of Sublingual Dosage Form

| Sample | | | | | | | Max | |
|---|---|---|---|---|---|---|---|---|
| Name | THC | CBD | CBN | THCa | CBDa | Total | THC | Max CBD |
| Control | 0.7% | 0.1% | <0.1% | 19.8% | 0.2% | 20.8% | 18.0% | 0.3% |
| Sample | | | | | | | | |
| (flower) | | | | | | | | |
| Sublingual | 11.9% | 0.1% | <0.1% | 4.3% | 0.2% | 16.5% | 15.6% | 0.3% |
| Dosage | | | | | | | | |
| Form | | | | | | | | |
| (flower) | | | | | | | | |
| Sublingual | 6.9% | 0.2% | <0.1% | 1.2% | 0.1% | 8.3% | 7.9% | 0.2% |
| Dosage | | | | | | | | |
| Form | | | | | | | | |
| (trim) | | | | | | | | |

The percentages displayed in Table 3 represent weight percentage of samples tested.

The total cannabinoid content in milligrams per gram and half gram of the control sample, sublingual dosage form (flower), and sublingual dosage form (trim), are shown in Table 4 below.

TABLE 4
Total Cannabinoid Content of Sublingual Dosage Form

| Sample | THC | | CBD | CBN | |
|---|---|---|---|---|---|
| 0.5 Grams Control | 3.5 | mg | 0.6 mg | <0.5 | mg |
| 0.5 Grams Sublingual Dosage Form | 59.5 | mg | 0.6 mg | <0.5 | mg |
| (flower) | | | | | |
| 0.5 Grams Sublingual Dosage Form | 34.5 | mg | 0.8 mg | <0.5 | mg |
| (trim) | | | | | |
| 1 Gram Control | 7.1 | mg | 1.2 mg | <1 | mg |
| 1 Gram Sublingual Dosage Form | 118.9 | mg | 1.2 mg | <1 | mg |
| (flower) | | | | | |
| 1 Gram Sublingual Dosage Form | 68.9 | mg | l.5 mg | <1 | mg |
| (trim) | | | | | |

The results of the HPLC testing demonstrate that an exemplary sublingual dosage form of the present invention contains greater than 15 fold more THC content compared to raw cannabis plant material. The results of the HPLC testing further demonstrate that approximately one half gram of the sublingual dosage form of the present invention (i.e., decarboxylated cannabis plant material) can contains at least 50 mg of THC when cannabis plant material (flower) is processed utilizing an apparatus of the present invention, approximately one half gram of the sublingual dosage form of the present invention (i.e., decarboxylated cannabis plant material) can contain at least about 34 mg of THC when cannabis plant material (trim) is processed using an apparatus of the present invention, a gram of the sublingual dosage form of the present invention can contain at least 115 mg of THC when cannabis plant material (flower) is processed using an apparatus of the present invention, and approximately one gram of the sublingual dosage form of the present invention can contain at least about 68 mg of THC when cannabis plant material (trim) is processed using an apparatus of the present invention.

### Example 5

### Cannabinoid Content of Sublingual Dosage Form Before and after Placement of Sublingual Use

High-performance liquid chromatography (HPLC) tests were performed on a sublingual dosage form of the present invention comprising decarboxylated cannabis plant material produced by processing the control cannabis plant material for about 75 minutes utilizing an apparatus of the present invention before and after the sublingual dosage form was placed in the sublingual cavity of a user for a period of 15 minutes to assess the cannabinoid content released from the sublingual dosage form into the user's sublingual cavity. The user reported experiencing effects consistent with the results described herein. The results of the HPLC tests are shown in Tables 5 and 6 below.

TABLE 5
Cannabinoid Content of Sublingual Dosage Form Before and
After Placement of Sublingual Dosage Form in Sublingual Cavity

| Sample | | | | | | | Max | Max |
|---|---|---|---|---|---|---|---|---|
| Name | THC | CBD | CBN | THCa | CBDa | Total | THC | CBD |
| Sublingual | 11.9% | 0.1% | <0.1% | 4.3% | 0.2% | 16.5% | 15.6% | 0.3% |
| Dosage | | | | | | | | |
| Form | | | | | | | | |
| Before | | | | | | | | |
| Sublingual | | | | | | | | |
| Use | | | | | | | | |
| Sublingual | 3.0% | 0.1% | <0.1% | 1.0% | <0.1% | 4.1% | 3.9% | 0.1% |
| Dosage | | | | | | | | |
| Form After | | | | | | | | |
| Sublingual | | | | | | | | |
| Use | | | | | | | | |

The percentages displayed in Table 5 represent weight percentage of samples tested.

The total cannabinoid content in milligrams per gram and half gram of the sublingual dosage form prior to use and sublingual dosage form after residing 15 minutes in the user's sublingual cavity are shown in Table 6 below.

TABLE 6
Total Cannabinoid Content Released Into Sublingual
Cavity From Sublingual Dosage Form

| Sample | THC | | CBD | CBN | |
|---|---|---|---|---|---|
| 0.5 Gram Sublingual Dosage Form | 59.5 | mg | 0.6 mg | <0.5 | mg |
| Before Use | | | | | |
| 0.5 Gram Sublingual Dosage Form | 15.1 | mg | 0.4 mg | <0.5 | |
| After Use | | | | | |
| 1 Gram Sublingual Dosage Form | 118.9 | mg | 1.2 mg | <1 | mg |
| Before Use | | | | | |
| 1 Gram Sublingual Dosage Form | 30.2 | mg | 0.9 mg | <1 | mg |
| After Use | | | | | |

The results of the HPLC testing demonstrate that the sublingual dosage form of the present invention releases significant amount of at least one pharmacologically active cannabinoid into the sublingual cavity of a user. Surprisingly, and unexpectedly, the sublingual dosage form of the present invention released approximately 75% of at least one pharmacologically active cannabinoid (e.g., THC) content into the user's sublingual cavity after a period of approximately 15 minutes. Remarkably, these results demonstrate that a significant amount of at least one pharmacologically active cannabinoid content remains in the sublingual dosage form after sublingual administration, and when a user ingests the sublingual dosage form after sublingual administration, the sublingual dosage form can be used to orally deliver the cannabinoid content remaining in the sublingual dosage form. This novel dosage form provides a dual mode of administration which is expected to provide a user with longer lasting multiphasic effects without the harmful side effects associated with smoking cannabis.

### Example 6

### Working Dispenser Constructed from Hemp Material

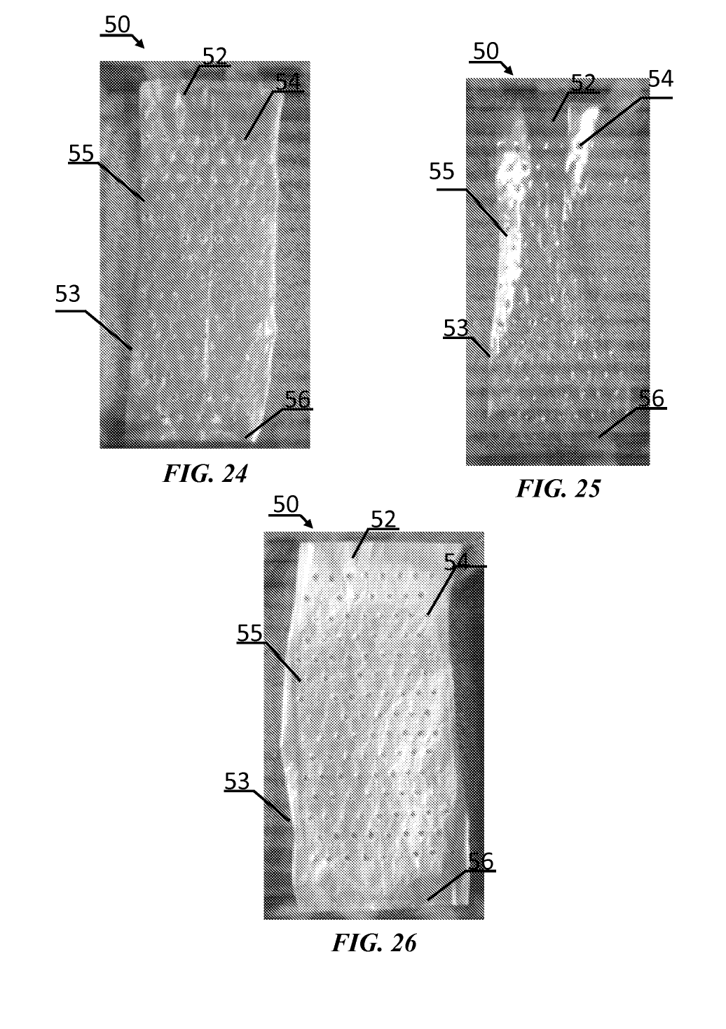

FIG. 24 illustrates a working dispenser 50 constructed from an ingestible hemp material in accordance with an example embodiment of the present invention. A wrapping 52 constructed from a sheet of hemp paper was sized and dimensioned to fold around 1 gm of cannabis plant material. The wrapping 52 thus sized and dimensioned comprised a rectangular shape having a length of approximately 2 inches, a width of approximately 1 inch, and a thickness of approximately 0.2 mm. A plurality of apertures 54 passing completely through the wrapping 52 from the first surface 55 to a second surface (not shown) opposite to the first surface 55 and vice versa were then formed, for example, by applying steam to wrapping 52 (e.g., to soften fibers in the hemp paper) and applying a sharp force to first surface 55 of wrapping 52. In the example shown, the sharp force was applied utilizing pins. The skilled artisan will appreciate, however, that any suitable technique for applying a sharp force to first surface 55 can be used, including without limitation, needles, presses, punches, die, and/or lasers. The sharp force can be applied manually or automated by a machine. It should be appreciated that the geometric shape of apertures 54 can be configured in any desired geometric shape, for example, by applying a sharp transverse force to first surface 55 of wrapping 52 utilizing a tool comprising a tip which matches the desired geometric shape (e.g., pin press, needle press, needle punch, etc.). In the example shown in FIG. 24, a sharp transverse force was applied to first surface 55 of wrapping 52 utilizing pins comprising circular tips to provide circular shaped apertures 54. Once the plurality of apertures 54 were formed passing through wrapping 52, at least one wax fastener 56 comprised of a paraffin wax was disposed proximal the entire perimeter 53 on a first surface 55 of wrapping 52, for example, by dipping the perimeter 53 of first surface 55 of wrapping 52 in paraffin wax and allowing it to solidify at ambient temperature. Preferably, the at least one wax fastener 56 is disposed proximal the perimeter 53 of wrapping 52 when first surface 55 of wrapping 52 is substantially free of moisture (i.e., dry).

The resulting dispenser 50 thus formed comprised a wrapping 52 comprising a sheet of ingestible material (e.g., hemp) having a perimeter 53, a first surface 55, and a second surface (not shown) on an opposite side of the sheet from the first surface, a plurality of apertures 54 passing completely through from the first surface 55 to the second surface (not shown), and at least one wax fastener 56 (e.g., paraffin wax) disposed proximal the perimeter and configured to secure the sheet of ingestible material to itself when the sheet is folded over and placed in contact with the at least one wax fastener.

### Example 7

### Working Dispenser Constructed from Cellulose Material

FIG. 25 illustrates a working dispenser 50 constructed from an ingestible cellulose material in accordance with an example embodiment of the present invention. A wrapping 52 constructed from a sheet of cellulose material was sized and dimensioned to fold around approximately 1 gm of cannabis plant material. The wrapping 52 thus sized and dimensioned comprised a rectangular shape having a length of approximately 2 inches, a width of approximately 1 inch, and a thickness of approximately 0.1 mm. Next, second surface (not shown) opposite to first surface 55 was uniformly coated with a thin layer of molten paraffin wax, for example, by dipping the second surface of wrapping 52 in the molten paraffin wax, and letting the wax solidify at ambient temperature. Any suitable technique can be used to coat second surface (e.g., spraying). Once second surface (not shown) of wrapping 52 was coated with paraffin wax, a plurality of apertures 54 passing completely through the wrapping 52 from the first surface 55 to a second surface (not shown) opposite to the first surface 55 and vice versa were formed, for example, by applying a puncturing force to first surface 55 of wrapping 52. In the example shown, the puncturing force was applied utilizing pins. The skilled artisan will appreciate, however, that any suitable technique for applying a puncturing force to first surface 55 can be used, including without limitation, needles, presses, punches, die, and/or lasers. The puncturing force can be applied manually or automated by a machine. It should be appreciated that the geometric shape of apertures 54 can be configured in any desired geometric shape, for example, by applying a sharp transverse force to first surface 55 of wrapping 52 utilizing a tool comprising a tip which matches the desired geometric shape (e.g., pin press, needle press, needle punch, etc.). In the example shown in FIG. 25, a puncturing transverse force was applied to first surface 55 of wrapping. 52 utilizing pins comprising circular tips to provide circular shaped apertures 54. Once the plurality of apertures 54 were formed passing through wrapping 52, at least one wax fastener 56 comprised of a paraffin was disposed proximal the entire perimeter 53 on a first surface 55 of wrapping 52, for example, by dipping the perimeter 53 of first surface 55 of wrapping 52 in molten paraffin wax and allowing it to solidify at ambient temperature. Preferably, the at least one wax fastener 56 is disposed proximal the perimeter 53 of wrapping 52 when first surface 55 of wrapping 52 is substantially free of moisture (i.e., dry).

The resulting dispenser 50 thus formed comprised a wrapping 52 comprising a sheet of ingestible material (e.g., cellulose) having a perimeter 53, a first surface 55, and a second surface (not shown) on an opposite side of the sheet from the first surface, a plurality of apertures 54 passing completely through from the first surface 55 to the second surface (not shown), at least one wax fastener 56 (e.g., paraffin wax) disposed proximal the perimeter and configured to secure the sheet of ingestible material to itself when the sheet is folded over and placed in contact with the at least one wax fastener, and a coating (not viewable) disposed on the second surface (e.g., configured to elicit a positive sensory response from a user).

### Example 8

### Working Dispenser Constructed from Rice Material

FIG. 26 illustrates a working dispenser 50 constructed from an ingestible rice material in accordance with an example embodiment of the present invention. A wrapping 52 constructed from a sheet of rice material (e.g., paper) was sized and dimensioned to fold around approximately 1 gm of cannabis plant material. The wrapping 52 thus sized and dimensioned comprised a rectangular shape having a length of approximately 2 inches, a width of 1 inch, and a thickness of approximately 0.2 mm. Next, second surface (not shown) opposite to first surface 55 was provided with a first coating (e.g., a uniform, thin layer) by dipping the second surface in molten beeswax, and letting the molten beeswax solidify at ambient temperature. The first coating configures the dispenser 50 to elicit a sense of smoothness, comfort and/or softness from a user when the dispenser is folded around decarboxylated cannabis plant material inside a user's sublingual cavity. Then, the second surface was provided with a second coating (e.g., uniform, thin layer) by spraying a mixture of glycerin and orange zest onto the second surface.

The second coating configures the dispenser 50 to elicit a sense of pleasant taste from a user when the dispenser is folded around decarboxylated cannabis plant material inside a user's sublingual cavity. Once second surface of wrapping 52 was provided with the first and second coatings, at least one wax fastener 56 comprised of a paraffin wax was disposed proximal the entire perimeter 53 on a first surface 55 of wrapping 52, for example, by dipping the perimeter 53 of first surface 55 of wrapping 52 in molten paraffin wax and allowing it to solidify at ambient temperature. Preferably, the at least one wax fastener 56 is disposed proximal the perimeter 53 of wrapping 52 when first surface 55 of wrapping 52 is substantially free of moisture (i.e., dry).

Once at least one wax fastener 56 comprised of a paraffin wax was disposed proximal the entire perimeter 53 on a first surface 55 of wrapping 52, a plurality of apertures 54 were formed passing completely through the wrapping 52 from the first surface 55 to a second surface (not shown) opposite to the first surface 55 and vice versa, for example, by applying a puncturing force to first surface 55 of wrapping 52. In the example shown, the puncturing force was applied utilizing pins. The skilled artisan will appreciate, however, that any suitable technique for applying a puncturing force to first surface 55 can be used, including without limitation, needles, presses, punches, die, and/or lasers. The puncturing force can be applied manually or automated by a machine. It should be appreciated that the geometric shape of apertures 54 can be configured in any desired geometric shape, for example, by applying a sharp transverse force to first surface 55 of wrapping 52 utilizing a tool comprising a tip which matches the desired geometric shape (e.g., pin press, needle press, needle punch, etc.). In the example shown in FIG. 26, a puncturing transverse force was applied to first surface 55 of wrapping 52 utilizing pins comprising circular tips to provide circular shaped apertures 54.

The resulting dispenser 50 thus formed comprised a wrapping 52 comprising a sheet of ingestible material (e.g., rice) having a perimeter 53, a first surface 55, and a second surface (not shown) on an opposite side of the sheet from the first surface, a plurality of apertures 54 passing completely through from the first surface 55 to the second surface (not shown), at least one wax fastener 56 (e.g., paraffin wax) disposed proximal the perimeter and configured to secure the sheet of ingestible material to itself when the sheet is folded over and placed in contact with the at least one wax fastener, a coating (not viewable) disposed on the second surface (e.g., configured to elicit a positive sensory response from a user), and a second coating (not viewable) disposed on the first coating (e.g., configured to elicit a positive sensory response from the user).

### Example 9

### Working Dispenser Comprising Visual Folding Indicator

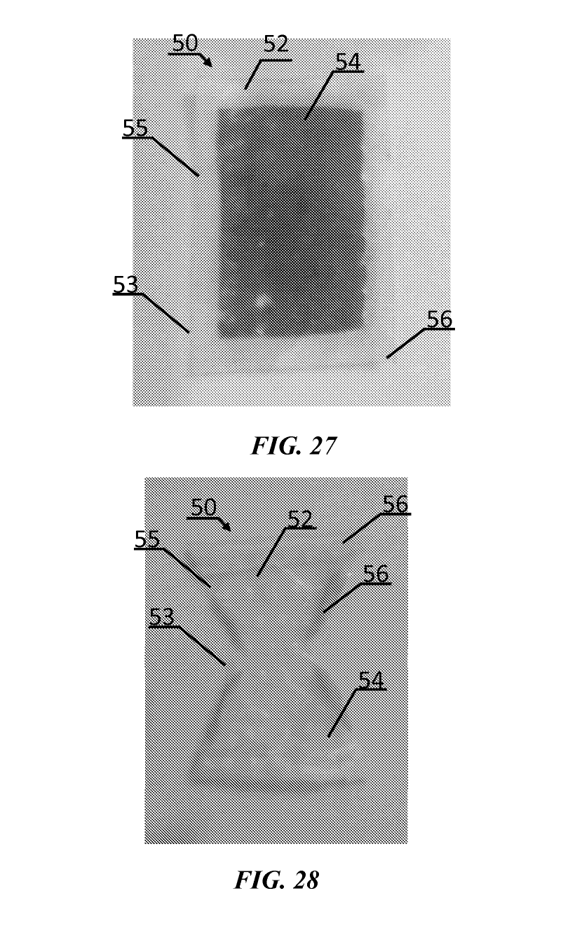

FIG. 27 illustrates a working dispenser 50 constructed from an ingestible rice material in accordance with an example embodiment of the present invention. A wrapping 52 constructed from a sheet of rice material (e.g., paper) was sized and dimensioned to fold around approximately 0.5 gram of cannabis plant material. The wrapping 52 thus sized and dimensioned comprised dual triangular shapes having a length of 1.5 inches, a width of 0.75, and a thickness of approximately 1 mm. Next, second surface (not shown) opposite to first surface 55 was uniformly coated with a thin layer of paraffin molten wax, for example, by dipping the second surface of wrapping 52 in the molten wax, and the wax was permitted to solidify at ambient temperature. The first coating causes the dispenser 50 to elicit a sense of smoothness, comfort and/or softness from a user when the dispenser is folded around decarboxylated cannabis plant material inside a user's sublingual cavity. Then, the second surface was provided with a second coating (e.g., uniform, thin layer) by spreading a thin layer of honey onto the second surface. The second coating configures the dispenser 50 to elicit a sense of pleasant taste from a user when the dispenser is folded around decarboxylated cannabis plant material inside a user's sublingual cavity. Once second surface of wrapping 52 was provided with the first and second coatings, at least one wax fastener 56 comprised of beeswax was disposed proximal the top and bottom perimeter 53 on a first surface 55 of wrapping 52, for example, by disposing segments of molten beeswax along the top and bottom perimeter 53 of first surface 55 of wrapping 52 and allowing the molten beeswax to solidify at ambient temperature.

Once at least one wax fastener 56 comprised of beeswax was disposed proximal the top and bottom perimeter 53 on a first surface 55 of wrapping 52, at least one wax fastener 56 comprised of colored paraffin wax was disposed proximal the side perimeters 53 of first surface 55 of wrapping 52, for example, by disposing segments of molten colored paraffin wax along the side perimeters 53 on first surface 55 of wrapping 52 and allowing the molten wax to solidify at ambient temperature. The colored paraffin wax resulted from heating the paraffin wax to a molten state and then mixing food coloring to the molten wax. The wax fastener disposed on the side perimeters 53 of first surface 55 of wrapping 52 configures dispenser 50 to provide a visual folding indicator. That is, the coloring (e.g., green and/or yellow) indicates how to optimally fold the wrapping 52 over and/or around decarboxylated cannabis plant material, i.e., the decarboxylated cannabis plant material is placed on the first surface 55 of

After the wax fasteners have been applied to dispenser 50, a plurality of apertures 54 were formed passing completely through the wrapping 52 from the first surface 55 to a second surface (not shown) opposite to the first surface 55 and vice versa, for example, by applying a puncturing force to first surface 55 of wrapping 52. In the example shown, the puncturing force was applied utilizing pins. The skilled artisan will appreciate, however, that any suitable technique for applying a puncturing force to first surface 55 can be used, including without limitation, needles, presses, punches, die, and/or lasers. The puncturing force can be applied manually or automated by a machine. It should be appreciated that the geometric shape of apertures 54 can be configured in any desired geometric shape, for example, by applying a sharp transverse force to first surface 55 of wrapping 52 utilizing a tool comprising a tip which matches the desired geometric shape (e.g., pin press, needle press, needle punch, etc). In the example shown in FIG. 27, a puncturing transverse force was applied to first surface 55 of wrapping 52 utilizing pins comprising circular tips to provide circular shaped apertures 54.

The resulting dispenser 50 thus formed comprised a wrapping 52 comprising a sheet of ingestible material (e.g., rice) having a perimeter 53, a first surface 55, and a second surface (not shown) on an opposite side of the sheet from the first surface, a plurality of apertures 54 passing completely through from the first surface 55 to the second surface (not shown), at least one wax fastener 56 (e.g. beeswax or paraffin wax) disposed proximal the top and bottom perimeter and configured to secure the sheet of ingestible material to itself when the sheet is folded over and the at least one wax fasteners are placed in contact with each other, at least one wax fastener 56 (e.g. beeswax or paraffin wax) disposed proximal the side perimeters and configured to secure the sheet of ingestible material to itself when the sheet is folded over and placed in contact with at least one wax fastener, a visible folding indicator, and a coating (not viewable) disposed on the second surface (e.g., configured to elicit a positive sensory response from a user, e.g., a sense of comfort, softness, and/or smoothness), and a second coating (not viewable) disposed on the first coating (e.g., configured to elicit a positive sensory response from the user, e.g., a sense of pleasant taste).

### Example 10

### Working Dispenser Comprising Visual Folding Indicator

FIG. 28 illustrates a working dispenser 50 constructed from an ingestible rice material in accordance with an example embodiment of the present invention. A wrapping 52 constructed from a sheet of rice material (e.g., paper) was sized and dimensioned to fold around approximately 0.4 gm of cannabis plant material. The wrapping 52 thus sized and dimensioned comprised a rectangular shape having a length of approximately 1 inch a width of approximately 0.75 inch, and a thickness of approximately 1 mm. Next, second surface (not shown) opposite to first surface 55 was provided with a first coating (e.g., a uniform, thin layer) by dipping the second surface in molten paraffin wax, and allowing the molten paraffin wax to solidify at ambient temperature. The first coating configures the dispenser 50 to elicit a sense of smoothness, comfort and/or softness from a user when the dispenser is folded around decarboxylated cannabis plant material inside a user's sublingual cavity. Then, a coating 58 (e.g., uniform, thin layer) was disposed on first surface 55, for example by spraying a mixture comprising sesame oil and blue food coloring (e.g., FDA approved) onto first surface 55 while avoiding perimeter 53.

The coating 58 on first surface 55 configures the dispenser 50 to aid in the transmission/absorption of cannabinoids when the dispenser is folded around decarboxylated cannabis plant material inside a user's sublingual cavity.

Next, at least one wax fastener 56 comprised of beeswax was disposed proximal the entire perimeter 53 on a first surface 55 of wrapping 52, for example, by dipping the perimeter 53 of first surface 55 of wrapping 52 in molten beeswax and allowing it to solidify at ambient temperature. Then, a plurality of apertures 54 were formed passing completely through the wrapping 52 from the first surface 55 to a second surface (not shown) opposite to the first surface 55 and vice versa, for example, by applying a puncturing force to first surface 55 of wrapping 52. In the example shown, the puncturing force was applied utilizing pins. The skilled artisan will appreciate, however, that any suitable technique for applying a puncturing force to first surface 55 can be used, including without limitation, needles, presses, punches, die, and/or lasers. The puncturing force can be applied manually or automated by a machine. It should be appreciated that the geometric shape of apertures 54 can be configured in any desired geometric shape, for example, by applying a sharp transverse force to first surface 55 of wrapping 52 utilizing a tool comprising a tip which matches the desired geometric shape (e.g., pin press, needle press, needle punch, etc.). In the example shown in FIG. 28, a puncturing transverse force was applied to first surface 55 of wrapping 52 utilizing pins comprising circular tips to provide circular shaped apertures 54.

The resulting dispenser 50 thus formed comprised a wrapping 52 comprising a sheet of ingestible material (e.g., rice) having a perimeter 53, a first surface 55, and a second surface (not shown) on an opposite side of the sheet from the first surface, a plurality of apertures 54 passing completely through from the first surface 55 to the second surface (not shown), at least one wax fastener 56 (e.g. beeswax) disposed proximal the perimeter and configured to secure the sheet of ingestible material to itself when the sheet is folded over and at least one wax fastener is placed into contact with itself, a coating disposed on the first surface (e.g. to aid in transmission and absorption of cannabinoids), and a coating disposed on the second surface (e.g., configured to elicit a positive sensory response from a user).

### Example 11

### Comparative Example

This comparative example demonstrates that the decarboxylation methods of the present invention surprisingly and unexpectedly achieve greater decarboxylation completeness in shorter time periods than the decarboxylation apparatus described in U.S. Pat. No. 4,279,824 (“the '824 patent”) when the apparatus is used in accordance with the method and operating parameters described therein. As used herein, “Decarboxylation completeness” refers the measured percentage of THC and/or CBD actually produced relative to the maximum amount of THC and/or CBD that theoretically could be produced based on the amount of THCA and/or CBDA present in the cannabis plant material. Max THC was calculated using the following formula: Max THC=THC+THCA*0.877. Max CBD was calculated using the same formula with CBDA and CBD substituted for THCA and THC, respectively. Decarboxylation completeness of both THCA and CBDA was measured by monitoring the decarboxylation of d9-tetrahydrocannabinolic acid (THCA) to d9-tetrahydrocannabinol (THC) and the decarboxylation of cannabidiolic acid (CBDA) to cannabidiol (CBD) using HPLC-UV via a validated method (MCR-TM-002).

Dried and cured flowers of OG, a cannabis sativa strain were decarboxylated using the apparatus described in U.S. Pat. No. 4,279,824 using the operating parameters described therein. In particular, the cannabis plant material was placed inside the inner chamber, water was used as the fluid medium in the outer chamber, and the thermostat was set to 110° C. so that decarboxylation would proceed using steam from boiling water as the external heat source and the apparatus would shut off rapidly after all of the water boiled away. As such, the estimated temperature range applied to the inner chamber in which the cannabis plant material resided did not exceed 100° C. at any point during the decarboxylation time period. It is worth noting that the '824 patent discloses decarboxylating cannabis using the apparatus under these conditions for up to 60 minutes (1h) to obtain the maximum amount of THC theoretically possible. In addition to decarboxylating the cannabis for 60 minutes, the inventors used the apparatus of the '824 patent under the above conditions for the time periods specified in Table 7 for not only THC but also for CBD. Table 7 below shows the results obtained for decarboxylation of THCA to THC and CBDA to CBD using the apparatus of the '824 patent according to the operating parameters described therein and reproduced above.

TABLE 7
Decarboxylation Completeness for THC/CBD Using Apparatus
of the ′824 Patent

| | | | | | % CBD | |
|---|---|---|---|---|---|---|
| | | | % THC | | decarboxyl- | |
| Time | Temp | Max | decarboxylation | Max | ation | % |
| (min) | (° C.) | THC | completeness | CBD | completeness | CBN |
| 60 | 100 | 14.0% | 64% | N/A | N/A | 0.3% |
| 75 | 100 | 15.6% | 76.3% | <1% | N/A | <0.1% |
| 90 | 100 | 14.0% | 82.1% | N/A | N/A | 0.4% |
| 105 | 100 | 4.8% | 89.6% | 8.3% | 66.26% | BQL |
| 120 | 100 | 5.1% | 92.4% | 9.1% | 35.70% | 0.1% |
| 180 | 100 | 5.2% | 97.7% | 8.2% | 60.30% | 0.1% |
| 240 | 100 | 5.2% | 96.6% | 8.0% | 65.70% | 0.1% |

*BQL = below quantifiable limits

As shown in Table 7 above, decarboxylating the cannabis for 60 minutes in accordance with the disclosure of the '824 patent achieved decarboxylation completeness for THC of only 64%. Moreover, the decarboxylation completeness for CBD did not exceed 67% even after four hours of decarboxylation using the apparatus. Thus, the data in Table 7 above demonstrate that the apparatus disclosed in the '824 patent falls short of obtaining maximum amounts of THC when decarboxylating cannabis in accordance with the operating parameters set forth therein.

The same strain of cannabis was decarboxylated in accordance with the methods of the present invention at the time periods and temperatures specified in Table 8 below, and decarboxylation completeness of THC and CBD was measured with the results set forth in Table 8 below

TABLE 8
Decarboxylation Completeness for THC/CBD Using Methods of
the Present Invention

| | | | % THC | | % CBD | % CBN |
|---|---|---|---|---|---|---|
| | | | decarboxyl- | | decarboxyl- | decarboxyl- |
| | | | ation | | ation | ation |
| Time | Temp | Max | com- | Max | com- | com- |
| (min) | (° C.) | THC | pleteness | CBD | pleteness | pleteness |
| 60 | 105 | 3.9% | 98.00% | 8.1% | 81.00% | 0.2% |
| 120 | 105 | 2.9% | 100.00% | 6.7% | 91.80% | 0.2% |
| 30 | 115 | <1% | N/A | 13.2% | 82.70% | BQL |
| 60 | 115 | <1% | N/A | 7.8% | 94.80% | BQL |
| 60 | 115 | 3.4% | 100.00% | 6.2% | 92.40% | 0.2% |
| 120 | 115 | 2.1% | 100.00% | 6.7% | 96.20% | 0.2% |

*BQL = below quantifiable limits

As shown in Table 8 above, surprisingly and unexpectedly, decarboxylating an amount of raw cannabis plant material by heating the amount of raw cannabis plant material in an oxygen controlled environment at a temperature from about 105° C. to about 115° C. for a period of from about 20 to 60 minutes results in the formation of decarboxylated cannabis plant material that contains at least 90% of the maximum amount of THC and/or at least 80% of the maximum amount of CBD that could theoretically be present as a result of decarboxylation of the maximum amount of both THCA and/or CBDA, respectively, present in the amount of raw cannabis plant material (i.e., a decarboxylation completeness of at least 90% and at least 80%, respectively). Similarly, decarboxylating an amount of raw cannabis plant material by heating it in an oxygen controlled environment at a temperature from about 105° C. to about 115° C. for a period of between 60 minutes and about 120 minutes results in the formation of decarboxylated cannabis plant material that contains the maximum amount of THC and/or at least 90% of the maximum amount of THC that could theoretically be present in the amount of raw cannabis plant material (i.e., a decarboxylation completeness of 100% and 90% respectively. This is in contrast to the apparatus described in U.S. Pat. No. 4,279,824 which only achieves decarboxylation completeness for THC of 64% when operated according to the parameters described therein and does not exceed a decarboxylation completeness of 67% even after 4 hours of decarboxylation using that apparatus.

### SOME DEFINITIONS

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Other than in the operating examples, or where otherwise indicated, all numbers expressing quantities of ingredients or reaction conditions used herein should be understood as modified in all instances by the term “about.” The term “about” when used in connection with percentages may mean±1%.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described herein. The term “comprises” means “includes.” The abbreviation, “e.g.” is derived from the Latin exempli gratia, and is used herein to indicate a non-limiting example. Thus, the abbreviation “e.g.” is synonymous with the term “for example.”

The terms “decrease”, “reduced”, “reduction”, “decrease” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90%, where the decrease is less than 100%. In one embodiment, the decrease includes a 100% decrease (e.g. absent level as compared to a reference sample), or any decrease between 10-100% as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

All patents and other publications identified are expressly incorporated herein by reference for the purpose of describing and disclosing, for example, the methodologies described in such publications that might be used in connection with the disclosure. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Numerous modifications and alternative embodiments of the present invention will be apparent to those skilled in the art in view of the foregoing description. Accordingly, this description is to be construed as illustrative only and is for the purpose of teaching those skilled in the art the best mode for carrying out the present invention. Details of the structure may vary substantially without departing from the spirit of the present invention, and exclusive use of all modifications that come within the scope of the appended claims is reserved. Within this specification embodiments have been described in a way which enables a clear and concise specification to be written, but it is intended and will be appreciated that embodiments may be variously combined or separated without parting from the invention. It is intended that the present invention be limited only to the extent required by the appended claims and the applicable rules of law.

It is also to be understood that the following claims are to cover all generic and specific features of the invention described herein, and all statements of the scope of the invention which, as a matter of language, might be said to fall therebetween.

1-384. (canceled)

385. A dispenser for delivering at least one pharmacologically active cannabinoid from decarboxylated cannabis plant material contained inside the dispenser into the sublingual cavity of a subject when the dispenser is placed within the subject's sub lingual cavity with the decarboxylated cannabis plant material contained therein.

386. The dispenser of claim 385, wherein the dispenser is ingestible.

387. The dispenser of claim 385, wherein the dispenser is constructed from an ingestible material.

388. The dispenser of claim 385, wherein the dispenser is constructed from an insoluble material.

389. The dispenser of claim 385, wherein the dispenser is constructed from a fibrous plant material.

390. The dispenser of claim 385, wherein the dispenser is constructed from a fibrous plant material comprising cellulose.

391. The dispenser of claim 385, wherein the dispenser is constructed from a fibrous plant material comprising at least one of hemp, rice, bamboo, corn husk, silk husk, fruit skin, straw, flax, soy, wood, a pulp of any thereof, and combinations thereof.

392. The dispenser of claim 385, wherein the dispenser comprises a wrapping folded around the decarboxylated cannabis plant material.

393. The dispenser of claim 385, wherein the dispenser comprises at least one aperture configured to retain the decarboxylated cannabis plant material inside the dispenser while delivering the at least one pharmacologically active cannabinoid from the decarboxylated cannabis plant material into the sublingual cavity of the subject.

394. The dispenser of claim 385, wherein the dispenser comprises a plurality of apertures configured to retain the decarboxylated cannabis plant material inside the dispenser while delivering the at least one pharmacologically active cannabinoid from the decarboxylated cannabis plant material into the sublingual cavity of the subject.

395. The dispenser of claim 385, wherein the dispenser comprises a fastener on a first portion of an interior surface of the dispenser which secures the first portion of the interior surface of the dispenser to at least a second portion of the interior surface of the dispenser when the dispenser is folded around the decarboxylated cannabis plant material in such a way that the fastener contacts the at least the second portion of the interior surface, thereby retaining the decarboxylated cannabis plant material inside the dispenser.

396. The dispenser of claim 395, wherein the fastener comprises a wax or gelatin.

397. The dispenser of claim 395, wherein the fastener comprises at least one of beeswax, candelilla wax, carnauba, ceresin wax, japan wax, microcrystalline wax, paraffin wax, sugarcane wax, and combinations thereof.

398. The dispenser of claim 395, wherein the dispenser is configured to elicit a positive sensory response from the subject when the dispenser is placed in the subject's sublingual cavity with the decarboxylated cannabis plant material contained therein.

399. The dispenser of claim 385, wherein the dispenser is configured to elicit a positive sensory response from the subject when the dispenser is ingested by the subject with the decarboxylated cannabis plant material contained therein.

400. The dispenser of claim 385, wherein the dispenser comprises at least a first coating on at least a portion of an exterior surface of the dispenser.

401. The dispenser of claim 386, wherein the dispenser comprises at least a first coating on the entire exterior surface of the dispenser.

402. The dispenser of claim 400 wherein the at least the first coating elicits a positive sensory response from the subject when the dispenser is placed in the subject's sublingual cavity or ingested by the subject with the decarboxylated cannabis plant material contained therein.

403. The dispenser of claim 398, wherein the positive sensory response comprises a sensation of smoothness, comfort, pleasant taste, and/or softness in the subject's sub lingual cavity or throat.

404. The dispenser of claim 398, wherein the at least the first coating comprises a wax or gelatin.

405. The dispenser of claim 398, wherein the at least the first coating comprises at least one of beeswax, candelilla wax, carnauba, ceresin wax, japan wax, microcrystalline wax, paraffin wax, shellac wax, sugarcane wax, and combinations thereof.

406. The dispenser of claim 398, wherein the at least the first coating comprises a flavorant.

407. The dispenser of claim 398, wherein the at least the first coating comprises an oil.

408. The dispenser of claim 385, wherein the dispenser is configured to deliver an effective amount of the at least one pharmacologically active cannabinoid from the decarboxylated cannabis plant material into the subject's sublingual cavity.

409. The dispenser of claim 385, wherein the dispenser is configured to deliver a first effective dose of the at least one pharmacologically active cannabinoid from the decarboxylated cannabis plant material into the subject's sublingual cavity when the dispenser is placed into the subject's sublingual cavity, and to deliver a second dose of the at least one pharmacologically active cannabinoid from the decarboxylated cannabis plant material into the subject's gastrointestinal tract when the dispenser is ingested by the subject after delivering the first effective dose.

410. The dispenser of claim 385, wherein the dispenser is configured to deliver a predetermined dose of the at least one pharmacologically active cannabinoid from the decarboxylated cannabis plant material into the subject's sublingual cavity.

411. The dispenser of claim 385, wherein the dispenser is configured to deliver a first predetermined dose of the at least one pharmacologically active cannabinoid from the decarboxylated cannabis plant material when the dispenser is placed into the subject's sub lingual cavity, and to deliver a second predetermined dose of the at least one pharmacologically active cannabinoid from the decarboxylated cannabis plant material when the dispenser is swallowed by the subject after delivering the first predetermined dose.

412. A wrapping comprising at least one aperture configured to retain cannabis plant material inside the wrapping while permitting an effective amount of at least one pharmacologically active agent released from the cannabis plant material upon exposure to saliva to flow with the saliva into a subject's sublingual cavity when the wrapping is folded around the cannabis plant material and placed in the subject's sublingual cavity.

413. A dispenser comprising:
a) a wrapping having a first surface, a second surface, and a perimeter defined by a first side, a second side, a third side, and a fourth side, the wrapping comprising at least one aperture configured to retain a decarboxylated cannabis plant material inside the wrapping while permitting a maximum dosage of at least one pharmacologically active cannabinoid present in the decarboxylated cannabis plant material to be released into a subject's sublingual cavity when the wrapping is folded around the decarboxylated cannabis plant material and placed into the subject's sublingual cavity; and
b) a first fastener on at least a portion of the first surface.

414. A wrapping, comprising:
d) a sheet of ingestible material having a perimeter, a first surface, and a second surface on an opposite side of the sheet from the first surface;
e) at least one aperture passing completely through from the first surface to the second surface and vice versa; and
f) at least one wax fastener disposed proximal the perimeter and configured to secure the sheet of ingestible material to itself when the sheet is folded over and placed in contact with the at least one wax fastener.

415. A wrapping, comprising:
d) a sheet of ingestible material having a perimeter, a first surface, and a second surface on an opposite side of the sheet from the first surface;
e) at least one aperture passing completely through from the first surface to the second surface and vice versa;
f) at least one wax fastener disposed proximal the perimeter and configured to secure the sheet of ingestible material to itself when the sheet is folded over and placed in contact with the at least one wax fastener;
wherein the sheet is configured to enclose decarboxylated cannabis plant material inside the wrapping when the sheet is folded over and placed in contact with the at least one wax fastener;
wherein when the wrapping is placed in the sub lingual cavity of a subject with decarboxylated cannabis plant material inside the wrapping, the at least one aperture permits cannabinoids present in the decarboxylated cannabis plant material to exit the wrapping while retaining the decarboxylated cannabis plant material inside the wrapping.
